(19) United States
(12) Patent Application Publication
(10) Pub. No.: US 2023/0235305 A1
(43) Pub. Date: Jul. 27, 2023

(54) CELLS MODIFIED BY A CAS12I POLYPEPTIDE

(71) Applicant: ARBOR BIOTECHNOLOGIES, INC., Cambridge, MA (US)

(72) Inventors: Noah Michael JAKIMO, Cambridge, MA (US); Quinton Norman WESSELLS, Cambridge, MA (US); Tia Marie DITOMMASO, Waltham, MA (US); Jeffrey Raymond HASWELL, Newton, MA (US); Anthony James GARRITY, Hingham, MA (US); Jason Michael CARTE, Cambridge, MA (US); Colin Alexander MCGAW, Boston, MA (US); Shaorong CHONG, Arlington, MA (US); David A. SCOTT, Somerville, MA (US)

(21) Appl. No.: 18/010,680

(22) PCT Filed: Jun. 16, 2021
(86) PCT No.: PCT/US2021/037670

§ 371 Date: Dec. 15, 2022

Related U.S. Application Data
(60) Provisional application No. 63/039,933, filed on Jun. 16, 2020.
(60) Provisional application No. 63/063,821, filed on Aug. 10, 2020.
(60) Provisional application No. 63/085,687, filed on Sep. 30, 2020.
(60) Provisional application No. 63/134,271, filed on Jan. 6, 2021.

(51) Int. Cl. C12N 9/22; C12N 15/90; C12N 15/11

(52) U.S. Cl. CPC C12N 9/22; C12N 15/907; C12N 15/11; C12N 2310/20; C12N 2800/80

(57) ABSTRACT

The present disclosure relates to cells modified by a Cas12i polypeptide, methods of modifying the cells, processes for characterizing the modified cells, compositions and formulations comprising the modified cells, and uses of the compositions and formulations comprising the modified cells.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY

It is against the above background that the present disclosure provides certain advantages and advancements over the prior art. The disclosure herein is not limited to specific advantages or functionalities.

Although this invention disclosed herein is not limited to specific advantages or functionalities, the invention provides a modified cell comprising a Cas12i-induced genomic deletion, wherein (a) the deletion starts within about 5 nucleotides to about 15 nucleotides downstream of a 5′-NTTN-3′ sequence, wherein N is any nucleotide, (b) wherein the deletion is greater than about 15 nucleotides in length, and (c) wherein the modified cell substantially lacks expression of the gene.

In one aspect of the modified cell, the 5′-NTTN-3′ sequence is on a sense strand of the gene.

In one aspect of the modified cell, the 5′-NTTN-3′ sequence is on an antisense strand of the gene.

In one aspect of the modified cell, the deletion starts within about 5 nucleotides to about 10 nucleotides downstream of the 5′-NTTN-3′ sequence.

In one aspect of the modified cell, the deletion starts within about 10 nucleotides to about 15 nucleotides downstream of the 5′-NTTN-3′ sequence.

In one aspect of the modified cell, the deletion ends within about 30 nucleotides to about 50 nucleotides downstream of the 5′-NTTN-3′ sequence.

In one aspect of the modified cell, the deletion ends within about 30 nucleotides to about 40 nucleotides downstream of the 5′-NTTN-3′ sequence.

In one aspect of the modified cell, the deletion ends within about 20 to about 30 nucleotides downstream of the 5′-NTTN-3′ sequence.

In one aspect of the modified cell, (a) the deletion starts within about 5 to about 15 nucleotides downstream of the 5′-NTTN-3′ sequence on the sense strand, (b) wherein the deletion ends within about 5 to about 15 nucleotides (e.g., upstream) of a 5′-NAAN-3′ sequence on the sense strand, wherein N is any nucleotide; and (c) wherein the 5′-NAAN-3′ sequence is downstream of the 5′-NTTN-3′ sequence.

In one aspect of the modified cell, (a) the deletion starts within about 5 to about 15 nucleotides downstream of the 5′-NTTN-3′ sequence on the antisense strand, (b) wherein the deletion ends within about 5 to about 15 nucleotides (e.g., upstream) of a 5′-NAAN-3′ sequence on the antisense strand, wherein N is any nucleotide, and (c) wherein the 5′-NAAN-3′ sequence is downstream of the 5′-NTTN-3′ sequence.

In one aspect of the modified cell, the deletion is greater than 40 nucleotides in length.

In another aspect of the modified cell, the deletion is in an exon of the gene.

In another aspect of the modified cell, the deletion overlaps with a mutation in the gene.

In another aspect of the modified cell, the deletion overlaps with an insertion in the gene.

In another aspect of the modified cell, the deletion removes at least a portion of a repeat expansion of the gene.

In another aspect of the modified cell, the deletion disrupts one or both alleles of the gene.

In another aspect of the modified cell, the modified cell comprises two or more deletions.

In another aspect of the modified cell, an unmodified cell lacks the deletion.

In another aspect of the modified cell, the unmodified cell expresses the gene.

In another aspect of the modified cell, the unmodified cell is a wild-type cell.

In another aspect of the modified cell, the 5′-NTTN-3′ sequence is 5′-NTTY-3′,5′-NTTC-3′,5′-NTTT-3′,5′-NTTA-3′,5′-NTTB-3′,5′-NTTG-3′,5′-CTTY-3′,5′-DTTR′3′,5′-CTTR-3′,5′-DTTT-3′, 5′-ATTN-3′, or 5′-GTTN-3′, wherein Y is C or T, B is any nucleotide except for A, D is any nucleotide except for C, and R is A or G.

In another aspect of the modified cell, the 5′-NTTN-3′ sequence is 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′, 5′-GTTG-3′,5′-GTTC-3′,5′-CTTA-3′,5′-CTTT-3′,5′-CTTG-3′, or 5′-CTTC-3′.

In another aspect of the modified cell, the modified cell is a eukaryotic cell or a prokaryotic cell.

In another aspect of the modified cell, the modified cell is an animal cell, a plant cell, or a fungal cell or the cell is derived from an animal cell, a plant cell, or a fungal cell.

In another aspect of the modified cell, the modified cell is a mammalian cell or derived from a mammalian cell.

In another aspect of the modified cell, the modified cell is a human cell or derived from a human cell.

In another aspect of the modified cell, the modified cell is a stem cell (e.g., a totipotent/omnipotent stem cell, a pluripotent stem cell, a multipotent stem cell, an oligopotent stem cell, or an unipotent stem cell), a differentiated cell, or a terminally differentiated cell.

In another aspect of the modified cell, the modified cell is a primary cell.

In another aspect of the modified cell, the modified cell is from a cell line.

In another aspect of the modified cell, the modified cell is a T cell, B cell, or NK cell.

In another aspect of the modified cell, the modified cell comprises a modification in a gene selected from the group consisting of: BCL11A intronic erythroid enhancer, CD3, B2M, TRAC, PDCD1, PDL1, CIITA, TTR, LDHA, and HAO1.

The invention yet further provides progeny of a modified cell described herein.

The invention yet further provides a method of obtaining a plurality of cells, wherein the method comprises isolating and culturing a modified cell described herein.

The invention yet further provides a method of obtaining a plurality of cells, wherein the method comprises culturing a modified cell described herein.

The invention yet further provides a plurality of cells comprising the modified cell or a plurality of a modified cell described herein.

The invention yet further provides a modified cell comprising a deletion, wherein the deletion is adjacent to a 5′-NTTN-3′ sequence, wherein N is any nucleotide.

In one aspect of the modified cell, the deletion is up to about 40 nucleotides in length.

In another aspect of the modified cell, the deletion is from about 4 nucleotides to about 40 nucleotides in length.

In another aspect of the modified cell, the deletion is from about 4 nucleotides to about 25 nucleotides in length.

In another aspect of the modified cell, the deletion is from about 10 nucleotides to about 25 nucleotides in length.

In another aspect of the modified cell, the deletion is from about 10 nucleotides to about 15 nucleotides in length.

In another aspect of the modified cell, the deletion is downstream of the 5′-NTTN-3′ sequence.

In another aspect of the modified cell, the deletion starts within about 5 nucleotides to about 15 nucleotides of the 5′-NTTN-3′ sequence.

In another aspect of the modified cell, the deletion starts within about 5 nucleotides to about 10 nucleotides of the 5′-NTTN-3′ sequence.

In another aspect of the modified cell, the deletion starts within about 10 nucleotides to about 15 nucleotides of the 5′-NTTN-3′ sequence.

In another aspect of the modified cell, the deletion starts within about 5 nucleotides to about 15 nucleotides downstream of the 5′-NTTN-3′ sequence.

In another aspect of the modified cell, the deletion starts within about 5 nucleotides to about 10 nucleotides downstream of the 5′-NTTN-3′ sequence.

In another aspect of the modified cell, the deletion starts within about 10 nucleotides to about 15 nucleotides downstream of the 5′-NTTN-3′ sequence.

In another aspect of the modified cell, the deletion ends within about 20 nucleotides to about 30 nucleotides of the 5′-NTTN-3′ sequence.

In another aspect of the modified cell, the deletion ends within about 20 nucleotides to about 25 nucleotides of the 5′-NTTN-3′ sequence.

In another aspect of the modified cell, the deletion ends within about 25 nucleotides to about 30 nucleotides of the 5′-NTTN-3′ sequence.

In another aspect of the modified cell, the deletion ends within about 20 nucleotides to about 30 nucleotides downstream of the 5′-NTTN-3′ sequence.

In another aspect of the modified cell, the deletion ends within about 20 nucleotides to about 25 nucleotides downstream of the 5′-NTTN-3′ sequence.

In another aspect of the modified cell, the deletion ends within about 25 nucleotides to about 30 nucleotides downstream of the 5′-NTTN-3′ sequence.

In another aspect of the modified cell, the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

In another aspect of the modified cell, the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

In another aspect of the modified cell, the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

In another aspect of the modified cell, the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

In another aspect of the modified cell, the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

In another aspect of the modified cell, the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

In another aspect of the modified cell, the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

In another aspect of the modified cell, the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

In another aspect of the modified cell, the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

In another aspect of the modified cell, the deletion is in a genome of the modified cell.

In another aspect of the modified cell, the deletion is in an exon of the gene.

In another aspect of the modified cell, the deletion overlaps with a mutation in the gene.

In another aspect of the modified cell, the deletion overlaps with an insertion in the gene.

In another aspect of the modified cell, the deletion removes at least a portion of a repeat expansion of the gene.

In another aspect of the modified cell, the deletion disrupts one or both alleles of the gene.

In another aspect of the modified cell, the modified cell comprises two or more deletions.

In another aspect of the modified cell, an unmodified cell lacks the deletion.

In another aspect of the modified cell, the unmodified cell is a wild-type cell.

In another aspect of the modified cell, a number of nucleotides deleted in the modified cell is greater than a number of nucleotides deleted in a second modified cell, wherein the second modified is generated by treating an unmodified cell with a Cas9 polypeptide of SEQ ID NO: 5.

The invention yet further provides a modified cell comprising a DNA insertion, wherein the DNA insertion is adjacent to a 5′-NTTN-3′ sequence, wherein N is any nucleotide.

In one aspect of the modified cell, the insertion is 1 nucleotide in length.

In another aspect of the modified cell, the insertion is from 2 nucleotides to about 9 nucleotides in length.

In another aspect of the modified cell, the insertion is greater than about 9 nucleotides in length.

In another aspect of the modified cell, the insertion is downstream of the 5′-NTTN-3′ sequence.

In another aspect of the modified cell, the insertion starts within about 15 nucleotides to about 35 nucleotides of the 5′-NTTN-3′ sequence.

In another aspect of the modified cell, the insertion starts within about 18 nucleotides to about 30 nucleotides of the 5′-NTTN-3′ sequence.

In another aspect of the modified cell, the insertion starts within about 20 nucleotides to about 28 nucleotides of the 5′-NTTN-3′ sequence.

In another aspect of the modified cell, the insertion starts within about 20 nucleotides to about 25 nucleotides downstream of the 5′-NTTN-3′ sequence.

In another aspect of the modified cell, the insertion is in a genome of the modified cell.

In another aspect of the modified cell, the insertion is in an exon of the gene.

In another aspect of the modified cell, the insertion overlaps with a mutation in the gene.

In another aspect of the modified cell, the insertion overlaps with a deletion in the gene.

In another aspect of the modified cell, the insertion corrects a frameshift in the gene.

In another aspect of the modified cell, the insertion disrupts one or both alleles of the gene.

In another aspect of the modified cell, an unmodified cell lacks the DNA insertion.

In another aspect of the modified cell, the unmodified cell is a wild-type cell.

In another aspect of the modified cell, the 5′-NTTN-3′ sequence is 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′, 5′-ATTN-3′, or 5′-GTTN-3′, wherein Y is C or T, B is any nucleotide except for A, D is any nucleotide except for C, and R is A or G.

In another aspect of the modified cell, the 5′-NTTN-3′ sequence is 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′, 5′-GTTG-3′,5′-GTTC-3′,5′-CTTA-3′,5′-CTTT-3′,5′-CTTG-3′, or 5′-CTTC-3′.

In another aspect of the modified cell, the modified cell is a eukaryotic cell or a prokaryotic cell.

In another aspect of the modified cell, the modified cell is an animal cell, a plant cell, or a fungal cell or the cell is derived from an animal cell, a plant cell, or a fungal cell.

In another aspect of the modified cell, the modified cell is a mammalian cell or derived from a mammalian cell.

In another aspect of the modified cell, the modified cell is a human cell or derived from a human cell.

In another aspect of the modified cell, the modified cell is a stem cell (e.g., a totipotent/omnipotent stem cell, a pluripotent stem cell, a multipotent stem cell, an oligopotent stem cell, or an unipotent stem cell), a differentiated cell, or a terminally differentiated cell.

In another aspect of the modified cell, the modified cell is a primary cell.

In another aspect of the modified cell, the modified cell is from a cell line.

In another aspect of the modified cell, the modified cell is a T cell, B cell, or NK cell.

In another aspect of the modified cell, the modified cell comprises a modification in a gene selected from the group consisting of: BCL11A intronic erythroid enhancer, CD3, B2M, TRAC, PDCD1, PDL1, CIITA, TTR, LDHA, and HAO1.

The invention yet further provides progeny of a modified cell described herein.

The invention yet further provides a method of obtaining a plurality of cells, wherein the method comprises isolating and culturing a modified cell described herein.

The invention yet further provides a method of obtaining a plurality of cells, wherein the method comprises culturing a modified cell described herein.

The invention yet further provides a plurality of cells comprising the modified cell or a plurality of a modified cell described herein.

The invention yet further provides a plurality of cells, wherein at least 70% of the cells comprise a deletion in a gene, wherein the deletion is adjacent to a 5′-NTTN-3′ sequence.

In one aspect of the plurality of cells, at least 80% of the cells comprise the deletion.

In another aspect of the plurality of cells, at least 90% of the cells comprise the deletion.

In another aspect of the plurality of cells, each of the cells comprises the deletion.

In another aspect of the plurality of cells, the deletion is at least about 5 nucleotides in length in about 90% of the cells having the deletion.

In another aspect of the plurality of cells, the deletion is from 4 nucleotides to 40 nucleotides in length in the cells having the deletion.

In another aspect of the plurality of cells, the deletion is at least about 10 nucleotides in length in about 75% of the cells having the deletion.

In another aspect of the plurality of cells, the deletion is at least about 15 nucleotides in length in about 50% of the cells having the deletion.

In another aspect of the plurality of cells, the deletion is at least about 20 nucleotides in length in about 25% of the cells having the deletion.

In another aspect of the plurality of cells, the deletion is at least about 25 nucleotides in length in about 25% of the cells having the deletion.

In another aspect of the plurality of cells, the deletion is at least about 5 nucleotides or longer in about 90% of the cells having the deletion.

In another aspect of the plurality of cells, the deletion is about 10 nucleotides or longer in about 75% of the cells having the deletion.

In another aspect of the plurality of cells, the deletion is about 15 nucleotides or longer in about 50% of the cells having the deletion.

In another aspect of the plurality of cells, the deletion is about 20 nucleotides or longer in about 25% of the cells having the deletion.

In another aspect of the plurality of cells, the deletion is about 25 nucleotides or longer in about 25% of the cells having the deletion.

In another aspect of the plurality of cells, the deletion is downstream of the 5′-NTTN-3′ sequence.

In another aspect of the plurality of cells, the deletion starts within about 5 nucleotides to about 15 nucleotides of the 5′-NTTN-3′ sequence.

In another aspect of the plurality of cells, the deletion starts within about 5 nucleotides to about 10 nucleotides of the 5′-NTTN-3′ sequence.

In another aspect of the plurality of cells, the deletion starts within about 10 nucleotides to about 15 nucleotides of the 5′-NTTN-3′ sequence.

In another aspect of the plurality of cells, the deletion starts within about 5 nucleotides to about 15 nucleotides downstream of the 5′-NTTN-3′ sequence.

In another aspect of the plurality of cells, the deletion starts within about 5 nucleotides to about 10 nucleotides downstream of the 5′-NTTN-3′ sequence.

In another aspect of the plurality of cells, the deletion starts within about 10 nucleotides to about 15 nucleotides downstream of the 5′-NTTN-3′ sequence.

In another aspect of the plurality of cells, the deletion ends within about 20 nucleotides to about 30 nucleotides of the 5′-NTTN-3′ sequence.

In another aspect of the plurality of cells, the deletion ends within about 20 nucleotides to about 25 nucleotides of the 5′-NTTN-3′ sequence.

In another aspect of the plurality of cells, the deletion ends within about 25 nucleotides to about 30 nucleotides of the 5′-NTTN-3′ sequence.

In another aspect of the plurality of cells, the deletion ends within about 20 nucleotides to about 30 nucleotides downstream of the 5′-NTTN-3′ sequence.

In another aspect of the plurality of cells, the deletion ends within about 20 nucleotides to about 25 nucleotides downstream of the 5′-NTTN-3′ sequence.

In another aspect of the plurality of cells, the deletion ends within about 25 nucleotides to about 30 nucleotides downstream of the 5′-NTTN-3′ sequence.

In another aspect of the plurality of cells, the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

In another aspect of the plurality of cells, the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

In another aspect of the plurality of cells, the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

In another aspect of the plurality of cells, the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

In another aspect of the plurality of cells, the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

In another aspect of the plurality of cells, the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

In another aspect of the plurality of cells, the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

In another aspect of the plurality of cells, the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

In another aspect of the plurality of cells, the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

In another aspect of the plurality of cells, the deletion is in an exon of the gene.

In another aspect of the plurality of cells, the deletion overlaps with a mutation in the gene.

In another aspect of the plurality of cells, the deletion overlaps with an insertion in the gene.

In another aspect of the plurality of cells, the deletion removes at least a portion of a repeat expansion of the gene.

In another aspect of the plurality of cells, the deletion disrupts one or both alleles of the gene.

The invention yet further provides a plurality of cells, wherein at least 70% of the cells comprise an insertion in a gene, wherein the insertion is adjacent to a 5′-NTTN-3′ sequence.

In one aspect of the plurality of cells, at least 80% of the cells comprise the insertion.

In another aspect of the plurality of cells, at least 90% of the cells comprise the insertion.

In another aspect of the plurality of cells, 100% of the cells comprises the insertion.

In another aspect of the plurality of cells, the insertion is 1 nucleotide in length.

In another aspect of the plurality of cells, the insertion is from 2 nucleotides to about 9 nucleotides in length.

In another aspect of the plurality of cells, the insertion is greater than about 9 nucleotides in length.

In another aspect of the plurality of cells, the insertion is downstream of the 5′-NTTN-3′ sequence.

In another aspect of the plurality of cells, the insertion starts within about 15 nucleotides to about 35 nucleotides of the 5′-NTTN-3′ sequence.

In another aspect of the plurality of cells, the insertion starts within about 18 nucleotides to about 30 nucleotides of the 5′-NTTN-3′ sequence.

In another aspect of the plurality of cells, the insertion starts within about 20 nucleotides to about 28 nucleotides of the 5′-NTTN-3′ sequence.

In another aspect of the plurality of cells, the insertion starts within about 20 nucleotides to about 25 nucleotides downstream of the 5′-NTTN-3′ sequence.

In another aspect of the plurality of cells, the insertion is in an exon of the gene.

In another aspect of the plurality of cells, the insertion overlaps with a mutation in the gene.

In another aspect of the plurality of cells, the insertion overlaps with a deletion in the gene.

In another aspect of the plurality of cells, the insertion corrects a frameshift in the gene.

In another aspect of the plurality of cells, the insertion disrupts one or both alleles of the gene.

The invention yet further provides a plurality of cells, wherein (a) at least about 20% of the cells comprise a deletion adjacent to a 5′-NTTN-3′ sequence, and (b) less than about 3% of the cells comprise an insertion adjacent to the 5′-NTTN-3′ sequence.

In one aspect of the plurality of cells, at least 30% of the cells comprise the deletion.

In another aspect of the plurality of cells, at least 40% of the cells comprise the deletion.

In another aspect of the plurality of cells, at least 50% of the cells comprise the deletion.

In another aspect of the plurality of cells, at least 60% of the cells comprise the deletion.

In another aspect of the plurality of cells, at least 70% of the cells comprise the deletion.

In another aspect of the plurality of cells, at least 80% of the cells comprise the deletion.

In another aspect of the plurality of cells, at least 90% of the cells comprise the deletion.

In another aspect of the plurality of cells, less than about 2% of the cells comprise the insertion.

In another aspect of the plurality of cells, less than about 1% of the cells comprise the insertion.

In another aspect of the plurality of cells, less than about 0.5% of the cells comprise the insertion.

In another aspect of the plurality of cells, less than about 0.1% of the cells comprise the insertion.

In another aspect of the plurality of cells, the deletion is downstream of the 5′-NTTN-3′ sequence.

In another aspect of the plurality of cells, the deletion starts within about 5 nucleotides to about 15 nucleotides of the 5′-NTTN-3′ sequence.

In another aspect of the plurality of cells, the deletion starts within about 5 nucleotides to about 10 nucleotides of the 5′-NTTN-3′ sequence.

In another aspect of the plurality of cells, the deletion starts within about 10 nucleotides to about 15 nucleotides of the 5′-NTTN-3′ sequence.

In another aspect of the plurality of cells, the deletion starts within about 5 nucleotides to about 15 nucleotides downstream of the 5′-NTTN-3′ sequence.

In another aspect of the plurality of cells, the deletion starts within about 5 nucleotides to about 10 nucleotides downstream of the 5′-NTTN-3′ sequence.

In another aspect of the plurality of cells, the deletion starts within about 10 nucleotides to about 15 nucleotides downstream of the 5′-NTTN-3′ sequence.

In another aspect of the plurality of cells, the deletion ends within about 20 nucleotides to about 30 nucleotides of the 5′-NTTN-3′ sequence.

In another aspect of the plurality of cells, the deletion ends within about 20 nucleotides to about 25 nucleotides of the 5′-NTTN-3′ sequence.

In another aspect of the plurality of cells, the deletion ends within about 25 nucleotides to about 30 nucleotides of the 5′-NTTN-3′ sequence.

In another aspect of the plurality of cells, the deletion ends within about 20 nucleotides to about 30 nucleotides downstream of the 5′-NTTN-3′ sequence.

In another aspect of the plurality of cells, the deletion ends within about 20 nucleotides to about 25 nucleotides downstream of the 5′-NTTN-3′ sequence.

In another aspect of the plurality of cells, the deletion ends within about 25 nucleotides to about 30 nucleotides downstream of the 5′-NTTN-3′ sequence.

In another aspect of the plurality of cells, the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

In another aspect of the plurality of cells, the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

In another aspect of the plurality of cells, the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

In another aspect of the plurality of cells, the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

In another aspect of the plurality of cells, the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

In another aspect of the plurality of cells, the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

In another aspect of the plurality of cells, the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

In another aspect of the plurality of cells, the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

In another aspect of the plurality of cells, the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

In another aspect of the plurality of cells, the insertion is 1 nucleotide in length.

In another aspect of the plurality of cells, the insertion is from 2 nucleotides to about 9 nucleotides in length.

In another aspect of the plurality of cells, the insertion is greater than about 9 nucleotides in length.

In another aspect of the plurality of cells, the insertion is downstream of the 5′-NTTN-3′ sequence.

In another aspect of the plurality of cells, the insertion starts within about 15 nucleotides to about 35 nucleotides of the 5′-NTTN-3′ sequence.

In another aspect of the plurality of cells, the insertion starts within about 18 nucleotides to about 30 nucleotides of the 5′-NTTN-3′ sequence.

In another aspect of the plurality of cells, the insertion starts within about 20 nucleotides to about 28 nucleotides of the 5′-NTTN-3′ sequence.

In another aspect of the plurality of cells, the insertion starts within about 20 nucleotides to about 25 nucleotides downstream of the 5′-NTTN-3′ sequence.

In another aspect of the plurality of cells, the 5′-NTTN-3′ sequence is 5′-NTTY-3′,5′-NTTC-3′, 5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′, wherein Y is C or T, B is any nucleotide except for A, D is any nucleotide except for C, and R is A or G.

In another aspect of the plurality of cells, the 5′-NTTN-3′ sequence is 5′-ATTA-3′,5′-ATTT-3′, 5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′.

In another aspect of the plurality of cells, the plurality of cells are eukaryotic cells or prokaryotic cells.

In another aspect of the plurality of cells, the plurality of cells are animal cells, plant cells, or fungal cells or the cells derived from animal cells, plant cells, or fungal cells.

In another aspect of the plurality of cells, the plurality of cells are mammalian cells or derived from mammalian cells.

In another aspect of the plurality of cells, the plurality of cells are human cells or derived from human cells.

In another aspect of the plurality of cells, the plurality of cells are stem cells (e.g., totipotent/omnipotent stem cells, pluripotent stem cells, multipotent stem cells, oligopotent stem cells, or unipotent stem cells), differentiated cells, or terminally differentiated cells.

In another aspect of the plurality of cells, the plurality of cells are primary cells.

In another aspect of the plurality of cells, the plurality of cells are cells of a cell line.

In another aspect of the plurality of cells, the plurality of cells comprise two or more cell types (e.g., are a co-culture of cells).

In another aspect of the plurality of cells, the plurality of cells are T cells, B cells, or NK cells.

In another aspect of the plurality of cells, the plurality of cells comprise a modification in a gene selected from the group consisting of: BCL11A intronic erythroid enhancer, CD3, B2M, TRAC, PDCD1, PDL1, CIITA, TTR, LDHA, and HAO1.

The invention yet further provides a plurality of modified cells, wherein at least about 0.1% of the modified cells comprise an insertion adjacent to a 5′-NTTN-3′ sequence, wherein N is any nucleotide.

In one aspect of the plurality of cells, at least about 0.5% of the modified cells comprise the insertion.

In another aspect of the plurality of modified cells, at least about 1.0% of the modified cells comprise the insertion.

In another aspect of the plurality of modified cells, at least about 2.0% of the modified cells comprise the insertion.

In another aspect of the plurality of modified cells, at least about 3.0% of the modified cells comprise the insertion.

In another aspect of the plurality of modified cells, the insertion is 1 nucleotide in length.

In another aspect of the plurality of modified cells, the insertion is from 2 nucleotides to about 9 nucleotides in length.

In another aspect of the plurality of modified cells, the insertion is greater than about 9 nucleotides in length.

In another aspect of the plurality of modified cells, the insertion is downstream of the 5′-NTTN-3′ sequence.

In another aspect of the plurality of modified cells, the insertion starts within about 15 nucleotides to about 35 nucleotides of the 5′-NTTN-3′ sequence.

In another aspect of the plurality of modified cells, the insertion starts within about 18 nucleotides to about 30 nucleotides of the 5′-NTTN-3′ sequence.

In another aspect of the plurality of modified cells, the insertion starts within about 20 nucleotides to about 28 nucleotides of the 5′-NTTN-3′ sequence.

In another aspect of the plurality of modified cells, the insertion starts within about 20 nucleotides to about 25 nucleotides downstream of the 5′-NTTN-3′ sequence.

In another aspect of the plurality of modified cells, the 5′-NTTN-3′ sequence is 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′, 5′-DTTT-3′,5′-ATTN-3′, or 5′-GTTN-3′, wherein Y is C or T, B is any nucleotide except for A, D is any nucleotide except for C, and R is A or G.

In another aspect of the plurality of modified cells, the 5′-NTTN-3′ sequence is 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′, 5′-GTTT-3′,5′-GTTG-3′,5′-GTTC-3′,5′-CTTA-3′,5′-CTTT-3′,5′-CTTG-3′, or 5′-CTTC-3′.

In another aspect of the plurality of modified cells, the insertion is in an exon of the gene.

In another aspect of the plurality of modified cells, the insertion overlaps with a mutation in the gene.

In another aspect of the plurality of modified cells, the insertion overlaps with a deletion in the gene.

In another aspect of the plurality of modified cells, the insertion corrects a frameshift in the gene.

In another aspect of the plurality of modified cells, the insertion disrupts one or both alleles of the gene.

In another aspect of the plurality of modified cells, the plurality of modified cells are eukaryotic cells or prokaryotic cells.

In another aspect of the plurality of modified cells, the plurality of modified cells are animal cells, plant cells, or fungal cells or the cells derived from animal cells, plant cells, or fungal cells.

In another aspect of the plurality of modified cells, the plurality of modified cells are mammalian cells or derived from mammalian cells.

In another aspect of the plurality of modified cells, the plurality of modified cells are human cells or derived from human cells.

In another aspect of the plurality of modified cells, the plurality of modified cells are stem cells (e.g., totipotent/omnipotent stem cells, pluripotent stem cells, multipotent stem cells, oligopotent stem cells, or unipotent stem cells), differentiated cells, or terminally differentiated cells.

In another aspect of the plurality of modified cells, the plurality of modified cells are primary cells.

In another aspect of the plurality of modified cells, the plurality of modified cells are cells of a cell line.

In another aspect of the plurality of modified cells, the plurality of modified cells comprise two or more cell types (e.g., are a co-culture of cells).

In yet another aspect, the invention provides a composition or formulation comprising a modified cell described herein, a plurality of cells described herein, or a plurality of modified cells described herein.

In yet another aspect, the invention provides a composition or formulation comprising a modified cell or a plurality of cells comprising a deletion, wherein the deletion is adjacent to a 5′-NTTN-3′ sequence, wherein N is any nucleotide.

In one aspect of the composition or formulation, at least 70% of the plurality of cells comprise the deletion.

In another aspect of the composition or formulation, at least 80% of the plurality of cells comprise the deletion.

In another aspect of the composition or formulation, at least 90% of the plurality of cells comprise the deletion.

In another aspect of the composition or formulation, 100% of the plurality of cells comprise the deletion.

In another aspect of the composition or formulation, the deletion is up to about 40 nucleotides in length.

In another aspect of the composition or formulation, the deletion is between about 4 nucleotides and 40 nucleotides in length.

In another aspect of the composition or formulation, the deletion is between about 4 nucleotides and 25 nucleotides in length.

In another aspect of the composition or formulation, the deletion is between about 10 nucleotides and 25 nucleotides in length.

In another aspect of the composition or formulation, the deletion is between about 10 nucleotides and 15 nucleotides in length.

In another aspect of the composition or formulation, the deletion starts within about 5 nucleotides to about 15 nucleotides of the 5′-NTTN-3′ sequence.

In another aspect of the composition or formulation, the deletion starts within about 5 nucleotides to about 10 nucleotides of the 5′-NTTN-3′ sequence.

In another aspect of the composition or formulation, the deletion starts within about 10 nucleotides to about 15 nucleotides of the 5′-NTTN-3′ sequence.

In another aspect of the composition or formulation, the deletion starts within about 5 nucleotides to about 15 nucleotides downstream of the 5′-NTTN-3′ sequence.

In another aspect of the composition or formulation, the deletion starts within about 5 nucleotides to about 10 nucleotides downstream of the 5′-NTTN-3′ sequence.

In another aspect of the composition or formulation, the deletion starts within about 10 nucleotides to about 15 nucleotides downstream of the 5′-NTTN-3′ sequence.

In another aspect of the composition or formulation, the deletion ends within about 20 nucleotides to about 30 nucleotides of the 5′-NTTN-3′ sequence.

In another aspect of the composition or formulation, the deletion ends within about 20 nucleotides to about 25 nucleotides of the 5′-NTTN-3′ sequence.

In another aspect of the composition or formulation, the deletion ends within about 25 nucleotides to about 30 nucleotides of the 5′-NTTN-3′ sequence.

In another aspect of the composition or formulation, the deletion ends within about 20 nucleotides to about 30 nucleotides downstream of the 5′-NTTN-3′ sequence.

In another aspect of the composition or formulation, the deletion ends within about 20 nucleotides to about 25 nucleotides downstream of the 5′-NTTN-3′ sequence.

In another aspect of the composition or formulation, the deletion ends within about 25 nucleotides to about 30 nucleotides downstream of the 5′-NTTN-3′ sequence.

In another aspect of the composition or formulation, the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

In another aspect of the composition or formulation, the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

In another aspect of the composition or formulation, the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

In another aspect of the composition or formulation, the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

In another aspect of the composition or formulation, the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

In another aspect of the composition or formulation, the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

In another aspect of the composition or formulation, the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

In another aspect of the composition or formulation, the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

In another aspect of the composition or formulation, the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

In another aspect of the composition or formulation, the deletion is in an exon of the gene.

In another aspect of the composition or formulation, the deletion overlaps with a mutation in the gene.

In another aspect of the composition or formulation, the deletion overlaps with an insertion in the gene.

In another aspect of the composition or formulation, the deletion removes at least a portion of a repeat expansion of the gene.

In another aspect of the composition or formulation, the deletion disrupts one or both alleles of the gene.

In yet another aspect of the composition or formulation, the invention provides a composition or formulation comprising a modified cell or a plurality of modified cells, wherein the insertion is adjacent to a 5′-NTTN-3′ sequence, wherein N is any nucleotide.

In one aspect of the composition or formulation, the insertion is 1 nucleotide in length.

In another aspect of the composition or formulation, the insertion is from 2 nucleotides to about 9 nucleotides in length.

In another aspect of the composition or formulation, the insertion is greater than about 9 nucleotides in length.

In another aspect of the composition or formulation, the insertion is downstream of the 5′-NTTN-3′ sequence.

In another aspect of the composition or formulation, the insertion starts within about 15 nucleotides to about 35 nucleotides of the 5′-NTTN-3′ sequence.

In another aspect of the composition or formulation, the insertion starts within about 18 nucleotides to about 30 nucleotides of the 5′-NTTN-3′ sequence.

In another aspect of the composition or formulation, the insertion starts within about 20 nucleotides to about 28 nucleotides of the 5′-NTTN-3′ sequence.

In another aspect of the composition or formulation, the insertion starts within about 20 nucleotides to about 25 nucleotides downstream of the 5′-NTTN-3′ sequence.

In another aspect of the composition or formulation, the insertion is in an exon of the gene.

In another aspect of the composition or formulation, the insertion overlaps with a mutation in the gene.

In another aspect of the composition or formulation, the insertion overlaps with a deletion in the gene.

In another aspect of the composition or formulation, the insertion corrects a frameshift in the gene.

In another aspect of the composition or formulation, the insertion disrupts one or both alleles of the gene.

In another aspect of the composition or formulation, the 5′-NTTN-3′ sequence is 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′, 5′-DTTT-3′,5′-ATTN-3′, or 5′-GTTN-3′, wherein Y is C or T, B is any nucleotide except for A, D is any nucleotide except for C, and R is A or G.

In another aspect of the composition or formulation, the 5′-NTTN-3′ sequence is 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′, 5′-GTTT-3′,5′-GTTG-3′,5′-GTTC-3′,5′-CTTA-3′,5′-CTTT-3′,5′-CTTG-3′, or 5′-CTTC-3′.

In another aspect of the composition or formulation, at least about 0.1% of the plurality of modified cells comprise the insertion.

In another aspect of the composition or formulation, at least about 0.5% of the plurality of modified cells comprise the insertion.

In another aspect of the composition or formulation, at least about 1.0% of the plurality of modified cells comprise the insertion.

In another aspect of the composition or formulation, at least about 2.0% of the plurality of modified cells comprise the insertion.

In another aspect of the composition or formulation, at least about 3.0% of the plurality of modified cells comprise the insertion.

In another aspect of the composition or formulation, at least about 70% of the plurality of modified cells comprise the insertion.

In another aspect of the composition or formulation, at least about 80% of the plurality of modified cells comprise the insertion.

In another aspect of the composition or formulation, at least about 90% of the plurality of modified cells comprise the insertion.

In another aspect of the composition or formulation, 100% of the plurality of modified cells comprise the insertion.

In another aspect of the composition or formulation, the cell is a eukaryotic cell or a prokaryotic cell.

In another aspect of the composition or formulation, the modified cell or a cell of the plurality is an animal cell, a plant cell, or a fungal cell or the cell is derived from an animal cell, a plant cell, or a fungal cell.

In another aspect of the composition or formulation, the modified cell or a cell of the plurality is a mammalian cell or derived from a mammalian cell.

In another aspect of the composition or formulation, the modified cell or a cell of the plurality is a human cell or derived from a human cell.

In another aspect of the composition or formulation, the modified cell or a cell of the plurality is a stem cell (e.g., a totipotent/omnipotent stem cell, a pluripotent stem cell, a multipotent stem cell, an oligopotent stem cell, or an unipotent stem cell), a differentiated cell, or a terminally differentiated cell.

In another aspect of the composition or formulation, the modified cell or a cell of the plurality is a primary cell.

In another aspect of the composition or formulation, the modified cell or a cell of the plurality is a cell from a cell line.

### Definitions

The present disclosure will be described with respect to particular embodiments and with reference to certain Figures, but the disclosure is not limited thereto but only by the claims. Terms as set forth hereinafter are generally to be understood in their common sense unless indicated otherwise.

As used herein, the term “adjacent to” refers to a sequence in close proximity to another sequence. In some embodiments, a sequence is adjacent to another sequence if no nucleotides separate the two sequences. In some embodiments, a sequence is adjacent to another sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides) In some embodiments, a first sequence is adjacent to a second sequence if the two sequences are separated by about 5, 6, 7, 8, 9, 10, 11, 12, 13, 14, or 15 nucleotides. In some embodiments, the term “adjacent to” is used to refer to the positioning of an indel in a modified cell of the disclosure. In some embodiments, the term “adjacent to a gene” refers to a genetic sequence that is in close proximity to a gene, including, but not limited to, a promoter, regulatory sequence, or intergenic sequence. A sequence that is adjacent to a gene can be a coding sequence or a non-coding sequence.

As used herein, the term “Cas12i polypeptide” (also referred to herein as Cas12i) refers to a polypeptide comprising at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 1-5 and SEQ ID NOs: 11-18 of U.S. Pat. No. 10,808,245, which is incorporated by reference herein in its entirety.

In some embodiments, a Cas12i polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 3, 5, 14, or 16 of U.S. Pat. No. 10,808,245. In some embodiments, a Cas12i2 polypeptide of the disclosure is a Cas12i2 polypeptide as described in PCT/US2021/025257. In some embodiments, a Cas12i2 polypeptide comprises at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with any one of SEQ ID NOs: 2-4 and SEQ ID NOs: 46-48. In some embodiments, a Cas12i2 polypeptide comprises a sequence of any one of SEQ ID NOs: 2-4 and SEQ ID NOs: 46-48. In some embodiments, a Cas12i2 polypeptide of the disclosure is encoded by a nucleic acid sequence comprising at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with SEQ ID NO: 1. In some embodiments, a Cas12i2 polypeptide of the disclosure is encoded by a nucleic acid sequence comprising the sequence of SEQ ID NO: 1.

As used herein, the term “Cas12i-induced” and the like in reference to a deletion or insertion refer to a deletion or insertion created upon cleavage of a target nucleic acid molecule by a Cas12i polypeptide (e.g., a deletion or insertion directly induced by Cas12i) or a deletion or insertion created following cleavage of a target nucleic acid molecule by Cas12i and DNA repair of the target nucleic acid molecule (e.g., a deletion or insertion indirectly induced by Cas12i).

As used herein, the term “deletion” refers to a loss or removal of nucleotides in a nucleic acid sequence relative to a reference sequence. The deletion can be a frameshift mutation or a non-frameshift mutation. A Cas12i-induced deletion described herein refers to a deletion of up to about 100 nucleotides, such as from about 4 nucleotides and 100 nucleotides, from about 4 nucleotides and 50 nucleotides, from about 4 nucleotides and 40 nucleotides, from about 4 nucleotides and 25 nucleotides, from about 10 nucleotides and 25 nucleotides, from about 10 nucleotides and 15 nucleotides, from a nucleic acid molecule. In some embodiments, a Cas12i-induced deletion described herein occurs downstream of a 5′-NTTN-3′ sequence.

As used herein, the term “insertion” refers to a gain of nucleotides in a nucleic acid sequence relative to a reference sequence. The nucleic acid sequence can be in a genome of an organism. The nucleic acid sequence can be in a cell. The nucleic acid sequence can be a DNA sequence. The nucleic acid sequence can be an RNA sequence. The insertion can be a frameshift mutation or a non-frameshift mutation. A Cas12i-induced insertion described herein refers to an insertion of up to about 10 nucleotides. In some embodiments, a Cas12i-induced insertion described herein occurs downstream of a 5′-NTTN-3′ sequence.

As used herein, the term “protospacer adjacent motif” or “PAM” refers to a DNA sequence adjacent to a target sequence to which a complex comprising a Cas12i polypeptide and an RNA guide binds. In some embodiments, a PAM sequence is required for enzyme activity. In the case of a double-stranded target, the RNA guide binds to a first strand of the target, and a PAM sequence as described herein is present in the second, complementary strand. For example, in some embodiments, the RNA guide binds to the target strand (e.g., the spacer-complementary strand), and the PAM sequence as described herein is present in the non-target strand (i.e., the non-spacer-complementary strand).

As used herein, the term “plurality” indicates “two or more.” In some embodiments with respect to cells, the term “plurality” refers to two or more cells, such as two or more modified cells. For instance, in some embodiments, a plurality of cells comprises at least about 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,0000, or more cells. In some embodiments, a plurality of cells refers to cells of a cell culture or cell line.

As used herein, the term “progeny” refers to daughter cells resulting from division of one or more parent cells (e.g., modified parent cells). In some embodiments, progeny (e.g., daughter cells) are modified cells. In some embodiments, progeny are daughter cells resulting from one or more modified parent cells. In some embodiments, progeny are multi-generational, e.g., daughter cells of modified parent cells can be used to generate further daughter cells, and so on. It will be understood that more than one generation of progeny are envisioned.

As used herein, the term “reference sequence” refers to an unmodified nucleic acid sequence. The reference sequence can be a nucleic acid sequence not modified by a Cas12i polypeptide (e.g., a deletion or insertion directly induced by Cas12i). The reference sequence can be an unmodified genome of an organism. The reference sequence can be an unmodified genome of an organism. The reference sequence can be an unmodified nucleic acid sequence in a cell. The reference sequence can be an unmodified DNA sequence. The nucleic acid sequence can be an unmodified RNA sequence.

As used herein, the term “substantial” refers to a measurable, considerable, or ample amount. In some embodiments, the term “substantial” is used to refer to the expression level of a gene. In some embodiments wherein a modified cell lacks substantial expression of a gene, expression of the gene in the modified cell is 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, or any integer therebetween) the expression of the gene in an unmodified cell. In some embodiments wherein a modified cell lacks substantial expression of a gene, the modified cell does not express the gene.

As used herein, the terms “upstream” and “downstream” refer to relative positions within a single nucleic acid (e.g., DNA) sequence in a nucleic acid molecule. “Upstream” and “downstream” relate to the 5′ to 3′ direction, respectively, in which RNA transcription occurs. A first sequence is upstream of a second sequence when the 3′ end of the first sequence occurs before the 5′ end of the second sequence. A first sequence is downstream of a second sequence when the 5′ end of the first sequence occurs after the 3′ end of the second sequence. In some embodiments, the 5′-NTTN-3′ sequence is upstream of an insertion or deletion described herein, and the insertion or deletion is downstream of the 5′-NTTN-3′ sequence. In some embodiments, “downstream” in reference to a deletion or insertion refers to the relative position in the non-target strand (i.e., the non-spacer-complementary strand). In embodiments wherein a deletion or insertion is downstream of a 5′-NTTN-3′ sequence of the non-target strand, the deletion or insertion can also be described as being upstream of a 5′-NAAN-3′ sequence on the target strand (i.e., the spacer complementary strand). In embodiments wherein a deletion or insertion is downstream of a 5′-NTTN-3′ sequence of the sense strand (e.g., coding strand), the deletion or insertion can also be described as being upstream of a 5′-NAAN-3′ sequence on the antisense strand (e.g., non-coding strand). In embodiments wherein a deletion or insertion is downstream of a 5′-NTTN-3′ sequence of the antisense strand (e.g., non-coding strand), the deletion or insertion can also be described as being upstream of a 5′-NAAN-3′ sequence on the sense strand (e.g., coding strand).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows AAVS1, EMX1, and VEGFA target loci for Cas12i2 and SpCas9. The sequences of the target loci are set forth in SEQ ID NOs: 6, 8, 10, 12, 14, 16, 18, 20, 22, 24, 26, 28, 30, 32, 34, 36, 38, 40, 42, and 44.

FIG. 2A shows indel size frequency (left column) and a cumulative density function (CDF) calculated for indel size frequency (right column) in cells modified at AAVS1 target locus 2 with variant Cas12i2 of SEQ ID NO: 3, variant Cas12i2 of SEQ ID NO: 4, or SpCas9 of SEQ ID NO: 5. FIG. 2B shows frequencies for indel start positions (left column) and end positions (right column) relative to the 5′ end of the AAVS1 target locus 2 sequence in cells modified with variant Cas12i2 of SEQ ID NO: 3, variant Cas12i2 of SEQ ID NO: 4, or SpCas9 of SEQ ID NO: 5.

FIG. 3A shows indel size frequency (left column) and a CDF calculated for indel size frequency (right column) in cells modified at AAVS1 target locus 3 with wild-type Cas12i2 of SEQ ID NO: 2, variant Cas12i2 of SEQ ID NO: 3, variant Cas12i2 of SEQ ID NO: 4, or SpCas9 (SEQ ID NO: 5). FIG. 3B shows frequencies for indel start positions (left column) and indel end positions (right column) relative to the 5′ end of the AAVS1 target locus 3 sequence in cells modified with wild-type Cas12i2 of SEQ ID NO: 2, variant Cas12i2 of SEQ ID NO: 3, variant Cas12i2 of SEQ ID NO: 4, or SpCas9 (SEQ ID NO: 5).

FIG. 4A shows indel size frequency (left column) and a CDF calculated for indel size frequency (right column) in cells modified at AAVS1 target locus 4 with variant Cas12i2 of SEQ ID NO: 3, variant Cas12i2 of SEQ ID NO: 4, or SpCas9 of SEQ ID NO: 5. FIG. 4B shows frequencies for indel start positions (left column) and indel end positions (right column) relative to the 5′ end of the AAVS1 target locus 4 sequence in cells modified with variant Cas12i2 of SEQ ID NO: 3, variant Cas12i2 of SEQ ID NO: 4, or SpCas9 of SEQ ID NO: 5.

FIG. 5A shows indel size frequency (left column) and a CDF calculated for indel size frequency (right column) in cells modified at AAVS1 target locus 6 with wild-type Cas12i2 of SEQ ID NO: 2, variant Cas12i2 of SEQ ID NO: 3, variant Cas12i2 of SEQ ID NO: 4, or SpCas9 (SEQ ID NO: 5). FIG. 5B shows frequencies for indel start positions (left column) and indel end positions (right column) relative to the 5′ end of the AAVS1 target locus 6 sequence in cells modified with wild-type Cas12i2 of SEQ ID NO: 2, variant Cas12i2 of SEQ ID NO: 3, variant Cas12i2 of SEQ ID NO: 4, or SpCas9 (SEQ ID NO: 5).

FIG. 6A shows indel size frequency (left column) and a CDF calculated for indel size frequency (right column) in cells modified at EMX1 target locus 3 with wild-type Cas12i2 of SEQ ID NO: 2, variant Cas12i2 of SEQ ID NO: 3, variant Cas12i2 of SEQ ID NO: 4, or SpCas9 (SEQ ID NO: 5). FIG. 6B shows frequencies for indel start positions (left column) and indel end positions (right column) relative to the 5′ end of the EMX1 target locus 3 sequence in cells modified with wild-type Cas12i2 of SEQ ID NO: 2, variant Cas12i2 of SEQ ID NO: 3, variant Cas12i2 of SEQ ID NO: 4, or SpCas9 (SEQ ID NO: 5).

FIG. 7A shows indel size frequency (left column) and a CDF calculated for indel size frequency (right column) in cells modified at EMX1 target locus 4 with variant Cas12i2 of SEQ ID NO: 3, variant Cas12i2 of SEQ ID NO: 4, or SpCas9 of SEQ ID NO: 5. FIG. 7B shows frequencies for indel start positions (left column) and indel end positions (right column) relative to the 5′ end of the EMX1 target locus 4 sequence in cells modified with variant Cas12i2 of SEQ ID NO: 3, variant Cas12i2 of SEQ ID NO: 4, or SpCas9 of SEQ ID NO: 5.

FIG. 8A shows indel size frequency (left column) and a CDF calculated for indel size frequency (right column) in cells modified at EMX1 target locus 5 with wild-type Cas12i2 of SEQ ID NO: 2, variant Cas12i2 of SEQ ID NO: 3, variant Cas12i2 of SEQ ID NO: 4, or SpCas9 (SEQ ID NO: 5). FIG. 8B shows frequencies for indel start positions (left column) and indel end positions (right column) relative to the 5′ end of the EMXI target locus 5 sequence in cells modified with wild-type Cas12i2 of SEQ ID NO: 2, variant Cas12i2 of SEQ ID NO: 3, variant Cas12i2 of SEQ ID NO: 4, or SpCas9 (SEQ ID NO: 5).

FIG. 9A shows indel size frequency (left column) and a CDF calculated for indel size frequency (right column) in cells modified at VEGFA target locus 1 with variant Cas12i2 of SEQ ID NO: 3, variant Cas12i2 of SEQ ID NO: 4, or SpCas9 of SEQ ID NO: 5. FIG. 9B shows frequencies for indel start positions (left column) and indel end positions (right column) relative to the 5′ end of the VEGFA target locus 1 sequence in cells modified with variant Cas12i2 of SEQ ID NO: 3, variant Cas12i2 of SEQ ID NO: 4, or SpCas9 of SEQ ID NO: 5.

FIG. 10A shows indel size frequency (left column) and a CDF calculated for indel size frequency (right column) in cells modified at VEGFA target locus 3 with wild-type Cas12i2 of SEQ ID NO: 2, variant Cas12i2 of SEQ ID NO: 3, variant Cas12i2 of SEQ ID NO: 4, or SpCas9 (SEQ ID NO: 5). FIG. 10B shows frequencies for indel start positions (left column) and indel end positions (right column) relative to the 5′ end of the VEGFA target locus 3 sequence in cells modified with wild-type Cas12i2 of SEQ ID NO: 2, variant Cas12i2 of SEQ ID NO: 3, variant Cas12i2 of SEQ ID NO: 4, or SpCas9 (SEQ ID NO: 5).

FIG. 11A shows indel size frequency (left column) and a CDF calculated for indel size frequency (right column) in cells modified at VEGFA target locus 4 with wild-type Cas12i2 of SEQ ID NO: 2, variant Cas12i2 of SEQ ID NO: 3, variant Cas12i2 of SEQ ID NO: 4, or SpCas9 (SEQ ID NO: 5). FIG. 11B shows frequencies for indel start positions (left column) and indel end positions (right column) relative to the 5′ end of the VEGFA target locus 4 sequence in cells modified with wild-type Cas12i2 of SEQ ID NO: 2, variant Cas12i2 of SEQ ID NO: 3, variant Cas12i2 of SEQ ID NO: 4, or SpCas9 (SEQ ID NO: 5).

FIG. 12 is a schematic showing the location of an insertion relative to a 5′-NTTN-3′ sequence for Cas12i2 or relative to a 5′-NGG-3′ sequence for Cas9.

FIG. 13 shows indel activity in primary T cells seven days after targeting B2M with a variant Cas12i2 of SEQ ID NO: 4 and different individual crRNAs (labeled B2M_4, B2M_8, B2M_10, or B2M_11) at different ribonucleoprotein (RNP) concentrations.

FIG. 14A shows reduced expression of B2M in primary T cells seven days after targeting of B2M with a variant Cas12i2 of SEQ ID NO: 4 in complex with different individual crRNAs (labeled B2M_4, B2M_8, B2M_10, or B2M_11) at different RNP concentrations. FIG. 14B shows viability of primary T cells, as measured by DAPI staining, seven days after targeting of B2M with a variant Cas12i2 of SEQ ID No: 4 in complex with different individual crRNAs (labeled B2M_4, B2M_8, B2M_10, or B2M_11) at different RNP concentrations.

FIG. 15A shows reduced expression of TRAC in primary T cells seven days after targeting of TRAC with a variant Cas12i2 of SEQ ID NO: 4 in complex with different individual crRNAs (labeled TRAC_1_3, TRAC_1_5, TRAC_2_4, and TRAC_34) at different RNP concentrations. FIG. 15B shows viability of primary T cells, as measured by DAPI staining, seven days after targeting of TRAC with a variant Cas12i2 of SEQ ID NO: 4 in complex with different individual crRNAs (labeled B2M_4, B2M_8, B2M_10, or B2M_11) at different RNP concentrations.

FIG. 16A shows reduced expression of PDCD1 in primary T cells following targeting of PDCD1 using a variant Cas12i2 of SEQ ID NO: 4 in complex with different individual crRNAs (labeled PDCD1_1_1, PDCD1_2_7, PDCD1_2_8, and PDCD1_2_9) at different RNP concentrations. FIG. 16B shows viability of primary T cells, as measured by DAPI staining, seven days after targeting of PDCD1 with a variant Cas12i2 of SEQ ID No: 4 in complex with different individual crRNAs (labeled B2M_4, B2M_8, B2M_10, or B2M_11) at different RNP concentrations.

FIG. 17 shows indel activity in primary T cells after targeting BCL11A intronic erythroid enhancer with different individual and multiplexed crRNAs in complex with a variant Cas12i2 of SEQ ID NO: 4 at various RNP concentrations. Error bars represent standard deviation of the mean of two bioreplicates (two individual donors).

FIG. 18 shows viability of modified CD34+ HSPC cells 72 hours following targeting of BCL11A intronic erythroid enhancer in primary CD34+ HSPCs. Different concentrations of BCL11A intronic erythroid enhancer targeting RNPs comprising variant Cas12i2 of SEQ ID NO: 4 and crRNAs were tested. crRNAs were tested individually and in multiplexed configuration. Error bars represent standard deviation of the mean of two bioreplicates (two individual donors).

### DETAILED DESCRIPTION

The present disclosure relates to a modified cell comprising a DNA deletion and/or DNA insertion induced by a Cas12i nuclease. In some aspects, a modified cell having one or more characteristics is described herein. In some aspects, a method of producing the modified cell is described. In some aspects, a composition or formulation comprises the modified cell described herein or a plurality of the modified cells described herein.

### Modified Cells

In some aspects, the disclosure described herein comprises a modified cell or a plurality of modified cells. In some embodiments, the modified cell is a genetically modified cell. In some embodiments, the modified cell is a cell comprising an indel. In some embodiments, the modified cell is a cell comprising a deletion. In some embodiments, the modified cell is a cell comprising an insertion. In some embodiments, the modified cell comprises a biochemical modification.

Cell Type

The modified cell or plurality of modified cells described herein can be a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a primate cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell. In some embodiments, the cell is a primary cell.

In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a mesenchymal stem cell. In some embodiments, the cell is an embryonic stem cell. In some embodiments, the cell is a hematopoietic stem cell. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is a glial cell. In some embodiments, the cell is a pancreatic islet cell, including an alpha cell, beta cell, delta cell, or enterochromaffin cell. In some embodiments, the cell is an immune cell. In some embodiments, the immune cell is a T cell. In some embodiments, the immune cell is a B cell. In some embodiments, the immune cell is a Natural Killer (NK) cell. In some embodiments, the immune cell is a Tumor Infiltrating Lymphocyte (TIL). In some embodiments, the cell is a mammalian cell, e.g., a human cell or primate cell or a murine cell. In some embodiments, the murine cell is derived from a wild-type mouse, an immunosuppressed mouse, or a disease-specific mouse model. In some embodiments, the cell is a cell within a living tissue, organ, or organism.

Genetic Characteristics

In some embodiments, the modified cell comprises a modification in a genomic region or a gene. In some embodiments, the modification is in an exon region of a gene. In some embodiments, the modification is in an intron region of a gene. In some embodiments, the modification is in a promoter region of a gene. In some embodiments, the modification is in an enhancer region of a gene. In some embodiments, the modification is in a silencer region of a gene. In some embodiments, the modification is in a terminator region of a gene. In some embodiments, the modification is in a region that regulates transcription of a gene. In some embodiments, the modification results in an altered expression (e.g., increase or decrease) of a gene product.

In some embodiments, the modified cell comprises two or more modifications (e.g., two or more desired or targeted modifications). In some embodiments, the modified cell comprises two or more deletions in the same gene. In some embodiments, the modified cell comprises a deletion in a first gene and a deletion in a second gene. In some embodiments, the modified cell comprises two or more insertions in the same gene. In some embodiments, the modified cell comprises an insertion in a first gene and an insertion in a second gene. In some embodiments, the modified cell comprises two or more indels (e.g., at least one deletion and at least one insertion) in the same gene. In some embodiments, the modified cell comprises an indel (e.g., deletion or insertion) in a first gene and an indel (e.g., deletion or insertion) in a second gene.

In some embodiments, the gene having the modification is present in the nucleus of a cell as described elsewhere herein. In some embodiments, the gene having the modification is endogenous to the cell. In some embodiments, the gene having the modification is a genomic DNA. In some embodiments, the gene having the modification is a chromosomal DNA. In some embodiments, the gene having the modification is a protein-coding gene or a functional region thereof, such as a coding region, or a regulatory element, such as a promoter, enhancer, a 5′ or 3′ untranslated region, etc. In some embodiments, the modification is in an exon or an intron. In some embodiments, the gene having the modification is a non-coding gene, such as transposon, miRNA, tRNA, ribosomal RNA, ribozyme, or lncRNA.

In some embodiments, the modification alters expression of the gene. In some embodiments, the modification alters function of the gene. In some embodiments, the modification inactivates the gene. In some embodiments, the modification is a frameshifting modification. In some embodiments, the modification is a non-frameshifting modification. In some embodiments, the modification leads to cell toxicity or cell death (e.g., apoptosis).

In some embodiments, the modification (e.g., deletion or insertion) overlaps with a mutation in the gene. In some embodiments, the modification (e.g., deletion) overlaps with an insertion within the gene. For example, in some embodiments, the modification (e.g., deletion) removes at least a portion of a repeat expansion of the gene. In some embodiments, the modification (e.g., insertion) overlaps with a deletion within the gene. In some embodiments, the modification (e.g., insertion) corrects a deletion in a gene. In some embodiments, the insertion corrects a frameshift in a gene. In some embodiments, the modification disrupts one allele of the gene. In some embodiments, the modification disrupts both alleles of the gene.

Deletion

In some embodiments, the deletion described herein is a genomic deletion of a cell.

In some embodiments, a modified cell comprises a deletion, wherein the deletion is adjacent to a 5′-NTTN-3′ sequence, wherein N is any nucleotide. In some embodiments, the modified cell comprises the deletion as described herein compared to an unmodified cell that lacks the deletion. In some embodiments a modified cell comprises a number of nucleotides deleted in the modified cell is greater than a number of nucleotides deleted in a second modified cell, wherein the second modified is generated by treating an unmodified cell with a Cas9 polypeptide of SEQ ID NO: 5. In some embodiments, the deletion is in a genome of the modified cell. In some embodiments, an unmodified cell is a wild-type cell.

In some embodiments, the modified cell comprises a deletion adjacent to a 5′-NTTN-3′ sequence, wherein N is any nucleotide. In some embodiments, the modified cell comprises a deletion adjacent to a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′, 5′-CTTR-3′,5′-DTTT-3′,5′-ATTN-3′, or 5′-GTTN-3′ sequence, wherein Y is C or T, B is any nucleotide except for A, D is any nucleotide except for C, and R is A or G. In some embodiments, the modified cell comprises a deletion adjacent to a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the modified cell comprises a deletion adjacent to a T/C-rich sequence.

In some embodiments, the modified cell comprises a deletion 3′ of a 5′-NTTN-3′ sequence on a sense strand within or adjacent to a gene, relative to a reference sequence. In some embodiments, the modified cell comprises a deletion 3′ of a 5′-NTTN-3′ sequence on an antisense strand within or adjacent to a gene, relative to a reference sequence. In some embodiments, the modified cell comprises a deletion that starts within about 5 to about 25 nucleotides of a 5′-NTTN-3′ sequence on a sense strand within or adjacent to a gene, relative to a reference sequence. In some embodiments, the modified cell comprises a deletion that starts within about 5 to about 25 nucleotides of a 5′-NTTN-3′ sequence on an antisense strand within or adjacent to a gene, relative to a reference sequence. In some embodiments, the modified cell comprises a deletion that starts within about 5 to about 25 nucleotides downstream or 3′ of a 5′-NTTN-3′ sequence on a sense strand within or adjacent to a gene, relative to a reference sequence and ends about 15 to about 50 nucleotides downstream of the 5′-NTTN-3′ sequence. In some embodiments, the modified cell comprises a deletion that starts within about 5 to about 25 nucleotides downstream of a 5′-NTTN-3′ sequence on an antisense strand within or adjacent to a gene, relative to a reference sequence and ends about 15 to about 50 nucleotides downstream of the 5′-NTTN-3′ sequence. In some embodiments, the modified cell comprises a deletion that starts within about 5 to about 25 nucleotides downstream of a 5′-NTTN-3′ sequence on a sense strand within or adjacent to a gene, relative to a reference sequence and ends within about 5 to about 25 nucleotides of a 5′-NTTN-3′ sequence on an antisense strand of the gene, wherein the antisense strand 5′-NTTN-3′ sequence relative to the sense strand 5′-NTTN-3′ sequence is downstream of the sense strand 5′-NTTN-3′ sequence. In some embodiments, the modified cell comprises a deletion that starts within about 5 to about 15 nucleotides downstream of a 5′-NTTN-3′ sequence on the antisense strand within or adjacent to a gene, relative to a reference sequence and ends within about 5 to about 15 nucleotides of a 5′-NTTN-3′ sequence on the sense strand of the gene, wherein the sense strand 5′-NTTN-3′ sequence relative to the antisense strand 5′-NTTN-3′ sequence is downstream of the antisense strand 5′-NTTN-3′ sequence. In some embodiments, the modified cell comprises a deletion that starts about 5 to about 25 nucleotides downstream of a 5′-NTTN-3′ sequence on a sense strand within or adjacent to a gene, relative to a reference sequence, and ends about 5 to about 25 nucleotides upstream of or 5′ to a complementary sequence of the 5′-NTTN-3′ sequence (e.g. upstream of a 5′-NAAN-3′ sequence on the antisense strand of the gene). In some embodiments, the modified cell comprises a deletion of about 5 to about 25 nucleotides downstream of a 5′-NTTN-3′ sequence on an antisense strand within or adjacent to a gene, relative to a reference sequence, and ends about 5 to about 25 nucleotides upstream of or 5′ to a complementary sequence of the 5′-NTTN-3′ sequence (e.g., upstream of a 5′-NAAN-3′ sequence on the on the sense strand of the gene).

In some embodiments, the modified cell comprises a deletion downstream or 3′ of a 5′-NTTN-3′ sequence. In some embodiments, the modified cell comprises a deletion downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′, 5′-DTTT-3′,5′-ATTN-3′, or 5′-GTTN-3′ sequence. In some embodiments, the modified cell comprises a deletion downstream of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the modified cell comprises a deletion downstream of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of the 5′-NTTN-3′ sequence within or adjacent to a gene, relative to a reference sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a 5′-NTTY-3′,5′-NTTC-3′, 5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a 5′-CTTT-3′, 5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence within or adjacent to a gene, relative to a reference sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′, 5′-CTTR-3′,5′-DTTT-3′,5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence.

In some embodiments, the deletion starts within about 5 to about 10 nucleotides (e.g., about 3, 4, 5, 6, 7, 8, 9, 10, 11, or 12 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) of the 5′-NTTN-3′ sequence within or adjacent to a gene, relative to a reference sequence. In some embodiments, the deletion starts within about 5 to about 10 nucleotides (e.g., about 3, 4, 5, 6, 7, 8, 9, 10, 11, or 12 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence within or adjacent to a gene, relative to a reference sequence. In some embodiments, the deletion starts within about 5 to about 10 nucleotides (e.g., about 3, 4, 5, 6, 7, 8, 9, 10, 11, or 12 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence.

In some embodiments, the deletion starts within about 10 to about 15 nucleotides (e.g., about 8, 9, 10, 11, 12, 13, 14, 15, 16, or 17 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) of the 5′-NTTN-3′ sequence within or adjacent to a gene, relative to a reference sequence. In some embodiments, the deletion starts within about 10 to about 15 nucleotides (e.g., about 8, 9, 10, 11, 12, 13, 14, 15, 16, or 17 nucleotides) of a 5′-NTTY-3′,5′-NTTC-3′,5′-NTTT-3′,5′-NTTA-3′,5′-NTTB-3′, 5′-NTTG-3′,5′-CTTY-3′,5‘-DTTR’3′,5′-CTTR-3′,5′-DTTT-3′,5′-ATTN-3′, or 5′-GTTN-3′ sequence.

In some embodiments, the deletion starts within about 10 to about 15 nucleotides (e.g., about 8, 9, 10, 11, 12, 13, 14, 15, 16, or 17 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence within or adjacent to a gene, relative to a reference sequence. In some embodiments, the deletion starts within about 10 to about 15 nucleotides (e.g., about 8, 9, 10, 11, 12, 13, 14, 15, 16, or 17 nucleotides) downstream of a 5′-NTTY-3′,5′-NTTC-3′, 5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence within or adjacent to a gene, relative to a reference sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence within or adjacent to a gene, relative to a reference sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion ends within about 20 to about 30 nucleotides (e.g., about 17, 18, 19, 20, 21, 22, 23, 24, 25, 26, 27, 28, 29, 30, 31, 32, or 33 nucleotides) downstream of a T/C-rich sequence.

In some embodiments, the deletion ends within about 30 to about 40 nucleotides (e.g., about 27, 28, 29, 30, 31, 32, 33, 34, 35, 36, 37, 38, 39, 40, 41, 42, or 43 nucleotides) of the 5′-NTTN-3′ sequence. In some embodiments, the deletion ends within about 30 to about 40 nucleotides (e.g., about 27, 28, 29, 30, 31, 32, 33, 34, 35, 36, 37, 38, 39, 40, 41, 42, or 43 nucleotides) of the 5′-NTTN-3′ sequence within or adjacent to a gene, relative to a reference sequence. In some embodiments, the deletion ends within about 30 to about 40 nucleotides (e.g., about 27, 28, 29, 30, 31, 32, 33, 34, 35, 36, 37, 38, 39, 40, 41, 42, or 43 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion ends within about 30 to about 40 nucleotides (e.g., about 27, 28, 29, 30, 31, 32, 33, 34, 35, 36, 37, 38, 39, 40, 41, 42, or 43 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion ends within about 30 to about 40 nucleotides (e.g., about 27, 28, 29, 30, 31, 32, 33, 34, 35, 36, 37, 38, 39, 40, 41, 42, or 43 nucleotides) of a T/C-rich sequence.

In some embodiments, the deletion ends within about 30 to about 40 nucleotides (e.g., about 27, 28, 29, 30, 31, 32, 33, 34, 35, 36, 37, 38, 39, 40, 41, 42, or 43 nucleotides) downstream of the 5′-NTTN-3′ sequence. In some embodiments, the deletion ends within about 30 to about 40 nucleotides (e.g., about 27, 28, 29, 30, 31, 32, 33, 34, 35, 36, 37, 38, 39, 40, 41, 42, or 43 nucleotides) downstream of the 5′-NTTN-3′ sequence within or adjacent to a gene, relative to a reference sequence. In some embodiments, the deletion ends within about 30 to about 40 nucleotides (e.g., about 27, 28, 29, 30, 31, 32, 33, 34, 35, 36, 37, 38, 39, 40, 41, 42, or 43 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion ends within about 30 to about 40 nucleotides (e.g., about 27, 28, 29, 30, 31, 32, 33, 34, 35, 36, 37, 38, 39, 40, 41, 42, or 43 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion ends within about 30 to about 40 nucleotides (e.g., about 27, 28, 29, 30, 31, 32, 33, 34, 35, 36, 37, 38, 39, 40, 41, 42, or 43 nucleotides) downstream of a T/C-rich sequence.

In some embodiments, the deletion ends within about 40 to about 50 nucleotides (e.g., about 37, 38, 39, 40, 41, 42, 43, 44, 45, 46, 47, 48, 49, 50, 51, 52, or 53 nucleotides) of the 5′-NTTN-3′ sequence. In some embodiments, the deletion ends within about 40 to about 50 nucleotides (e.g., about 37, 38, 39, 40, 41, 42, 43, 44, 45, 46, 47, 48, 49, 50, 51, 52, or 53 nucleotides) of the 5′-NTTN-3′ sequence within or adjacent to a gene, relative to a reference sequence. In some embodiments, the deletion ends within about 40 to about 50 nucleotides (e.g., about 37, 38, 39, 40, 41, 42, 43, 44, 45, 46, 47, 48, 49, 50, 51, 52, or 53 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion ends within about 40 to about 50 nucleotides (e.g., about 37, 38, 39, 40, 41, 42, 43, 44, 45, 46, 47, 48, 49, 50, 51, 52, or 53 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion ends within about 40 to about 50 nucleotides (e.g., about 37, 38, 39, 40, 41, 42, 43, 44, 45, 46, 47, 48, 49, 50, 51, 52, or 53 nucleotides) of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) of the 5′-NTTN-3′ sequence within or adjacent to a gene, relative to a reference sequence. In some embodiments, the deletion ends within about 20 to about 25 nucleotides (e.g., about 17, 18, 19, 20, 21, 22, 23, 24, 25, 26, 27, or 28 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion ends within about 20 to about 25 nucleotides (e.g., about 17, 18, 19, 20, 21, 22, 23, 24, 25, 26, 27, or 28 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion ends within about 20 to about 25 nucleotides (e.g., about 17, 18, 19, 20, 21, 22, 23, 24, 25, 26, 27, or 28 nucleotides) downstream of the 5′-NTTN-3′ sequence within or adjacent to a gene, relative to a reference sequence. In some embodiments, the deletion ends within about 20 to about 25 nucleotides (e.g., about 17, 18, 19, 20, 21, 22, 23, 24, 25, 26, 27, or 28 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′, 5′-CTTR-3′,5′-DTTT-3′,5′-ATTN-3′, or 5′-GTTN-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion ends within about 20 to about 25 nucleotides (e.g., about 17, 18, 19, 20, 21, 22, 23, 24, 25, 26, 27, or 28 nucleotides) downstream of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) of the 5′-NTTN-3′ sequence within or adjacent to a gene, relative to a reference sequence. In some embodiments, the deletion ends within about 25 to about 30 nucleotides (e.g., about 22, 23, 24, 25, 26, 27, 28, 29, 30, 31, 32, or 33 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion ends within about 25 to about 30 nucleotides (e.g., about 22, 23, 24, 25, 26, 27, 28, 29, 30, 31, 32, or 33 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion ends within about 25 to about 30 nucleotides (e.g., about 22, 23, 24, 25, 26, 27, 28, 29, 30, 31, 32, or 33 nucleotides) downstream of the 5′-NTTN-3′ sequence within or adjacent to a gene, relative to a reference sequence. In some embodiments, the deletion ends within about 25 to about 30 nucleotides (e.g., about 22, 23, 24, 25, 26, 27, 28, 29, 30, 31, 32, or 33 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′, 5′-CTTR-3′,5′-DTTT-3′,5′-ATTN-3′, or 5′-GTTN-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion ends within about 25 to about 30 nucleotides (e.g., about 22, 23, 24, 25, 26, 27, 28, 29, 30, 31, 32, or 33 nucleotides) downstream of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence within or adjacent to a gene, relative to a reference sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-NTTY-3′,5′-NTTC-3′, 5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-NTTN-3′ sequence on a sense strand within or adjacent to a gene, relative to a reference sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-NTTN-3′ sequence on an antisense strand in or adjacent to a gene and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on a sense strand of a gene and ends within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on an antisense strand of the gene or upstream of a complementary sequence to a 5′-NTTN-3′ sequence on the sense strand of the gene (e.g., upstream of a 5′-NAAN-3′ sequence on the sense strand). In some embodiments, the deletion starts within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on an antisense strand of a gene and ends within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on a sense strand of the gene or upstream of a complementary sequence to a 5′-NTTN-3′ sequence on the antisense strand of the gene (e.g., a 5′-NAAN-3′ sequence on the antisense strand). In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′, 5′-CTTR-3′,5′-DTTT-3′,5′-ATTN-3′, or 5′-GTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of the 5′-NTTN-3′ sequence within or adjacent to a gene, relative to a reference sequence and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence within or adjacent to a gene, relative to a reference sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ends within about 20 to about 25 nucleotides (e.g., about 17, 18, 19, 20, 21, 22, 23, 24, 25, 26, 27, or 28 nucleotides) downstream of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence and ends within about 20 to about 25 nucleotides (e.g., about 17, 18, 19, 20, 21, 22, 23, 24, 25, 26, 27, or 28 nucleotides) downstream of the 5′-CTTY-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) of the 5′-NTTN-3′ sequence within or adjacent to a gene, relative to a reference sequence and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of the 5′-NTTN-3′ sequence within or adjacent to a gene, relative to a reference sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ends within about 25 to about 30 nucleotides (e.g., about 22, 23, 24, 25, 26, 27, 28, 29, 30, 31, 32, or 33 nucleotides) downstream of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence and ends within about 25 to about 30 nucleotides (e.g., about 22, 23, 24, 25, 26, 27, 28, 29, 30, 31, 32, or 33 nucleotides) downstream of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 15 nucleotides (e.g., about 3, 4, 5, 6, 7, 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence within or adjacent to a gene, relative to a reference sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-CTTT-3′,5′-CTTC-3′,5′-GTTT-3′,5′-GTTC-3′,5′-TTTC-3′, 5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence within or adjacent to a gene, relative to a reference sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-CTTT-3′, 5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence within or adjacent to a gene, relative to a reference sequence. In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) and ends within about 20 to about 25 nucleotides (e.g., about 17, 18, 19, 20, 21, 22, 23, 24, 25, 26, 27, or 28 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 10 nucleotides and ends within about 20 to about 25 nucleotides (e.g., about 17, 18, 19, 20, 21, 22, 23, 24, 25, 26, 27, or 28 nucleotides) (e.g., about 3, 4, 5, 6, 7, 8, 9, 10, 11, or 12 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence within or adjacent to a gene, relative to a reference sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ends within about 20 to about 25 nucleotides (e.g., about 17, 18, 19, 20, 21, 22, 23, 24, 25, 26, 27, or 28 nucleotides) downstream of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′, 5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence and ends within about 20 to about 25 nucleotides (e.g., about 17, 18, 19, 20, 21, 22, 23, 24, 25, 26, 27, or 28 nucleotides) downstream of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence within or adjacent to a gene, relative to a reference sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of the 5′-NTTN-3′ sequence within or adjacent to a gene, relative to a reference sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ends within about 25 to about 30 nucleotides (e.g., about 22, 23, 24, 25, 26, 27, 28, 29, 30, 31, 32, or 33 nucleotides) downstream of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′, 5′-ATTN-3′, or 5′-GTTN-3′ sequence. In some embodiments, the deletion starts within about 5 to about 10 nucleotides (e.g., about 3, 4, 5, 6, 7, 8, 9, 10, 11, or 12 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence and ends within about 25 to about 30 nucleotides (e.g., about 22, 23, 24, 25, 26, 27, 28, 29, 30, 31, 32, or 33 nucleotides) downstream of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 5 to about 10 nucleotides (e.g., about 3, 4, 5, 6, 7, 8, 9, 10, 11, or 12 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence within or adjacent to a gene, relative to a reference sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence within or adjacent to a gene, relative to a reference sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-CTTT-3′,5′-CTTC-3′,5′-GTTT-3′,5′-GTTC-3′, 5′-TTTC-3′,5′-GTTA-3′, or 5′-GTTG-3′ sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of the 5′-NTTN-3′ sequence within or adjacent to a gene, relative to a reference sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-NTTY-3′, 5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) and ends within about 20 to about 25 nucleotides (e.g., about 17, 18, 19, 20, 21, 22, 23, 24, 25, 26, 27, or 28 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence within or adjacent to a gene, relative to a reference sequence and ends within about 20 to about 25 nucleotides (e.g., about 17, 18, 19, 20, 21, 22, 23, 24, 25, 26, 27, or 28 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-NTTY-3′, 5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ends within about 20 to about 25 nucleotides (e.g., about 17, 18, 19, 20, 21, 22, 23, 24, 25, 26, 27, or 28 nucleotides) downstream of the 5′-NTTY-3′, 5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence and ends within about 20 to about 25 nucleotides (e.g., about 17, 18, 19, 20, 21, 22, 23, 24, 25, 26, 27, or 28 nucleotides) downstream of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 20 to about 25 nucleotides (e.g., about 17, 18, 19, 20, 21, 22, 23, 24, 25, 26, 27, or 28 nucleotides) downstream of the T/C-rich sequence.

In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of the 5′-NTTN-3′ sequence within or adjacent to a gene, relative to a reference sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-NTTY-3′, 5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) and ends within about 25 to about 30 nucleotides (e.g., about 22, 23, 24, 25, 26, 27, 28, 29, 30, 31, 32, or 33 nucleotides) of a T/C-rich sequence.

In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of the 5′-NTTN-3′ sequence within or adjacent to a gene, relative to a reference sequence and ends within about 25 to about 30 nucleotides (e.g., about 22, 23, 24, 25, 26, 27, 28, 29, 30, 31, 32, or 33 nucleotides) downstream of the 5′-NTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-NTTY-3′, 5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ends within about 25 to about 30 nucleotides (e.g., about 22, 23, 24, 25, 26, 27, 28, 29, 30, 31, 32, or 33 nucleotides) downstream of the 5′-NTTY-3′, 5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the deletion starts within about 10 to about 15 nucleotides (e.g., about 8, 9, 10, 11, 12, 13, 14, 15, 16, or 17 nucleotides) downstream of a 5′-CTTT-3′,5′-CTTC-3′,5′-GTTT-3′,5′-GTTC-3′,5′-TTTC-3′,5′-GTTA-3′, or 5′-GTTG-3′ sequence and ends within about 25 to about 30 nucleotides (e.g., about 22, 23, 24, 25, 26, 27, 28, 29, 30, 31, 32, or 33 nucleotides) downstream of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion starts within about 10 to about 15 nucleotides (e.g., about 8, 9, 10, 11, 12, 13, 14, 15, 16, or 17 nucleotides) downstream of a T/C-rich sequence and ends within about 25 to about 30 nucleotides (e.g., about 22, 23, 24, 25, 26, 27, 28, 29, 30, 31, 32, or 33 nucleotides) downstream of the T/C-rich sequence.

In some embodiments, the deletion is up to about 40 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides).

In some embodiments, the deletion is between about 4 nucleotides and about 40 nucleotides in length (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides).

In some embodiments, the deletion is between about 4 nucleotides and about 25 nucleotides in length (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides).

In some embodiments, the deletion is between about 10 nucleotides and about 25 nucleotides in length (e.g., about 7, 8, 9, 10, 11, 12, 13, 14, 15, 16, 17, 18, 19, 20, 21, 22, 23, 24, 25, 26, 27, or 28 nucleotides).

In some embodiments, the deletion is between about 10 nucleotides and about 15 nucleotides in length (e.g., about 7, 8, 9, 10, 11, 12, 13, 14, 15, 16, or 17 nucleotides).

In some embodiments, a deletion starts within about 5 to about 15 nucleotides downstream of a first 5′-NTTN-3′ sequence and ends adjacent to a second 5′-NTTN-3′ sequence. In some embodiments, the deletion ends about 20 to about 30 nucleotides downstream of the second 5′-NTTN-3′ sequence. In some embodiments, the second 5′-NTTN-3′ sequence is on the same strand (sense or antisense strand) as the first 5′-NTTN-3′ sequence. In some embodiments, the second 5′-NTTN-3′ sequence is on a different strand than the first 5′-NTTN-3′ sequence. In some embodiments, a deletion starts within about 5 to about 15 nucleotides downstream of a first 5′-NTTN-3′ sequence on a sense strand and ends adjacent to a second 5′-NTTN-3′ sequence on the sense strand. In some embodiments, a deletion starts within about 5 to about 15 nucleotides downstream of a first 5′-NTTN-3′ sequence on a sense strand and ends about 20 to about 30 nucleotides downstream of a second 5′-NTTN-3′ sequence on the sense strand. In some embodiments, a deletion starts within about 5 to about 15 nucleotides downstream of a first 5′-NTTN-3′ sequence on an antisense strand and ends adjacent to a second 5′-NTTN-3′ sequence on the antisense strand. In some embodiments, the deletion may be greater than about 40 nucleotides in length (e.g., about 30, 35, 40, 45, 50, 55, 60, 65, 70, 75, 80, 85, 90, 95, 100 nucleotides or greater or any integer therebetween).

In some embodiments, a deletion starts within about 5 to about 15 nucleotides downstream of a first 5′-NTTN-3′ sequence on an antisense strand and ends about 20 to about 30 nucleotides downstream of a second 5′-NTTN-3′ sequence on the antisense strand. In some embodiments, a deletion starts within about 5 to about 15 nucleotides downstream of a 5′-NTTN-3′ sequence on a sense strand and ends adjacent to a 5′-NAAN-3′ sequence on the sense strand. In some embodiments, a deletion starts within about 5 to about 15 nucleotides downstream of a 5′-NTTN-3′ sequence on a sense strand and ends about 5 to about 15 nucleotides upstream of a 5′-NAAN-3′ sequence on the sense strand. In some embodiments, a deletion starts within about 5 to about 15 nucleotides downstream of a 5′-NTTN-3′ sequence on an antisense strand and ends adjacent to a 5′-NAAN-3′ sequence on the antisense strand. In some embodiments, a deletion starts within about 5 to about 15 nucleotides downstream of a 5′-NTTN-3′ sequence on an antisense strand and ends about 5 to about 15 nucleotides upstream of a 5′-NAAN-3′ sequence on the antisense strand. In some embodiments, the deletion may be greater than about 40 nucleotides in length (e.g., about 30, 35, 40, 45, 50, 55, 60, 65, 70, 75, 80, 85, 90, 95, 100 nucleotides or greater or any integer therebetween). In some embodiments, a deletion starts within about 5 to about 15 nucleotides downstream of a first 5′-NTTN-3′ sequence and ends about 5 to about 25 nucleotides upstream or 5′ of a complementary sequence of the second 5′-NTTN-3′ sequence (e.g., upstream of a 5′-NAAN-3′ sequence). In these embodiments, the deletion may be greater than about 40 nucleotides in length (e.g., about 30, 35, 40, 45, 50, 55, 60, 65, 70, 75, 80, 85, 90, 95, 100 nucleotides or greater or any integer therebetween).

Insertion

In some embodiments, a modified cell comprises a DNA insertion, wherein the DNA insertion is adjacent to a 5′-NTTN-3′ sequence, wherein N is any nucleotide. In some embodiments, the modified cell comprises the insertion as described herein compared to an unmodified cell that lacks the DNA insertion.

In some embodiments, the insertion is in a genome of the modified cell. In some embodiments, an unmodified cell is a wild-type cell.

In some embodiments, the modified cell comprises a DNA insertion adjacent to a 5′-NTTN-3′ sequence, wherein N is any nucleotide. In some embodiments, the modified cell comprises an insertion adjacent to a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence, wherein Y is C or T, B is any nucleotide except for A, D is any nucleotide except for C, and R is A or G. In some embodiments, the modified cell comprises an insertion adjacent to a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the modified cell comprises an insertion adjacent to a T/C-rich sequence.

In some embodiments, the modified cell comprises an insertion 3′ of a 5′-NTTN-3′ sequence on a sense strand within or adjacent to a gene, relative to a reference sequence. In some embodiments, the modified cell comprises an insertion 3′ of a 5′-NTTN-3′ sequence on an antisense strand within or adjacent to a gene, relative to a reference sequence. In some embodiments, the modified cell comprises an insertion that starts within about 5 to about 25 nucleotides of a 5′-NTTN-3′ sequence on a sense strand within or adjacent to a gene, relative to a reference sequence. In some embodiments, the modified cell comprises an insertion that starts within about 5 to about 25 nucleotides of a 5′-NTTN-3′ sequence on an antisense strand within or adjacent to a gene, relative to a reference sequence.

In some embodiments, the modified cell comprises an insertion downstream or 3′ of a 5′-NTTN-3′ sequence. In some embodiments, the modified cell comprises an insertion downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the modified cell comprises an insertion downstream of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the modified cell comprises an insertion downstream of a T/C-rich sequence.

In some embodiments, the insertion starts within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of the 5′-NTTN-3′ sequence. In some embodiments, the insertion starts within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of the 5′-NTTN-3′ sequence within or adjacent to a gene, relative to a reference sequence. In some embodiments, the insertion starts within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of a 5′-NTTY-3′,5′-NTTC-3′,5′-NTTT-3′, 5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the insertion starts within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′, 5′-CTTG-3′, or 5′-CTTC-3′ sequence. In some embodiments, the insertion starts within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of a T/C-rich sequence.

In some embodiments, the insertion starts within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence. In some embodiments, the insertion starts within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence within or adjacent to a gene, relative to a reference sequence. In some embodiments, the insertion starts within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the insertion starts within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′, 5′-GTTG-3′,5′-GTTC-3′,5′-CTTA-3′,5′-CTTT-3′,5′-CTTG-3′, or 5′-CTTC-3′ sequence. In some embodiments, the insertion starts within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of a T/C-rich sequence.

In some embodiments, the insertion starts within about 18 to about 30 nucleotides (e.g., about 18, 19, 20, 21, 22, 23, 24, 25, 26, 27, 28, 29, or 30 nucleotides) of the 5′-NTTN-3′ sequence. In some embodiments, the insertion starts within about 18 to about 30 nucleotides (e.g., about 18, 19, 20, 21, 22, 23, 24, 25, 26, 27, 28, 29, or 30 nucleotides) of the 5′-NTTN-3′ sequence within or adjacent to a gene, relative to a reference sequence. In some embodiments, the insertion starts within about 18 to about 30 nucleotides (e.g., about 18, 19, 20, 21, 22, 23, 24, 25, 26, 27, 28, 29, or 30 nucleotides) of a 5′-NTTY-3′,5′-NTTC-3′, 5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the insertion starts within about 18 to about 30 nucleotides (e.g., about 18, 19, 20, 21, 22, 23, 24, 25, 26, 27, 28, 29, or 30 nucleotides) of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′, 5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the insertion starts within about 18 to about 30 nucleotides (e.g., about 18, 19, 20, 21, 22, 23, 24, 25, 26, 27, 28, 29, or 30 nucleotides) of a T/C-rich sequence.

In some embodiments, the insertion starts within about 18 to about 30 nucleotides (e.g., about 18, 19, 20, 21, 22, 23, 24, 25, 26, 27, 28, 29, or 30 nucleotides) downstream of the 5′-NTTN-3′ sequence. In some embodiments, the insertion starts within about 18 to about 30 nucleotides (e.g., about 18, 19, 20, 21, 22, 23, 24, 25, 26, 27, 28, 29, or 30 nucleotides) downstream of the 5′-NTTN-3′ sequence within or adjacent to a gene, relative to a reference sequence. In some embodiments, the insertion starts within about 18 to about 30 nucleotides (e.g., about 18, 19, 20, 21, 22, 23, 24, 25, 26, 27, 28, 29, or 30 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the insertion starts within about 18 to about 30 nucleotides (e.g., about 18, 19, 20, 21, 22, 23, 24, 25, 26, 27, 28, 29, or 30 nucleotides) downstream of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the insertion starts within about 18 to about 30 nucleotides (e.g., about 18, 19, 20, 21, 22, 23, 24, 25, 26, 27, 28, 29, or 30 nucleotides) downstream of a T/C-rich sequence.

In some embodiments, the insertion starts within about 20 to about 28 nucleotides (e.g., about 20, 21, 22, 23, 24, 25, 26, 27, or 28 nucleotides) of the 5′-NTTN-3′ sequence. In some embodiments, the insertion starts within about 20 to about 28 nucleotides (e.g., about 20, 21, 22, 23, 24, 25, 26, 27, or 28 nucleotides) of the 5′-NTTN-3′ sequence within or adjacent to a gene, relative to a reference sequence. In some embodiments, the insertion starts within about 20 to about 28 nucleotides (e.g., about 20, 21, 22, 23, 24, 25, 26, 27, or 28 nucleotides) of a 5′-NTTY-3′,5′-NTTC-3′,5′-NTTT-3′,5′-NTTA-3′,5′-NTTB-3′, 5′-NTTG-3′,5′-CTTY-3′,5‘-DTTR’3′,5′-CTTR-3′,5′-DTTT-3′,5′-ATTN-3′, or 5′-GTTN-3′ sequence.

In some embodiments, the insertion starts within about 20 to about 28 nucleotides (e.g., about 20, 21, 22, 23, 24, 25, 26, 27, or 28 nucleotides) of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the insertion starts within about 20 to about 28 nucleotides (e.g., about 20, 21, 22, 23, 24, 25, 26, 27, or 28 nucleotides) of a T/C-rich sequence.

In some embodiments, the insertion starts within about 20 to about 28 nucleotides (e.g., about 20, 21, 22, 23, 24, 25, 26, 27, or 28 nucleotides) downstream of the 5′-NTTN-3′ sequence. In some embodiments, the insertion starts within about 20 to about 28 nucleotides (e.g., about 20, 21, 22, 23, 24, 25, 26, 27, or 28 nucleotides) downstream of the 5′-NTTN-3′ sequence within or adjacent to a gene, relative to a reference sequence. In some embodiments, the insertion starts within about 20 to about 28 nucleotides (e.g., about 20, 21, 22, 23, 24, 25, 26, 27, or 28 nucleotides) downstream of a 5′-NTTY-3′,5′-NTTC-3′, 5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the insertion starts within about 20 to about 28 nucleotides (e.g., about 20, 21, 22, 23, 24, 25, 26, 27, or 28 nucleotides) downstream of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′, 5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the insertion starts within about 20 to about 28 nucleotides (e.g., about 20, 21, 22, 23, 24, 25, 26, 27, or 28 nucleotides) downstream of a T/C-rich sequence.

In some embodiments, the insertion starts within about 20 to about 25 nucleotides (e.g., about 20, 21, 22, 23, 24, or 25 nucleotides) of the 5′-NTTN-3′ sequence. In some embodiments, the insertion starts within about 20 to about 25 nucleotides (e.g., about 20, 21, 22, 23, 24, or 25 nucleotides) of the 5′-NTTN-3′ sequence within or adjacent to a gene, relative to a reference sequence. In some embodiments, the insertion starts within about 20 to about 25 nucleotides (e.g., about 20, 21, 22, 23, 24, or 25 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the insertion starts within about 20 to about 25 nucleotides (e.g., about 20, 21, 22, 23, 24, or 25 nucleotides) of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the insertion starts within about 20 to about 25 nucleotides (e.g., about 20, 21, 22, 23, 24, or 25 nucleotides) of a T/C-rich sequence.

In some embodiments, the insertion starts within about 20 to about 25 nucleotides (e.g., about 20, 21, 22, 23, 24, or 25 nucleotides) downstream of the 5′-NTTN-3′ sequence. In some embodiments, the insertion starts within about 20 to about 25 nucleotides (e.g., about 20, 21, 22, 23, 24, or 25 nucleotides) downstream of the 5′-NTTN-3′ sequence within or adjacent to a gene, relative to a reference sequence. In some embodiments, the insertion starts within about 20 to about 25 nucleotides (e.g., about 20, 21, 22, 23, 24, or 25 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the insertion starts within about 20 to about 25 nucleotides (e.g., about 20, 21, 22, 23, 24, or 25 nucleotides) downstream of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the insertion starts within about 20 to about 25 nucleotides (e.g., about 20, 21, 22, 23, 24, or 25 nucleotides) downstream of a T/C-rich sequence.

In some embodiments, the insertion starts within about 5 to about 15 nucleotides (e.g., about 3, 4, 5, 6, 7, 8, 9, 10, 11, 12, 13, 14, 15, 16, or 17 nucleotides) downstream of a 5′-NTTN-3′ sequence on a sense strand within or adjacent to a gene, relative to a reference sequence and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the insertion starts within about 5 to about 15 nucleotides (e.g., about 3, 4, 5, 6, 7, 8, 9, 10, 11, 12, 13, 14, 15, 16, or 17 nucleotides) downstream of a 5′-NTTN-3′ sequence on an antisense strand in or adjacent to a gene and ends within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the insertion starts within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on a sense strand of a gene and ends within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on an antisense strand of the gene or upstream of a complementary sequence to a 5′-NTTN-3′ sequence on the sense strand of the gene (e.g., a 5′-NAAN-3′ sequence on the sense strand). In some embodiments, the insertion starts within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on an antisense strand of a gene and ends within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on a sense strand of the gene or upstream of a complementary sequence to a 5′-NTTN-3′ sequence on the antisense strand of the gene (e.g., a 5′-NAAN-3′ sequence on the antisense strand).

In some embodiments, the insertion is up to about 9 nucleotides (e.g., about 1, 2, 3, 4, 5, 6, 7, 8, or 9 nucleotides) in length. In some embodiments, the insertion is a 1-nucleotide insertion. In some embodiments, the insertion is a 2-nucleotide insertion. In some embodiments, the insertion is a 3-nucleotide insertion. In some embodiments, the insertion is a 4-nucleotide insertion. In some embodiments, the insertion is a 5-nucleotide insertion. In some embodiments, the insertion is a 6-nucleotide insertion. In some embodiments, the insertion is a 7-nucleotide insertion. In some embodiments, the insertion is an 8-nucleotide insertion. In some embodiments, the insertion is a 9-nucleotide insertion. In some embodiments, the insertion is longer than 9 nucleotides.

Biochemical Characteristics

In some embodiments, a modified cell described herein is further characterized by a biochemical change, as compared to an unmodified cell. In some embodiments, the biochemical change that occurs is transient. For example, in some embodiments, the biochemical change occurs while the cell is being modified or after the cell has been modified.

In some embodiments, the biochemical change occurs at the initiation of DNA repair, during DNA repair, or after DNA repair. In some embodiments, the modified cell of the disclosure is characterized by a stimulated cellular endogenous DNA repair pathway. In some embodiments, the stimulated DNA repair pathway is Non-Homologous End Joining (NHEJ), Alternative Non-Homologues End-Joining (A-NHEJ), or Homology Directed Recombination (HDR). NHEJ can repair cleaved target sequence without the need for a homologous template. NHEJ can result in the indel as described herein. In some embodiments, NHEJ results in insertion of one or more nucleotides at the target sequence. HDR can occur with a homologous template, such as the donor DNA. The homologous template can comprise sequences that are homologous to sequences flanking the target sequence cleavage site. In some cases, HDR can insert an exogenous polynucleotide sequence into the cleaved target sequence. The modifications of the target DNA due to NHEJ and/or HDR can further lead to, for example, mutations, deletions, alterations, integrations, gene correction, gene replacement, gene tagging, transgene knock-in, gene disruption, and/or gene knock-outs.

In some embodiments, the modified cell of the disclosure is characterized by recruitment of one or more endogenous cellular molecules. In some embodiments, the modified cell is characterized by recruitment of one or more molecules not involved in a DNA repair pathway. In some embodiments, the one or more recruited molecules associate with the genomic DNA of the cell. In some embodiments, one or more signal transduction pathways of a modified cell differ from those of an unmodified cell. For example, in some embodiments, the modified cell is characterized by a release of one or more secondary messengers.

In some embodiments, the gene expression profile of a modified cell described herein is altered, as compared to an unmodified cell. In some embodiments, expression of the modified gene is decreased by about 0.5%, 1%, 5%, 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or any integer therebetween) as compared to expression of a reference gene (e.g., an unmodified gene in an unmodified cell). In some embodiments, expression of the modified gene is 95%, 90%, 85%, 80%, 75%, 70%, 65%, 60%, 55%, 50%, 45%, 40%, 35%, 30%, 25%, 20%, 15%, 10%, or 5% the expression of a reference gene (e.g., an unmodified gene in an unmodified cell). In some embodiments, expression of the modified gene is increased by at least about 0.5%, 1%, 5%, 10%, 15%, 20%, 25%, 30%, 35%, 40%, 45%, 50%, 55%, 60%, 65%, 70%, 75%, 80%, 85%, 90%, 95%, 100%, 200%, or 300%, as compared to expression of a reference gene (e.g., an unmodified gene in an unmodified cell).

In some embodiments, the modified gene is an immune-related gene, for example, a gene that is involved in an immune response in a subject. In some embodiments, the modified gene is an immune checkpoint gene. In some embodiments, the modified gene is selected from the group consisting of: BCL11A intronic erythroid enhancer, CD3, Beta-2 microglobulin (B2M), T Cell Receptor Alpha Constant (TRAC), Programmed Cell Death 1 (PDCD1), T-cell receptor alpha, T-cell receptor beta, B-cell lymphoma/leukemia 11A (BCL11A), Cytotoxic T-Lymphocyte Antigen 4 (CTLA-4), chemokine (C—C motif) receptor 5 (gene/pseudogene) (CCRS), CXCR4 gene, CD160 molecule (CD160), adenosine A2a receptor (ADORA), CD276, B7-H3, B7-H4, BTLA, nicotinamide adenine dinucleotide phosphate NADPH oxidase isoform 2 (NOX2), V-domain Ig suppressor of T cell activation (VISTA), Sialic acid-binding immunoglobulin-type lectin 7 (SIGLEC7), Sialic acid-binding immunoglobulin-type lectin 9 (SIGLEC9), SIGLEC10, V-set domain containing T cell activation inhibitor 1 (VTCN1), B and T lymphocyte associated (BTLA), Indoleamine 2,3-dioxygenase (IDO), indoleamine 2,3-dioxygenase 1 (IDO1), Killer-cell Immunoglobulin-like Receptor (KIR), killer cell immunoglobulin-like receptor, three domains, long cytoplasmic tail, 1 (KIR3DL1), lymphocyte-activation gene 3 (LAG3), T-cell Immunoglobulin domain and Mucin domain 3 (TIM3), hepatitis A virus cellular receptor 2 (HAVCR2), natural killer cell receptor 2B4 (CD244), hypoxanthine phosphoribosyltransferase 1 (HPRT), T-cell immunoreceptor with Ig and ITIM domains (TIGIT), CD96 molecule (CD96), cytotoxic and regulatory T-cell molecule (CRTAM), leukocyte associated immunoglobulin like receptor 1 (LAIRI), adeno-associated virus integration site 1 (AAVS1), AAVS 2, AAVS3, AAVS4, AAVS5, AAVS6, AAVS7, AAVS8, transforming growth factor beta receptor II (TGFBRII), transforming growth factor beta receptor 1 (TGFBR1), SMAD family member 2 (SMAD2), SMAD family member 3 (SMAD3), SMAD family member 4 (SMAD4), SKI proto-oncogene (SKI), SKI-like proto-oncogene (SKIL), egl-9 family hypoxia-inducible factor 1 (EGLN1), egl-9 family hypoxia-inducible factor 2 (EGLN2), egl-9 family hypoxia-inducible factor 3 (EGLN3), protein phosphatase 1 regulatory subunit 12C (PPP1R12C), TGFB induced factor homeobox 1 (TGIF1), tumor necrosis factor receptor superfamily member, tumor necrosis factor receptor superfamily member 10b (TNFRSF10B), tumor necrosis factor receptor superfamily member 10a (TNFRSF10A), BY55, B7H5, caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1 (PAG1), guanylate cyclase 1, soluble, beta 3 (GUCY1B3), signaling threshold regulating transmembrane adaptor 1 (SIT1), forkhead box P3 (FOXP3), PR domain 1 (PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2 (GUCY1B2), prolyl hydroxylase domain (PHD1, PHD2, PHD3) family of proteins, CD27, CD28, CD40, CD122, CD137, OX40, GITR, and ICOS. In some embodiments, the modified gene is Programmed Death Ligand 1 (PD-LI), Class II Major Histocompatibility Complex Transactivator (CIITA), Adeno-Associated Virus Integration Site 1 (AAVS1), Citramalyl-CoA lyase (CLYBL), Transthyretin (TTR), Lactate Dehydrogenase-A (LDHA), Hydroxyacid Oxidase-1 (HAO1), Alanine-Glyoxylate and Serine-Pyruvate Aminotransferase (AGXT), Glyoxylate Reductase/Hydroxypyruvate Reductase (GRHPR), or 4-Hydroxy-2-Oxoglutarate Aldolase (HOGA).

In some embodiments, the modified cell is a T cell and comprises a modification in a gene selected from the group consisting of: BCL11A intronic erythroid enhancer, CD3, B2M, TRAC, or PDCD1. In some embodiments, the modified cell is a T cell and comprises a modification in a BCL11A intronic erythroid enhancer gene. In some embodiments, the modified cell is a T cell and comprises a modification in a CD3 gene. In some embodiments, the modified cell is a T cell and comprises a modification in a B2M gene. In some embodiments, the modified cell is a T cell and comprises a modification in a TRAC gene. In some embodiments, the modified cell is a T cell and comprises a modification in a PDCD1 gene.

Plurality of Cells

In some embodiments, a cell of a plurality of cells comprises at least one deletion and/or at least one insertion.

In some embodiments, a plurality of cells comprise a deletion described herein. In some embodiments, each cell of the plurality of cells comprises an identical deletion. In some embodiments, the plurality of cells comprises non-identical deletions (e.g., the deletions range in size and position relative to a 5′-NTTN-3′ sequence, as disclosed herein. For example, a first cell of a plurality can have a deletion from about 4 nucleotides to about 40 nucleotides in length, a second cell of a plurality can have a deletion from about 4 nucleotides to about 25 nucleotides in length, a third cell of a plurality can have a deletion from about 10 nucleotides to about 25 nucleotides in length, and/or a fourth cell of a plurality can have a deletion from about 10 nucleotides to about 15 nucleotides in length. In another example, a first cell of a plurality can have a deletion that starts within about 5 nucleotides to about 15 nucleotides of a 5′-NTTN-3′ sequence, a second cell of a plurality can have a deletion that starts within about 5 nucleotides to about 10 nucleotides of a 5′-NTTN-3′ sequence, a third cell of a plurality can have a deletion that starts within about 10 nucleotides to about 15 nucleotides of a 5′-NTTN-3′ sequence, a fourth cell of a plurality can have a deletion that starts within about 5 nucleotides to about 15 nucleotides downstream of a 5′-NTTN-3′ sequence, a fifth cell of a plurality can have a deletion that starts within about 5 nucleotides to about 10 nucleotides downstream of a 5′-NTTN-3′ sequence, and a sixth cell of a plurality can have a deletion that starts the deletion starts within about 10 nucleotides to about 15 nucleotides downstream of the 5′-NTTN-3′ sequence. In another example, a first cell of a plurality can have a deletion that ends within about 20 nucleotides to about 30 nucleotides of a 5′-NTTN-3′ sequence, a second cell of a plurality can have a deletion that ends within about 20 nucleotides to about 25 nucleotides of a 5′-NTTN-3′ sequence, a third cell of a plurality can have a deletion that ends within about 25 nucleotides to about 30 nucleotides of a 5′-NTTN-3′ sequence, a fourth cell of a plurality can have a deletion that ends within about 20 nucleotides to about 30 nucleotides downstream of a 5′-NTTN-3′ sequence, a fifth cell of a plurality can have a deletion that ends within about 20 nucleotides to about 25 nucleotides downstream of a 5′-NTTN-3′ sequence, and a sixth cell of a plurality can have a deletion that ends within about 25 nucleotides to about 30 nucleotides downstream of the 5′-NTTN-3′ sequence.

In some embodiments, at least 10%, 15%, 20%, 25%, 30%, 35%, 40%, 45%, 50%, 55%, 60%, 65%, 70% of the cells in the plurality (e.g., at least about 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the cells) comprise a deletion. In some embodiments the plurality of cells comprises a deletion in a gene, wherein the deletion is from about 4 to about 40 nucleotides in length.

In some embodiments, a plurality of cells comprises a deletion in a gene. In some embodiments, each of the cells of the plurality of cells comprising a deletion comprises a deletion in the same gene. In some embodiments, at least 70% of the cells (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the cells) comprise the deletion. In some embodiments, at least 80% of the cells (e.g., at least about 80%, 81%, 82%, 83%, 84%, 85%, 86%, 87%, 88%, 89%, 90%, 91%, 92%, 93%, 94%, 95%, 96%, 97%, 98%, 99%, or 100% of the cells) comprise the deletion. In some embodiments, at least 90% of the cells (e.g., at least about 90%, 91%, 92%, 93%, 94%, 95%, 96%, 97%, 98%, 99%, or 100% of the cells) comprise the deletion. In some embodiments, each of the cells comprises the deletion.

In some embodiments wherein a plurality of cells comprises a deletion in a gene, the deletion is at least about 5 nucleotides in length (e.g., at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides) in at least about 90% of the cells (e.g., at least about 90%, 91%, 92%, 93%, 94%, 95%, 96%, 97%, 98%, 99%, or 100% of the cells).

In some embodiments wherein a plurality of cells comprises a deletion in a gene, the deletion is at least about 10 nucleotides in length (e.g., at least about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides) in at least about 75% of the cells (e.g., at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the cells).

In some embodiments wherein a plurality of cells comprises a deletion in a gene, the deletion is at least about 15 nucleotides in length (e.g., at least about 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides) in at least about 50% of the cells (e.g., at least about 50%, 51%, 52%, 53%, 54%, 55%, 56%, 57%, 58%, 59%, 60%, 65%, 70%, 75%, 80%, 85%, 90%, 95%, or 100% of the cells).

In some embodiments wherein a plurality of cells comprises a deletion in a gene, the deletion is at least about 20 nucleotides in length (e.g., at least about 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides) in at least about 25% of the cells (e.g., at least about 25%, 26%, 27%, 28%, 29%, 30%, 35%, 40%, 45%, 50%, 55%, 60%, 65%, 70%, 75%, 80%, 85%, 90%, 95%, or 100% of the cells).

In some embodiments wherein a plurality of cells comprises a deletion in a gene, the deletion is at least about 25 nucleotides in length (e.g., at least about 22, 23, 24, 25, 26, 27, 28, 29, 30, 31, 32, 33, 34, 35, 36, 37, 38, 39, 40, 41, 42, 43, 44, or 45 nucleotides) in at least about 25% of the cells (e.g., at least about 25%, 26%, 27%, 28%, 29%, 30%, 35%, 40%, 45%, 50%, 55%, 60%, 65%, 70%, 75%, 80%, 85%, 90%, 95%, or 100% of the cells).

In some embodiments wherein a plurality of cells comprises a deletion in a gene, the deletion is from 4 to 40 nucleotides in length in at least about 25% of the cells (e.g., at least about 25%, 26%, 27%, 28%, 29%, 30%, 35%, 40%, 45%, 50%, 55%, 60%, 65%, 70%, 75%, 80%, 85%, 90%, 95%, or 100% of the cells).

In some embodiments, a cell of a plurality of cells comprises an insertion. In some embodiments, two or more cells of a plurality of cells comprise an insertion. In some embodiments, the insertion is at least one nucleotide (e.g., at least 1, 2, 3, 4, 5, 6, 7, 8, or 9 nucleotides) in length.

In some embodiments, in a plurality of cells comprising indels (e.g., a plurality of modified cells), at least about 3.0% of the indels are insertions. In some embodiments, in a plurality of modified cells, at least about 2.0% of the indels are insertions. In some embodiments, in a plurality of modified cells, at least about 1.0% of the indels are insertions. In some embodiments, in a plurality of modified cells, at least about 0.5% of the indels are insertions. In some embodiments, in a plurality of modified cells, at least about 0.4% of the indels are insertions. In some embodiments, in a plurality of modified cells, at least about 0.3% of the indels are insertions. In some embodiments, in a plurality of modified cells, at least about 0.2% of the indels are insertions. In some embodiments, in a plurality of modified cells, at least about 0.1% of the indels are insertions.

In some embodiments, in a plurality of modified cells, less than about 3.0% of the indels (e.g., less than about 3%, 2.9%, 2.8%, 2.7%, 2.6%, 2.5%, 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the indels) are insertions. In some embodiments, in a plurality of modified cells, less than about 2.0% of the indels (e.g., less than about 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the indels) are insertions. In some embodiments, in a plurality of modified cells, less than about 1.0% of the indels (e.g., less than about 1.0%, 0.9%, 0.8%, 0.7%, 0.6%, 0.5%, 0.4%, 0.3%, 0.2%, or 0.1% of the indels) are insertions. In some embodiments, in a plurality of modified cells, less than about 0.5% of the indels (e.g., less than about 0.5%, 0.4%, 0.3%, 0.2%, or 0.1% of the indels) are insertions. In some embodiments, in a plurality of modified cells, less than about 0.4% of the indels (e.g., less than about 0.4%, 0.3%, 0.2%, or 0.1% of the indels) are insertions. In some embodiments, in a plurality of modified cells, less than about 0.3% of the indels (e.g., less than about 0.3%, 0.2%, or 0.1% of the indels) are insertions. In some embodiments, in a plurality of modified cells, less than about 0.2% of the indels (e.g., less than about 0.2% or 0.1% of the indels) are insertions. In some embodiments, in a plurality of modified cells, less than about 0.1% of the indels (e.g., less than about 0.1%, 0.09%, 0.08%, 0.07%, 0.06%, 0.05%, 0.04%, 0.03%, 0.02%, or 0.01% of the indels) are insertions.

In some embodiments, in a plurality of modified cells, less than about 1.0% of the indels (e.g., less than about 1.0%, 0.9%, 0.8%, 0.7%, 0.6%, 0.5%, 0.4%, 0.3%, 0.2%, or 0.1% of the indels) are 1-nucleotide insertions. In some embodiments, in a plurality of modified cells, less than about 1.0% of the indels (e.g., less than about 1.0%, 0.9%, 0.8%, 0.7%, 0.6%, 0.5%, 0.4%, 0.3%, 0.2%, or 0.1% of the indels) are 2-nucleotide insertions. In some embodiments, in a plurality of modified cells, less than about 1.0% of the indels (e.g., less than about 1.0%, 0.9%, 0.8%, 0.7%, 0.6%, 0.5%, 0.4%, 0.3%, 0.2%, or 0.1% of the indels) are 3-nucleotide insertions. In some embodiments, in a plurality of modified cells, less than about 1.0% of the indels (e.g., less than about 1.0%, 0.9%, 0.8%, 0.7%, 0.6%, 0.5%, 0.4%, 0.3%, 0.2%, or 0.1% of the indels) are 4-nucleotide insertions. In some embodiments, in a plurality of modified cells, less than about 1.0% of the indels (e.g., less than about 1.0%, 0.9%, 0.8%, 0.7%, 0.6%, 0.5%, 0.4%, 0.3%, 0.2%, or 0.1% of the indels) are 5-nucleotide insertions. In some embodiments, in a plurality of modified cells, less than about 1.0% of the indels (e.g., less than about 1.0%, 0.9%, 0.8%, 0.7%, 0.6%, 0.5%, 0.4%, 0.3%, 0.2%, or 0.1% of the indels) are 6-nucleotide insertions. In some embodiments, in a plurality of modified cells, less than about 1.0% of the indels (e.g., less than about 1.0%, 0.9%, 0.8%, 0.7%, 0.6%, 0.5%, 0.4%, 0.3%, 0.2%, or 0.1% of the indels) are 7-nucleotide insertions. In some embodiments, in a plurality of modified cells, less than about 1.0% of the indels (e.g., less than about 1.0%, 0.9%, 0.8%, 0.7%, 0.6%, 0.5%, 0.4%, 0.3%, 0.2%, or 0.1% of the indels) are 8-nucleotide insertions. In some embodiments, in a plurality of modified cells, less than about 1.0% of the indels (e.g., less than about 1.0%, 0.9%, 0.8%, 0.7%, 0.6%, 0.5%, 0.4%, 0.3%, 0.2%, or 0.1% of the indels) are 9-nucleotide insertions.

In some embodiments, in a plurality of modified cells, less than about 0.5% of the indels (e.g., less than about 0.5%, 0.4%, 0.3%, 0.2%, or 0.1% of the indels) are 1-nucleotide insertions. In some embodiments, in a plurality of modified cells, less than about 0.5% of the indels (e.g., less than about 0.5%, 0.4%, 0.3%, 0.2%, or 0.1% of the indels) are 2-nucleotide insertions. In some embodiments, in a plurality of modified cells, less than about 0.5% of the indels (e.g., less than about 0.5%, 0.4%, 0.3%, 0.2%, or 0.1% of the indels) are 3-nucleotide insertions. In some embodiments, in a plurality of modified cells, less than about 0.5% of the indels (e.g., less than about 0.5%, 0.4%, 0.3%, 0.2%, or 0.1% of the indels) are 4-nucleotide insertions. In some embodiments, in a plurality of modified cells, less than about 0.5% of the indels (e.g., less than about 0.5%, 0.4%, 0.3%, 0.2%, or 0.1% of the indels) are 5-nucleotide insertions. In some embodiments, in a plurality of modified cells, less than about 0.5% of the indels (e.g., less than about 0.5%, 0.4%, 0.3%, 0.2%, or 0.1% of the indels) are 6-nucleotide insertions. In some embodiments, in a plurality of modified cells, less than about 0.5% of the indels (e.g., less than about 0.5%, 0.4%, 0.3%, 0.2%, or 0.1% of the indels) are 7-nucleotide insertions. In some embodiments, in a plurality of modified cells, less than about 0.5% of the indels (e.g., less than about 0.5%, 0.4%, 0.3%, 0.2%, or 0.1% of the indels) are 8-nucleotide insertions. In some embodiments, in a plurality of modified cells, less than about 0.5% of the indels (e.g., less than about 0.5%, 0.4%, 0.3%, 0.2%, or 0.1% of the indels) are 9-nucleotide insertions.

In some embodiments, in a plurality of modified cells, less than about 0.4% of the indels (e.g., less than about 0.4%, 0.3%, 0.2%, or 0.1% of the indels) are 1-nucleotide insertions. In some embodiments, in a plurality of modified cells, less than about 0.4% of the indels (e.g., less than about 0.4%, 0.3%, 0.2%, or 0.1% of the indels) are 2-nucleotide insertions. In some embodiments, in a plurality of modified cells, less than about 0.4% of the indels (e.g., less than about 0.4%, 0.3%, 0.2%, or 0.1% of the indels) are 3-nucleotide insertions. In some embodiments, in a plurality of modified cells, less than about 0.4% of the indels (e.g., less than about 0.4%, 0.3%, 0.2%, or 0.1% of the indels) are 4-nucleotide insertions. In some embodiments, in a plurality of modified cells, less than about 0.4% of the indels (e.g., less than about 0.4%, 0.3%, 0.2%, or 0.1% of the indels) are 5-nucleotide insertions. In some embodiments, in a plurality of modified cells, less than about 0.4% of the indels (e.g., less than about 0.4%, 0.3%, 0.2%, or 0.1% of the indels) are 6-nucleotide insertions. In some embodiments, in a plurality of modified cells, less than about 0.4% of the indels (e.g., less than about 0.4%, 0.3%, 0.2%, or 0.1% of the indels) are 7-nucleotide insertions. In some embodiments, in a plurality of modified cells, less than about 0.4% of the indels (e.g., less than about 0.4%, 0.3%, 0.2%, or 0.1% of the indels) are 8-nucleotide insertions. In some embodiments, in a plurality of modified cells, less than about 0.4% of the indels (e.g., less than about 0.4%, 0.3%, 0.2%, or 0.1% of the indels) are 9-nucleotide insertions.

In some embodiments, in a plurality of modified cells, less than about 0.3% of the indels (e.g., less than about 0.3%, 0.2%, or 0.1% of the indels) are 1-nucleotide insertions. In some embodiments, in a plurality of modified cells, less than about 0.3% of the indels (e.g., less than about 0.3%, 0.2%, or 0.1% of the indels) are 2-nucleotide insertions. In some embodiments, in a plurality of modified cells, less than about 0.3% of the indels (e.g., less than about 0.3%, 0.2%, or 0.1% of the indels) are 3-nucleotide insertions.

In some embodiments, in a plurality of modified cells, less than about 0.3% of the indels (e.g., less than about 0.3%, 0.2%, or 0.1% of the indels) are 4-nucleotide insertions. In some embodiments, in a plurality of modified cells, less than about 0.3% of the indels (e.g., less than about 0.3%, 0.2%, or 0.1% of the indels) are 5-nucleotide insertions. In some embodiments, in a plurality of modified cells, less than about 0.3% of the indels (e.g., less than about 0.3%, 0.2%, or 0.1% of the indels) are 6-nucleotide insertions. In some embodiments, in a plurality of modified cells, less than about 0.3% of the indels (e.g., less than about 0.3%, 0.2%, or 0.1% of the indels) are 7-nucleotide insertions. In some embodiments, in a plurality of modified cells, less than about 0.3% of the indels (e.g., less than about 0.3%, 0.2%, or 0.1% of the indels) are 8-nucleotide insertions. In some embodiments, in a plurality of modified cells, less than about 0.3% of the indels (e.g., less than about 0.3%, 0.2%, or 0.1% of the indels) are 9-nucleotide insertions.

In some embodiments, in a plurality of modified cells, less than about 0.2% of the indels (e.g., less than about 0.2% or 0.1% of the indels) are 1-nucleotide insertions. In some embodiments, in a plurality of modified cells, less than about 0.2% of the indels (e.g., less than about 0.2% or 0.1% of the indels) are 2-nucleotide insertions. In some embodiments, in a plurality of modified cells, less than about 0.2% of the indels (e.g., less than about 0.2% or 0.1% of the indels) are 3-nucleotide insertions. In some embodiments, in a plurality of modified cells, less than about 0.2% of the indels (e.g., less than about 0.2% or 0.1% of the indels) are 4-nucleotide insertions. In some embodiments, in a plurality of modified cells, less than about 0.2% of the indels (e.g., less than about 0.2% or 0.1% of the indels) are 5-nucleotide insertions. In some embodiments, in a plurality of modified cells, less than about 0.2% of the indels (e.g., less than about 0.2% or 0.1% of the indels) are 6-nucleotide insertions. In some embodiments, in a plurality of modified cells, less than about 0.2% of the indels (e.g., less than about 0.2% or 0.1% of the indels) are 7-nucleotide insertions. In some embodiments, in a plurality of modified cells, less than about 0.2% of the indels (e.g., less than about 0.2% or 0.1% of the indels) are 8-nucleotide insertions. In some embodiments, in a plurality of modified cells, less than about 0.2% of the indels (e.g., less than about 0.2% or 0.1% of the indels) are 9-nucleotide insertions.

In some embodiments, in a plurality of modified cells, less than about 0.1% of the indels (e.g., less than about 0.1%, 0.09%, 0.08%, 0.07%, 0.06%, 0.05%, 0.04%, 0.03%, 0.02%, or 0.01% of the indels) are 1-nucleotide insertions. In some embodiments, in a plurality of modified cells, less than about 0.1% of the indels (e.g., less than about 0.1%, 0.09%, 0.08%, 0.07%, 0.06%, 0.05%, 0.04%, 0.03%, 0.02%, or 0.01% of the indels) are 2-nucleotide insertions. In some embodiments, in a plurality of modified cells, less than about 0.1% of the indels (e.g., less than about 0.1%, 0.09%, 0.08%, 0.07%, 0.06%, 0.05%, 0.04%, 0.03%, 0.02%, or 0.01% of the indels) are 3-nucleotide insertions. In some embodiments, in a plurality of modified cells, less than about 0.1% of the indels (e.g., less than about 0.1%, 0.09%, 0.08%, 0.07%, 0.06%, 0.05%, 0.04%, 0.03%, 0.02%, or 0.01% of the indels) are 4-nucleotide insertions. In some embodiments, in a plurality of modified cells, less than about 0.1% of the indels (e.g., less than about 0.1%, 0.09%, 0.08%, 0.07%, 0.06%, 0.05%, 0.04%, 0.03%, 0.02%, or 0.01% of the indels) are 5-nucleotide insertions. In some embodiments, in a plurality of modified cells, less than about 0.1% of the indels (e.g., less than about 0.1%, 0.09%, 0.08%, 0.07%, 0.06%, 0.05%, 0.04%, 0.03%, 0.02%, or 0.01% of the indels) are 6-nucleotide insertions. In some embodiments, in a plurality of modified cells, less than about 0.1% of the indels (e.g., less than about 0.1%, 0.09%, 0.08%, 0.07%, 0.06%, 0.05%, 0.04%, 0.03%, 0.02%, or 0.01% of the indels) are 7-nucleotide insertions. In some embodiments, in a plurality of modified cells, less than about 0.1% of the indels (e.g., less than about 0.1%, 0.09%, 0.08%, 0.07%, 0.06%, 0.05%, 0.04%, 0.03%, 0.02%, or 0.01% of the indels) are 8-nucleotide insertions. In some embodiments, in a plurality of modified cells, less than about 0.1% of the indels (e.g., less than about 0.1%, 0.09%, 0.08%, 0.07%, 0.06%, 0.05%, 0.04%, 0.03%, 0.02%, or 0.01% of the indels) are 9-nucleotide insertions.

In some embodiments wherein a first plurality of modified cells comprises an insertion, the percentage of cells of the first plurality comprising an insertion is less than the percentage of cells of a second plurality comprising an insertion, wherein the second plurality is generated by treating an unmodified plurality of cells with a Cas9 polypeptide of SEQ ID NO: 5.

In some embodiments, a plurality of cells is obtained by culturing a modified cell comprising an insertion described herein. In some embodiments, a plurality of cells is obtained by isolating and culturing a modified cell comprising an insertion described herein. In some embodiments, a plurality of cells is obtained by culturing one or more cells comprising an indel. In some embodiments, a plurality of cells is obtained by culturing one or more modified cells.

In some embodiments, at least 10% of the cells of the plurality (e.g., at least about 10%, 15%, 20%, 25%, 30%, 35%, 40%, 45%, 50%, 55%, 60%, 65%, 70%, 75%, 80%, 85%, 90%, 95%, or 100% of the cells) comprise an insertion in a gene. In some embodiments, at least 20% of the cells of the plurality (e.g., at least about 20%, 25%, 30%, 35%, 40%, 45%, 50%, 55%, 60%, 65%, 70%, 75%, 80%, 85%, 90%, 95%, or 100% of the cells) comprise an insertion in a gene. In some embodiments, at least 30% of the cells of the plurality (e.g., at least about 30%, 35%, 40%, 45%, 50%, 55%, 60%, 65%, 70%, 75%, 80%, 85%, 90%, 95%, or 100% of the cells) comprise an insertion in a gene. In some embodiments, at least 40% of the cells of the plurality (e.g., at least about 40%, 45%, 50%, 55%, 60%, 65%, 70%, 75%, 80%, 85%, 90%, 95%, or 100% of the cells) comprise an insertion in a gene. In some embodiments, at least 50% of the cells of the plurality (e.g., at least about 50%, 55%, 60%, 65%, 70%, 75%, 80%, 85%, 90%, 95%, or 100% of the cells) comprise an insertion in a gene. In some embodiments, at least 60% of the cells of the plurality (e.g., at least about 60%, 65%, 70%, 75%, 80%, 85%, 90%, 95%, or 100% of the cells) comprise an insertion in a gene. In some embodiments, at least 70% of the cells of the plurality (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the cells) comprise an insertion in a gene. In some embodiments, at least 80% of the cells of the plurality (e.g., at least about 80%, 81%, 82%, 83%, 84%, 85%, 86%, 87%, 88%, 89%, 90%, 91%, 92%, 93%, 94%, 95%, 96%, 97%, 98%, 99%, or 100% of the cells) comprise an insertion in a gene. In some embodiments, at least 90% of the cells of the plurality (e.g., at least about 90%, 91%, 92%, 93%, 94%, 95%, 96%, 97%, 98%, 99%, or 100% of the cells) comprise an insertion in a gene. In some embodiments, each of the cells of the plurality (e.g., 100% of the cells) comprises an insertion in a gene.

In some embodiments, at least 10% of the cells of the plurality (e.g., at least about 10%, 15%, 20%, 25%, 30%, 35%, 40%, 45%, 50%, 55%, 60%, 65%, 70%, 75%, 80%, 85%, 90%, 95%, or 100% of the cells) comprise an insertion in the same gene. In some embodiments, at least 20% of the cells of the plurality (e.g., at least about 20%, 25%, 30%, 35%, 40%, 45%, 50%, 55%, 60%, 65%, 70%, 75%, 80%, 85%, 90%, 95%, or 100% of the cells) comprise an insertion in the same gene. In some embodiments, at least 30% of the cells of the plurality (e.g., at least about 30%, 35%, 40%, 45%, 50%, 55%, 60%, 65%, 70%, 75%, 80%, 85%, 90%, 95%, or 100% of the cells) comprise an insertion in the same gene. In some embodiments, at least 40% of the cells of the plurality (e.g., at least about 40%, 45%, 50%, 55%, 60%, 65%, 70%, 75%, 80%, 85%, 90%, 95%, or 100% of the cells) comprise an insertion in the same gene. In some embodiments, at least 50% of the cells of the plurality (e.g., at least about 50%, 55%, 60%, 65%, 70%, 75%, 80%, 85%, 90%, 95%, or 100% of the cells) comprise an insertion in the same gene. In some embodiments, at least 60% of the cells of the plurality (e.g., at least about 60%, 65%, 70%, 75%, 80%, 85%, 90%, 95%, or 100% of the cells) comprise an insertion in the same gene. In some embodiments, at least 70% of the cells of the plurality (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the cells) comprise an insertion in the same gene. In some embodiments, at least 80% of the cells of the plurality (e.g., at least about 80%, 81%, 82%, 83%, 84%, 85%, 86%, 87%, 88%, 89%, 90%, 91%, 92%, 93%, 94%, 95%, 96%, 97%, 98%, 99%, or 100% of the cells) comprise an insertion in the same gene. In some embodiments, at least 90% of the cells of the plurality (e.g., at least about 90%, 91%, 92%, 93%, 94%, 95%, 96%, 97%, 98%, 99%, or 100% of the cells) comprise an insertion in the same gene. In some embodiments, each of the cells of the plurality (e.g., 100% of the cells) comprises an insertion in the same gene.

In some embodiments, two or more cells of a plurality of cells comprise an identical insertion (e.g., the same insertion). In some embodiments, at least 10% of the cells of the plurality (e.g., at least about 10%, 15%, 20%, 25%, 30%, 35%, 40%, 45%, 50%, 55%, 60%, 65%, 70%, 75%, 80%, 85%, 90%, 95%, or 100% of the cells) comprise the same insertion. In some embodiments, at least 20% of the cells of the plurality (e.g., at least about 20%, 25%, 30%, 35%, 40%, 45%, 50%, 55%, 60%, 65%, 70%, 75%, 80%, 85%, 90%, 95%, or 100% of the cells) comprise the same insertion. In some embodiments, at least 30% of the cells of the plurality (e.g., at least about 30%, 35%, 40%, 45%, 50%, 55%, 60%, 65%, 70%, 75%, 80%, 85%, 90%, 95%, or 100% of the cells) comprise the same insertion. In some embodiments, at least 40% of the cells of the plurality (e.g., at least about 40%, 45%, 50%, 55%, 60%, 65%, 70%, 75%, 80%, 85%, 90%, 95%, or 100% of the cells) comprise the same insertion. In some embodiments, at least 50% of the cells of the plurality (e.g., at least about 50%, 55%, 60%, 65%, 70%, 75%, 80%, 85%, 90%, 95%, or 100% of the cells) comprise the same insertion. In some embodiments, at least 60% of the cells of the plurality (e.g., at least about 60%, 65%, 70%, 75%, 80%, 85%, 90%, 95%, or 100% of the cells) comprise the same insertion. In some embodiments, at least 70% of the cells of the plurality (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the cells) comprise the same insertion. In some embodiments, at least 80% of the cells of the plurality (e.g., at least about 80%, 81%, 82%, 83%, 84%, 85%, 86%, 87%, 88%, 89%, 90%, 91%, 92%, 93%, 94%, 95%, 96%, 97%, 98%, 99%, or 100% of the cells) comprise the same insertion. In some embodiments, at least 90% of the cells of the plurality (e.g., at least about 90%, 91%, 92%, 93%, 94%, 95%, 96%, 97%, 98%, 99%, or 100% of the cells) comprise the same insertion. In some embodiments, each of the cells of the plurality (e.g., 100% of the cells) comprises the same insertion.

### Preparation

The disclosure also provides methods of obtaining a modified cell of the disclosure. In some embodiments, the methods comprise introducing a Cas12i polypeptide and an RNA guide into a cell. For example, the Cas12i polypeptide can be introduced as a ribonucleoprotein complex (e.g., Cas12i ribonucleoprotein (RNP)) with an RNA guide into a cell. The Cas12i and/or RNA guide can be introduced on a nucleic acid vector. The Cas12i can be introduced as an mRNA. The RNA guide can be introduced directly into the cell.

In some embodiments, the RNA guide is designed as described in U.S. Pat. No. 10,808,245 and PCT/US2021/025257, which are incorporated by reference herein in their entirety. See, e.g., the “RNA Guides” and “RNA Guide Modifications” sections of U.S. Pat. No. 10,808,245 and the “Targeting Moiety” section of PCT/US2021/025257. The Cas12i polypeptide and RNA guide can further be delivered as described in PCT/US2021/025257.

In some embodiments wherein the Cas12i polypeptide has at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with SEQ ID NO: 3 of U.S. Pat. No. 10,808,245, the direct repeat is an RNA molecule having at least 90%, at least 95%, or 100% identity to SEQ ID NO: 7 or SEQ ID NO: 24 of U.S. Pat. No. 10,808,245 or a portion of SEQ ID NO: 7 or SEQ ID NO: 24 of U.S. Pat. No. 10,808,245. In some embodiments, the RNA guide comprises the sequence of SEQ ID NO: 156 or SEQ ID NO: 157 of U.S. Pat. No. 10,808,245 or a portion of the sequence of SEQ ID NO: 156 or SEQ ID NO: 157 of U.S. Pat. No. 10,808,245.

In some embodiments wherein the Cas12i polypeptide has at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with SEQ ID NO: 5 of U.S. Pat. No. 10,808,245 or any one of SEQ ID NOs: 2-4 or SEQ ID NOs: 46-48 of the present disclosure, the direct repeat is an RNA molecule having at least 90%, at least 95%, or 100% identity to SEQ ID NO: 9 or SEQ ID NO: 10 of U.S. Pat. No. 10,808,245 or a portion of SEQ ID NO: 9 or SEQ ID NO: 10 of U.S. Pat. No. 10,808,245. In some embodiments, the RNA guide comprises the sequence of SEQ ID NO: 162 or SEQ ID NO: 163 of U.S. Pat. No. 10,808,245 or a portion of the sequence of SEQ ID NO: 162 or SEQ ID NO: 163 of U.S. Pat. No. 10,808,245.

In some embodiments wherein the Cas12i polypeptide has at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with SEQ ID NO: 14 of U.S. Pat. No. 10,808,245, the direct repeat is an RNA molecule having at least 90%, at least 95%, or 100% identity to SEQ ID NO: 19 or SEQ ID NO: 21 of U.S. Pat. No. 10,808,245 or a portion of SEQ ID NO: 19 or SEQ ID NO: 21 of U.S. Pat. No. 10,808,245. In some embodiments, the RNA guide comprises the sequence of any one of SEQ ID NOs: 150, 151, or 153 of U.S. Pat. No. 10,808,245 or a portion of the sequence of any one of SEQ ID NOs: 150, 151, or 153 of U.S. Pat. No. 10,808,245.

In some embodiments wherein the Cas12i polypeptide has at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% sequence identity with SEQ ID NO: 16 of U.S. Pat. No. 10,808,245, the direct repeat of the is an RNA molecule having at least 90%, at least 95%, or 100% identity to SEQ ID NO: 7 or SEQ ID NO: 24 of U.S. Pat. No. 10,808,245 or a portion of SEQ ID NO: 6 or SEQ ID NO: 24 of U.S. Pat. No. 10,808,245. In some embodiments, the RNA guide comprises the sequence of SEQ ID NO: 152 or SEQ ID NO: 158 of U.S. Pat. No. 10,808,245 or a portion of the sequence of SEQ ID NO: 152 or SEQ ID NO: 158 of U.S. Pat. No. 10,808,245.

The RNA guide forms a complex with the Cas12i polypeptide and directs the Cas12i polypeptide to a target sequence adjacent to a 5′-NTTN-3′ sequence (e.g., PAM sequence). The RNA guide forms a complex with the Cas12i polypeptide and directs the Cas12i polypeptide to a target sequence adjacent to a 5′-NTTN-3′ sequence (e.g., PAM sequence) within or adjacent to a gene. In some embodiments, the 5′-NTTN-3′ sequence (e.g., PAM sequence) is a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the 5′-NTTN-3′ sequence is a 5′-CTTT-3′,5′-CTTC-3′,5′-GTTT-3′, 5′-GTTC-3′,5′-TTTC-3′,5′-GTTA-3′, or 5′-GTTG-3′ sequence.

In some embodiments, two or more (e.g., 2, 3, 4, 5, 6, 7, 8, 9, or more) RNA guides are used to introduce indels (e.g., deletions or insertions) into one or more (e.g., 1, 2, 3, 4, 5, 6, 7, 8, 9, or more) genes of a cell. For example, a first RNA guide can be designed to target a first gene, and a second RNA guide can be designed to target a second gene. In another example, a first RNA guide can be designed to target a first portion of a gene, and a second RNA guide can be designed to target a second portion of the gene.

In some embodiments, the Cas12i polypeptide has enzymatic activity (e.g., nuclease activity). In some embodiments, the Cas12i polypeptide induces one or more DNA double-stranded breaks in the cell.

In some embodiments, the Cas12i polypeptide induces one or more DNA single-stranded breaks in the cell.

In some embodiments, the Cas12i polypeptide induces one or more DNA nicks in the cell. In some embodiments, DNA breaks and/or nicks result in formation of one or more indels (e.g., one or more deletions or one or more insertions).

In some embodiments, the Cas12i polypeptide induces a deletion 3′ of a 5′-NTTN-3′ sequence on a target strand within or adjacent to a gene, relative to a reference sequence. In some embodiments, the Cas12i polypeptide induces a deletion that starts within about 5 to about 25 nucleotides of a 5′-NTTN-3′ sequence on a target strand within or adjacent to a gene, relative to a reference sequence. In some embodiments, the Cas12i polypeptide induces a deletion that starts within about 5 to about 25 nucleotides downstream or 3′ of a 5′-NTTN-3′ sequence on a target strand within or adjacent to a gene, relative to a reference sequence and ends about 15 to about 50 nucleotides downstream of the 5′-NTTN-3′ sequence. In some embodiments, the Cas12i polypeptide induces a deletion that starts within about 5 to about 25 nucleotides downstream of a 5′-NTTN-3′ sequence on a target strand within or adjacent to a gene, relative to a reference sequence and ends within about 5 to about 25 nucleotides of a 5′-NTTN-3′ sequence on the other strand, wherein the other strand 5′-NTTN-3′ sequence relative to the target strand 5′-NTTN-3′ sequence is downstream of the target strand 5′-NTTN-3′ sequence. In some embodiments, the Cas12i polypeptide induces a deletion that starts about 5 to about 25 nucleotides downstream of a 5′-NTTN-3′ sequence on a target strand within or adjacent to a gene, relative to a reference sequence and ends within about 5 to about 25 nucleotides upstream or 5′ to a complementary sequence of a 5′-NTTN-3′ sequence on the target strand, wherein the complementary 5′-NTTN-3′ sequence relative to the target strand 5′-NTTN-3′ sequence is downstream of the target strand 5′-NTTN-3′ sequence.

In some embodiments, the Cas12i polypeptide induces a deletion adjacent to (e.g., downstream of or 3′ of) a 5′-NTTN-3′ sequence, wherein N is any nucleotide. In some embodiments, the Cas12i polypeptide induces a deletion adjacent to (e.g., downstream of) a 5′-NTTN-3′ sequence within or adjacent to a gene, relative to a reference sequence, wherein N is any nucleotide. In some embodiments, the deletion is adjacent to (e.g., downstream of) a 5′-NTTY-3′,5′-NTTC-3′,5′-NTTT-3′,5′-NTTA-3′,5′-NTTB-3′, 5′-NTTG-3′,5′-CTTY-3′,5‘-DTTR’3′,5′-CTTR-3′,5′-DTTT-3′,5′-ATTN-3′, or 5′-GTTN-3′ sequence.

In some embodiments, the deletion is adjacent to (e.g., downstream of) a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the deletion is adjacent to (e.g., downstream of) a T/C-rich sequence.

In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence within or adjacent to a gene, relative to a reference sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptide induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a T/C-rich sequence.

In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence within or adjacent to a gene, relative to a reference sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptide induces a deletion starting within (e.g., downstream of) about 5 to about 10 nucleotides (e.g., about 3, 4, 5, 6, 7, 8, 9, 10, 11, or 12 nucleotides) of a T/C-rich sequence.

In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence within or adjacent to a gene, relative to a reference sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a 5′-NTTY-3′, 5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptide induces a deletion starting within (e.g., downstream of) about 10 to about 15 nucleotides (e.g., about 8, 9, 10, 11, 12, 13, 14, 15, 16, or 17 nucleotides) of a T/C-rich sequence.

In some embodiments, the Cas12i polypeptide induces a deletion starting within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptide induces a deletion starting within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence within or adjacent to a gene, relative to a reference sequence. In some embodiments, the Cas12i polypeptides induces a deletion starting within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion starting within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion starting within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of a T/C-rich sequence.

In some embodiments, the Cas12i polypeptide induces a deletion ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptide induces a deletion ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence within or adjacent to a gene, relative to a reference sequence. In some embodiments, the Cas12i polypeptides induces a deletion ending within (e.g., downstream of) about 20 to about 25 nucleotides (e.g., about 17, 18, 19, 20, 21, 22, 23, 24, 25, 26, 27, or 28 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion ending within (e.g., downstream of) about 20 to about 25 nucleotides (e.g., about 17, 18, 19, 20, 21, 22, 23, 24, 25, 26, 27, or 28 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion ending within (e.g., downstream of) about 20 to about 25 nucleotides (e.g., about 17, 18, 19, 20, 21, 22, 23, 24, 25, 26, 27, or 28 nucleotides) of a T/C-rich sequence.

In some embodiments, the Cas12i polypeptides induces a deletion ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptides induces a deletion ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence within or adjacent to a gene, relative to a reference sequence. In some embodiments, the Cas12i polypeptides induces a deletion ending within (e.g., downstream of) about 25 to about 30 nucleotides (e.g., about 22, 23, 24, 25, 26, 27, 28, 29, 30, 31, 32, or 33 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion ending within (e.g., downstream of) about 25 to about 30 nucleotides (e.g., about 22, 23, 24, 25, 26, 27, 28, 29, 30, 31, 32, or 33 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion ending within (e.g., downstream of) about 25 to about 30 nucleotides (e.g., about 22, 23, 24, 25, 26, 27, 28, 29, 30, 31, 32, or 33 nucleotides) of a T/C-rich sequence.

In some embodiments, the Cas12i polypeptides induces a deletion starting within about 5 to about 15 nucleotides (e.g., about 3, 4, 5, 6, 7, 8, 9, 10, 11, 12, 13, 14, 15, 16, or 17 nucleotides) downstream of a 5′-NTTN-3′ sequence on a target strand within or adjacent to a gene, relative to a reference sequence and ending within about 20 to about 30 nucleotides (e.g., about 17, 18, 19, 20, 21, 22, 23, 24, 25, 26, 27, 28, 29, 30, 31, 32, or 33 nucleotides) downstream of the 5′-NTTN-3′ sequence. In some embodiments, the Cas12i polypeptides induces a deletion starting within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on a target strand within or adjacent to a gene, relative to a reference sequence and ending within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on the other strand or upstream of a complementary sequence to a 5′-NTTN-3′ sequence on the target strand, wherein the other strand 5′-NTTN-3′ sequence or the complementary 5′-NTTN-3′ sequence relative to the target strand 5′-NTTN-3′ sequence is downstream of the target strand 5′-NTTN-3′ sequence.

In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′, 5′-CTTR-3′,5′-DTTT-3′,5′-ATTN-3′, or 5′-GTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-CTTY-3′,5′-CTTC-3′,5′-GTTT-3′,5′-GTTC-3′,5′-TTTC-3′,5′-GTTA-3′, or 5′-GTTG-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a T/C-rich sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the T/C-rich sequence.

In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a T/C-rich sequence and ending within (e.g., downstream of) about 20 to about 25 nucleotides (e.g., about 17, 18, 19, 20, 21, 22, 23, 24, 25, 26, 27, or 28 nucleotides) of the T/C-rich sequence.

In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion starting within (e.g., downstream of) about 5 to about 15 nucleotides (e.g., about 3, 4, 5, 6, 7, 8, 9, 10, 11, 12, 13, 14, 15, 16, or 17 nucleotides) of a T/C-rich sequence and ending within (e.g., downstream of) about 25 to about 30 nucleotides (e.g., about 22, 23, 24, 25, 26, 27, 28, 29, 30, 31, 32, or 33 nucleotides) of the T/C-rich sequence.

In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence within or adjacent to a gene, relative to a reference sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTY-3′,5′-NTTC-3′,5′-NTTT-3′, 5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of a T/C-rich sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the T/C-rich sequence.

In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence within or adjacent to a gene, relative to a reference sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion starting within (e.g., downstream of) about 5 to about 10 nucleotides of a T/C-rich sequence and ending within (e.g., downstream of) about 20 to about 25 nucleotides (e.g., about 17, 18, 19, 20, 21, 22, 23, 24, 25, 26, 27, or 28 nucleotides) (e.g., about 3, 4, 5, 6, 7, 8, 9, 10, 11, or 12 nucleotides) of the T/C-rich sequence.

In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of the 5′-NTTN-3′ sequence within or adjacent to a gene, relative to a reference sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of a 5′-NTTY-3′,5′-NTTC-3′,5′-NTTT-3′,5′-NTTA-3′,5′-NTTB-3′,5′-NTTG-3′,5′-CTTY-3′,5′-DTTR′3′,5′-CTTR-3′,5′-DTTT-3′,5′-ATTN-3′, or 5′-GTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion starting within (e.g., downstream of) about 5 to about 10 nucleotides (e.g., about 3, 4, 5, 6, 7, 8, 9, 10, 11, or 12 nucleotides) of a T/C-rich sequence and ending within (e.g., downstream of) about 25 to about 30 nucleotides (e.g., about 22, 23, 24, 25, 26, 27, 28, 29, 30, 31, 32, or 33 nucleotides) of the T/C-rich sequence.

In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-NTTY-3′, 5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of a 5′-CTTT-3′,5′-CTTC-3′,5′-GTTT-3′,5′-GTTC-3′,5′-TTTC-3′,5′-GTTA-3′, or 5′-GTTG-3′ sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of a T/C-rich sequence and ending within (e.g., downstream of) about 20 to about 30 nucleotides (e.g., about 17, 18, 19, 20, 21, 22, 23, 24, 25, 26, 27, 28, 29, 30, 31, 32, or 33 nucleotides) of the T/C-rich sequence.

In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 20 to about 25 nucleotides (e.g., about 17, 18, 19, 20, 21, 22, 23, 24, 25, 26, 27, or 28 nucleotides) of the 5′-NTTN-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of a 5′-NTTY-3′,5′-NTTC-3′,5′-NTTT-3′,5′-NTTA-3′,5′-NTTB-3′,5′-NTTG-3′, 5′-CTTY-3′,5‘-DTTR’3′,5′-CTTR-3′,5′-DTTT-3′,5′-ATTN-3′, or 5′-GTTN-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of a 5′-CTTT-3′, 5′-CTTC-3′,5′-GTTT-3′,5′-GTTC-3′,5′-TTTC-3′,5′-GTTA-3′, or 5′-GTTG-3′ sequence and ending within (e.g., downstream of) about 20 to about 25 nucleotides (e.g., about 17, 18, 19, 20, 21, 22, 23, 24, 25, 26, 27, or 28 nucleotides) of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of a T/C-rich sequence and ending within (e.g., downstream of) about 20 to about 25 nucleotides (e.g., about 17, 18, 19, 20, 21, 22, 23, 24, 25, 26, 27, or 28 nucleotides) of the T/C-rich sequence.

In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of the 5′-NTTN-3′ sequence within or adjacent to a gene, relative to a reference sequence and ending within (e.g., downstream of) about 25 to about 30 nucleotides (e.g., about 22, 23, 24, 25, 26, 27, 28, 29, 30, 31, 32, or 33 nucleotides) of the 5′-NTTN-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of a 5′-NTTY-3′,5′-NTTC-3′,5′-NTTT-3′,5′-NTTA-3′,5′-NTTB-3′,5′-NTTG-3′, 5′-CTTY-3′,5‘-DTTR’3′,5′-CTTR-3′,5′-DTTT-3′,5′-ATTN-3′, or 5′-GTTN-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of a 5′-CTTT-3′, 5′-CTTC-3′,5′-GTTT-3′,5′-GTTC-3′,5′-TTTC-3′,5′-GTTA-3′, or 5′-GTTG-3′ sequence and ending within (e.g., downstream of) about 25 to about 30 nucleotides (e.g., about 22, 23, 24, 25, 26, 27, 28, 29, 30, 31, 32, or 33 nucleotides) of the 5′-CTTT-3′,5′-CTTC-3′,5′-GTTT-3′,5′-GTTC-3′,5′-TTTC-3′,5′-GTTA-3′, or 5′-GTTG-3′ sequence. In some embodiments, the Cas12i polypeptides induces a deletion starting within (e.g., downstream of) about 10 to about 15 nucleotides (e.g., about 8, 9, 10, 11, 12, 13, 14, 15, 16, or 17 nucleotides) of a T/C-rich sequence and ending within (e.g., downstream of) about 25 to about 30 nucleotides (e.g., about 22, 23, 24, 25, 26, 27, 28, 29, 30, 31, 32, or 33 nucleotides) of the T/C-rich sequence.

In some embodiments, the Cas12i polypeptides induces a deletion up to about 40 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides).

In some embodiments, the Cas12i polypeptides induces a deletion of between about 4 nucleotides and about 40 nucleotides in length (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides).

In some embodiments, the Cas12i polypeptides induces a deletion of between about 4 nucleotides and about 25 nucleotides in length (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides).

In some embodiments, the Cas12i polypeptides induces a deletion of between about 10 nucleotides and about 25 nucleotides in length (e.g., about 7, 8, 9, 10, 11, 12, 13, 14, 15, 16, 17, 18, 19, 20, 21, 22, 23, 24, 25, 26, 27, or 28 nucleotides).

In some embodiments, the Cas12i polypeptides induces a deletion of between about 10 nucleotides and about 15 nucleotides in length (e.g., about 7, 8, 9, 10, 11, 12, 13, 14, 15, 16, or 17 nucleotides).

In some embodiments, the Cas12i polypeptide induces an insertion 3′ of a 5′-NTTN-3′ sequence on a target strand within or adjacent to a gene, relative to a reference sequence. In some embodiments, the Cas12i polypeptide induces an insertion that starts within about 5 to about 25 nucleotides of a 5′-NTTN-3′ sequence on a target strand within or adjacent to a gene, relative to a reference sequence.

In some embodiments, the Cas12i polypeptide induces an insertion adjacent to a 5′-NTTN-3′ sequence, wherein N is any nucleotide. In some embodiments, the Cas12i polypeptide induces an insertion adjacent to a 5′-NTTN-3′ sequence within or adjacent to a gene, relative to a reference sequence, wherein N is any nucleotide. In some embodiments, the Cas12i polypeptide-induced insertion is adjacent to a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′, 5′-CTTR-3′,5′-DTTT-3′,5′-ATTN-3′, or 5′-GTTN-3′ sequence. In some embodiments, the Cas12i polypeptide-induced insertion is adjacent to a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′, 5′-CTTA-3′,5′-CTTT-3′,5′-CTTG-3′, or 5′-CTTC-3′ sequence. In some embodiments, the Cas12i polypeptide-induced insertion is adjacent to a T/C-rich sequence.

In some embodiments, the Cas12i polypeptide induces an insertion downstream or 3′ of a 5′-NTTN-3′ sequence, wherein N is any nucleotide. In some embodiments, the Cas12i polypeptide induces an insertion downstream of a 5′-NTTN-3′ sequence within or adjacent to a gene, relative to a reference sequence, wherein N is any nucleotide. In some embodiments, the Cas12i polypeptide-induced insertion is downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide-induced insertion is downstream of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′, 5′-GTTG-3′,5′-GTTC-3′,5′-CTTA-3′,5′-CTTT-3′,5′-CTTG-3′, or 5′-CTTC-3′ sequence. In some embodiments, the Cas12i polypeptide-induced insertion is downstream of a T/C-rich sequence.

In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of the 5′-NTTN-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of the 5′-NTTN-3′ sequence within or adjacent to a gene, relative to a reference sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of a 5′-NTTY-3′,5′-NTTC-3′,5′-NTTT-3′,5′-NTTA-3′, 5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of a 5′-ATTA-3′,5′-ATTT-3′,5′-ATTG-3′,5′-ATTC-3′,5′-TTTA-3′, 5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) of a T/C-rich sequence.

In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of the 5′-NTTN-3′ sequence within or adjacent to a gene, relative to a reference sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′, 5′-GTTG-3′,5′-GTTC-3′,5′-CTTA-3′,5′-CTTT-3′,5′-CTTG-3′, or 5′-CTTC-3′ sequence. In some embodiments, the Cas12i polypeptide induces an insertion starting within about 15 to about 35 nucleotides (e.g., about 15, 16, 17, 18, 19, 20, 21, 22, 23, 24, 25, 26, 27, 28, 29, 30, 31, 32, 33, 34, or 35 nucleotides) downstream of a T/C-rich sequence.

In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) of the 5′-NTTN-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) of the 5′-NTTN-3′ sequence within or adjacent to a gene, relative to a reference sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) of a 5′-NTTY-3′,5′-NTTC-3′,5′-NTTT-3′, 5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′, 5′-CTTG-3′, or 5′-CTTC-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) of a T/C-rich sequence.

In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) downstream of the 5′-NTTN-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) downstream of the 5′-NTTN-3′ sequence within or adjacent to a gene, relative to a reference sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) downstream of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′, 5′-GTTG-3′,5′-GTTC-3′,5′-CTTA-3′,5′-CTTT-3′,5′-CTTG-3′, or 5′-CTTC-3′ sequence. In some embodiments, the Cas12i polypeptide induces an insertion starting within about 18 to about 30 nucleotides (e.g., about 18, 19, 20, 21, 22, 23, 24, 25, 26, 27, 28, 29, or 30 nucleotides) downstream of a T/C-rich sequence.

In some embodiments, the Cas12i polypeptide induces an insertion starting within about 20 to about 28 nucleotides (e.g., about 20, 21, 22, 23, 24, 25, 26, 27, or 28 nucleotides) of the 5′-NTTN-3′ sequence. In some embodiments, the Cas12i polypeptide induces an insertion starting within about 20 to about 28 nucleotides (e.g., about 20, 21, 22, 23, 24, 25, 26, 27, or 28 nucleotides) of the 5′-NTTN-3′ sequence within or adjacent to a gene, relative to a reference sequence. In some embodiments, the Cas12i polypeptide induces an insertion starting within about 20 to about 28 nucleotides (e.g., about 20, 21, 22, 23, 24, 25, 26, 27, or 28 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide induces an insertion starting within about 20 to about 28 nucleotides (e.g., about 20, 21, 22, 23, 24, 25, 26, 27, or 28 nucleotides) of a 5′-ATTA-3′,5′-ATTT-3′,5′-ATTG-3′, 5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the Cas12i polypeptide induces an insertion starting within about 20 to about 28 nucleotides (e.g., about 20, 21, 22, 23, 24, 25, 26, 27, or 28 nucleotides) of a T/C-rich sequence.

In some embodiments, the Cas12i polypeptide induces an insertion starting within about 20 to about 28 nucleotides (e.g., about 20, 21, 22, 23, 24, 25, 26, 27, or 28 nucleotides) downstream of the 5′-NTTN-3′ sequence. In some embodiments, the Cas12i polypeptide induces an insertion starting within about 20 to about 28 nucleotides (e.g., about 20, 21, 22, 23, 24, 25, 26, 27, or 28 nucleotides) downstream of the 5′-NTTN-3′ sequence within or adjacent to a gene, relative to a reference sequence. In some embodiments, the Cas12i polypeptide induces an insertion starting within about 20 to about 28 nucleotides (e.g., about 20, 21, 22, 23, 24, 25, 26, 27, or 28 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide induces an insertion starting within about 20 to about 28 nucleotides (e.g., about 20, 21, 22, 23, 24, 25, 26, 27, or 28 nucleotides) downstream of a 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′, 5′-CTTG-3′, or 5′-CTTC-3′ sequence. In some embodiments, the Cas12i polypeptide induces an insertion starting within about 20 to about 28 nucleotides (e.g., about 20, 21, 22, 23, 24, 25, 26, 27, or 28 nucleotides) downstream of a T/C-rich sequence.

In some embodiments, the Cas12i polypeptide induces an insertion starting within about 20 to about 25 nucleotides (e.g., about 20, 21, 22, 23, 24, or 25 nucleotides) of the 5′-NTTN-3′ sequence. In some embodiments, the Cas12i polypeptide induces an insertion starting within about 20 to about 25 nucleotides (e.g., about 20, 21, 22, 23, 24, or 25 nucleotides) of the 5′-NTTN-3′ sequence within or adjacent to a gene, relative to a reference sequence. In some embodiments, the Cas12i polypeptide induces an insertion starting within about 20 to about 25 nucleotides (e.g., about 20, 21, 22, 23, 24, or 25 nucleotides) of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide induces an insertion starting within about 20 to about 25 nucleotides (e.g., about 20, 21, 22, 23, 24, or 25 nucleotides) of a 5′-ATTA-3′,5′-ATTT-3′,5′-ATTG-3′,5′-ATTC-3′,5′-TTTA-3′, 5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the Cas12i polypeptide induces an insertion starting within about 20 to about 25 nucleotides (e.g., about 20, 21, 22, 23, 24, or 25 nucleotides) of a T/C-rich sequence.

In some embodiments, the Cas12i polypeptide induces an insertion starting within about 20 to about 25 nucleotides (e.g., about 20, 21, 22, 23, 24, or 25 nucleotides) downstream of the 5′-NTTN-3′ sequence. In some embodiments, the Cas12i polypeptide induces an insertion starting within about 20 to about 25 nucleotides (e.g., about 20, 21, 22, 23, 24, or 25 nucleotides) downstream of the 5′-NTTN-3′ sequence within or adjacent to a gene, relative to a reference sequence. In some embodiments, the Cas12i polypeptide induces an insertion starting within about 20 to about 25 nucleotides (e.g., about 20, 21, 22, 23, 24, or 25 nucleotides) downstream of a 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′ sequence. In some embodiments, the Cas12i polypeptide induces an insertion starting within about 20 to about 25 nucleotides (e.g., about 20, 21, 22, 23, 24, or 25 nucleotides) downstream of a 5′-ATTA-3′,5′-ATTT-3′, 5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′ sequence. In some embodiments, the Cas12i polypeptide induces an insertion starting within about 20 to about 25 nucleotides (e.g., about 20, 21, 22, 23, 24, or 25 nucleotides) downstream of a T/C-rich sequence.

In some embodiments, the Cas12i polypeptides induces an insertion starting within about 5 to about 15 nucleotides (e.g., about 3, 4, 5, 6, 7, 8, 9, 10, 11, 12, 13, 14, 15, 16, or 17 nucleotides) downstream of a 5′-NTTN-3′ sequence on a target strand within or adjacent to a gene, relative to a reference sequence. In some embodiments, the Cas12i polypeptides induces an insertion starting within about 5 to about 25 nucleotides (e.g., about 3, 4, 5, 6, 7, 8, 9, 10, 11, 12, 13, 14, 15, 16, 17, 18, 19, 20, 21, 22, 23, 24, 25, 26, 27, or 28 nucleotides) downstream of a 5′-NTTN-3′ sequence on target strand within or adjacent to a gene, relative to a reference sequence. In some embodiments, the Cas12i polypeptides induces an insertion starting within about 5 to about 30 nucleotides (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 nucleotides) downstream of a 5′-NTTN-3′ sequence on target strand within or adjacent to a gene, relative to a reference sequence.

In some embodiments, the Cas12i polypeptide induces an insertion of up to about 9 nucleotides (e.g., about 1, 2, 3, 4, 5, 6, 7, 8, or 9 nucleotides) in length. In some embodiments, the Cas12i polypeptide induces a 1-nucleotide insertion. In some embodiments, the Cas12i polypeptide induces a 2-nucleotide insertion. In some embodiments, the Cas12i polypeptide induces a 3-nucleotide insertion. In some embodiments, the Cas12i polypeptide induces a 4-nucleotide insertion. In some embodiments, the Cas12i polypeptide induces a 5-nucleotide insertion. In some embodiments, the Cas12i polypeptide induces a 6-nucleotide insertion. In some embodiments, the Cas12i polypeptide induces a 7-nucleotide insertion. In some embodiments, the Cas12i polypeptide induces an 8-nucleotide insertion. In some embodiments, the Cas12i polypeptide induces a 9-nucleotide insertion. In some embodiments, the Cas12i polypeptide induces an insertion having a length greater than 9 nucleotides.

The disclosure also provides methods of obtaining a plurality of modified cells of the disclosure. In some embodiments, the modified cell described above is identified, isolated and cultured to produce a plurality of identical modified cells. The modified cell can be isolated using methods known in the art, e.g., by immunomagnetic cell separation, fluorescence-activated cell sorting, density gradient centrifugation, immunodensity cell separation, sedimentation, adhesion, or microfluidic cell separation. In some embodiments, a plurality of modified cells comprising the deletion and/or insertion described above is produced via introduction of the Cas12i polypeptide and RNA guide at high frequency, such that the modified cells represent at least 10%, 15%, 20%, 25%, 30%, 35%, 40%, 45%, 50%, 55%, 60%, 65%, 70%, or more of the cells present.

### Compositions and Formulations

The disclosure also provides a composition or formulation comprising the modified cell or plurality of modified cells described herein. In some embodiments, the composition or formulation includes a cell or plurality of cells modified by Cas12i. In some embodiments, the composition or formulation includes a cell or plurality of cells comprising a deletion described herein. In some embodiments, the composition or formulation includes a cell line modified by Cas12i. In some embodiments, the composition or formulation includes a cell line comprising a deletion described herein. The composition or formulation can additionally include, optionally, media and/or instructions for use of the modified cell or cell line.

In some embodiments, the composition or formulation comprises a plurality of cells that include at least 10% modified cells described herein, e.g., at least 10%, 15%, 20%, 25%, 30%, 35%, 40%, 45%, 50%, 55%, 60%, 65%, 70%, or more of the plurality are the modified cells. In some embodiments, the composition or formulation comprises a plurality of cells that include at least 70% modified cells described herein, e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more of the plurality are the modified cells. In some embodiments, the composition or formulation comprises a plurality of cells that include at least 80% modified cells described herein, e.g., at least about 80%, 81%, 82%, 83%, 84%, 85%, 86%, 87%, 88%, 89%, 90%, 91%, 92%, 93%, 94%, 95%, 96%, 97%, 98%, 99%, or more of the plurality are the modified cells. In some embodiments, the composition or formulation comprises a plurality of cells that include at least 90% modified cells described herein, e.g., at least about 90%, 91%, 92%, 93%, 94%, 95%, 96%, 97%, 98%, 99%, or more of the plurality are the modified cells.

In some embodiments, the composition is a pharmaceutical composition. A pharmaceutical composition that is useful may be prepared, packaged, or sold in a formulation suitable for oral, rectal, vaginal, parenteral, topical, pulmonary, intranasal, intra-lesional, buccal, ophthalmic, intravenous, intra-organ or another route of administration. A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined number of cells. The number of cells is generally equal to the dosage of the cells which would be administered to a subject or a convenient fraction of such a dosage such as, for example, one-half or one-third of such a dosage.

A formulation of a pharmaceutical composition suitable for parenteral administration may comprise the cells combined with a pharmaceutically acceptable carrier, such as sterile water or sterile isotonic saline. Such a formulation may be prepared, packaged, or sold in a form suitable for bolus administration or for continuous administration. Some injectable formulations may be prepared, packaged, or sold in unit dosage form, such as in ampules or in multi-dose containers containing a preservative. Some formulations for parenteral administration include, but are not limited to, suspensions, solutions, emulsions in oily or aqueous vehicles, pastes, and implantable sustained-release or biodegradable formulations. Some formulations may further comprise one or more additional ingredients including, but not limited to, suspending, stabilizing, or dispersing agents.

The pharmaceutical composition may be prepared, packaged, or sold in the form of a sterile injectable aqueous or oily suspension or solution. This suspension or solution may be formulated according to the known art, and may comprise, in addition to the cells, additional ingredients such as the dispersing agents, wetting agents, or suspending agents described herein. Such sterile injectable formulation may be prepared using a non-toxic parenterally-acceptable diluent or solvent, such as water or saline. Other acceptable diluents and solvents include, but are not limited to, Ringer's solution, isotonic sodium chloride solution, and fixed oils such as synthetic mono- or di-glycerides. Other parentally-administrable formulations which that are useful include those which may comprise the cells in a packaged form, in a liposomal preparation, or as a component of a biodegradable polymer system. Some compositions for sustained release or implantation may comprise pharmaceutically acceptable polymeric or hydrophobic materials such as an emulsion, an ion exchange resin, a sparingly soluble polymer, or a sparingly soluble salt.

### Uses

In some embodiments, the composition or formulation comprising the modified cell or a plurality of the modified cells as described herein may be useful for research purposes. In some embodiments, the composition or formulation comprising the modified cell or a plurality of the modified cells as described herein may be useful to study gene function. In some embodiments, the composition or formulation comprising the modified cell or a plurality of the modified cells as described herein may be useful as an expression system to manufacture biomolecules. For example, in some embodiments, the composition or formulation comprising the modified cell or a plurality of the modified cells as described herein may be useful to produce biomolecules such as proteins (e.g., cytokines, antibodies, antibody-based molecules), peptides, lipids, carbohydrates, nucleic acids, amino acids, and vitamins. In other embodiments, the composition or formulation comprising the modified cell or a plurality of the modified cells as described herein may be useful in the production of a viral vector such as a lentivirus, adenovirus, adeno-associated virus, and oncolytic virus vector. In some embodiments, the composition or formulation comprising the modified cell or a plurality of the modified cells as described herein may be useful in cytotoxicity studies. In some embodiments, the composition or formulation comprising the modified cell or a plurality of the modified cells as described herein may be useful as a disease model. In some embodiments, the composition or formulation comprising the modified cell or a plurality of the modified cells as described herein may be useful in vaccine production. In some embodiments, the composition or formulation comprising the modified cell or a plurality of the modified cells as described herein may be useful in therapeutics. For example, in some embodiments, the composition or formulation comprising the modified cell or a plurality of the modified cells as described herein may be useful in cellular therapies such as transfusions and transplantations.

In some embodiments, the composition or formulation comprising the modified cell or a plurality of the modified cells as described herein may be useful to establish a new cell line comprising a modified genomic sequence. In some embodiments, a modified cell of the disclosure is a modified stem cell (e.g., a modified totipotent/omnipotent stem cell, a modified pluripotent stem cell, a modified multipotent stem cell, a modified oligopotent stem cell, or a modified unipotent stem cell) that differentiates into one or more cell lineages comprising the deletion of the modified stem cell. The disclosure further provides organisms (such as animals, plants, or fungi) comprising or produced from a modified cell of the disclosure.

All references and publications cited herein are hereby incorporated by reference.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Characterization of Deletions Induced by Cas12i2

This Example describes a method for characterizing Cas12i2-induced deletions in mammalian cells.

Wild-type Cas12i2 of SEQ ID NO: 2, variant Cas12i2 of SEQ ID NO: 3, and variant Cas72i2 of SEQ ID NO: 4 were cloned into a modified pET28 backbone (EMD Millipore). The plasmids were then transformed into E. coli BL21 (DE3) (Thermo Fisher). Cas12i2 proteins were expressed by culturing BL21 (DE3) cells in bacterial growth media (Teknova) to OD₆₀₀ of about 0.7, and protein expression was induced with the addition of 0.5 mM IPTG (Teknova) for 12-14 hours at 18° C. with 250 rpm shaking. Cells were harvested by centrifugation, resuspended in Extraction Buffer (20 mM Bis-Tris, pH 6.5, 500 mM NaCl, 0.5 mM TCEP, and 5% glycerol), and lysed by passing the cell slurry through a pressurized cell (Cell Systems) at 20 kPa. Insoluble material was removed by centrifugation, and nucleic acids were depleted with the addition of polyethyleneimine (Sigma) to 0.2%, followed by centrifugation. The clarified lysates were then purified by ion exchange chromatography, and the flow through was collected. Peak fractions were pooled, and proteins were concentrated using centrifugal filter units (30 kDa NMWL, Sigma). Cas12i2 protein concentrations were determined by A₂₈₀ (Nanodrop, Thermo Fisher), adjusted to 50% glycerol, and stored at −20° C. SpCas9 protein (SEQ ID NO: 5) was purchased from Aldevron.

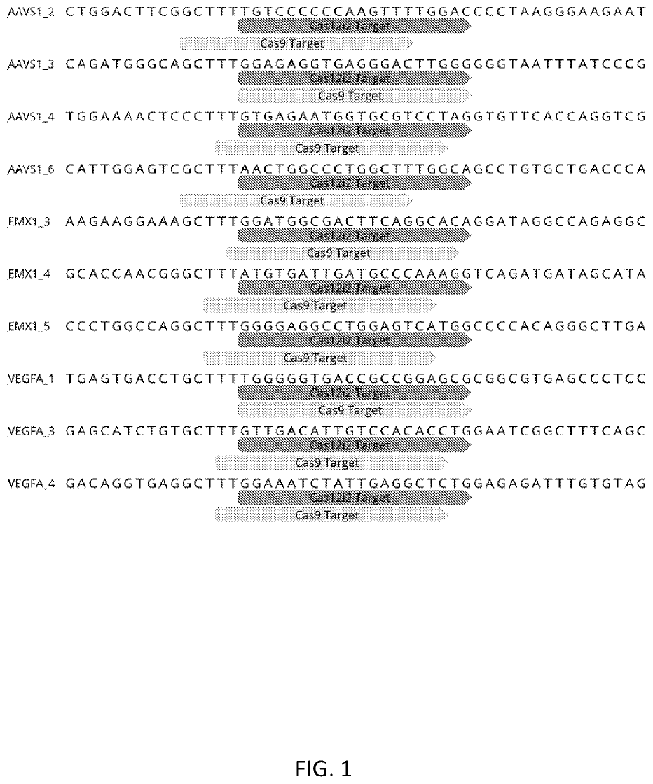

Cas12i2 and SpCas9 RNA guides were ordered as synthetic RNA oligos (IDT), and RNA guide sequences and their corresponding target DNA sequences are shown in Table 7. The target DNA sequences are also shown in FIG. 1.

TABLE 1
Target and RNA Guide Sequences.

| Locus | Nuclease | Target Sequence | RNA Guide Sequence |
|---|---|---|---|
| AAVS1_2 | Cas12i2 | TGTCCCCCCAAGTTTTGGAC | AGAAAUCCGUCUUUCAUUGACGGUGUCCC |
| | | (SEQ ID NO: 6) | CCCAAGUUUUGGAC (SEQ ID NO: 7) |
| AAVS1_2 | SpCas9 | GCTTTTGTCCCCCCAAGTTT | GCUUUUGUCCCCCCAAGUUUGTTTTAGAG |
| | | (SEQ ID NO: 8) | CTAGAAATAGCAAGTTAAAATAAGGCTAG |
| | | | TCCGTTATCAACTTGAAAAAGTGGCACCG |
| | | | AGTCGGTGCUUUUUU (SEQ ID NO: |
| | | | 9) |
| AAVS1_3 | Cas12i2 | GGAGAGGTGAGGGACTTGGG | AGAAAUCCGUCUUUCAUUGACGGGGAGAG |
| | | (SEQ ID NO: 10) | GUGAGGGACUUGGG (SEQ ID NO: |
| | | | 11) |
| AAVS1_3 | SpCas9 | GGAGAGGTGAGGGACTTGGG | GGAGAGGUGAGGGACUUGGGGTTTTAGAG |
| | | (SEQ ID NO: 12) | CTAGAAATAGCAAGTTAAAATAAGGCTAG |
| | | | TCCGTTATCAACTTGAAAAAGTGGCACCG |
| | | | AGTCGGTGCUUUUUU (SEQ ID NO: |
| | | | 13) |
| AAVS1_4 | Cas12i2 | GTGAGAATGGTGCGTCCTAG | AGAAAUCCGUCUUUCAUUGACGGGUGAGA |
| | | (SEQ ID NO: 14) | AUGGUGCGUCCUAG (SEQ ID NO: |
| | | | 15) |
| AAVS1_4 | SpCas9 | TTGTGAGAATGGTGCGTCCT | UUGUGAGAAUGGUGCGUCCUGTTTTAGAG |
| | | (SEQ ID NO: 16) | CTAGAAATAGCAAGTTAAAATAAGGCTAG |
| | | | TCCGTTATCAACTTGAAAAAGTGGCACCG |
| | | | AGTCGGTGCUUUUUU (SEQ ID NO: |
| | | | 17) |
| AAVS1_6 | Cas12i2 | AACTGGCCCTGGCTTTGGCA | AGAAAUCCGUCUUUCAUUGACGGAACUGG |
| | | (SEQ ID NO: 18) | CCCUGGCUUUGGCA (SEQ ID NO: |
| | | | 19) |
| AAVS1_6 | SpCas9 | GCTTTAACTGGCCCTGGCTT | GCUUUAACUGGCCCUGGCUUGTTTTAGAG |
| | | (SEQ ID NO: 20) | CTAGAAATAGCAAGTTAAAATAAGGCTAG |
| | | | TCCGTTATCAACTTGAAAAAGTGGCACCG |
| | | | AGTCGGTGCUUUUUU (SEQ ID NO: |
| | | | 21) |
| EMX1_3 | Cas12i2 | GGATGGCGACTTCAGGCACA | AGAAAUCCGUCUUUCAUUGACGGGGAUGG |
| | | (SEQ ID NO: 22) | CGACUUCAGGCACA (SEQ ID NO: |
| | | | 23) |
| EMX1_3 | SpCas9 | TGGATGGCGACTTCAGGCAC | UGGAUGGCGACUUCAGGCACGTTTTAGAG |
| | | (SEQ ID NO: 24) | CTAGAAATAGCAAGTTAAAATAAGGCTAG |
| | | | TCCGTTATCAACTTGAAAAAGTGGCACCG |
| | | | AGTCGGTGCUUUUUU (SEQ ID NO: |
| | | | 25) |
| EMX1_4 | Cas12i2 | ATGTGATTGATGCCCAAAGG | AGAAAUCCGUCUUUCAUUGACGGAUGUGA |
| | | (SEQ ID NO: 26) | UUGAUGCCCAAAGG (SEQ ID NO: |
| | | | 27) |
| EMX1_4 | SpCas9 | TTTATGTGATTGATGCCCAA | UUUAUGUGAUUGAUGCCCAAGTTTTAGAG |
| | | (SEQ ID NO: 28) | CTAGAAATAGCAAGTTAAAATAAGGCTAG |
| | | | TCCGTTATCAACTTGAAAAAGTGGCACCG |
| | | | AGTCGGTGCUUUUUU (SEQ ID NO: |
| | | | 29) |
| EMX1_5 | Cas12i2 | GGGGAGGCCTGGAGTCATGG | AGAAAUCCGUCUUUCAUUGACGGGGGGAG |
| | | (SEQ ID NO: 30) | GCCUGGAGUCAUGG (SEQ ID NO: |
| | | | 31) |
| EMX1_5 | SpCas9 | TTTGGGGAGGCCTGGAGTCA | UUUGGGGAGGCCUGGAGUCAGTTTTAGAG |
| | | (SEQ ID NO: 32) | CTAGAAATAGCAAGTTAAAATAAGGCTAG |
| | | | TCCGTTATCAACTTGAAAAAGTGGCACCG |
| | | | AGTCGGTGCUUUUUU (SEQ ID NO: |
| | | | 33) |
| VEGFA_1 | Cas12i2 | TGGGGGTGACCGCCGGAGCG | AGAAAUCCGUCUUUCAUUGACGGUGGGGG |
| | | (SEQ ID NO: 34) | UGACCGCCGGAGCG (SEQ ID NO: |
| | | | 35) |
| VEGFA_1 | SpCas9 | TGGGGGTGACCGCCGGAGCG | UGGGGGUGACCGCCGGAGCGGTTTTAGAG |
| | | (SEQ ID NO: 36) | CTAGAAATAGCAAGTTAAAATAAGGCTAG |
| | | | TCCGTTATCAACTTGAAAAAGTGGCACCG |
| | | | AGTCGGTGCUUUUUU (SEQ ID NO: |
| | | | 37) |
| VEGFA_3 | Cas12i2 | GTTGACATTGTCCACACCTG | AGAAAUCCGUCUUUCAUUGACGGGUUGAC |
| | | (SEQ ID NO: 38) | AUUGUCCACACCUG (SEQ ID NO: 39) |
| VEGFA_3 | SpCas9 | TTGTTGACATTGTCCACACC | UUGUUGACAUUGUCCACACCGTTTTAGAG |
| | | (SEQ ID NO: 40) | CTAGAAATAGCAAGTTAAAATAAGGCTAG |
| | | | TCCGTTATCAACTTGAAAAAGTGGCACCG |
| | | | AGTCGGTGCUUUUUU (SEQ ID NO: |
| | | | 41) |
| VEGFA_4 | Cas12i2 | GGAAATCTATTGAGGCTCTG | AGAAAUCCGUCUUUCAUUGACGGGGAAAU |
| | | (SEQ ID NO: 42) | CUAUUGAGGCUCUG (SEQ ID NO: |
| | | | 43) |
| VEGFA_4 | SpCas9 | TTGGAAATCTATTGAGGCTC | UUGGAAAUCUAUUGAGGCUCGTTTTAGAG |
| | | (SEQ ID NO: 44) | CTAGAAATAGCAAGTTAAAATAAGGCTAG |
| | | | TCCGTTATCAACTTGAAAAAGTGGCACCG |
| | | | AGTCGGTGCUUUUUU (SEQ ID NO: |
| | | | 45) |

Prior to nucleofection, 25 μM Cas12i2 and SpCas9 ribonucleoprotein (RNP) complexes were formed by mixing protein with crRNA/sgRNA at a 2.5:1 molar ratio of RNA:protein and incubation at 37° C. for 30-60 minutes. During the RNP complexation, 293T cells were harvested by treatment with a recombinant enzyme to dissociate adherent cells (Thermo Fisher) and centrifugation. Cells were washed once with PBS, counted, and re-suspended at 15,000,000 cells per mL in nucleofection buffer (Lonza). Twenty microliters of 293T cell slurry (300,000 cells) was added to 5 μL of 25 μM RNPs along with 1 μL of 100 μM enhancer DNA oligo (IDT). Twenty microliters of the mixtures were then transferred to nucleofection cuvettes (Lonza) and RNPs were nucleofected using a nucleofector system (Lonza). Nucleofected cells were then diluted to 100 μL with DMEM+10% FBS (Thermo Fisher) and 10 μL was transferred to 96-well plates containing 100 μL of DMEM+10% FBS. Cells were grown at 37° C. with 5% CO₂ for 72 hours. Cells were washed once with PBS and resuspended in 50 μL of DNA extraction solution (Lucigen) and transferred to 96-well PCR plates. Cells were lysed by incubation at 65° C. for 15 minutes and 98° C. for 10 minutes in a thermal cycler (Thermo Fisher).

Samples for next generation sequencing (NGS) were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled, loaded onto a 2% E-gel EX for 10 minutes and gel extracted. Sequencing runs were done with a 150 cycle NGS output kit (Illumina).

Sample-loaded kits were run on a sequencing instrument (Illumina), which was used to output single-end read fastq files corresponding to specific RNP samples. Reads (˜50-150 nt in length) were compared to genomic reference sequences (˜120-140 nit in length) to identify alignment gaps that indicate the position and size of deletion (indel) edits mediated by RNP. The indel percentage was calculated as the number of indel-containing reads divided by the number of reads analyzed (up to 50,000). The QC standard for the minimum number of reads was 10,000.

For each deletion length, indel size frequencies among edited reads were calculated as the number of indel-containing reads of a certain deletion length divided by the number of reads containing indels. Frequencies for indel start/end positions (relative to the target sequence programmed into a sample's guide RNA) were calculated per indel size as the number of reads with indels of a certain size starting/ending at each position divided by the number of reads containing indels. A cumulative density function (CDF) was calculated for indel size frequency by summing the frequency of deletions longer than or equal to each possible deletion length (numerically less than or equal to in terms of indel size).

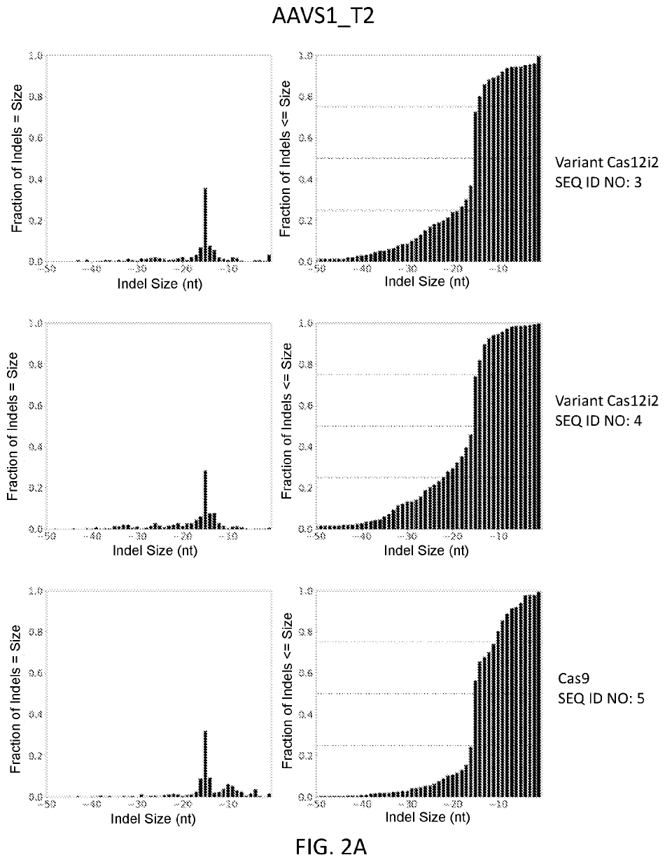

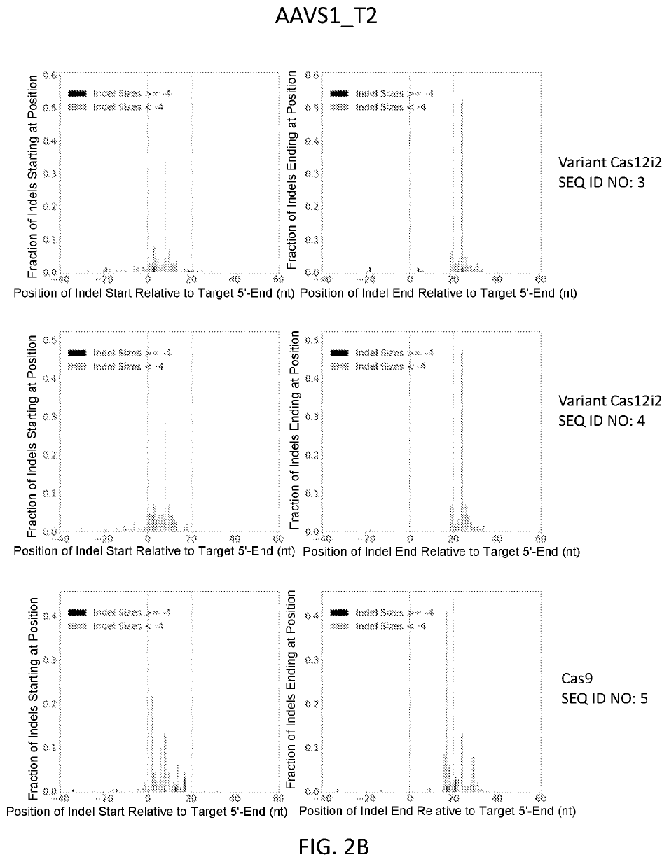

In FIGS. 2-11, rows of plots generated in the python matplotlib library represent indel size and position profiles corresponding to individual RNP samples. The first column includes histograms of indel size frequencies among edited reads. The second column includes bar graphs representing the indel size CDF function. Horizontal lines serve as visual cues to assess the broadness of the CDF function. The third and fourth columns includes histograms of indel start and end positions, respectively. The colors of the bars in the third and fourth columns are split based on the fraction of indels of sizes>=−4 that start or end at a position. Indels greater than or equal to 4 (e.g., deletions having a length of M to 4 nucleotides) are depicted in black and indels less than −4 (e.g., deletions having a length of at least 5 nucleotides) are depicted in gray. Vertical lines serve as visual cues for the beginning and end of the target sequence programmed into a sample's RNA guide. For the Cas12i2 plots, the 5′-NTTN-3′ (e.g., 5′-CTTT-3′) PAM is directly upstream of the left vertical line. The position of the 5′-NTTN-3′ sequence is thus from −4 to −1 according to the numbering of Cas2i2 plots in FIGS. 2-11. For the SpCas9 plots, the 5′-NGG-3′ PAM starts at the right vertical line. The position of the 5′-NGG-3′ sequence is thus from 19 to 21 according to the numbering of SpCas9 plots in FIGS. 2-11. Positions 1 to 20 represent the target sequence to which the Cas12i25 RNA guide hinds, and positions 0 to 19 represent the target sequence to which the SpCas9 RNA guide hinds.

The indel data from FIGS. 2-11 is further presented quantitatively in Tables 2-8 below. In Table 2, the minimum indel size was calculated such that that CDF of the indel size exceeded X, wherein X 0.25, 0.50 and 0.75. This is represented as Min(CDF(Indel Size)>=X) in Table 2. The mean and mode of indel start and end positions for both the collection of indels with sizes>=−4 and the collection of indels with sizes<−4 were calculated, as were the fraction of indels with sizes>=−4 and with sizes<−4 were calculated, as shown in Tables 3-6.

TABLE 2
Cumulative Distribution Function Values of Indel Size for Cas12i2 and SpCas9.

| | | | Min (CDF(Indel | Min (CDF(Indel | Min (CDF(Indel |
|---|---|---|---|---|---|
| Gene | Locus | Nuclease | Size) > 0.75 | Size) > 0.50 | Size) > 0.25 |
| AAVS1 | T2 | variant Cas12i2 | −14 | −15 | −18 |
| | | (SEQ ID NO: 3) | | | |
| | | variant Cas12i2 | −14 | −15 | −22 |
| | | (SEQ ID NO: 4) | | | |
| | | WT Cas9 | −10 | −15 | −15 |
| | | (SEQ ID NO: 5) | | | |
| AAVS1 | T3 | WT Cas12i2 | −2 | −8 | −12 |
| | | (SEQ ID NO: 2) | | | |
| | | variant Cas12i2 | −2 | −8 | −14 |
| | | (SEQ ID NO: 3) | | | |
| | | variant Cas12i2 | −10 | −14 | −26 |
| | | (SEQ ID NO: 4) | | | |
| | | WT Cas9 | −1 | −3 | −10 |
| | | (SEQ ID NO: 5) | | | |
| AAVS1 | T4 | variant Cas12i2 | −7 | −11 | −16 |
| | | (SEQ ID NO: 3) | | | |
| | | variant Cas12i2 | −9 | −13 | −18 |
| | | (SEQ ID NO: 4) | | | |
| | | WT Cas9 | −2 | −2 | −7 |
| | | (SEQ ID NO: 5) | | | |
| AAVS1 | T6 | WT Cas12i2 | −1 | −4 | −9 |
| | | (SEQ ID NO: 2) | | | |
| | | variant Cas12i2 | −12 | −15 | −25 |
| | | (SEQ ID NO: 3) | | | |
| | | variant Cas12i2 | −13 | −18 | −25 |
| | | (SEQ ID NO: 4) | | | |
| | | WT Cas9 | −4 | −6 | −14 |
| | | (SEQ ID NO: 5) | | | |
| EMX1 | T3 | WT Cas12i2 | −3 | −9 | −20 |
| | | (SEQ ID NO: 2) | | | |
| | | variant Cas12i2 | −4 | −9 | −16 |
| | | (SEQ ID NO: 3) | | | |
| | | variant Cas12i2 | −7 | −11 | −21 |
| | | (SEQ ID NO: 4) | | | |
| | | WT Cas9 | −4 | −6 | −6 |
| | | (SEQ ID NO: 5) | | | |
| EMX1 | T4 | variant Cas12i2 | −3 | −11 | −18 |
| | | (SEQ ID NO: 3) | | | |
| | | variant Cas12i2 | −9 | −15 | −19 |
| | | (SEQ ID NO: 4) | | | |
| | | WT Cas9 | −1 | −1 | −1 |
| | | (SEQ ID NO: 5) | | | |
| EMX1 | T5 | WT Cas12i2 | −6 | −12 | −23 |
| | | (SEQ ID NO: 2) | | | |
| | | variant Cas12i2 | −10 | −16 | −25 |
| | | (SEQ ID NO: 3) | | | |
| | | variant Cas12i2 | −12 | −23 | −31 |
| | | (SEQ ID NO: 4) | | | |
| | | WT Cas9 | −4 | −22 | −31 |
| | | (SEQ ID NO: 5) | | | |
| VEGFA | T1 | variant Cas12i2 | −2 | −8 | −13 |
| | | (SEQ ID NO: 3) | | | |
| | | variant Cas12i2 | −7 | −12 | −20 |
| | | (SEQ ID NO: 4) | | | |
| | | WT Cas9 | −6 | −8 | −18 |
| | | (SEQ ID NO: 5) | | | |
| VEGFA | T3 | WT Cas12i2 | −2 | −3 | −7 |
| | | (SEQ ID NO: 2) | | | |
| | | variant Cas12i2 | −3 | −10 | −20 |
| | | (SEQ ID NO: 3) | | | |
| | | variant Cas12i2 | −8 | −16 | −26 |
| | | (SEQ ID NO: 4) | | | |
| | | WT Cas9 | −2 | −4 | −11 |
| | | (SEQ ID NO: 5) | | | |
| VEGFA | T4 | WT Cas12i2 | −2 | −6 | −11 |
| | | (SEQ ID NO: 2) | | | |
| | | variant Cas12i2 | −13 | −15 | −20 |
| | | (SEQ ID NO: 3) | | | |
| | | variant Cas12i2 | −12 | −16 | −20 |
| | | (SEQ ID NO: 4) | | | |
| | | WT Cas9 | −4 | −5 | −9 |
| | | (SEQ ID NO: 5) | | | |

TABLE 3
Mode Indel Start and End Positions for Indels of Sizes >= −4
(1-4 Nucleotides).

| | | | Fraction | | |
|---|---|---|---|---|---|
| | | | Indels 1-4 | Mode | Mode |
| Gene | Locus | Nuclease | Nucleotides | (Start) | (End) |
| AAVS1 | T2 | variant Cas12i2 | 0.05 | 3 | −18 |
| | | (SEQ ID NO: 3) | | | |
| | | variant Cas12i2 | 0.01 | −19 | −18 |
| | | (SEQ ID NO: 4) | | | |
| | | WT Cas9 | 0.06 | 17 | 21 |
| | | (SEQ ID NO: 5) | | | |
| AAVS1 | T3 | WT Cas12i2 | 0.42 | 23 | 24 |
| | | (SEQ ID NO: 2) | | | |
| | | variant Cas12i2 | 0.44 | 23 | 24 |
| | | (SEQ ID NO: 3) | | | |
| | | variant Cas12i2 | 0.11 | 23 | 24 |
| | | (SEQ ID NO: 4) | | | |
| | | WT Cas9 | 0.58 | 15 | 16 |
| | | (SEQ ID NO: 5) | | | |
| AAVS1 | T4 | variant Cas12i2 | 0.19 | 20 | 24 |
| | | (SEQ ID NO: 3) | | | |
| | | variant Cas12i2 | 0.12 | 20 | 22 |
| | | (SEQ ID NO: 4) | | | |
| | | WT Cas9 | 0.69 | 17 | 19 |
| | | (SEQ ID NO: 5) | | | |
| AAVS1 | T6 | WT Cas12i2 | 0.58 | 23 | 24 |
| | | (SEQ ID NO: 2) | | | |
| | | variant Cas12i2 | 0.04 | 23 | 26 |
| | | (SEQ ID NO: 3) | | | |
| | | variant Cas12i2 | 0.00 | −5 | −5 |
| | | (SEQ ID NO: 4) | | | |
| | | WT Cas9 | 0.30 | 17 | 19 |
| | | (SEQ ID NO: 5) | | | |
| EMX1 | T3 | WT Cas12i2 | 0.30 | 19 | 22 |
| | | (SEQ ID NO: 2) | | | |
| | | variant Cas12i2 | 0.26 | 19 | 24 |
| | | (SEQ ID NO: 3) | | | |
| | | variant Cas12i2 | 0.13 | 19 | 24 |
| | | (SEQ ID NO: 4) | | | |
| | | WT Cas9 | 0.26 | 15 | 17 |
| | | (SEQ ID NO: 5) | | | |
| EMX1 | T4 | variant Cas12i2 | 0.28 | 23 | 26 |
| | | (SEQ ID NO: 3) | | | |
| | | variant Cas12i2 | 0.14 | 23 | 24 |
| | | (SEQ ID NO: 4) | | | |
| | | WT Cas9 | 0.84 | 15 | 16 |
| | | (SEQ ID NO: 5) | | | |
| EMX1 | T5 | WT Cas12i2 | 0.19 | 20 | 21 |
| | | (SEQ ID NO: 2) | | | |
| | | variant Cas12i2 | 0.13 | 20 | 21 |
| | | (SEQ ID NO: 3) | | | |
| | | variant Cas12i2 | 0.04 | 20 | 21 |
| | | (SEQ ID NO: 4) | | | |
| | | WT Cas9 | 0.28 | 17 | 16 |
| | | (SEQ ID NO: 5) | | | |
| VEGFA | T1 | variant Cas12i2 | 0.39 | 22 | 24 |
| | | (SEQ ID NO: 3) | | | |
| | | variant Cas12i2 | 0.17 | 23 | 24 |
| | | (SEQ ID NO: 4) | | | |
| | | WT Cas9 | 0.12 | 14 | 17 |
| | | (SEQ ID NO: 5) | | | |
| VEGFA | T3 | WT Cas12i2 | 0.62 | 23 | 25 |
| | | (SEQ ID NO: 2) | | | |
| | | variant Cas12i2 | 0.34 | 23 | 25 |
| | | (SEQ ID NO: 3) | | | |
| | | variant Cas12i2 | 0.18 | 23 | 25 |
| | | (SEQ ID NO: 4) | | | |
| | | WT Cas9 | 0.51 | 14 | 16 |
| | | (SEQ ID NO: 5) | | | |
| VEGFA | T4 | WT Cas12i2 | 0.43 | 20 | 22 |
| | | (SEQ ID NO: 2) | | | |
| | | variant Cas12i2 | 0.05 | 20 | 22 |
| | | (SEQ ID NO: 3) | | | |
| | | variant Cas12i2 | 0.00 | 17 | 21 |
| | | (SEQ ID NO: 4) | | | |
| | | WT Cas9 | 0.40 | 13 | 17 |
| | | (SEQ ID NO: 5) | | | |

TABLE 4
Mean Indel Start and End Positions for Indels Having a
Size of 1-4 Nucleotides.

| | | | Fraction | | |
|---|---|---|---|---|---|
| | | | Indels 1-4 | Mean | Mean |
| Gene | Locus | Nuclease | Nucleotides | (Start) | (End) |
| AAVS1 | T2 | variant Cas12i2 | 0.05 | 2.46 | 4.06 |
| | | (SEQ ID NO: 3) | | | |
| | | variant Cas12i2 | 0.01 | 3.17 | −1.87 |
| | | (SEQ ID NO: 4) | | | |
| | | WT Cas9 | 0.06 | 9.39 | 12.28 |
| | | (SEQ ID NO: 5) | | | |
| AAVS1 | T3 | WT Cas12i2 | 0.42 | 21.63 | 23.43 |
| | | (SEQ ID NO: 2) | | | |
| | | variant Cas12i2 | 0.44 | 21.87 | 23.73 |
| | | (SEQ ID NO: 3) | | | |
| | | variant Cas12i2 | 0.11 | 22.31 | 24.09 |
| | | (SEQ ID NO: 4) | | | |
| | | WT Cas9 | 0.58 | 14.81 | 16.52 |
| | | (SEQ ID NO: 5) | | | |
| AAVS1 | T4 | variant Cas12i2 | 0.19 | 21.49 | 24.05 |
| | | (SEQ ID NO: 3) | | | |
| | | variant Cas12i2 | 0.12 | 21.22 | 23.85 |
| | | (SEQ ID NO: 4) | | | |
| | | WT Cas9 | 0.69 | 16.66 | 18.78 |
| | | (SEQ ID NO: 5) | | | |
| AAVS1 | T6 | WT Cas12i2 | 0.58 | 22.38 | 24.07 |
| | | (SEQ ID NO: 2) | | | |
| | | variant Cas12i2 | 0.04 | 22.11 | 24.33 |
| | | (SEQ ID NO: 3) | | | |
| | | variant Cas12i2 | 0.00 | 0.00 | 0.00 |
| | | (SEQ ID NO: 4) | | | |
| | | WT Cas9 | 0.30 | 15.68 | 17.92 |
| | | (SEQ ID NO: 5) | | | |
| EMX1 | T3 | WT Cas12i2 | 0.30 | 4.94 | 5.99 |
| | | (SEQ ID NO: 2) | | | |
| | | variant Cas12i2 | 0.26 | 21.08 | 23.32 |
| | | (SEQ ID NO: 3) | | | |
| | | variant Cas12i2 | 0.13 | 20.65 | 23.03 |
| | | (SEQ ID NO: 4) | | | |
| | | WT Cas9 | 0.26 | 15.00 | 17.07 |
| | | (SEQ ID NO: 5) | | | |
| EMX1 | T4 | variant Cas12i2 | 0.28 | 22.98 | 25.16 |
| | | (SEQ ID NO: 3) | | | |
| | | variant Cas12i2 | 0.14 | 22.74 | 24.86 |
| | | (SEQ ID NO: 4) | | | |
| | | WT Cas9 | 0.84 | 14.99 | 16.15 |
| | | (SEQ ID NO: 5) | | | |
| EMX1 | T5 | WT Cas12i2 | 0.19 | 18.89 | 20.28 |
| | | (SEQ ID NO: 2) | | | |
| | | variant Cas12i2 | 0.13 | 19.74 | 21.59 |
| | | (SEQ ID NO: 3) | | | |
| | | variant Cas12i2 | 0.04 | 19.14 | 20.83 |
| | | (SEQ ID NO: 4) | | | |
| | | WT Cas9 | 0.28 | 15.00 | 16.83 |
| | | (SEQ ID NO: 5) | | | |
| VEGFA | T1 | variant Cas12i2 | 0.39 | 22.24 | 24.21 |
| | | (SEQ ID NO: 3) | | | |
| | | variant Cas12i2 | 0.17 | 21.69 | 23.73 |
| | | (SEQ ID NO: 4) | | | |
| | | WT Cas9 | 0.12 | 26.44 | 28.09 |
| | | (SEQ ID NO: 5) | | | |
| VEGFA | T3 | WT Cas12i2 | 0.62 | 19.28 | 21.36 |
| | | (SEQ ID NO: 2) | | | |
| | | variant Cas12i2 | 0.34 | 22.31 | 24.62 |
| | | (SEQ ID NO: 3) | | | |
| | | variant Cas12i2 | 0.18 | 22.11 | 24.56 |
| | | (SEQ ID NO: 4) | | | |
| | | WT Cas9 | 0.51 | 14.32 | 16.30 |
| | | (SEQ ID NO: 5) | | | |
| VEGFA | T4 | WT Cas12i2 | 0.43 | 22.37 | 24.69 |
| | | (SEQ ID NO: 2) | | | |
| | | variant Cas12i2 | 0.05 | 21.14 | 23.58 |
| | | (SEQ ID NO: 3) | | | |
| | | variant Cas12i2 | 0.00 | 22.55 | 24.67 |
| | | (SEQ ID NO: 4) | | | |
| | | WT Cas9 | 0.40 | 14.15 | 16.92 |
| | | (SEQ ID NO: 5) | | | |

TABLE 5
Mode Indel Start and End Positions for Indels Having a Size of
5 Nucleotides or Longer.

| | | | Fraction | | |
|---|---|---|---|---|---|
| | | | Indels 5+ | Mean | Mean |
| Gene | Locus | Nuclease | Nucleotides | (Start) | (End) |
| AAVS1 | T2 | variant Cas12i2 | 0.95 | 9 | 24 |
| | | (SEQ ID NO: 3) | | | |
| | | variant Cas12i2 | 0.99 | 9 | 23 |
| | | (SEQ ID NO: 4) | | | |
| | | WT Cas9 | 0.94 | 2 | 17 |
| | | (SEQ ID NO: 5) | | | |
| AAVS1 | T3 | WT Cas12i2 | 0.58 | 13 | 24 |
| | | (SEQ ID NO: 2) | | | |
| | | variant Cas12i2 | 0.56 | 13 | 24 |
| | | (SEQ ID NO: 3) | | | |
| | | variant Cas12i2 | 0.89 | 13 | 24 |
| | | (SEQ ID NO: 4) | | | |
| | | WT Cas9 | 0.42 | 10 | 17 |
| | | (SEQ ID NO: 5) | | | |
| AAVS1 | T4 | variant Cas12i2 | 0.81 | 13 | 24 |
| | | (SEQ ID NO: 3) | | | |
| | | variant Cas12i2 | 0.88 | 13 | 24 |
| | | (SEQ ID NO: 4) | | | |
| | | WT Cas9 | 0.31 | 17 | 21 |
| | | (SEQ ID NO: 5) | | | |
| AAVS1 | T6 | WT Cas12i2 | 0.42 | 15 | 23 |
| | | (SEQ ID NO: 2) | | | |
| | | variant Cas12i2 | 0.96 | 11 | 26 |
| | | (SEQ ID NO: 3) | | | |
| | | variant Cas12i2 | 1 | 11 | −5 |
| | | (SEQ ID NO: 4) | | | |
| | | WT Cas9 | 0.7 | 14 | 20 |
| | | (SEQ ID NO: 5) | | | |
| EMX1 | T3 | WT Cas12i2 | 0.7 | 13 | 24 |
| | | (SEQ ID NO: 2) | | | |
| | | variant Cas12i2 | 0.74 | 13 | 24 |
| | | (SEQ ID NO: 3) | | | |
| | | variant Cas12i2 | 0.87 | 13 | 22 |
| | | (SEQ ID NO: 4) | | | |
| | | WT Cas9 | 0.74 | 13 | 19 |
| | | (SEQ ID NO: 5) | | | |
| EMX1 | T4 | variant Cas12i2 | 0.72 | 11 | 25 |
| | | (SEQ ID NO: 3) | | | |
| | | variant Cas12i2 | 0.86 | 11 | 25 |
| | | (SEQ ID NO: 4) | | | |
| | | WT Cas9 | 0.16 | 11 | 26 |
| | | (SEQ ID NO: 5) | | | |
| EMX1 | T5 | WT Cas12i2 | 0.81 | 5 | 24 |
| | | (SEQ ID NO: 2) | | | |
| | | variant Cas12i2 | 0.87 | 12 | 28 |
| | | (SEQ ID NO: 3) | | | |
| | | variant Cas12i2 | 0.96 | 5 | 28 |
| | | (SEQ ID NO: 4) | | | |
| | | WT Cas9 | 0.72 | 8 | 31 |
| | | (SEQ ID NO: 5) | | | |
| VEGFA | T1 | variant Cas12i2 | 0.61 | 14 | 22 |
| | | (SEQ ID NO: 3) | | | |
| | | variant Cas12i2 | 0.83 | 11 | 24 |
| | | (SEQ ID NO: 4) | | | |
| | | WT Cas9 | 0.88 | 13 | 20 |
| | | (SEQ ID NO: 5) | | | |
| VEGFA | T3 | WT Cas12i2 | 0.38 | 19 | 25 |
| | | (SEQ ID NO: 2) | | | |
| | | variant Cas12i2 | 0.66 | 10 | 25 |
| | | (SEQ ID NO: 3) | | | |
| | | variant Cas12i2 | 0.82 | 10 | 25 |
| | | (SEQ ID NO: 4) | | | |
| | | WT Cas9 | 0.49 | 17 | 17 |
| | | (SEQ ID NO: 5) | | | |
| VEGFA | T4 | WT Cas12i2 | 0.57 | 11 | 24 |
| | | (SEQ ID NO: 2) | | | |
| | | variant Cas12i2 | 0.95 | 11 | 24 |
| | | (SEQ ID NO: 3) | | | |
| | | variant Cas12i2 | 1 | 11 | 24 |
| | | (SEQ ID NO: 4) | | | |
| | | WT Cas9 | 0.6 | 12 | 17 |
| | | (SEQ ID NO: 5) | | | |

TABLE 6
Mean Indel Start and End Positions for Indels Having a Size of
5 Nucleotides or Longer.

| | | | Fraction | | |
|---|---|---|---|---|---|
| | | | Indels 5+ | Mean | Mean |
| Gene | Locus | Nuclease | Nucleotides | (Start) | (End) |
| AAVS1 | T2 | variant Cas12i2 | 0.95 | 5.73 | 22.2 |
| | | (SEQ ID NO: 3) | | | |
| | | variant Cas12i2 | 0.99 | 5.17 | 5.39 |
| | | (SEQ ID NO: 4) | | | |
| | | WT Cas9 | 0.94 | 5.62 | 20.1 |
| | | (SEQ ID NO: 5) | | | |
| AAVS1 | T3 | WT Cas12i2 | 0.58 | 11.9 | 26.51 |
| | | (SEQ ID NO: 2) | | | |
| | | variant Cas12i2 | 0.56 | 11 | 27.75 |
| | | (SEQ ID NO: 3) | | | |
| | | variant Cas12i2 | 0.89 | 9.32 | 29.44 |
| | | (SEQ ID NO: 4) | | | |
| | | WT Cas9 | 0.42 | 5.27 | 20.03 |
| | | (SEQ ID NO: 5) | | | |
| AAVS1 | T4 | variant Cas12i2 | 0.81 | 13.99 | 28.72 |
| | | (SEQ ID NO: 3) | | | |
| | | variant Cas12i2 | 0.88 | 13.19 | 28.74 |
| | | (SEQ ID NO: 4) | | | |
| | | WT Cas9 | 0.31 | 14.55 | 27.18 |
| | | (SEQ ID NO: 5) | | | |
| AAVS1 | T6 | WT Cas12i2 | 0.42 | 11.94 | 24.3 |
| | | (SEQ ID NO: 2) | | | |
| | | variant Cas12i2 | 0.96 | 10.03 | 13.06 |
| | | (SEQ ID NO: 3) | | | |
| | | variant Cas12i2 | 1 | 9.24 | 0 |
| | | (SEQ ID NO: 4) | | | |
| | | WT Cas9 | 0.7 | 11.42 | 9.75 |
| | | (SEQ ID NO: 5) | | | |
| EMX1 | T3 | WT Cas12i2 | 0.7 | 9.66 | 25.59 |
| | | (SEQ ID NO: 2) | | | |
| | | variant Cas12i2 | 0.74 | 11.95 | 26.2 |
| | | (SEQ ID NO: 3) | | | |
| | | variant Cas12i2 | 0.87 | 10.73 | 26.16 |
| | | (SEQ ID NO: 4) | | | |
| | | WT Cas9 | 0.74 | 10.78 | 20.34 |
| | | (SEQ ID NO: 5) | | | |
| EMX1 | T4 | variant Cas12i2 | 0.72 | 11.53 | 27.63 |
| | | (SEQ ID NO: 3) | | | |
| | | variant Cas12i2 | 0.86 | 10.68 | 27.66 |
| | | (SEQ ID NO: 4) | | | |
| | | WT Cas9 | 0.16 | 7.65 | 24.08 |
| | | (SEQ ID NO: 5) | | | |
| EMX1 | T5 | WT Cas12i2 | 0.81 | 8.38 | 27.99 |
| | | (SEQ ID NO: 2) | | | |
| | | variant Cas12i2 | 0.87 | 7.04 | 28.14 |
| | | (SEQ ID NO: 3) | | | |
| | | variant Cas12i2 | 0.96 | 5.25 | 28.72 |
| | | (SEQ ID NO: 4) | | | |
| | | WT Cas9 | 0.72 | 3.83 | 30.12 |
| | | (SEQ ID NO: 5) | | | |
| VEGFA | T1 | variant Cas12i2 | 0.61 | 12.21 | 25.72 |
| | | (SEQ ID NO: 3) | | | |
| | | variant Cas12i2 | 0.83 | 10.5 | 27.31 |
| | | (SEQ ID NO: 4) | | | |
| | | WT Cas9 | 0.88 | 9.27 | 23.2 |
| | | (SEQ ID NO: 5) | | | |
| VEGFA | T3 | WT Cas12i2 | 0.38 | 14.45 | 26.65 |
| | | (SEQ ID NO: 2) | | | |
| | | variant Cas12i2 | 0.66 | 11.85 | 31.48 |
| | | (SEQ ID NO: 3) | | | |
| | | variant Cas12i2 | 0.82 | 9.65 | 30.97 |
| | | (SEQ ID NO: 4) | | | |
| | | WT Cas9 | 0.49 | 9.38 | 22.4 |
| | | (SEQ ID NO: 5) | | | |
| VEGFA | T4 | WT Cas12i2 | 0.57 | 13.33 | 24.36 |
| | | (SEQ ID NO: 2) | | | |
| | | variant Cas12i2 | 0.95 | 11.27 | 29.03 |
| | | (SEQ ID NO: 3) | | | |
| | | variant Cas12i2 | 1 | 10.67 | 28.59 |
| | | (SEQ ID NO: 4) | | | |
| | | WT Cas9 | 0.6 | 10.54 | 19.92 |
| | | (SEQ ID NO: 5) | | | |

TABLE 7
Fraction of Indels with a Size of 1-4 Nucleotides with Cas12i2
Variant of SEQ ID NO: 4 and WT SpCas9 (SEQ ID NO: 5).

| | | Fraction Indels for | Fraction Indels for |
|---|---|---|---|
| | | Variant Cas12i2 | WT SpCas9 |
| Gene | Locus | (SEQ ID NO: 4) | (SEQ ID NO: 5) |
| AAVS1 | T2 | 0.01 | 0.06 |
| AAVS1 | T3 | 0.11 | 0.58 |
| AAVS1 | T4 | 0.12 | 0.69 |
| AAVS1 | T6 | 0.00 | 0.30 |
| EMX1 | T3 | 0.13 | 0.26 |
| EMX1 | T4 | 0.14 | 0.84 |
| EMX1 | T5 | 0.04 | 0.28 |
| VEGFA | T1 | 0.17 | 0.12 |
| VEGFA | T3 | 0.18 | 0.51 |
| VEGFA | T4 | 0.00 | 0.40 |

TABLE 8
Fraction of Indels with a Size of at Least 5 Nucleotides with
Cas12i2 Variant of SEQ ID NO: 4 and WT SpCas9
(SEQ ID NO: 5).

| | | Fraction Indels for | Fraction Indels for |
|---|---|---|---|
| | | Variant Cas12i2 | WT SpCas9 |
| Gene | Locus | (SEQ ID NO: 4) | (SEQ ID NO: 5) |
| AAVS1 | T2 | 0.99 | 0.94 |
| AAVS1 | T3 | 0.89 | 0.42 |
| AAVS1 | T4 | 0.88 | 0.31 |
| AAVS1 | T6 | 1.00 | 0.70 |
| EMX1 | T3 | 0.87 | 0.74 |
| EMX1 | T4 | 0.86 | 0.16 |
| EMX1 | T5 | 0.96 | 0.72 |
| VEGFA | T1 | 0.83 | 0.88 |
| VEGFA | T3 | 0.82 | 0.49 |
| VEGFA | T4 | 1.00 | 0.60 |

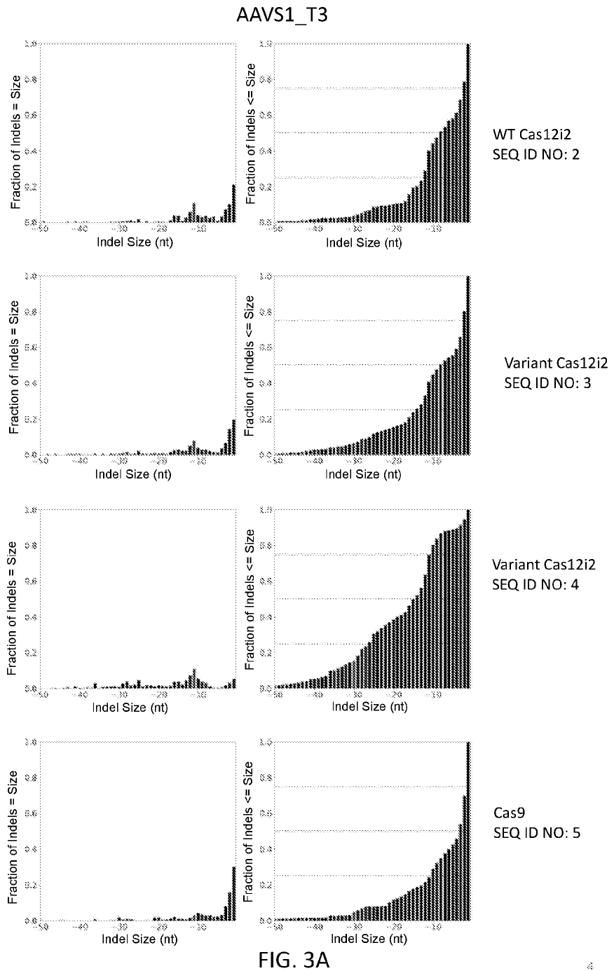

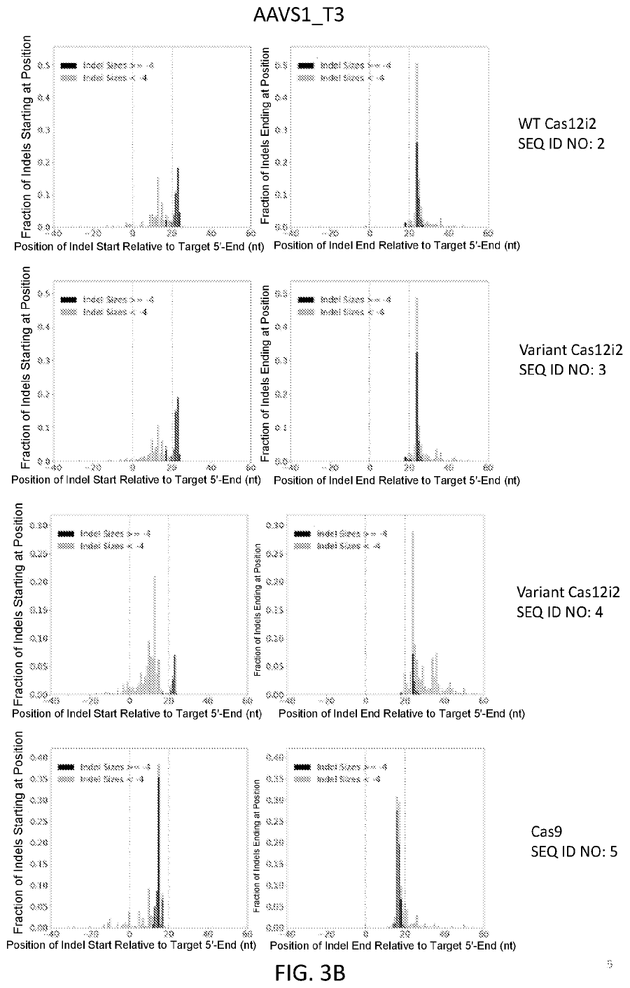

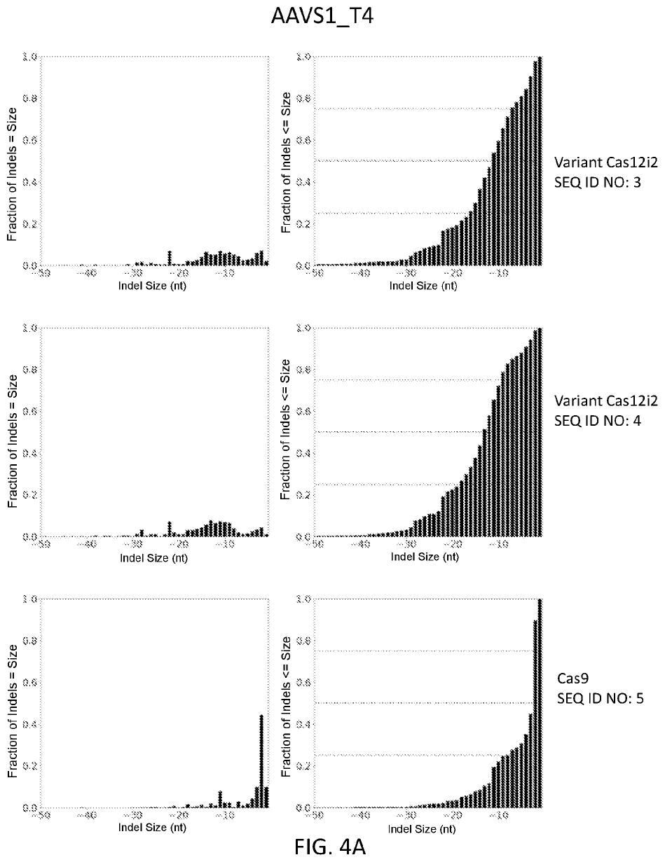

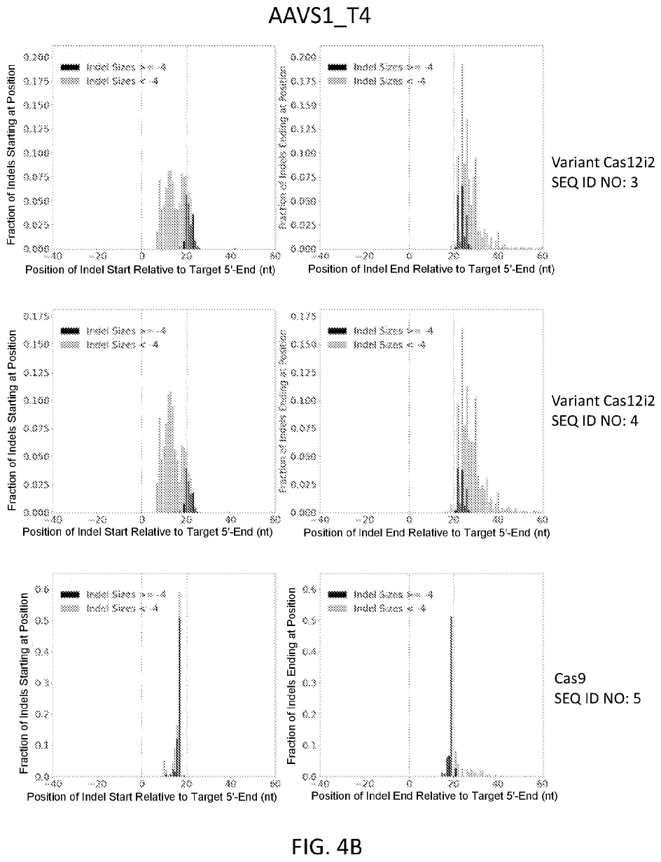

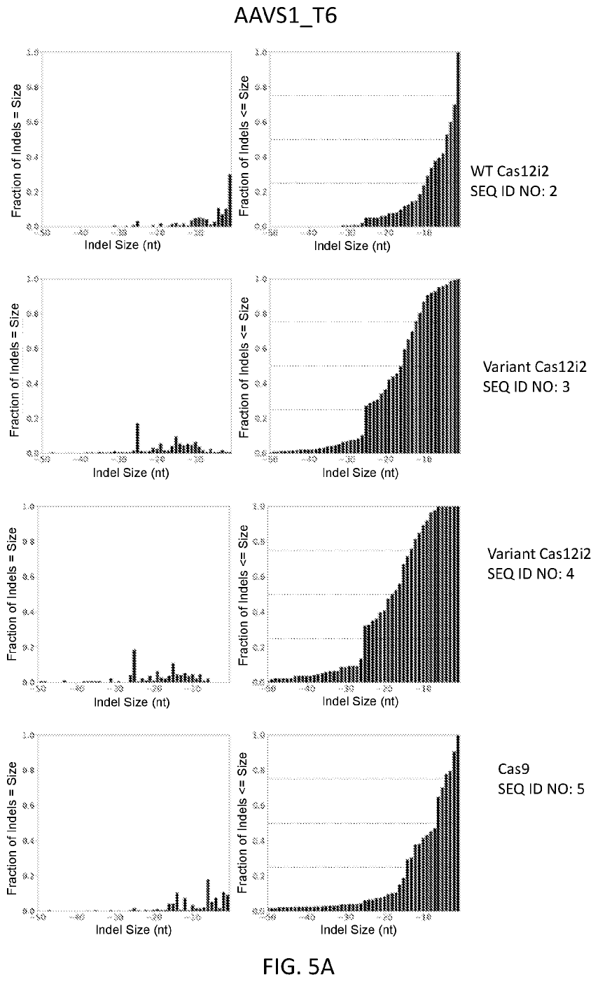

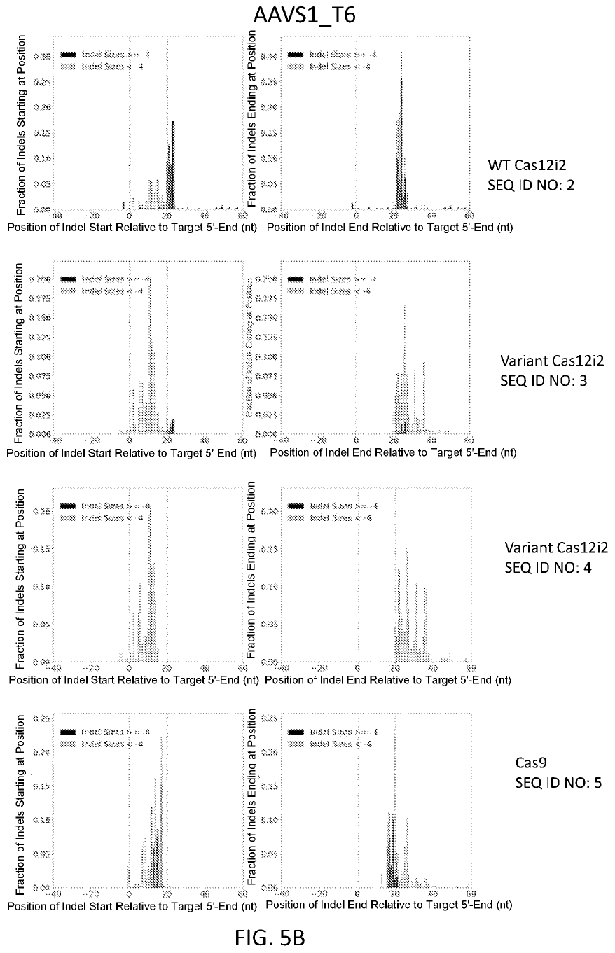

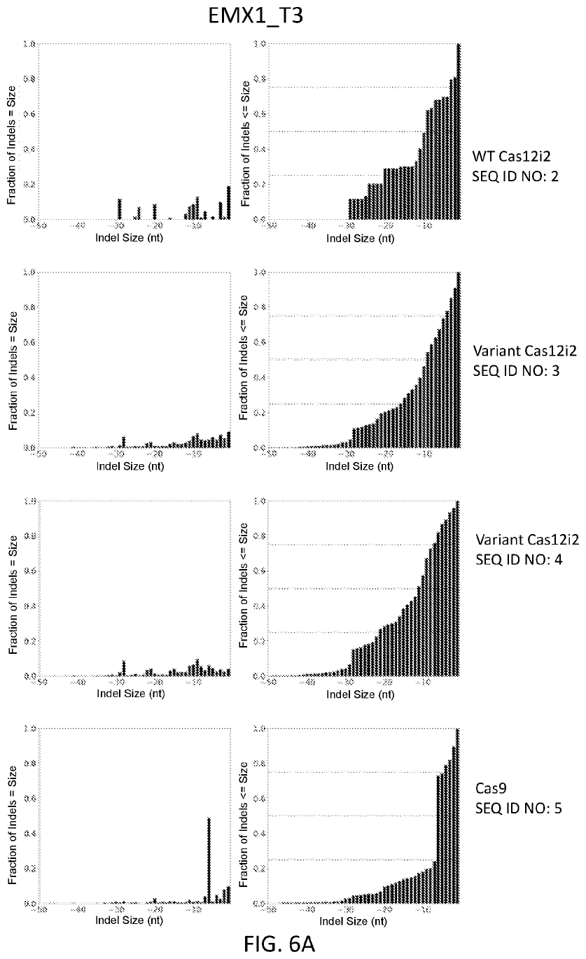

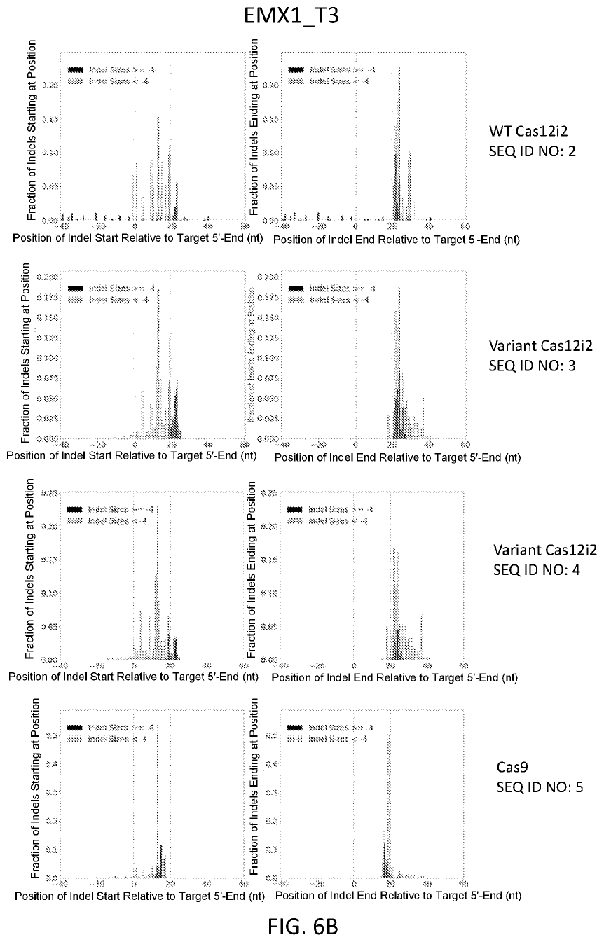

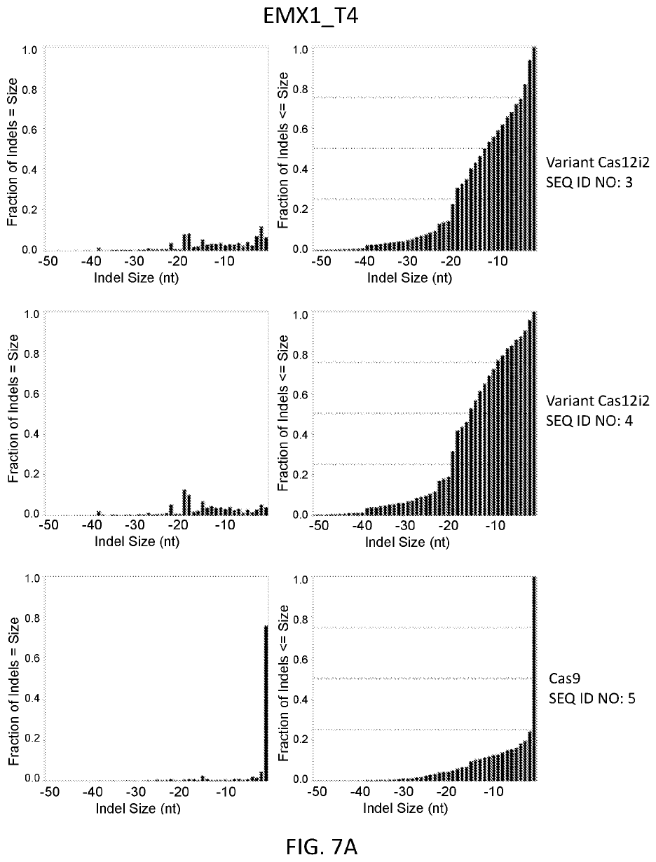

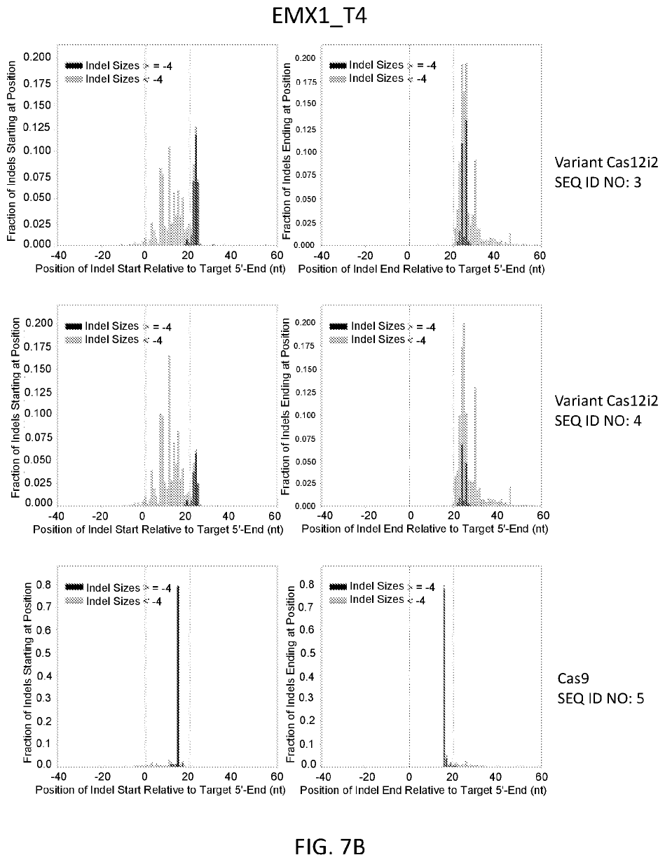

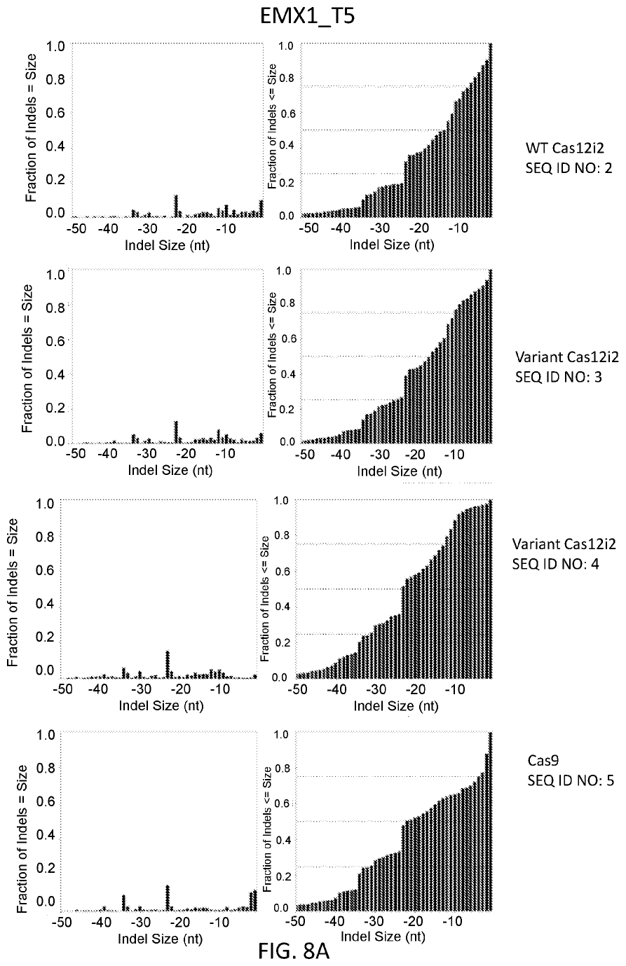

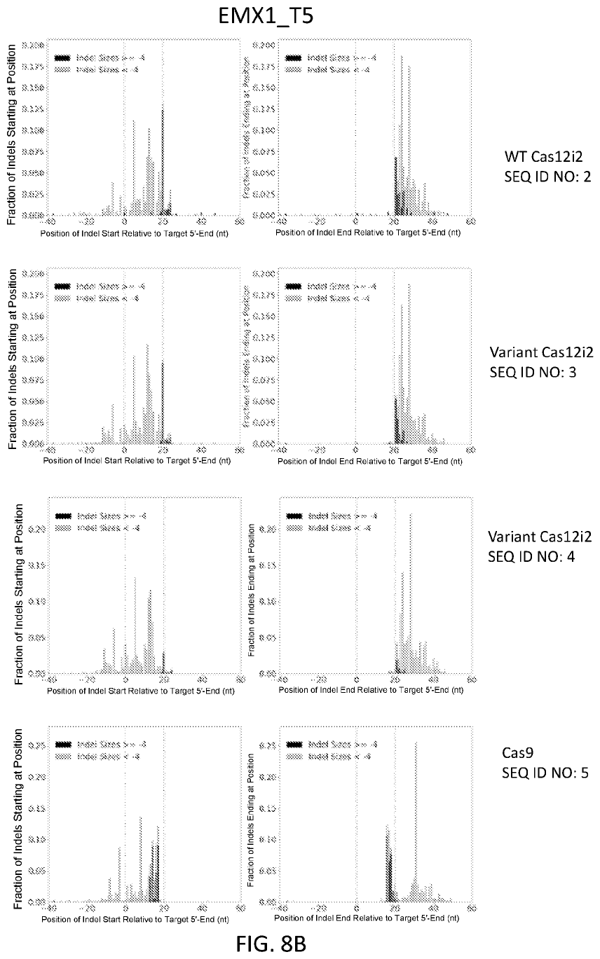

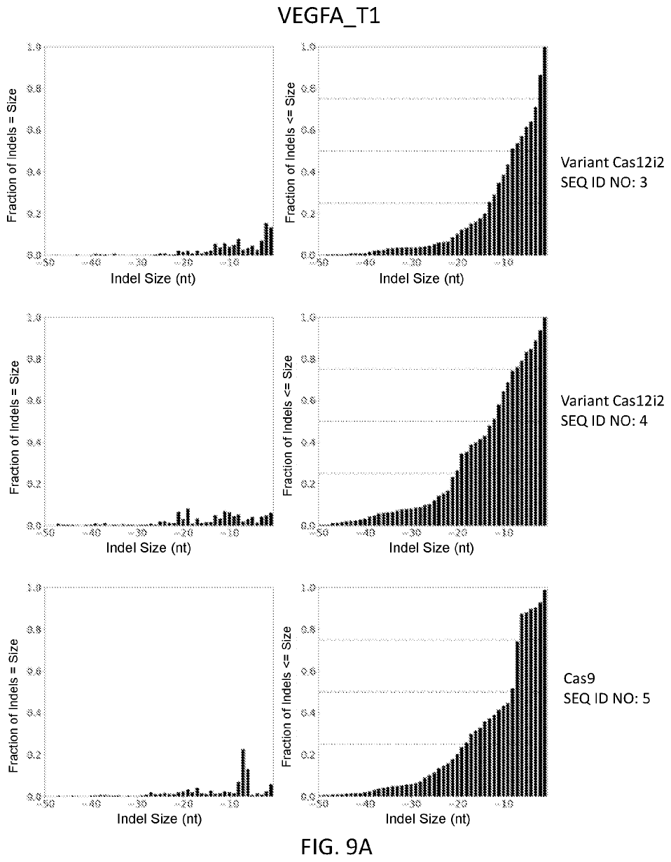

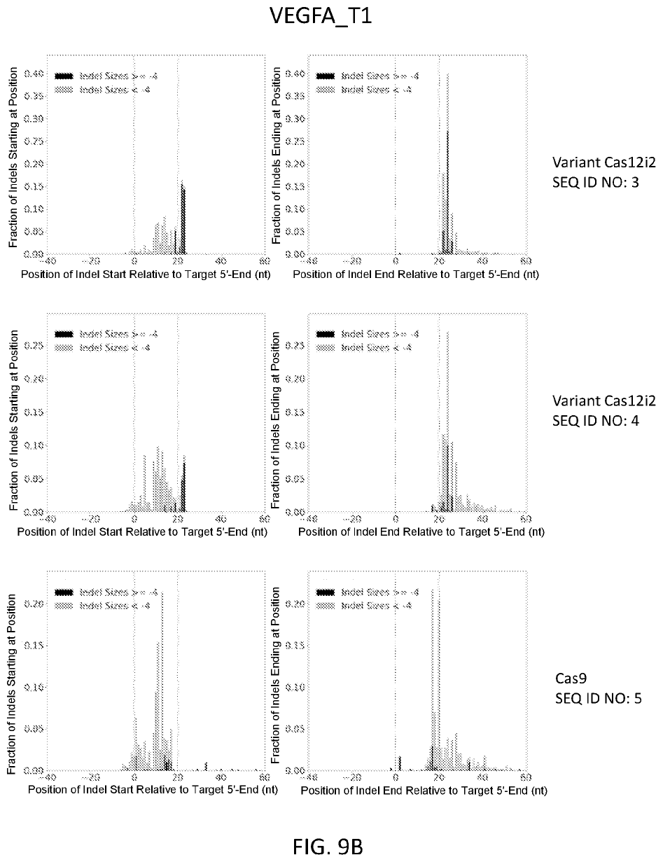

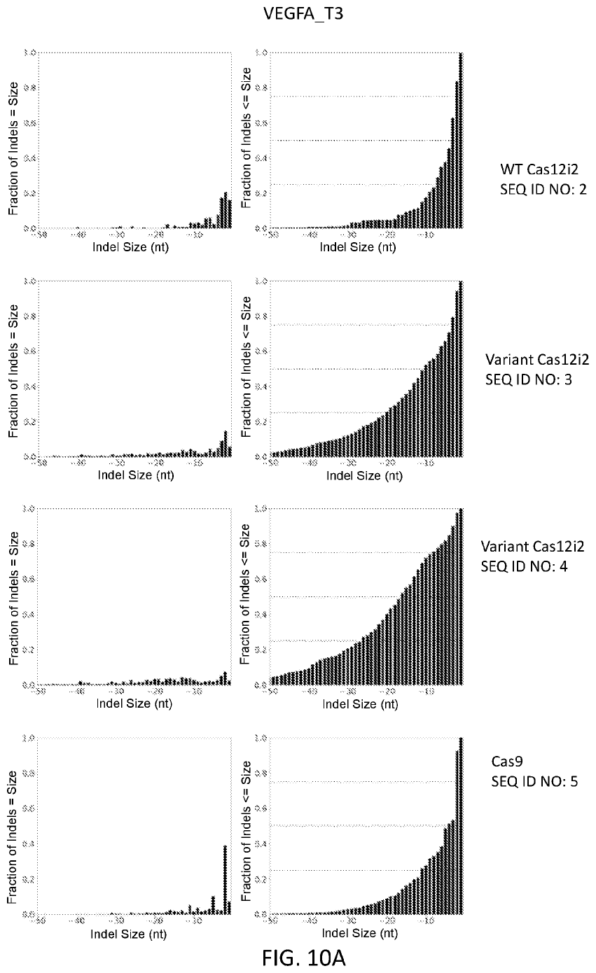

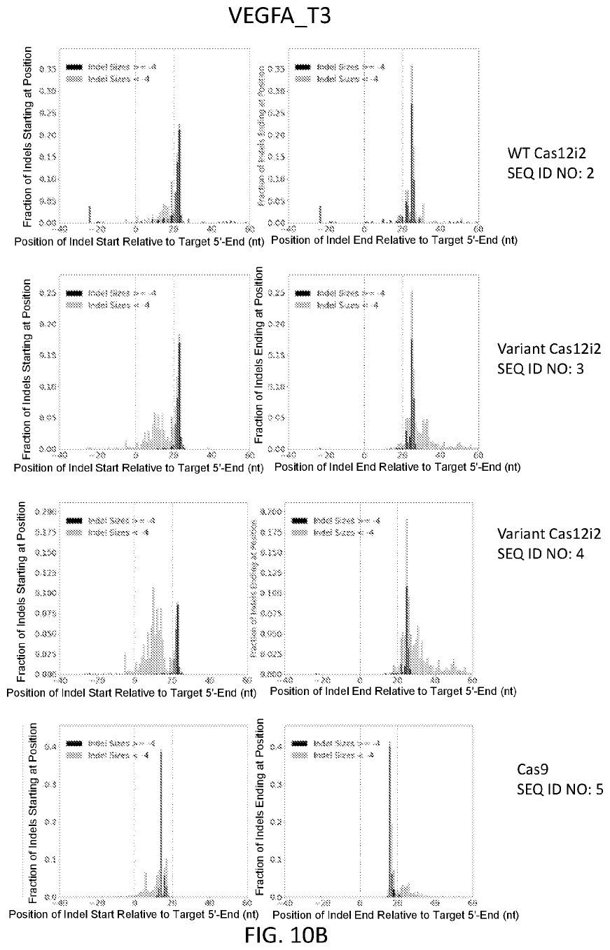

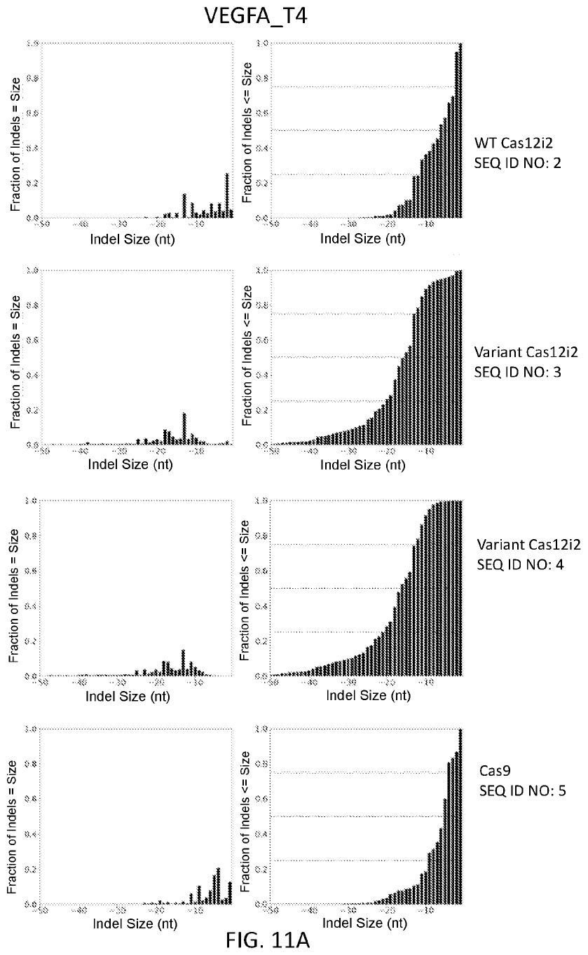

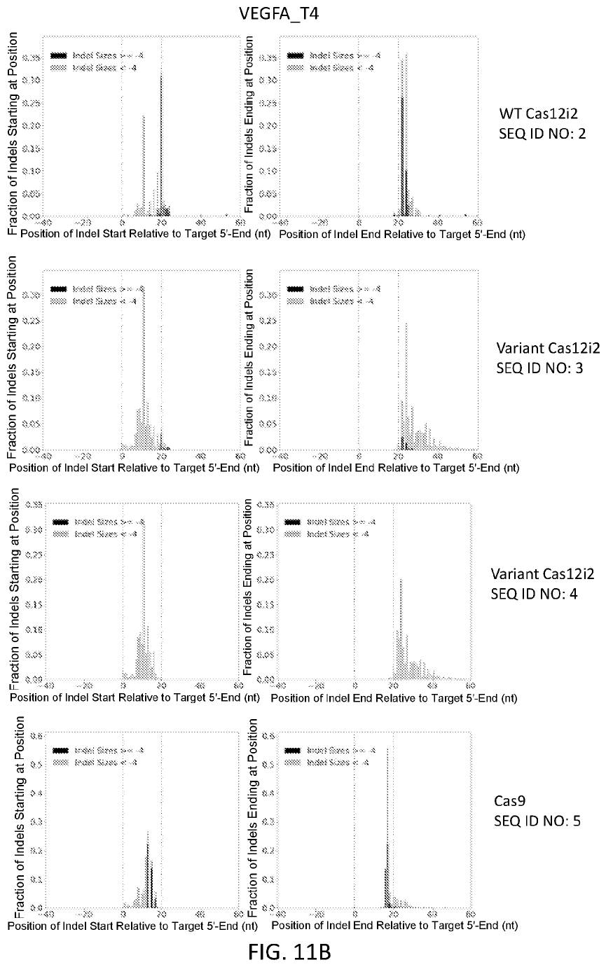

FIG. 2, FIG. 3, FIG. 4, and FIG. 5 show the sizes and positions of Cas12i2-induced and SpCas9-induced indels at AAVS 1 target loci. FIG. 6, FIG. 7, and FIG. 8 show the sizes and positions of Cas12i2-induced and SpCas9-induced indels at EMX1 target loci. FIG. 9, FIG. 10, and FIG. 11 show the sizes and 10 positions of Cas12i2-induced and SpCas9-induced Indels at VEGFA target loci. The sequences of each AAVS1, EMX1, and VEGFA target locus for Cas12i2 and SpCas9 are shown in FIG. 1.

As shown in the first column of FIGS. 2-11, wild-type and variant Cas12i2 induced larger deletions across multiple target loci, as compared to those induced by SpCas9. This is confirmed by Tables 2-7, which show fractions of indels>=−4 (deletions of 1-4 nucleotides in size) and fractions of indels<−4 (deletions of 5 nucleotides or larger in size). As shown in Table 7, cells edited by SpCas9 were characterized by a higher fraction of indels>=−4 at nine of the ten target loci tested, compared to cells edited by variant Cas12i2 of SEQ ID NO: 4. In agreement with this, as shown in Table 8, cells edited by variant Cas12i2 of SEQ ID NO:4 were characterized by a higher fraction of indels<−4 at nine of the ten target loci tested, as compared to cells edited by SpCas9. Furthermore, as shown in the second column of FIGS. 2-11 and Table 2, a broader distribution of indel sizes was observed for wild-type and variant Cas12i2, as compared to SpCas9.

As shown in the third and fourth columns of FIGS. 2-11, Cas12i2-induced indels>=−4 (deletions of 1-4 nucleotides in size), which are less frequent than longer deletions in Cas12i2-modified cells, tend to be positioned directly downstream of the target sequence (e.g., starting around position 20 and ending around position 25). Cas12i2-induced indels<−4 (deletions of 5 nucleotides or larger in size), which are more frequent than shorter deletions in Cas12i2-modified cells, tend to start within the target sequence (e.g., between position 5 and position 15) and ending downstream of the target sequence (e.g., between position 22 and 30). See, also, the mode indel start and end positions for wild-type and variant Cas12i2 in Table 3 and Table 4 and the mean indel start and end positions for wild-type and variant Cas12i2 in Table 5 and Table 6. Conversely, SpCas9-induced indels>=−4 (deletions of 1-4 nucleotides in size), which are more frequent than longer deletions in SpCas9-modified cells, tend to start and end within the target sequence. SpCas9-induced indels<−4 (deletions of 5 nucleotides or larger in size), which are less frequent than shorter deletions in SpCas9-modified cells, also tend to start and end within the target sequence or start within the target sequence and end directly downstream of the target sequence. See, also, the mode indel start and end positions for SpCas9 in Table 3 and Table 4 and the mean indel start and end positions for SpCas9 in Table 5 and Table 6.

As shown in FIG. 1, the target sequences for AAVS1_3 and VEGFA_1 are identical for Cas12i2 and SpCas9. Therefore, these two sequences can be used to directly compare the indel positions relative to the Cas12i2 PAM, 5‘-NTTN’3′ (e.g., 5‘-CTTT’3′). These positions are summarized in Table 9 below. As shown for AAVS1_T3, the Cas12i2-induced indels>=−4 were downstream of the target sequence and therefore further from the Cas12i2 PAM, compared to SpCas9-induced indels>=−4. Furthermore, Cas12i2-induced indels<−4 end further from the Cas12i2 PAM, compared to SpCas9-induced indels<−4.

TABLE 9
Mode Indel Start and End Positions for AAVS1_T3 and VEGFA_T1 Indels

| | | Mode Start and | Mode Start and | Mode Start and | Mode Start and |
|---|---|---|---|---|---|
| | | End Range | End Range | End Range | End Range |
| | | Variant Cas12i2 | SpCas9 (SEQ ID | Variant Cas12i2 | SpCas9 (SEQ ID |
| | | (SEQ ID NO: 4) | NO: 5) | (SEQ ID NO: 4) | NO: 5) |
| Gene | Locus | Indels >= −4 | Indels >= −4 | Indels < −4 | Indels < −4 |
| AAVS1 | T3 | 23 to 24 | 15 to 16 | 13 to 24 | 10 to 17 |
| VEGFA | T1 | 23 to 24 | 14 to 17 | 11 to 24 | 13 to 20 |

Therefore, this Example shows that cells modified by Cas12i2 (wild-type and variant) have characteristic indel sizes and positions that allow for Cas12i2-modified cells to be distinguished from SpCas9-modified cells.

### Example 2—Characterization of Insertions Induced by Cas12i2

This Example describes characterization of Cas12i2-induced insertions in mammalian cells.

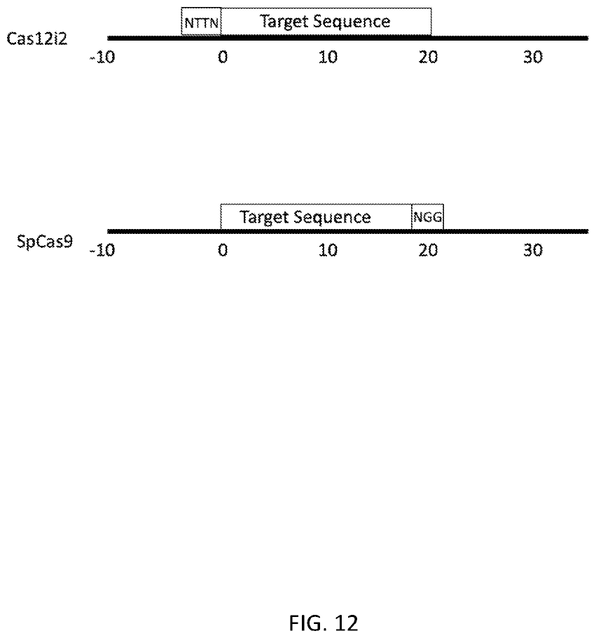

NGS samples from Example 1 were analyzed for insertions. For each insertion length, indel size frequencies among edited reads were calculated as the number of indel-containing reads of a certain insertion length divided by the number of reads containing indels. Sequencing reads are aligned to a genomic reference sequence to which provides size and positional information about the indel. Average insertion starting positions (relative to the target sequence programmed into a sample's guide RNA) were also calculated for insertions of up to 9 nucleotides in length. FIG. 12 shows how the insertion start positions in Tables 2-10 are measured. For the position of Cas12i2-induced insertions, the target sequence (e.g., the sequence to which the RNA guide hinds) spans from a position 1 through position 20; the position of the 5′-NTTN-3′ sequence is from position −4 to position −1. For the position of the SpCas9-induced indels, the target sequence (e.g., The sequence to which the RNA guide binds) spans from position 0 through position 19; the position of the 5′-NGG-3′ sequence is from position 19 to position 21. Tables 2-10 show the frequency and average start positions for 1-nucleotide, 2-nucleotide, 3-nucleotide, 4-nucleotide, 5-nucleotide, 6-nucleotide, 7-nucleotide, 8-nucleotide, and 9-nucleotide insertions induced by variant Cas12i2 of SEQ I3 NO: 4 or SpCas9 (SEQ ID NO: 5).

TABLE 10
Characterization of 1-nucleotide insertions.

| | | | | | Average |
|---|---|---|---|---|---|
| | | | | | Start |
| | | | Total | Frequency | position |
| | | Repli- | Indel | of 1- | of 1- |
| | | cate | Fre- | Nucleotide | Nucleotide |
| Target | Nuclease | No. | quency | Insertion | Insertion |
| AAVS1_T2 | Cas12i2 | 1 | 0.38 | 0.0016 | 25.65 |
| AAVS1_T2 | Cas12i2 | 2 | 0.32 | 0.0025 | 24.95 |
| AAVS1_T2 | Cas12i2 | 3 | 0.39 | 0.0026 | 24.73 |
| AAVS1_T2 | Cas12i2 | 4 | 0.32 | 0.0027 | 23.96 |
| AAVS1_T2 | SpCas9 | 1 | 0.54 | 0.1019 | 27.44 |
| AAVS1_T2 | SpCas9 | 2 | 0.53 | 0.1099 | 27.48 |
| AAVS1_T2 | SpCas9 | 3 | 0.44 | 0.1164 | 27.30 |
| AAVS1_T2 | SpCas9 | 4 | 0.49 | 0.1461 | 27.60 |
| AAVS1_T3 | Cas12i2 | 1 | 0.90 | 0.0035 | 24.62 |
| AAVS1_T3 | Cas12i2 | 2 | 0.90 | 0.0045 | 23.87 |
| AAVS1_T3 | Cas12i2 | 3 | 0.89 | 0.0061 | 25.07 |
| AAVS1_T3 | Cas12i2 | 4 | 0.89 | 0.0075 | 24.17 |
| AAVS1_T3 | SpCas9 | 1 | 0.88 | 0.0476 | 15.76 |
| AAVS1_T3 | SpCas9 | 2 | 0.90 | 0.0584 | 16.08 |
| AAVS1_T3 | SpCas9 | 3 | 0.91 | 0.1136 | 15.65 |
| AAVS1_T3 | SpCas9 | 4 | 0.91 | 0.1267 | 15.67 |
| AAVS1_T4 | Cas12i2 | 1 | 0.79 | 0.0013 | 30.63 |
| AAVS1_T4 | Cas12i2 | 2 | 0.68 | 0.0021 | 25.52 |
| AAVS1_T4 | Cas12i2 | 3 | 0.69 | 0.0022 | 24.82 |
| AAVS1_T4 | Cas12i2 | 4 | 0.78 | 0.0024 | 23.93 |
| AAVS1_T4 | SpCas9 | 1 | 0.97 | 0.3710 | 16.39 |
| AAVS1_T4 | SpCas9 | 2 | 0.97 | 0.3728 | 16.43 |
| AAVS1_T4 | SpCas9 | 3 | 0.94 | 0.4063 | 16.36 |
| AAVS1_T4 | SpCas9 | 4 | 0.95 | 0.4188 | 16.35 |
| AAVS1_T6 | Cas12i2 | 1 | 0.90 | 0.0010 | 30.46 |
| AAVS1_T6 | Cas12i2 | 2 | 0.90 | 0.0013 | 29.59 |
| AAVS1_T6 | Cas12i2 | 3 | 0.88 | 0.0015 | 23.00 |
| AAVS1_T6 | Cas12i2 | 4 | 0.85 | 0.0058 | 23.00 |
| AAVS1_T6 | SpCas9 | 1 | 0.79 | 0.0424 | 18.80 |
| AAVS1_T6 | SpCas9 | 2 | 0.69 | 0.0578 | 18.15 |
| AAVS1_T6 | SpCas9 | 3 | 0.72 | 0.0620 | 17.97 |
| AAVS1_T6 | SpCas9 | 4 | 0.81 | 0.0622 | 19.31 |
| EMX1_T3 | Cas12i2 | 1 | 0.59 | 0.0110 | 22.73 |
| EMX1_T3 | Cas12i2 | 2 | 0.58 | 0.0112 | 23.00 |
| EMX1_T3 | Cas12i2 | 3 | 0.66 | 0.0134 | 22.80 |
| EMX1_T3 | Cas12i2 | 4 | 0.65 | 0.0135 | 22.81 |
| EMX1_T3 | SpCas9 | 1 | 0.81 | 0.0239 | 16.91 |
| EMX1_T3 | SpCas9 | 2 | 0.85 | 0.0254 | 16.28 |
| EMX1_T3 | SpCas9 | 3 | 0.85 | 0.0255 | 16.76 |
| EMX1_T3 | SpCas9 | 4 | 0.81 | 0.0276 | 16.59 |
| EMX1_T4 | Cas12i2 | 1 | 0.52 | 0.0048 | 24.57 |
| EMX1_T4 | Cas12i2 | 2 | 0.52 | 0.0050 | 27.06 |
| EMX1_T4 | Cas12i2 | 3 | 0.77 | 0.0053 | 24.05 |
| EMX1_T4 | Cas12i2 | 4 | 0.77 | 0.0054 | 26.48 |
| EMX1_T4 | SpCas9 | 1 | 0.98 | 0.1839 | 15.41 |
| EMX1_T4 | SpCas9 | 2 | 0.98 | 0.1847 | 15.45 |
| EMX1_T4 | SpCas9 | 3 | 0.98 | 0.1851 | 15.57 |
| EMX1_T4 | SpCas9 | 4 | 0.98 | 0.1931 | 15.42 |
| EMX1_T5 | Cas12i2 | 1 | 0.67 | 0.0101 | 22.15 |
| EMX1_T5 | Cas12i2 | 2 | 0.74 | 0.0106 | 21.92 |
| EMX1_T5 | Cas12i2 | 3 | 0.67 | 0.0107 | 21.54 |
| EMX1_T5 | SpCas9 | 1 | 0.57 | 0.0324 | 17.72 |
| EMX1_T5 | SpCas9 | 2 | 0.57 | 0.0349 | 17.72 |
| EMX1_T5 | SpCas9 | 3 | 0.58 | 0.0366 | 17.70 |
| EMX1_T5 | SpCas9 | 4 | 0.58 | 0.0388 | 17.52 |
| VEGFA_T1 | Cas12i2 | 1 | 0.84 | 0.0044 | 22.81 |
| VEGFA_T1 | Cas12i2 | 2 | 0.83 | 0.0077 | 23.42 |
| VEGFA_T1 | Cas12i2 | 3 | 0.82 | 0.0080 | 23.74 |
| VEGFA_T1 | Cas12i2 | 4 | 0.84 | 0.0082 | 23.55 |
| VEGFA_T1 | SpCas9 | 1 | 0.17 | 0.0377 | 16.13 |
| VEGFA_T1 | SpCas9 | 2 | 0.17 | 0.0427 | 16.50 |
| VEGFA_T1 | SpCas9 | 3 | 0.16 | 0.0616 | 16.43 |
| VEGFA_T1 | SpCas9 | 4 | 0.17 | 0.0750 | 16.43 |
| VEGFA_T3 | Cas12i2 | 1 | 0.77 | 0.0024 | 24.03 |
| VEGFA_T3 | Cas12i2 | 2 | 0.78 | 0.0027 | 24.62 |
| VEGFA_T3 | Cas12i2 | 3 | 0.79 | 0.0029 | 25.86 |
| VEGFA_T3 | Cas12i2 | 4 | 0.78 | 0.0036 | 21.90 |
| VEGFA_T3 | SpCas9 | 1 | 0.94 | 0.2231 | 16.83 |
| VEGFA_T3 | SpCas9 | 2 | 0.93 | 0.2309 | 16.85 |
| VEGFA_T3 | SpCas9 | 3 | 0.94 | 0.2314 | 16.78 |
| VEGFA_T3 | SpCas9 | 4 | 0.93 | 0.2338 | 16.87 |
| VEGFA_T4 | Cas12i2 | 1 | 0.98 | 0.0001 | 61.00 |
| VEGFA_T4 | Cas12i2 | 2 | 0.99 | 0.0006 | 30.22 |
| VEGFA_T4 | Cas12i2 | 3 | 0.99 | 0.0008 | 29.98 |
| VEGFA_T4 | Cas12i2 | 4 | 0.98 | 0.0009 | 31.71 |
| VEGFA_T4 | SpCas9 | 1 | 0.97 | 0.0253 | 16.81 |
| VEGFA_T4 | SpCas9 | 2 | 0.98 | 0.0266 | 16.68 |
| VEGFA_T4 | SpCas9 | 3 | 0.97 | 0.0334 | 16.77 |
| VEGFA_T4 | SpCas9 | 4 | 0.97 | 0.0381 | 16.91 |

TABLE 11
Characterization of 2-nucleotide insertions.

| | | | | | Average Start |
|---|---|---|---|---|---|
| | | | | Frequency of | position of 2- |
| | | | Total Indel | 2-Nucleotide | Nucleotide |
| Target | Nuclease | Replicate No. | Frequency | Insertion | Insertion |
| AAVS1_T2 | Cas12i2 | 1 | 0.38 | 0.0017 | 26.22 |
| AAVS1_T2 | Cas12i2 | 2 | 0.32 | 0.0010 | 27.69 |
| AAVS1_T2 | Cas12i2 | 3 | 0.39 | 0.0009 | 27.51 |
| AAVS1_T2 | Cas12i2 | 4 | 0.32 | 0.0012 | 26.00 |
| AAVS1_T2 | SpCas9 | 1 | 0.54 | 0.0163 | 28.92 |
| AAVS1_T2 | SpCas9 | 2 | 0.53 | 0.0196 | 29.95 |
| AAVS1_T2 | SpCas9 | 3 | 0.44 | 0.0232 | 30.55 |
| AAVS1_T2 | SpCas9 | 4 | 0.49 | 0.0289 | 30.46 |
| AAVS1_T3 | Cas12i2 | 1 | 0.90 | 0.0007 | 25.67 |
| AAVS1_T3 | Cas12i2 | 2 | 0.90 | 0.0015 | 24.91 |
| AAVS1_T3 | Cas12i2 | 3 | 0.89 | 0.0018 | 24.77 |
| AAVS1_T3 | Cas12i2 | 4 | 0.89 | 0.0018 | 26.73 |
| AAVS1_T3 | SpCas9 | 1 | 0.88 | 0.0676 | 16.06 |
| AAVS1_T3 | SpCas9 | 2 | 0.90 | 0.0698 | 16.14 |
| AAVS1_T3 | SpCas9 | 3 | 0.91 | 0.0734 | 16.11 |
| AAVS1_T3 | SpCas9 | 4 | 0.91 | 0.0791 | 16.04 |
| AAVS1_T4 | Cas12i2 | 1 | 0.79 | 0.0016 | 26.41 |
| AAVS1_T4 | Cas12i2 | 2 | 0.68 | 0.0010 | 31.56 |
| AAVS1_T4 | Cas12i2 | 3 | 0.69 | 0.0009 | 25.95 |
| AAVS1_T4 | Cas12i2 | 4 | 0.78 | 0.0000 | 0.00 |
| AAVS1_T4 | SpCas9 | 1 | 0.97 | 0.0248 | 16.04 |
| AAVS1_T4 | SpCas9 | 2 | 0.97 | 0.0263 | 16.54 |
| AAVS1_T4 | SpCas9 | 3 | 0.94 | 0.0216 | 16.27 |
| AAVS1_T4 | SpCas9 | 4 | 0.95 | 0.0191 | 16.10 |
| AAVS1_T6 | Cas12i2 | 1 | 0.90 | 0.0009 | 31.00 |
| AAVS1_T6 | Cas12i2 | 2 | 0.90 | 0.0006 | 28.17 |
| AAVS1_T6 | Cas12i2 | 3 | 0.88 | 0.0015 | 22.00 |
| AAVS1_T6 | Cas12i2 | 4 | 0.85 | 0.0000 | 0.00 |
| AAVS1_T6 | SpCas9 | 1 | 0.79 | 0.0198 | 18.57 |
| AAVS1_T6 | SpCas9 | 2 | 0.69 | 0.0154 | 18.45 |
| AAVS1_T6 | SpCas9 | 3 | 0.72 | 0.0147 | 18.55 |
| AAVS1_T6 | SpCas9 | 4 | 0.81 | 0.0175 | 17.27 |
| EMX1_T3 | Cas12i2 | 1 | 0.59 | 0.0017 | 23.29 |
| EMX1_T3 | Cas12i2 | 2 | 0.58 | 0.0015 | 24.14 |
| EMX1_T3 | Cas12i2 | 3 | 0.66 | 0.0029 | 23.23 |
| EMX1_T3 | Cas12i2 | 4 | 0.65 | 0.0033 | 22.88 |
| EMX1_T3 | SpCas9 | 1 | 0.81 | 0.0071 | 17.54 |
| EMX1_T3 | SpCas9 | 2 | 0.85 | 0.0066 | 17.59 |
| EMX1_T3 | SpCas9 | 3 | 0.85 | 0.0098 | 18.00 |
| EMX1_T3 | SpCas9 | 4 | 0.81 | 0.0081 | 17.53 |
| EMX1_T4 | Cas12i2 | 1 | 0.52 | 0.0021 | 23.63 |
| EMX1_T4 | Cas12i2 | 2 | 0.52 | 0.0008 | 20.63 |
| EMX1_T4 | Cas12i2 | 3 | 0.77 | 0.0013 | 28.73 |
| EMX1_T4 | Cas12i2 | 4 | 0.77 | 0.0014 | 26.10 |
| EMX1_T4 | SpCas9 | 1 | 0.98 | 0.0162 | 16.47 |
| EMX1_T4 | SpCas9 | 2 | 0.98 | 0.0120 | 17.06 |
| EMX1_T4 | SpCas9 | 3 | 0.98 | 0.0119 | 16.69 |
| EMX1_T4 | SpCas9 | 4 | 0.98 | 0.0098 | 16.52 |
| EMX1_T5 | Cas12i2 | 1 | 0.67 | 0.0029 | 22.71 |
| EMX1_T5 | Cas12i2 | 2 | 0.74 | 0.0028 | 24.14 |
| EMX1_T5 | Cas12i2 | 3 | 0.67 | 0.0032 | 23.15 |
| EMX1_T5 | SpCas9 | 1 | 0.57 | 0.0042 | 17.44 |
| EMX1_T5 | SpCas9 | 2 | 0.57 | 0.0028 | 23.72 |
| EMX1_T5 | SpCas9 | 3 | 0.58 | 0.0063 | 18.47 |
| EMX1_T5 | SpCas9 | 4 | 0.58 | 0.0063 | 18.71 |
| VEGFA_T1 | Cas12i2 | 1 | 0.84 | 0.0025 | 21.75 |
| VEGFA_T1 | Cas12i2 | 2 | 0.83 | 0.0020 | 24.74 |
| VEGFA_T1 | Cas12i2 | 3 | 0.82 | 0.0026 | 25.21 |
| VEGFA_T1 | Cas12i2 | 4 | 0.84 | 0.0025 | 24.62 |
| VEGFA_T1 | SpCas9 | 1 | 0.17 | 0.0020 | 19.67 |
| VEGFA_T1 | SpCas9 | 2 | 0.17 | 0.0012 | 17.14 |
| VEGFA_T1 | SpCas9 | 3 | 0.16 | 0.0027 | 16.94 |
| VEGFA_T1 | SpCas9 | 4 | 0.17 | 0.0022 | 17.30 |
| VEGFA_T3 | Cas12i2 | 1 | 0.77 | 0.0022 | 23.13 |
| VEGFA_T3 | Cas12i2 | 2 | 0.78 | 0.0020 | 26.67 |
| VEGFA_T3 | Cas12i2 | 3 | 0.79 | 0.0012 | 24.91 |
| VEGFA_T3 | Cas12i2 | 4 | 0.78 | 0.0020 | 24.92 |
| VEGFA_T3 | SpCas9 | 1 | 0.94 | 0.0374 | 16.00 |
| VEGFA_T3 | SpCas9 | 2 | 0.93 | 0.0437 | 16.13 |
| VEGFA_T3 | SpCas9 | 3 | 0.94 | 0.0382 | 16.05 |
| VEGFA_T3 | SpCas9 | 4 | 0.93 | 0.0383 | 16.06 |
| VEGFA_T4 | Cas12i2 | 1 | 0.98 | 0.0000 | 28.50 |
| VEGFA_T4 | Cas12i2 | 2 | 0.99 | 0.0005 | 31.52 |
| VEGFA_T4 | Cas12i2 | 3 | 0.99 | 0.0008 | 28.48 |
| VEGFA_T4 | Cas12i2 | 4 | 0.98 | 0.0004 | 33.27 |
| VEGFA_T4 | SpCas9 | 1 | 0.97 | 0.0054 | 16.78 |
| VEGFA_T4 | SpCas9 | 2 | 0.98 | 0.0068 | 19.35 |
| VEGFA_T4 | SpCas9 | 3 | 0.97 | 0.0079 | 17.02 |
| VEGFA_T4 | SpCas9 | 4 | 0.97 | 0.0078 | 17.71 |

TABLE 12
Characterization of 3-nucleotide insertions.

| | | | | | Average Start |
|---|---|---|---|---|---|
| | | | | Frequency of | position of 3- |
| | | | Total Indel | 3-Nucleotide | Nucleotide |
| Target | Nuclease | Replicate No. | Frequency | Insertion | Insertion |
| AAVS1_T2 | Cas12i2 | 1 | 0.38 | 0.0003 | 26.47 |
| AAVS1_T2 | Cas12i2 | 2 | 0.32 | 0.0006 | 25.86 |
| AAVS1_T2 | Cas12i2 | 3 | 0.39 | 0.0008 | 22.95 |
| AAVS1_T2 | Cas12i2 | 4 | 0.32 | 0.0005 | 24.08 |
| AAVS1_T2 | SpCas9 | 1 | 0.54 | 0.0035 | 24.19 |
| AAVS1_T2 | SpCas9 | 2 | 0.53 | 0.0048 | 23.18 |
| AAVS1_T2 | SpCas9 | 3 | 0.44 | 0.0016 | 23.73 |
| AAVS1_T2 | SpCas9 | 4 | 0.49 | 0.0031 | 24.93 |
| AAVS1_T3 | Cas12i2 | 1 | 0.90 | 0.0032 | 25.83 |
| AAVS1_T3 | Cas12i2 | 2 | 0.90 | 0.0027 | 26.94 |
| AAVS1_T3 | Cas12i2 | 3 | 0.89 | 0.0010 | 19.85 |
| AAVS1_T3 | Cas12i2 | 4 | 0.89 | 0.0011 | 29.26 |
| AAVS1_T3 | SpCas9 | 1 | 0.88 | 0.0248 | 16.42 |
| AAVS1_T3 | SpCas9 | 2 | 0.90 | 0.0292 | 15.40 |
| AAVS1_T3 | SpCas9 | 3 | 0.91 | 0.0282 | 16.42 |
| AAVS1_T3 | SpCas9 | 4 | 0.91 | 0.0354 | 16.13 |
| AAVS1_T4 | Cas12i2 | 1 | 0.79 | 0.0006 | 28.00 |
| AAVS1_T4 | Cas12i2 | 2 | 0.68 | 0.0008 | 29.05 |
| AAVS1_T4 | Cas12i2 | 3 | 0.69 | 0.0005 | 27.78 |
| AAVS1_T4 | Cas12i2 | 4 | 0.78 | 0.0008 | 27.19 |
| AAVS1_T4 | SpCas9 | 1 | 0.97 | 0.0122 | 15.65 |
| AAVS1_T4 | SpCas9 | 2 | 0.97 | 0.0112 | 16.74 |
| AAVS1_T4 | SpCas9 | 3 | 0.94 | 0.0095 | 16.11 |
| AAVS1_T4 | SpCas9 | 4 | 0.95 | 0.0087 | 15.27 |
| AAVS1_T6 | Cas12i2 | 1 | 0.90 | 0.0008 | 32.57 |
| AAVS1_T6 | Cas12i2 | 2 | 0.90 | 0.0012 | 29.56 |
| AAVS1_T6 | Cas12i2 | 3 | 0.88 | 0.0000 | 0.00 |
| AAVS1_T6 | Cas12i2 | 4 | 0.85 | 0.0000 | 0.00 |
| AAVS1_T6 | SpCas9 | 1 | 0.79 | 0.0141 | 16.80 |
| AAVS1_T6 | SpCas9 | 2 | 0.69 | 0.0061 | 19.05 |
| AAVS1_T6 | SpCas9 | 3 | 0.72 | 0.0060 | 20.31 |
| AAVS1_T6 | SpCas9 | 4 | 0.81 | 0.0064 | 18.00 |
| EMX1_T3 | Cas12i2 | 1 | 0.59 | 0.0013 | 23.26 |
| EMX1_T3 | Cas12i2 | 2 | 0.58 | 0.0011 | 23.35 |
| EMX1_T3 | Cas12i2 | 3 | 0.66 | 0.0020 | 21.39 |
| EMX1_T3 | Cas12i2 | 4 | 0.65 | 0.0012 | 24.10 |
| EMX1_T3 | SpCas9 | 1 | 0.81 | 0.0043 | 17.65 |
| EMX1_T3 | SpCas9 | 2 | 0.85 | 0.0044 | 17.63 |
| EMX1_T3 | SpCas9 | 3 | 0.85 | 0.0043 | 19.42 |
| EMX1_T3 | SpCas9 | 4 | 0.81 | 0.0044 | 18.50 |
| EMX1_T4 | Cas12i2 | 1 | 0.52 | 0.0011 | 27.72 |
| EMX1_T4 | Cas12i2 | 2 | 0.52 | 0.0006 | 24.00 |
| EMX1_T4 | Cas12i2 | 3 | 0.77 | 0.0013 | 25.11 |
| EMX1_T4 | Cas12i2 | 4 | 0.77 | 0.0010 | 25.55 |
| EMX1_T4 | SpCas9 | 1 | 0.98 | 0.0020 | 17.99 |
| EMX1_T4 | SpCas9 | 2 | 0.98 | 0.0047 | 17.11 |
| EMX1_T4 | SpCas9 | 3 | 0.98 | 0.0020 | 17.27 |
| EMX1_T4 | SpCas9 | 4 | 0.98 | 0.0023 | 16.96 |
| EMX1_T5 | Cas12i2 | 1 | 0.67 | 0.0038 | 23.40 |
| EMX1_T5 | Cas12i2 | 2 | 0.74 | 0.0032 | 23.78 |
| EMX1_T5 | Cas12i2 | 3 | 0.67 | 0.0036 | 22.20 |
| EMX1_T5 | SpCas9 | 1 | 0.57 | 0.0021 | 20.36 |
| EMX1_T5 | SpCas9 | 2 | 0.57 | 0.0022 | 22.06 |
| EMX1_T5 | SpCas9 | 3 | 0.58 | 0.0020 | 24.88 |
| EMX1_T5 | SpCas9 | 4 | 0.58 | 0.0016 | 20.17 |
| VEGFA_T1 | Cas12i2 | 1 | 0.84 | 0.0006 | 22.80 |
| VEGFA_T1 | Cas12i2 | 2 | 0.83 | 0.0013 | 26.48 |
| VEGFA_T1 | Cas12i2 | 3 | 0.82 | 0.0011 | 20.41 |
| VEGFA_T1 | Cas12i2 | 4 | 0.84 | 0.0016 | 25.27 |
| VEGFA_T1 | SpCas9 | 1 | 0.17 | 0.0003 | 17.00 |
| VEGFA_T1 | SpCas9 | 2 | 0.17 | 0.0013 | 17.67 |
| VEGFA_T1 | SpCas9 | 3 | 0.16 | 0.0014 | 17.24 |
| VEGFA_T1 | SpCas9 | 4 | 0.17 | 0.0013 | 17.52 |
| VEGFA_T3 | Cas12i2 | 1 | 0.77 | 0.0023 | 28.26 |
| VEGFA_T3 | Cas12i2 | 2 | 0.78 | 0.0012 | 26.48 |
| VEGFA_T3 | Cas12i2 | 3 | 0.79 | 0.0014 | 23.84 |
| VEGFA_T3 | Cas12i2 | 4 | 0.78 | 0.0013 | 22.75 |
| VEGFA_T3 | SpCas9 | 1 | 0.94 | 0.0118 | 17.47 |
| VEGFA_T3 | SpCas9 | 2 | 0.93 | 0.0123 | 17.06 |
| VEGFA_T3 | SpCas9 | 3 | 0.94 | 0.0119 | 17.07 |
| VEGFA_T3 | SpCas9 | 4 | 0.93 | 0.0135 | 16.88 |
| VEGFA_T4 | Cas12i2 | 1 | 0.98 | 0.0001 | 33.00 |
| VEGFA_T4 | Cas12i2 | 2 | 0.99 | 0.0005 | 27.92 |
| VEGFA_T4 | Cas12i2 | 3 | 0.99 | 0.0008 | 31.45 |
| VEGFA_T4 | Cas12i2 | 4 | 0.98 | 0.0000 | 0.00 |
| VEGFA_T4 | SpCas9 | 1 | 0.97 | 0.0046 | 17.38 |
| VEGFA_T4 | SpCas9 | 2 | 0.98 | 0.0018 | 17.67 |
| VEGFA_T4 | SpCas9 | 3 | 0.97 | 0.0027 | 17.38 |
| VEGFA_T4 | SpCas9 | 4 | 0.97 | 0.0041 | 21.78 |

TABLE 13
Characterization of 4-nucleotide insertions.

| | | | | | Average Start |
|---|---|---|---|---|---|
| | | | | Frequency of | position of 4- |
| | | | Total Indel | 4-Nucleotide | Nucleotide |
| Target | Nuclease | Replicate No. | Frequency | Insertion | Insertion |
| AAVS1_T2 | Cas12i2 | 1 | 0.38 | 0.0004 | 24.55 |
| AAVS1_T2 | Cas12i2 | 2 | 0.32 | 0.0002 | 24.33 |
| AAVS1_T2 | Cas12i2 | 3 | 0.39 | 0.0009 | 23.89 |
| AAVS1_T2 | Cas12i2 | 4 | 0.32 | 0.0005 | 25.54 |
| AAVS1_T2 | SpCas9 | 1 | 0.54 | 0.0024 | 22.44 |
| AAVS1_T2 | SpCas9 | 2 | 0.53 | 0.0024 | 20.75 |
| AAVS1_T2 | SpCas9 | 3 | 0.44 | 0.0036 | 21.53 |
| AAVS1_T2 | SpCas9 | 4 | 0.49 | 0.0022 | 22.52 |
| AAVS1_T3 | Cas12i2 | 1 | 0.90 | 0.0011 | 35.26 |
| AAVS1_T3 | Cas12i2 | 2 | 0.90 | 0.0006 | 31.25 |
| AAVS1_T3 | Cas12i2 | 3 | 0.89 | 0.0010 | 27.47 |
| AAVS1_T3 | Cas12i2 | 4 | 0.89 | 0.0017 | 26.69 |
| AAVS1_T3 | SpCas9 | 1 | 0.88 | 0.0118 | 15.81 |
| AAVS1_T3 | SpCas9 | 2 | 0.90 | 0.0055 | 16.68 |
| AAVS1_T3 | SpCas9 | 3 | 0.91 | 0.0093 | 17.84 |
| AAVS1_T3 | SpCas9 | 4 | 0.91 | 0.0106 | 16.83 |
| AAVS1_T4 | Cas12i2 | 1 | 0.79 | 0.0002 | 20.60 |
| AAVS1_T4 | Cas12i2 | 2 | 0.68 | 0.0006 | 28.75 |
| AAVS1_T4 | Cas12i2 | 3 | 0.69 | 0.0003 | 24.14 |
| AAVS1_T4 | Cas12i2 | 4 | 0.78 | 0.0003 | 27.82 |
| AAVS1_T4 | SpCas9 | 1 | 0.97 | 0.0030 | 16.40 |
| AAVS1_T4 | SpCas9 | 2 | 0.97 | 0.0040 | 15.36 |
| AAVS1_T4 | SpCas9 | 3 | 0.94 | 0.0033 | 17.71 |
| AAVS1_T4 | SpCas9 | 4 | 0.95 | 0.0036 | 17.44 |
| AAVS1_T6 | Cas12i2 | 1 | 0.90 | 0.0005 | 30.96 |
| AAVS1_T6 | Cas12i2 | 2 | 0.90 | 0.0004 | 31.55 |
| AAVS1_T6 | Cas12i2 | 3 | 0.88 | 0.0015 | 36.00 |
| AAVS1_T6 | Cas12i2 | 4 | 0.85 | 0.0000 | 0.00 |
| AAVS1_T6 | SpCas9 | 1 | 0.79 | 0.0000 | 0.00 |
| AAVS1_T6 | SpCas9 | 2 | 0.69 | 0.0047 | 20.28 |
| AAVS1_T6 | SpCas9 | 3 | 0.72 | 0.0031 | 19.50 |
| AAVS1_T6 | SpCas9 | 4 | 0.81 | 0.0064 | 17.00 |
| EMX1_T3 | Cas12i2 | 1 | 0.59 | 0.0011 | 23.44 |
| EMX1_T3 | Cas12i2 | 2 | 0.58 | 0.0007 | 18.61 |
| EMX1_T3 | Cas12i2 | 3 | 0.66 | 0.0015 | 26.00 |
| EMX1_T3 | Cas12i2 | 4 | 0.65 | 0.0011 | 23.58 |
| EMX1_T3 | SpCas9 | 1 | 0.81 | 0.0037 | 17.25 |
| EMX1_T3 | SpCas9 | 2 | 0.85 | 0.0031 | 18.25 |
| EMX1_T3 | SpCas9 | 3 | 0.85 | 0.0060 | 17.92 |
| EMX1_T3 | SpCas9 | 4 | 0.81 | 0.0033 | 17.47 |
| EMX1_T4 | Cas12i2 | 1 | 0.52 | 0.0002 | 26.67 |
| EMX1_T4 | Cas12i2 | 2 | 0.52 | 0.0001 | 22.00 |
| EMX1_T4 | Cas12i2 | 3 | 0.77 | 0.0013 | 27.27 |
| EMX1_T4 | Cas12i2 | 4 | 0.77 | 0.0008 | 26.45 |
| EMX1_T4 | SpCas9 | 1 | 0.98 | 0.0029 | 16.24 |
| EMX1_T4 | SpCas9 | 2 | 0.98 | 0.0026 | 19.10 |
| EMX1_T4 | SpCas9 | 3 | 0.98 | 0.0020 | 18.28 |
| EMX1_T4 | SpCas9 | 4 | 0.98 | 0.0021 | 16.70 |
| EMX1_T5 | Cas12i2 | 1 | 0.67 | 0.0005 | 22.48 |
| EMX1_T5 | Cas12i2 | 2 | 0.74 | 0.0014 | 19.66 |
| EMX1_T5 | Cas12i2 | 3 | 0.67 | 0.0021 | 23.69 |
| EMX1_T5 | SpCas9 | 1 | 0.57 | 0.0003 | 15.87 |
| EMX1_T5 | SpCas9 | 2 | 0.57 | 0.0003 | 16.29 |
| EMX1_T5 | SpCas9 | 3 | 0.58 | 0.0010 | 19.96 |
| EMX1_T5 | SpCas9 | 4 | 0.58 | 0.0017 | 21.43 |
| VEGFA_T1 | Cas12i2 | 1 | 0.84 | 0.0015 | 60.50 |
| VEGFA_T1 | Cas12i2 | 2 | 0.83 | 0.0012 | 58.98 |
| VEGFA_T1 | Cas12i2 | 3 | 0.82 | 0.0020 | 41.57 |
| VEGFA_T1 | Cas12i2 | 4 | 0.84 | 0.0015 | 45.39 |
| VEGFA_T1 | SpCas9 | 1 | 0.17 | 0.0012 | 62.57 |
| VEGFA_T1 | SpCas9 | 2 | 0.17 | 0.0017 | 39.79 |
| VEGFA_T1 | SpCas9 | 3 | 0.16 | 0.0018 | 51.93 |
| VEGFA_T1 | SpCas9 | 4 | 0.17 | 0.0017 | 45.17 |
| VEGFA_T3 | Cas12i2 | 1 | 0.77 | 0.0012 | 23.88 |
| VEGFA_T3 | Cas12i2 | 2 | 0.78 | 0.0012 | 24.40 |
| VEGFA_T3 | Cas12i2 | 3 | 0.79 | 0.0015 | 26.05 |
| VEGFA_T3 | Cas12i2 | 4 | 0.78 | 0.0013 | 25.03 |
| VEGFA_T3 | SpCas9 | 1 | 0.94 | 0.0050 | 18.22 |
| VEGFA_T3 | SpCas9 | 2 | 0.93 | 0.0061 | 18.03 |
| VEGFA_T3 | SpCas9 | 3 | 0.94 | 0.0036 | 17.29 |
| VEGFA_T3 | SpCas9 | 4 | 0.93 | 0.0060 | 17.77 |
| VEGFA_T4 | Cas12i2 | 1 | 0.98 | 0.0001 | 25.60 |
| VEGFA_T4 | Cas12i2 | 2 | 0.99 | 0.0005 | 26.79 |
| VEGFA_T4 | Cas12i2 | 3 | 0.99 | 0.0003 | 32.00 |
| VEGFA_T4 | Cas12i2 | 4 | 0.98 | 0.0007 | 30.39 |
| VEGFA_T4 | SpCas9 | 1 | 0.97 | 0.0020 | 17.68 |
| VEGFA_T4 | SpCas9 | 2 | 0.98 | 0.0028 | 17.04 |
| VEGFA_T4 | SpCas9 | 3 | 0.97 | 0.0038 | 18.08 |
| VEGFA_T4 | SpCas9 | 4 | 0.97 | 0.0034 | 17.11 |

TABLE 14
Characterization of 5-nucleotide insertions.

| | | | | | Average Start |
|---|---|---|---|---|---|
| | | | | Frequency of | position of 5- |
| | | | Total Indel | 5-Nucleotide | Nucleotide |
| Target | Nuclease | Replicate No. | Frequency | Insertion | Insertion |
| AAVS1_T2 | Cas12i2 | 1 | 0.38 | 0.00016 | 22.00 |
| AAVS1_T2 | Cas12i2 | 2 | 0.32 | 0.00036 | 26.17 |
| AAVS1_T2 | Cas12i2 | 3 | 0.39 | 0.00024 | 25.67 |
| AAVS1_T2 | Cas12i2 | 4 | 0.32 | 0.00030 | 19.93 |
| AAVS1_T2 | SpCas9 | 1 | 0.54 | 0.00206 | 21.83 |
| AAVS1_T2 | SpCas9 | 2 | 0.53 | 0.00268 | 21.89 |
| AAVS1_T2 | SpCas9 | 3 | 0.44 | 0.00168 | 22.62 |
| AAVS1_T2 | SpCas9 | 4 | 0.49 | 0.00156 | 20.63 |
| AAVS1_T3 | Cas12i2 | 1 | 0.90 | 0.00114 | 27.58 |
| AAVS1_T3 | Cas12i2 | 2 | 0.90 | 0.00123 | 35.95 |
| AAVS1_T3 | Cas12i2 | 3 | 0.89 | 0.00050 | 28.92 |
| AAVS1_T3 | Cas12i2 | 4 | 0.89 | 0.00062 | 27.81 |
| AAVS1_T3 | SpCas9 | 1 | 0.88 | 0.00191 | 16.44 |
| AAVS1_T3 | SpCas9 | 2 | 0.90 | 0.00389 | 16.52 |
| AAVS1_T3 | SpCas9 | 3 | 0.91 | 0.00266 | 18.55 |
| AAVS1_T3 | SpCas9 | 4 | 0.91 | 0.00386 | 19.30 |
| AAVS1_T4 | Cas12i2 | 1 | 0.79 | 0.00004 | 85.00 |
| AAVS1_T4 | Cas12i2 | 2 | 0.68 | 0.00072 | 43.42 |
| AAVS1_T4 | Cas12i2 | 3 | 0.69 | 0.00018 | 22.00 |
| AAVS1_T4 | Cas12i2 | 4 | 0.78 | 0.00012 | 32.67 |
| AAVS1_T4 | SpCas9 | 1 | 0.97 | 0.00088 | 16.61 |
| AAVS1_T4 | SpCas9 | 2 | 0.97 | 0.00346 | 17.97 |
| AAVS1_T4 | SpCas9 | 3 | 0.94 | 0.00254 | 18.19 |
| AAVS1_T4 | SpCas9 | 4 | 0.95 | 0.00212 | 16.25 |
| AAVS1_T6 | Cas12i2 | 1 | 0.90 | 0.00046 | 34.57 |
| AAVS1_T6 | Cas12i2 | 2 | 0.90 | 0.00044 | 29.05 |
| AAVS1_T6 | Cas12i2 | 3 | 0.88 | 0.00000 | 0.00 |
| AAVS1_T6 | Cas12i2 | 4 | 0.85 | 0.00000 | 0.00 |
| AAVS1_T6 | SpCas9 | 1 | 0.79 | 0.00282 | 16.00 |
| AAVS1_T6 | SpCas9 | 2 | 0.69 | 0.00249 | 22.88 |
| AAVS1_T6 | SpCas9 | 3 | 0.72 | 0.00354 | 20.77 |
| AAVS1_T6 | SpCas9 | 4 | 0.81 | 0.00000 | 0.00 |
| EMX1_T3 | Cas12i2 | 1 | 0.59 | 0.00088 | 22.36 |
| EMX1_T3 | Cas12i2 | 2 | 0.58 | 0.00014 | 30.00 |
| EMX1_T3 | Cas12i2 | 3 | 0.66 | 0.00102 | 23.27 |
| EMX1_T3 | Cas12i2 | 4 | 0.65 | 0.00106 | 23.64 |
| EMX1_T3 | SpCas9 | 1 | 0.81 | 0.00238 | 18.08 |
| EMX1_T3 | SpCas9 | 2 | 0.85 | 0.00368 | 18.14 |
| EMX1_T3 | SpCas9 | 3 | 0.85 | 0.00486 | 19.00 |
| EMX1_T3 | SpCas9 | 4 | 0.81 | 0.00288 | 20.06 |
| EMX1_T4 | Cas12i2 | 1 | 0.52 | 0.00000 | 0.00 |
| EMX1_T4 | Cas12i2 | 2 | 0.52 | 0.00002 | 80.00 |
| EMX1_T4 | Cas12i2 | 3 | 0.77 | 0.00080 | 29.55 |
| EMX1_T4 | Cas12i2 | 4 | 0.77 | 0.00094 | 26.09 |
| EMX1_T4 | SpCas9 | 1 | 0.98 | 0.00066 | 19.09 |
| EMX1_T4 | SpCas9 | 2 | 0.98 | 0.00204 | 18.08 |
| EMX1_T4 | SpCas9 | 3 | 0.98 | 0.00112 | 18.00 |
| EMX1_T4 | SpCas9 | 4 | 0.98 | 0.00090 | 18.51 |
| EMX1_T5 | Cas12i2 | 1 | 0.67 | 0.00088 | 19.89 |
| EMX1_T5 | Cas12i2 | 2 | 0.74 | 0.00046 | 27.26 |
| EMX1_T5 | Cas12i2 | 3 | 0.67 | 0.00140 | 21.00 |
| EMX1_T5 | SpCas9 | 1 | 0.57 | 0.00040 | 23.40 |
| EMX1_T5 | SpCas9 | 2 | 0.57 | 0.00086 | 29.14 |
| EMX1_T5 | SpCas9 | 3 | 0.58 | 0.00100 | 24.44 |
| EMX1_T5 | SpCas9 | 4 | 0.58 | 0.00060 | 25.03 |
| VEGFA_T1 | Cas12i2 | 1 | 0.84 | 0.00049 | 28.50 |
| VEGFA_T1 | Cas12i2 | 2 | 0.83 | 0.00056 | 24.65 |
| VEGFA_T1 | Cas12i2 | 3 | 0.82 | 0.00068 | 26.81 |
| VEGFA_T1 | Cas12i2 | 4 | 0.84 | 0.00076 | 24.00 |
| VEGFA_T1 | SpCas9 | 1 | 0.17 | 0.00017 | 16.00 |
| VEGFA_T1 | SpCas9 | 2 | 0.17 | 0.00079 | 18.56 |
| VEGFA_T1 | SpCas9 | 3 | 0.16 | 0.00048 | 17.96 |
| VEGFA_T1 | SpCas9 | 4 | 0.17 | 0.00084 | 20.14 |
| VEGFA_T3 | Cas12i2 | 1 | 0.77 | 0.00134 | 31.46 |
| VEGFA_T3 | Cas12i2 | 2 | 0.78 | 0.00044 | 21.77 |
| VEGFA_T3 | Cas12i2 | 3 | 0.79 | 0.00076 | 29.34 |
| VEGFA_T3 | Cas12i2 | 4 | 0.78 | 0.00078 | 25.49 |
| VEGFA_T3 | SpCas9 | 1 | 0.94 | 0.00404 | 17.69 |
| VEGFA_T3 | SpCas9 | 2 | 0.93 | 0.00264 | 18.48 |
| VEGFA_T3 | SpCas9 | 3 | 0.94 | 0.00358 | 15.98 |
| VEGFA_T3 | SpCas9 | 4 | 0.93 | 0.00416 | 16.38 |
| VEGFA_T4 | Cas12i2 | 1 | 0.98 | 0.00022 | 27.00 |
| VEGFA_T4 | Cas12i2 | 2 | 0.99 | 0.00018 | 27.67 |
| VEGFA_T4 | Cas12i2 | 3 | 0.99 | 0.00064 | 32.13 |
| VEGFA_T4 | Cas12i2 | 4 | 0.98 | 0.00090 | 24.44 |
| VEGFA_T4 | SpCas9 | 1 | 0.97 | 0.00230 | 19.99 |
| VEGFA_T4 | SpCas9 | 2 | 0.98 | 0.00418 | 17.41 |
| VEGFA_T4 | SpCas9 | 3 | 0.97 | 0.00266 | 17.92 |
| VEGFA_T4 | SpCas9 | 4 | 0.97 | 0.00246 | 17.82 |

TABLE 15
Characterization of 6-nucleotide insertions.

| | | | | | Average Start |
|---|---|---|---|---|---|
| | | | | Frequency of | position of 6- |
| | | | Total Indel | 6-Nucleotide | Nucleotide |
| Target | Nuclease | Replicate No. | Frequency | Insertion | Insertion |
| AAVS1_T2 | Cas12i2 | 1 | 0.38 | 0.00058 | 25.07 |
| AAVS1_T2 | Cas12i2 | 2 | 0.32 | 0.00032 | 22.75 |
| AAVS1_T2 | Cas12i2 | 3 | 0.39 | 0.00042 | 20.10 |
| AAVS1_T2 | Cas12i2 | 4 | 0.32 | 0.00030 | 24.67 |
| AAVS1_T2 | SpCas9 | 1 | 0.54 | 0.00238 | 23.66 |
| AAVS1_T2 | SpCas9 | 2 | 0.53 | 0.00154 | 22.96 |
| AAVS1_T2 | SpCas9 | 3 | 0.44 | 0.00126 | 22.42 |
| AAVS1_T2 | SpCas9 | 4 | 0.49 | 0.00148 | 22.28 |
| AAVS1_T3 | Cas12i2 | 1 | 0.90 | 0.00064 | 26.91 |
| AAVS1_T3 | Cas12i2 | 2 | 0.90 | 0.00075 | 27.24 |
| AAVS1_T3 | Cas12i2 | 3 | 0.89 | 0.00120 | 28.55 |
| AAVS1_T3 | Cas12i2 | 4 | 0.89 | 0.00100 | 30.30 |
| AAVS1_T3 | SpCas9 | 1 | 0.88 | 0.00168 | 36.00 |
| AAVS1_T3 | SpCas9 | 2 | 0.90 | 0.00071 | 16.25 |
| AAVS1_T3 | SpCas9 | 3 | 0.91 | 0.00218 | 17.24 |
| AAVS1_T3 | SpCas9 | 4 | 0.91 | 0.00007 | 17.00 |
| AAVS1_T4 | Cas12i2 | 1 | 0.79 | 0.00032 | 30.00 |
| AAVS1_T4 | Cas12i2 | 2 | 0.68 | 0.00128 | 30.02 |
| AAVS1_T4 | Cas12i2 | 3 | 0.69 | 0.00020 | 27.20 |
| AAVS1_T4 | Cas12i2 | 4 | 0.78 | 0.00024 | 32.50 |
| AAVS1_T4 | SpCas9 | 1 | 0.97 | 0.00042 | 16.14 |
| AAVS1_T4 | SpCas9 | 2 | 0.97 | 0.00072 | 15.72 |
| AAVS1_T4 | SpCas9 | 3 | 0.94 | 0.00162 | 23.00 |
| AAVS1_T4 | SpCas9 | 4 | 0.95 | 0.00078 | 18.26 |
| AAVS1_T6 | Cas12i2 | 1 | 0.90 | 0.00072 | 33.31 |
| AAVS1_T6 | Cas12i2 | 2 | 0.90 | 0.00076 | 30.63 |
| AAVS1_T6 | Cas12i2 | 3 | 0.88 | 0.00000 | 0.00 |
| AAVS1_T6 | Cas12i2 | 4 | 0.85 | 0.00000 | 0.00 |
| AAVS1_T6 | SpCas9 | 1 | 0.79 | 0.00847 | 16.00 |
| AAVS1_T6 | SpCas9 | 2 | 0.69 | 0.00307 | 22.90 |
| AAVS1_T6 | SpCas9 | 3 | 0.72 | 0.00306 | 22.12 |
| AAVS1_T6 | SpCas9 | 4 | 0.81 | 0.00319 | 26.50 |
| EMX1_T3 | Cas12i2 | 1 | 0.59 | 0.00028 | 26.00 |
| EMX1_T3 | Cas12i2 | 2 | 0.58 | 0.00064 | 25.31 |
| EMX1_T3 | Cas12i2 | 3 | 0.66 | 0.00042 | 22.67 |
| EMX1_T3 | Cas12i2 | 4 | 0.65 | 0.00092 | 25.61 |
| EMX1_T3 | SpCas9 | 1 | 0.81 | 0.00320 | 18.02 |
| EMX1_T3 | SpCas9 | 2 | 0.85 | 0.00332 | 20.73 |
| EMX1_T3 | SpCas9 | 3 | 0.85 | 0.00316 | 19.96 |
| EMX1_T3 | SpCas9 | 4 | 0.81 | 0.00238 | 17.63 |
| EMX1_T4 | Cas12i2 | 1 | 0.52 | 0.00000 | 0.00 |
| EMX1_T4 | Cas12i2 | 2 | 0.52 | 0.00000 | 0.00 |
| EMX1_T4 | Cas12i2 | 3 | 0.77 | 0.00032 | 26.06 |
| EMX1_T4 | Cas12i2 | 4 | 0.77 | 0.00058 | 28.76 |
| EMX1_T4 | SpCas9 | 1 | 0.98 | 0.00046 | 15.52 |
| EMX1_T4 | SpCas9 | 2 | 0.98 | 0.00108 | 18.72 |
| EMX1_T4 | SpCas9 | 3 | 0.98 | 0.00178 | 19.63 |
| EMX1_T4 | SpCas9 | 4 | 0.98 | 0.00096 | 16.42 |
| EMX1_T5 | Cas12i2 | 1 | 0.67 | 0.00062 | 29.68 |
| EMX1_T5 | Cas12i2 | 2 | 0.74 | 0.00182 | 23.93 |
| EMX1_T5 | Cas12i2 | 3 | 0.67 | 0.00128 | 28.39 |
| EMX1_T5 | SpCas9 | 1 | 0.57 | 0.00094 | 29.40 |
| EMX1_T5 | SpCas9 | 2 | 0.57 | 0.00002 | -3.00 |
| EMX1_T5 | SpCas9 | 3 | 0.58 | 0.00068 | 23.35 |
| EMX1_T5 | SpCas9 | 4 | 0.58 | 0.00048 | 26.04 |
| VEGFA_T1 | Cas12i2 | 1 | 0.84 | 0.00099 | 26.25 |
| VEGFA_T1 | Cas12i2 | 2 | 0.83 | 0.00095 | 25.38 |
| VEGFA_T1 | Cas12i2 | 3 | 0.82 | 0.00081 | 21.62 |
| VEGFA_T1 | Cas12i2 | 4 | 0.84 | 0.00146 | 24.74 |
| VEGFA_T1 | SpCas9 | 1 | 0.17 | 0.00066 | 20.00 |
| VEGFA_T1 | SpCas9 | 2 | 0.17 | 0.00079 | 13.00 |
| VEGFA_T1 | SpCas9 | 3 | 0.16 | 0.00072 | 18.08 |
| VEGFA_T1 | SpCas9 | 4 | 0.17 | 0.00052 | 16.12 |
| VEGFA_T3 | Cas12i2 | 1 | 0.77 | 0.00022 | 40.36 |
| VEGFA_T3 | Cas12i2 | 2 | 0.78 | 0.00080 | 25.42 |
| VEGFA_T3 | Cas12i2 | 3 | 0.79 | 0.00074 | 27.76 |
| VEGFA_T3 | Cas12i2 | 4 | 0.78 | 0.00104 | 26.92 |
| VEGFA_T3 | SpCas9 | 1 | 0.94 | 0.00350 | 19.14 |
| VEGFA_T3 | SpCas9 | 2 | 0.93 | 0.00254 | 17.66 |
| VEGFA_T3 | SpCas9 | 3 | 0.94 | 0.00264 | 17.64 |
| VEGFA_T3 | SpCas9 | 4 | 0.93 | 0.00246 | 18.92 |
| VEGFA_T4 | Cas12i2 | 1 | 0.98 | 0.00026 | 38.69 |
| VEGFA_T4 | Cas12i2 | 2 | 0.99 | 0.00040 | 27.95 |
| VEGFA_T4 | Cas12i2 | 3 | 0.99 | 0.00040 | 38.05 |
| VEGFA_T4 | Cas12i2 | 4 | 0.98 | 0.00042 | 24.10 |
| VEGFA_T4 | SpCas9 | 1 | 0.97 | 0.00154 | 17.91 |
| VEGFA_T4 | SpCas9 | 2 | 0.98 | 0.00082 | 17.44 |
| VEGFA_T4 | SpCas9 | 3 | 0.97 | 0.00184 | 24.04 |
| VEGFA_T4 | SpCas9 | 4 | 0.97 | 0.00124 | 18.44 |

TABLE 16
Characterization of 7-nucleotide insertions.

| | | | | | Average Start |
|---|---|---|---|---|---|
| | | | | Frequency of | position of 7- |
| | | | Total Indel | 7-Nucleotide | Nucleotide |
| Target | Nuclease | Replicate No. | Frequency | Insertion | Insertion |
| AAVS1_T2 | Cas12i2 | 1 | 0.38 | 0.00016 | 25.00 |
| AAVS1_T2 | Cas12i2 | 2 | 0.32 | 0.00038 | 25.42 |
| AAVS1_T2 | Cas12i2 | 3 | 0.39 | 0.00016 | 19.12 |
| AAVS1_T2 | Cas12i2 | 4 | 0.32 | 0.00032 | 24.06 |
| AAVS1_T2 | SpCas9 | 1 | 0.54 | 0.00142 | 21.73 |
| AAVS1_T2 | SpCas9 | 2 | 0.53 | 0.00212 | 20.43 |
| AAVS1_T2 | SpCas9 | 3 | 0.44 | 0.00073 | 20.57 |
| AAVS1_T2 | SpCas9 | 4 | 0.49 | 0.00122 | 21.89 |
| AAVS1_T3 | Cas12i2 | 1 | 0.90 | 0.00110 | 25.96 |
| AAVS1_T3 | Cas12i2 | 2 | 0.90 | 0.00034 | 22.88 |
| AAVS1_T3 | Cas12i2 | 3 | 0.89 | 0.00054 | 32.67 |
| AAVS1_T3 | Cas12i2 | 4 | 0.89 | 0.00120 | 22.12 |
| AAVS1_T3 | SpCas9 | 1 | 0.88 | 0.00053 | 19.00 |
| AAVS1_T3 | SpCas9 | 2 | 0.90 | 0.00248 | 17.04 |
| AAVS1_T3 | SpCas9 | 3 | 0.91 | 0.00041 | 16.37 |
| AAVS1_T3 | SpCas9 | 4 | 0.91 | 0.00046 | 16.90 |
| AAVS1_T4 | Cas12i2 | 1 | 0.79 | 0.00022 | 29.09 |
| AAVS1_T4 | Cas12i2 | 2 | 0.68 | 0.00048 | 28.50 |
| AAVS1_T4 | Cas12i2 | 3 | 0.69 | 0.00022 | 25.18 |
| AAVS1_T4 | Cas12i2 | 4 | 0.78 | 0.00046 | 29.96 |
| AAVS1_T4 | SpCas9 | 1 | 0.97 | 0.00078 | 24.56 |
| AAVS1_T4 | SpCas9 | 2 | 0.97 | 0.00134 | 21.39 |
| AAVS1_T4 | SpCas9 | 3 | 0.94 | 0.00090 | 16.89 |
| AAVS1_T4 | SpCas9 | 4 | 0.95 | 0.00086 | 19.12 |
| AAVS1_T6 | Cas12i2 | 1 | 0.90 | 0.00074 | 31.86 |
| AAVS1_T6 | Cas12i2 | 2 | 0.90 | 0.00030 | 42.67 |
| AAVS1_T6 | Cas12i2 | 3 | 0.88 | 0.00000 | 0.00 |
| AAVS1_T6 | Cas12i2 | 4 | 0.85 | 0.00000 | 0.00 |
| AAVS1_T6 | SpCas9 | 1 | 0.79 | 0.00565 | 23.00 |
| AAVS1_T6 | SpCas9 | 2 | 0.69 | 0.00176 | 17.33 |
| AAVS1_T6 | SpCas9 | 3 | 0.72 | 0.00212 | 28.22 |
| AAVS1_T6 | SpCas9 | 4 | 0.81 | 0.00159 | 19.00 |
| EMX1_T3 | Cas12i2 | 1 | 0.59 | 0.00114 | 28.26 |
| EMX1_T3 | Cas12i2 | 2 | 0.58 | 0.00072 | 24.44 |
| EMX1_T3 | Cas12i2 | 3 | 0.66 | 0.00058 | 28.03 |
| EMX1_T3 | Cas12i2 | 4 | 0.65 | 0.00052 | 26.08 |
| EMX1_T3 | SpCas9 | 1 | 0.81 | 0.00216 | 18.69 |
| EMX1_T3 | SpCas9 | 2 | 0.85 | 0.00202 | 18.55 |
| EMX1_T3 | SpCas9 | 3 | 0.85 | 0.00226 | 19.27 |
| EMX1_T3 | SpCas9 | 4 | 0.81 | 0.00286 | 17.37 |
| EMX1_T4 | Cas12i2 | 1 | 0.52 | 0.00008 | 29.50 |
| EMX1_T4 | Cas12i2 | 2 | 0.52 | 0.00028 | 25.14 |
| EMX1_T4 | Cas12i2 | 3 | 0.77 | 0.00078 | 24.95 |
| EMX1_T4 | Cas12i2 | 4 | 0.77 | 0.00048 | 27.42 |
| EMX1_T4 | SpCas9 | 1 | 0.98 | 0.00156 | 18.22 |
| EMX1_T4 | SpCas9 | 2 | 0.98 | 0.00174 | 19.80 |
| EMX1_T4 | SpCas9 | 3 | 0.98 | 0.00078 | 20.15 |
| EMX1_T4 | SpCas9 | 4 | 0.98 | 0.00044 | 20.14 |
| EMX1_T5 | Cas12i2 | 1 | 0.67 | 0.00060 | 21.20 |
| EMX1_T5 | Cas12i2 | 2 | 0.74 | 0.00044 | 26.68 |
| EMX1_T5 | Cas12i2 | 3 | 0.67 | 0.00100 | 27.76 |
| EMX1_T5 | SpCas9 | 1 | 0.57 | 0.00122 | 24.21 |
| EMX1_T5 | SpCas9 | 2 | 0.57 | 0.00012 | 21.00 |
| EMX1_T5 | SpCas9 | 3 | 0.58 | 0.00068 | 19.44 |
| EMX1_T5 | SpCas9 | 4 | 0.58 | 0.00086 | 24.79 |
| VEGFA_T1 | Cas12i2 | 1 | 0.84 | 0.00025 | 32.50 |
| VEGFA_T1 | Cas12i2 | 2 | 0.83 | 0.00063 | 25.12 |
| VEGFA_T1 | Cas12i2 | 3 | 0.82 | 0.00066 | 24.42 |
| VEGFA_T1 | Cas12i2 | 4 | 0.84 | 0.00019 | 43.00 |
| VEGFA_T1 | SpCas9 | 1 | 0.17 | 0.00000 | 0.00 |
| VEGFA_T1 | SpCas9 | 2 | 0.17 | 0.00053 | 18.67 |
| VEGFA_T1 | SpCas9 | 3 | 0.16 | 0.00012 | 19.00 |
| VEGFA_T1 | SpCas9 | 4 | 0.17 | 0.00046 | 18.96 |
| VEGFA_T3 | Cas12i2 | 1 | 0.77 | 0.00098 | 23.53 |
| VEGFA_T3 | Cas12i2 | 2 | 0.78 | 0.00070 | 25.97 |
| VEGFA_T3 | Cas12i2 | 3 | 0.79 | 0.00076 | 34.79 |
| VEGFA_T3 | Cas12i2 | 4 | 0.78 | 0.00012 | 22.17 |
| VEGFA_T3 | SpCas9 | 1 | 0.94 | 0.00150 | 17.68 |
| VEGFA_T3 | SpCas9 | 2 | 0.93 | 0.00210 | 18.30 |
| VEGFA_T3 | SpCas9 | 3 | 0.94 | 0.00206 | 17.76 |
| VEGFA_T3 | SpCas9 | 4 | 0.93 | 0.00184 | 18.65 |
| VEGFA_T4 | Cas12i2 | 1 | 0.98 | 0.00022 | 23.73 |
| VEGFA_T4 | Cas12i2 | 2 | 0.99 | 0.00016 | 41.13 |
| VEGFA_T4 | Cas12i2 | 3 | 0.99 | 0.00036 | 32.11 |
| VEGFA_T4 | Cas12i2 | 4 | 0.98 | 0.00000 | 0.00 |
| VEGFA_T4 | SpCas9 | 1 | 0.97 | 0.00210 | 17.80 |
| VEGFA_T4 | SpCas9 | 2 | 0.98 | 0.00320 | 25.47 |
| VEGFA_T4 | SpCas9 | 3 | 0.97 | 0.00128 | 17.63 |
| VEGFA_T4 | SpCas9 | 4 | 0.97 | 0.00164 | 17.96 |

TABLE 17
Characterization of 8-nucleotide insertions.

| | | | | | Average Start |
|---|---|---|---|---|---|
| | | | | Frequency of | position of 8- |
| | | | Total Indel | 8-Nucleotide | Nucleotide |
| Target | Nuclease | Replicate No. | Frequency | Insertion | Insertion |
| AAVS1_T2 | Cas12i2 | 1 | 0.38 | 0.00046 | 28.78 |
| AAVS1_T2 | Cas12i2 | 2 | 0.32 | 0.00032 | 25.63 |
| AAVS1_T2 | Cas12i2 | 3 | 0.39 | 0.00102 | 25.59 |
| AAVS1_T2 | Cas12i2 | 4 | 0.32 | 0.00004 | 25.00 |
| AAVS1_T2 | SpCas9 | 1 | 0.54 | 0.00288 | 20.92 |
| AAVS1_T2 | SpCas9 | 2 | 0.53 | 0.00218 | 19.92 |
| AAVS1_T2 | SpCas9 | 3 | 0.44 | 0.00157 | 23.93 |
| AAVS1_T2 | SpCas9 | 4 | 0.49 | 0.00160 | 21.79 |
| AAVS1_T3 | Cas12i2 | 1 | 0.90 | 0.00082 | 27.66 |
| AAVS1_T3 | Cas12i2 | 2 | 0.90 | 0.00053 | 33.15 |
| AAVS1_T3 | Cas12i2 | 3 | 0.89 | 0.00066 | 31.64 |
| AAVS1_T3 | Cas12i2 | 4 | 0.89 | 0.00084 | 30.31 |
| AAVS1_T3 | SpCas9 | 1 | 0.88 | 0.00099 | 17.00 |
| AAVS1_T3 | SpCas9 | 2 | 0.90 | 0.00000 | 0.00 |
| AAVS1_T3 | SpCas9 | 3 | 0.91 | 0.00020 | 18.33 |
| AAVS1_T3 | SpCas9 | 4 | 0.91 | 0.00135 | 17.48 |
| AAVS1_T4 | Cas12i2 | 1 | 0.79 | 0.00008 | 24.00 |
| AAVS1_T4 | Cas12i2 | 2 | 0.68 | 0.00040 | 30.35 |
| AAVS1_T4 | Cas12i2 | 3 | 0.69 | 0.00048 | 37.75 |
| AAVS1_T4 | Cas12i2 | 4 | 0.78 | 0.00090 | 31.07 |
| AAVS1_T4 | SpCas9 | 1 | 0.97 | 0.00014 | 17.00 |
| AAVS1_T4 | SpCas9 | 2 | 0.97 | 0.00088 | 17.34 |
| AAVS1_T4 | SpCas9 | 3 | 0.94 | 0.00094 | 17.96 |
| AAVS1_T4 | SpCas9 | 4 | 0.95 | 0.00072 | 20.67 |
| AAVS1_T6 | Cas12i2 | 1 | 0.90 | 0.00062 | 29.35 |
| AAVS1_T6 | Cas12i2 | 2 | 0.90 | 0.00056 | 33.61 |
| AAVS1_T6 | Cas12i2 | 3 | 0.88 | 0.00304 | 24.00 |
| AAVS1_T6 | Cas12i2 | 4 | 0.85 | 0.00000 | 0.00 |
| AAVS1_T6 | SpCas9 | 1 | 0.79 | 0.00565 | 16.00 |
| AAVS1_T6 | SpCas9 | 2 | 0.69 | 0.00293 | 21.00 |
| AAVS1_T6 | SpCas9 | 3 | 0.72 | 0.00224 | 21.89 |
| AAVS1_T6 | SpCas9 | 4 | 0.81 | 0.00159 | 26.00 |
| EMX1_T3 | Cas12i2 | 1 | 0.59 | 0.00042 | 31.14 |
| EMX1_T3 | Cas12i2 | 2 | 0.58 | 0.00058 | 27.34 |
| EMX1_T3 | Cas12i2 | 3 | 0.66 | 0.00032 | 23.63 |
| EMX1_T3 | Cas12i2 | 4 | 0.65 | 0.00058 | 29.14 |
| EMX1_T3 | SpCas9 | 1 | 0.81 | 0.00118 | 17.97 |
| EMX1_T3 | SpCas9 | 2 | 0.85 | 0.00314 | 18.78 |
| EMX1_T3 | SpCas9 | 3 | 0.85 | 0.00246 | 18.16 |
| EMX1_T3 | SpCas9 | 4 | 0.81 | 0.00144 | 18.08 |
| EMX1_T4 | Cas12i2 | 1 | 0.52 | 0.00000 | 0.00 |
| EMX1_T4 | Cas12i2 | 2 | 0.52 | 0.00060 | 24.77 |
| EMX1_T4 | Cas12i2 | 3 | 0.77 | 0.00058 | 25.17 |
| EMX1_T4 | Cas12i2 | 4 | 0.77 | 0.00070 | 32.57 |
| EMX1_T4 | SpCas9 | 1 | 0.98 | 0.00000 | 0.00 |
| EMX1_T4 | SpCas9 | 2 | 0.98 | 0.00064 | 20.31 |
| EMX1_T4 | SpCas9 | 3 | 0.98 | 0.00092 | 18.80 |
| EMX1_T4 | SpCas9 | 4 | 0.98 | 0.00112 | 23.09 |
| EMX1_T5 | Cas12i2 | 1 | 0.67 | 0.00148 | 25.99 |
| EMX1_T5 | Cas12i2 | 2 | 0.74 | 0.00142 | 25.62 |
| EMX1_T5 | Cas12i2 | 3 | 0.67 | 0.00044 | 23.50 |
| EMX1_T5 | SpCas9 | 1 | 0.57 | 0.00054 | 23.30 |
| EMX1_T5 | SpCas9 | 2 | 0.57 | 0.00056 | 24.64 |
| EMX1_T5 | SpCas9 | 3 | 0.58 | 0.00056 | 25.00 |
| EMX1_T5 | SpCas9 | 4 | 0.58 | 0.00070 | 30.31 |
| VEGFA_T1 | Cas12i2 | 1 | 0.84 | 0.00025 | 24.00 |
| VEGFA_T1 | Cas12i2 | 2 | 0.83 | 0.00034 | 28.71 |
| VEGFA_T1 | Cas12i2 | 3 | 0.82 | 0.00051 | 26.70 |
| VEGFA_T1 | Cas12i2 | 4 | 0.84 | 0.00032 | 26.80 |
| VEGFA_T1 | SpCas9 | 1 | 0.17 | 0.00000 | 0.00 |
| VEGFA_T1 | SpCas9 | 2 | 0.17 | 0.00167 | 19.63 |
| VEGFA_T1 | SpCas9 | 3 | 0.16 | 0.00052 | 21.81 |
| VEGFA_T1 | SpCas9 | 4 | 0.17 | 0.00030 | 20.60 |
| VEGFA_T3 | Cas12i2 | 1 | 0.77 | 0.00056 | 32.39 |
| VEGFA_T3 | Cas12i2 | 2 | 0.78 | 0.00098 | 27.06 |
| VEGFA_T3 | Cas12i2 | 3 | 0.79 | 0.00080 | 26.02 |
| VEGFA_T3 | Cas12i2 | 4 | 0.78 | 0.00066 | 40.24 |
| VEGFA_T3 | SpCas9 | 1 | 0.94 | 0.00198 | 19.78 |
| VEGFA_T3 | SpCas9 | 2 | 0.93 | 0.00250 | 17.71 |
| VEGFA_T3 | SpCas9 | 3 | 0.94 | 0.00342 | 19.16 |
| VEGFA_T3 | SpCas9 | 4 | 0.93 | 0.00144 | 21.35 |
| VEGFA_T4 | Cas12i2 | 1 | 0.98 | 0.00000 | 0.00 |
| VEGFA_T4 | Cas12i2 | 2 | 0.99 | 0.00016 | 25.38 |
| VEGFA_T4 | Cas12i2 | 3 | 0.99 | 0.00038 | 21.11 |
| VEGFA_T4 | Cas12i2 | 4 | 0.98 | 0.00018 | 27.00 |
| VEGFA_T4 | SpCas9 | 1 | 0.97 | 0.00106 | 18.23 |
| VEGFA_T4 | SpCas9 | 2 | 0.98 | 0.00070 | 20.66 |
| VEGFA_T4 | SpCas9 | 3 | 0.97 | 0.00108 | 20.83 |
| VEGFA_T4 | SpCas9 | 4 | 0.97 | 0.00130 | 23.92 |

TABLE 18
Characterization of 9-nucleotide insertions.

| | | | | | Average Start |
|---|---|---|---|---|---|
| | | | | Frequency of | position of 9- |
| | | | Total Indel | 9-Nucleotide | Nucleotide |
| Target | Nuclease | Replicate No. | Frequency | Insertion | Insertion |
| AAVS1_T2 | Cas12i2 | 1 | 0.38 | 0.00008 | 23.50 |
| AAVS1_T2 | Cas12i2 | 2 | 0.32 | 0.00024 | 26.17 |
| AAVS1_T2 | Cas12i2 | 3 | 0.39 | 0.00032 | 23.94 |
| AAVS1_T2 | Cas12i2 | 4 | 0.32 | 0.00046 | 23.30 |
| AAVS1_T2 | SpCas9 | 1 | 0.54 | 0.00274 | 22.15 |
| AAVS1_T2 | SpCas9 | 2 | 0.53 | 0.00188 | 18.19 |
| AAVS1_T2 | SpCas9 | 3 | 0.44 | 0.00084 | 22.00 |
| AAVS1_T2 | SpCas9 | 4 | 0.49 | 0.00176 | 23.23 |
| AAVS1_T3 | Cas12i2 | 1 | 0.90 | 0.00018 | 28.44 |
| AAVS1_T3 | Cas12i2 | 2 | 0.90 | 0.00051 | 32.16 |
| AAVS1_T3 | Cas12i2 | 3 | 0.89 | 0.00080 | 30.67 |
| AAVS1_T3 | Cas12i2 | 4 | 0.89 | 0.00086 | 28.40 |
| AAVS1_T3 | SpCas9 | 1 | 0.88 | 0.00084 | 19.18 |
| AAVS1_T3 | SpCas9 | 2 | 0.90 | 0.00071 | 14.00 |
| AAVS1_T3 | SpCas9 | 3 | 0.91 | 0.00074 | 17.38 |
| AAVS1_T3 | SpCas9 | 4 | 0.91 | 0.00049 | 16.86 |
| AAVS1_T4 | Cas12i2 | 1 | 0.79 | 0.00030 | 26.07 |
| AAVS1_T4 | Cas12i2 | 2 | 0.68 | 0.00032 | 25.63 |
| AAVS1_T4 | Cas12i2 | 3 | 0.69 | 0.00054 | 26.74 |
| AAVS1_T4 | Cas12i2 | 4 | 0.78 | 0.00000 | 0.00 |
| AAVS1_T4 | SpCas9 | 1 | 0.97 | 0.00026 | 19.15 |
| AAVS1_T4 | SpCas9 | 2 | 0.97 | 0.00082 | 15.90 |
| AAVS1_T4 | SpCas9 | 3 | 0.94 | 0.00060 | 20.17 |
| AAVS1_T4 | SpCas9 | 4 | 0.95 | 0.00052 | 17.77 |
| AAVS1_T6 | Cas12i2 | 1 | 0.90 | 0.00048 | 35.13 |
| AAVS1_T6 | Cas12i2 | 2 | 0.90 | 0.00066 | 30.67 |
| AAVS1_T6 | Cas12i2 | 3 | 0.88 | 0.00000 | 0.00 |
| AAVS1_T6 | Cas12i2 | 4 | 0.85 | 0.00000 | 0.00 |
| AAVS1_T6 | SpCas9 | 1 | 0.79 | 0.00565 | 16.00 |
| AAVS1_T6 | SpCas9 | 2 | 0.69 | 0.00146 | 27.60 |
| AAVS1_T6 | SpCas9 | 3 | 0.72 | 0.00259 | 22.41 |
| AAVS1_T6 | SpCas9 | 4 | 0.81 | 0.00159 | 16.00 |
| EMX1_T3 | Cas12i2 | 1 | 0.59 | 0.00046 | 21.48 |
| EMX1_T3 | Cas12i2 | 2 | 0.58 | 0.00034 | 25.65 |
| EMX1_T3 | Cas12i2 | 3 | 0.66 | 0.00056 | 28.43 |
| EMX1_T3 | Cas12i2 | 4 | 0.65 | 0.00048 | 23.29 |
| EMX1_T3 | SpCas9 | 1 | 0.81 | 0.00134 | 18.70 |
| EMX1_T3 | SpCas9 | 2 | 0.85 | 0.00110 | 17.35 |
| EMX1_T3 | SpCas9 | 3 | 0.85 | 0.00198 | 17.99 |
| EMX1_T3 | SpCas9 | 4 | 0.81 | 0.00138 | 16.43 |
| EMX1_T4 | Cas12i2 | 1 | 0.52 | 0.00010 | −10.00 |
| EMX1_T4 | Cas12i2 | 2 | 0.52 | 0.00086 | 24.05 |
| EMX1_T4 | Cas12i2 | 3 | 0.77 | 0.00072 | 25.03 |
| EMX1_T4 | Cas12i2 | 4 | 0.77 | 0.00076 | 24.03 |
| EMX1_T4 | SpCas9 | 1 | 0.98 | 0.00114 | 21.23 |
| EMX1_T4 | SpCas9 | 2 | 0.98 | 0.00064 | 18.06 |
| EMX1_T4 | SpCas9 | 3 | 0.98 | 0.00132 | 18.12 |
| EMX1_T4 | SpCas9 | 4 | 0.98 | 0.00086 | 18.09 |
| EMX1_T5 | Cas12i2 | 1 | 0.67 | 0.00118 | 24.63 |
| EMX1_T5 | Cas12i2 | 2 | 0.74 | 0.00234 | 27.98 |
| EMX1_T5 | Cas12i2 | 3 | 0.67 | 0.00086 | 24.72 |
| EMX1_T5 | SpCas9 | 1 | 0.57 | 0.00024 | 33.08 |
| EMX1_T5 | SpCas9 | 2 | 0.57 | 0.00062 | 26.19 |
| EMX1_T5 | SpCas9 | 3 | 0.58 | 0.00028 | 26.86 |
| EMX1_T5 | SpCas9 | 4 | 0.58 | 0.00044 | 21.14 |
| VEGFA_T1 | Cas12i2 | 1 | 0.84 | 0.00037 | 29.00 |
| VEGFA_T1 | Cas12i2 | 2 | 0.83 | 0.00066 | 23.44 |
| VEGFA_T1 | Cas12i2 | 3 | 0.82 | 0.00053 | 29.24 |
| VEGFA_T1 | Cas12i2 | 4 | 0.84 | 0.00057 | 24.44 |
| VEGFA_T1 | SpCas9 | 1 | 0.17 | 0.00017 | 26.00 |
| VEGFA_T1 | SpCas9 | 2 | 0.17 | 0.00026 | 16.00 |
| VEGFA_T1 | SpCas9 | 3 | 0.16 | 0.00034 | 17.65 |
| VEGFA_T1 | SpCas9 | 4 | 0.17 | 0.00028 | 24.36 |
| VEGFA_T3 | Cas12i2 | 1 | 0.77 | 0.00032 | 20.38 |
| VEGFA_T3 | Cas12i2 | 2 | 0.78 | 0.00054 | 25.26 |
| VEGFA_T3 | Cas12i2 | 3 | 0.79 | 0.00052 | 23.00 |
| VEGFA_T3 | Cas12i2 | 4 | 0.78 | 0.00032 | 39.19 |
| VEGFA_T3 | SpCas9 | 1 | 0.94 | 0.00116 | 17.36 |
| VEGFA_T3 | SpCas9 | 2 | 0.93 | 0.00166 | 20.81 |
| VEGFA_T3 | SpCas9 | 3 | 0.94 | 0.00232 | 18.28 |
| VEGFA_T3 | SpCas9 | 4 | 0.93 | 0.00110 | 18.05 |
| VEGFA_T4 | Cas12i2 | 1 | 0.98 | 0.00000 | 0.00 |
| VEGFA_T4 | Cas12i2 | 2 | 0.99 | 0.00020 | 24.50 |
| VEGFA_T4 | Cas12i2 | 3 | 0.99 | 0.00020 | 39.10 |
| VEGFA_T4 | Cas12i2 | 4 | 0.98 | 0.00026 | 33.23 |
| VEGFA_T4 | SpCas9 | 1 | 0.97 | 0.00068 | 17.71 |
| VEGFA_T4 | SpCas9 | 2 | 0.98 | 0.00058 | 17.00 |
| VEGFA_T4 | SpCas9 | 3 | 0.97 | 0.00152 | 16.91 |
| VEGFA_T4 | SpCas9 | 4 | 0.97 | 0.00156 | 18.46 |

As shown in Tables 10-18, insertions were more frequently observed in SpCas9 samples than in Cas12i2 samples. For example, as shown in Table 10, 1-nucleotide insertions induced by SpCas9 were observed with a maximum indel frequency of 0.4188 (for target AAVS1_T4), whereas 1-nucleotide insertions induced by Cas12i2 were observed with a maximum indel frequency of 0.0135 (for target EMX1_T3) Additionally, SpCas9-induced insertions were most frequently observed within the target sequence (e.g., around position 15 to position 19), whereas Cas12i2-induced insertions were most frequently observed downstream of the target sequence (e.g., around position 20 to position 25). A direct comparison of the insertion positions can be conducted using AAVS1_T3 and VEGFA_T1, which have overlapping SpCas9 and Cas12i2 target regions, as shown in FIG. 1. Similar total indel frequencies are observed in SpCas9 and Cas12i2 AAVS1-T3 samples (approximately 0.90 for each nuclease); however, the frequency of 1-nucleotide insertions induced by SpCas9 exceeds the frequency of 1-nucleotide insertions induced by Cas12i2. Additionally, the average start position of SpCas9-induced 1-nucleotide insertions in AAVS1_T3 ranges from position 15 to position 16, whereas the average start position of Cas12i2-induced 1-nucleotide insertions in AAVS1_T3 ranges from position 23 to position 25.

Therefore, this Example shows that cells modified by Cas12i2 have characteristic indel sizes and positions that allow for Cas12i2-modified cells to be distinguished from SpCas9-modified cells.

### Example 3—Efficient Immune Cell Editing with Engineered CRISPR-Cas12i

CRISPR RNA-guided nucleases have gained considerable interest for their role in revolutionizing existing ex vivo approaches to engineered cell therapies. This Example describes a novel engineered Type V CRISPR-Cas variant, Cas12i, as an alternative to the widely used Cas9 and Cpf1 CRISPR nuclease systems. Although effectors of subtypes V-A (e.g, Cas12a, also known as Cpf1) and V-B (e.g., Cas12b) have been studied in detail, the distinct domain architectures and diverged RuvC sequences of uncharacterized Cas12 proteins suggest unexplored functional diversity. In an effort to uncover such functional diversity, rigorous characterization of Cas12i revealed therapeutically differentiating attributes over classical gene modification nucleases, including compact size (1054 amino acid protein, 43 nucleotide tracr-less guide RNA), the validation of an optimal T-rich PAM in mammalian cells, distinct editing outcomes, and potential for multiplexing. A high-throughput evaluation of engineered variants of Cas12i was performed that resulted in a Cas12i variant that yielded dramatically increased (˜40-fold) activity over the parent Cas12i and comprised enhanced specificity as compared to SpCas9. RNP-mediated delivery of the Cas12i variant to T cells targeting therapeutically relevant loci revealed robust editing (>90% indel across multiple targets and donors). See Examples 4-6 below. Additionally, specificity studies using in silico prediction coupled with targeted NGS sequencing demonstrated the robust activity and specificity of the Cas12i variant. See Example 4-7 below. Taken together, these data suggested that this variant of Cas12i is uniquely differentiated to enable robust and precise engineered cell therapies.

### Example 4—Modified T Cells Generated by Cas12i2 Editing of B2M in Primary T Cells

This example demonstrates generation of modified T cells with variant Cas12i2. For this study, human primary T cells were transfected with B2M-targeting RNPs comprising variant Cas12i2 of SEQ ID NO: 4 and different crRNAs. The modified T cells were analyzed by fluorescence-activated cell sorting (FACS) staining and indel assessment at the B2M target. RNPs comprising SpCas9 protein (SEQ ID NO: 5) and B2M-targeting sgRNA were used as controls.

CD3+ T cells from three individual human donors were collected and counted using an automated cell counter. A sample from each donor was collected and stained for CD3E and DAPI for flow cytometry analysis of surface expression and viability, respectively. Cell density was adjusted to 1e6 cells/mL and cells were stimulated for 3 days with a cocktail of anti-CD3:CD28 antibodies.

RNP Complexation Reactions: Variant Cas12i2 RNP complexes were prepared by mixing purified variant Cas12i2 of SEQ ID NO: 4 (400 μM) with different crRNAs (1 mM in 250 mM NaCl) at a 1:1 Cas12i2 effector:crRNA volume ratio (corresponding to 2.5:1 crRNA:Cas12i2 effector molar ratio). SpCas9 RNP complexes were prepared by mixing purified SpCas9 (62 μM) with single guide RNA (sgRNA) (1 mM in water) at a 6.45:1 SpCas9 effector: sgRNA volume ratio (corresponding to 2.5:1 sgRNA: SpCas9 effector molar ratio). SpCas9 protein (SEQ ID NO: 5) was purchased from Aldevron. Sequences of crRNAs and sgRNA for targeting B2M are shown in Table 19.

TABLE 19
Sequences for B2M-targeting crRNAs (for variant Cas12i2) and sgRNA (for SpCas9)
used for RNP complexes.

| | | | | DNA | |
|---|---|---|---|---|---|
| Guide Name | Gene | Effector | PAM | Strand | crRNA/sgRNA |
| Cas12i2_B2M | B2M | Cas12i2 | CTTC | Sense | rArGrArArArUrCrCrGrUrCrUrUrUrCrArUrUr |
| exon2_target4 | | | | | GrArCrGrGrArArUrGrUrCrGrGrArUrGrGrAr |
| | | | | | UrGrArArArCrC (SEQ ID NO: 49) |
| Cas12i2_B2M | B2M | Cas12i2 | CTTT | Anti- | rArGrArArArUrCrCrGrUrCrUrUrUrCrArUrUr |
| exon2_target8 | | | | sense | GrArCrGrGrCrUrArUrCrUrCrUrUrGrUrArCrU |
| | | | | | rArCrArCrUrG (SEQ ID NO: 50) |
| Cas12i2_B2M | B2M | Cas12i2 | GTTC | Sense | rArGrArArArUrCrCrGrUrCrUrUrUrCrArUrUr |
| exon2_targetl0 | | | | | GrArCrGrGrArCrArCrGrGrCrArGrGrCrArUrA |
| | | | | | rCrUrCrArUrC (SEQ ID NO: 51) |
| Cas12i2_B2M | B2M | Cas12i2 | CTTT | Anti- | rArGrArArArUrCrCrGrUrCrUrUrUrCrArUrUr |
| exon2_target11 | | | | sense | GrArCrGrGrGrUrCrArCrArGrCrCrCrArArGrA |
| | | | | | rUrArGrUrUrA (SEQ ID NO: 52) |
| SpCas9_B2M | B2M | SpCas9 | TGG | Anti- | mG*mG*mC*rCrGrArGrArUrGrUrCrUrCrGrC |
| exon1_target1 | | | | sense | rUrCrCrGrGrUrUrUrUrArGrArGrCrUrArGrAr |
| | | | | | ArArUrArGrCrArArGrUrUrArArArArUrArAr |
| | | | | | GrGrCrUrArGrUrCrCrGrUrUrArUrCrArArCrU |
| | | | | | TUrGrArArArArArGrUrGrGrCrArCrCrGrArGr |
| | | | | | UrCrGrGrUrGrCmU*mU*mU*rU (SEQ ID |
| | | | | | NO: 53) |

r-RNA base
*-phosphorothioated
m-2’ O-methyl

For “effector only” controls, variant Cas12i2 of SEQ ID NO: 4 or SpCas9 of SEQ ID NO: 5 were mixed with Protein Storage Buffer (25 mM Tris, pH 7.5, 250 mM NaCl, 1 mM TCEP, 50% glycerol) at the same volume ratio as the crRNA or sgRNA, respectively. Additional controls were also included such as SpCas9 (purchased from Aldevron) with either transfection control guide Lethal #1, pooled CD3, or ROSA26 sgRNAs and SpCas9 (purchased from Horizon) with either transfection control guide Lethal #1, pooled CD3, or ROSA26 sgRNAs. RNP complexations were incubated at 37 degrees Celsius for 30-60 minutes. Following incubation, RNPs were diluted to 20 μM, 50 μM, 100 μM, or 160 μM effector concentration for variant Cas12i2 of SEQ ID NO: 4 and 20 μM or 50 μM for SpCas9.

Diluted complexed reactions were dispensed at 2 μL per well into a 384 electroporation plate. Cell suspensions were collected and counted using an automated cell counter. Cell density was adjusted to 1.1e7 cells/mL in P3 primary cell buffer (from Lonza #VXP-3032) and was dispensed at 2e5 cells/reaction (18 μL). Final concentration of variant Cas12i2 RNPs was 2 μM, 5 μM, 10 μM, or 16 μM. Final concentration of SpCas9 RNPs was 2 or 5 μM. The following controls were set up: unelectroporated cells only, cells in P3 primary cell buffer (from Lonza #VXP-3032) only, cells in Protein Storage Buffer only. The plate was electroporated using an electroporation device (program EO-115-AA, Lonza HT), excluding the unelectroporated conditions. Each well was split into four 96-well editing plates (containing 200 μL total volume) using robotics (from StarLab Hamilton). Editing plates were incubated for 7 days at 37 degrees Celsius with 100 μL media replacement at day 4.

After 7 days, plates were spun down and the supernatant was removed. Pellets were resuspended in 200 μL of PBS. 100 μL of sample was collected and stained with either anti-B2M antibody or anti-CD3E antibody (transfection control guide lethal #1, pooled CD3E, ROSA26, Protein Storage Buffer and unelectroporated for SpCas9 (SEQ ID NO: 5) controls). All cells were stained with DAPI to assess viability. Remaining cell suspension was transferred to a 96-well PCR plate and pelleted at 500× g for 5 min.

Supernatants were removed and pellets were frozen at −80 degrees Celsius.

For genomic DNA extraction, pellets were thawed to room temperature and resuspended in appropriate volume of DNA extraction buffer (from QuickExtract) to give final concentration of 1000 cells/μL. Samples were then cycled in PCR machine at 65 degrees Celsius for 15 min, 68 degrees Celsius for 15 min, 98 degrees Celsius for 10 min. Samples were then frozen at −20 degrees Celsius.

Samples for Next Generation Sequencing (NGS) were prepared by rounds of PCR. The first round (PCR I) was used to amplify the genomic regions flanking the target site and add NGS adapters. The second round (PCR II) was used to add NGS indexes. Reactions were then pooled, purified by column purification, and quantified on a fluorometer (Qubit). Sequencing runs were done using a 150 cycle NGS instrument (NextSeq v2.5) mid or high output kit and run on an NGS instrument (NextSeq 550).

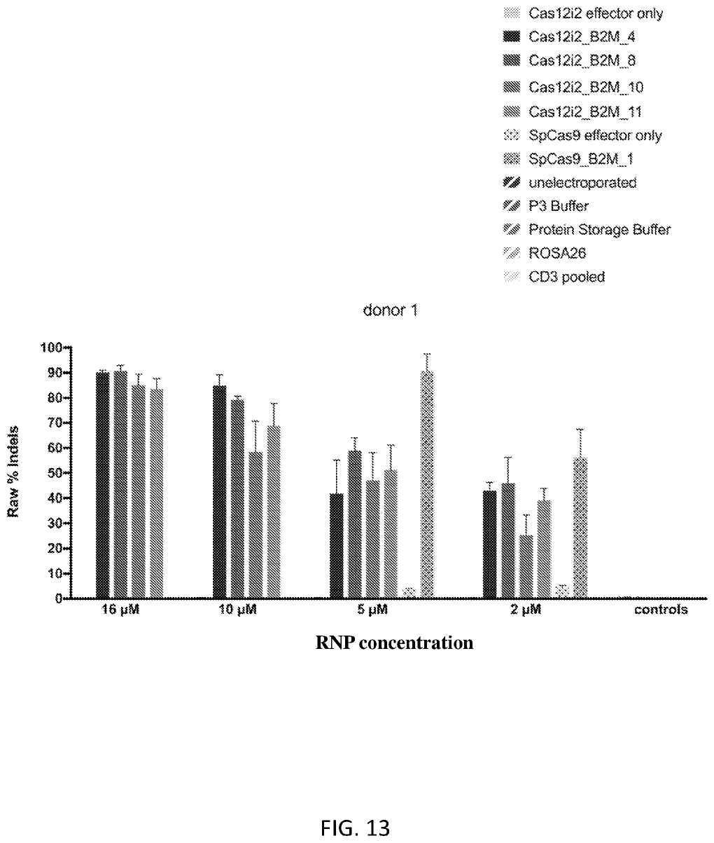

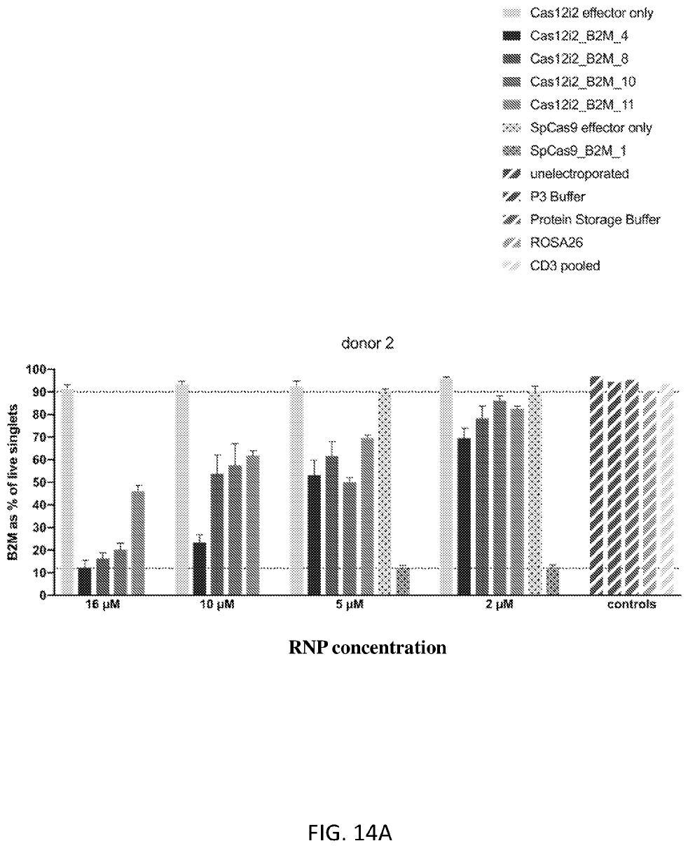

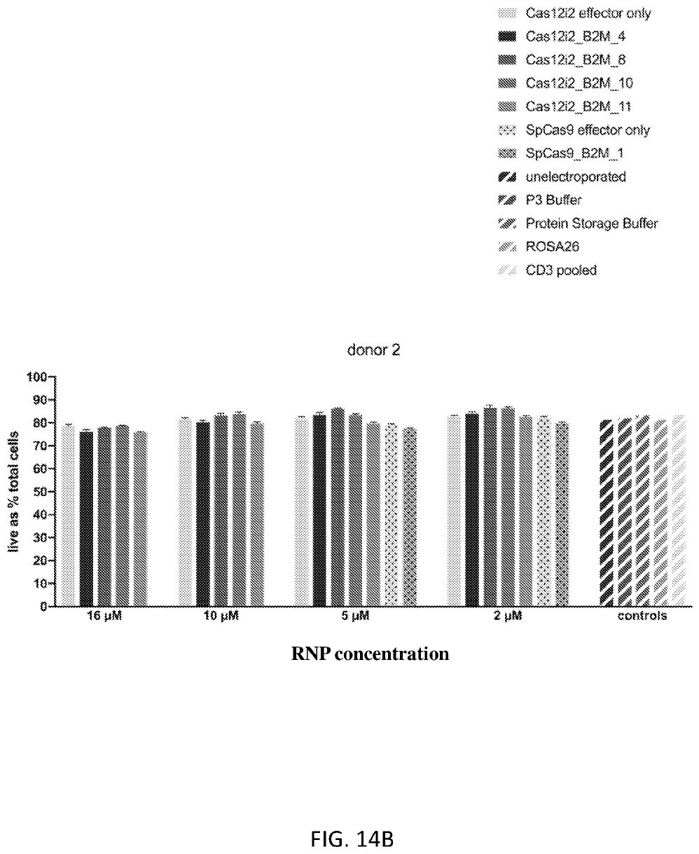

FIG. 13, FIG. 14A, and FIG. 14B illustrate the results of this example. As shown in FIG. 13, B2M-targeting RNP complexes comprising variant Cas12i2 and different crRNAs resulted in indel activity in primary T cells. The indel measurement was performed seven days after B2M targeting in the primary T cells. FIG. 14A shows that the modified T cells had reduced expression of B2M at least seven days after the targeting of B2M in primary T cells by the variant Cas12i2. FIG. 14B shows the viability of modified cells, as measured by DAPI staining, seven days after the targeting of B2M in the primary T cells by the variant Cas12i2.

The study in this example showed that variant Cas12i2 of the disclosure comprises gene editing activity in primary T cells and can be used to generate modified T cells.

### Example 5—Modified T Cells Generated by Cas12i2 Editing of TRAC in Primary T Cells

This example demonstrates generation of modified T cells with variant Cas12i2. For this study, human primary T cells were transfected with TRAC-targeting RNPs comprising variant Cas12i2 of SEQ ID NO: 4 and different crRNAs. The modified T cells were analyzed by FACS staining and indel assessment at the TRAC target. RNPs comprising SpCas9 protein (SEQ ID NO: 5) and TRAC-targeting sgRNA were used as controls.

CD3+ T cells from three individual human donors were collected and counted using an automated cell counter. A sample from each donor was collected and stained for CD3E and DAPI for flow cytometry analysis of surface expression and viability, respectively. Cell density was adjusted to 1e6 cells/mL and cells were stimulated for 3 days with a cocktail of anti-CD3:CD28 antibodies.

RNP Complexation Reactions: Variant Cas12i2 RNP complexes were prepared by mixing purified variant Cas12i2 of SEQ ID NO: 4 (400 μM) with different crRNAs (1 mM in 250 mM NaCl) at a 1:1 Cas12i2 effector:crRNA volume ratio (corresponding to 2.5:1 crRNA:Cas12i2 effector molar ratio). SpCas9 RNP complexes were prepared by mixing purified SpCas9 (62 μM) with single guide RNA (sgRNA) (1 mM in water) at a 6.45:1 SpCas9 effector: sgRNA volume ratio (corresponding to 2.5:1 sgRNA: SpCas9 effector molar ratio). SpCas9 protein (SEQ ID NO: 5) was purchased from Aldevron. Sequences of crRNAs and sgRNA are shown in Table 20.

TABLE 20
Sequences of TRAC-targeting crRNAs (for variant Cas12i2) and sgRNA (for SpCas9)
used for RNP complexes

| | | | | DNA | |
|---|---|---|---|---|---|
| Guide Name | Gene | Effector | PAM | Strand | crRNA/sgRNA |
| Cas12i2_TRAC_ | TRAC | Cas12i2 | CTTC | Sense | rArGrArArArUrCrCrGrUrCrUrUrUrCrArUr |
| exon1_target3 | | | | | UrGrArCrGrGrArArGrArGrCrArArCrArGrU |
| | | | | | rGrCrUrGrUrGrGrC (SEQ ID NO: 54) |
| Cas12i2_TRAC_ | TRAC | Cas12i2 | CTTC | Anti- | rArGrArArArUrCrCrGrUrCrUrUrUrCrArUr |
| exon1_target5 | | | | sense | UrGrArCrGrGrArArCrArArCrArGrCrArUrU |
| | | | | | rArUrUrCrCrArGrA (SEQ ID NO: 55) |
| Cas12i2_TRAC_ | TRAC | Cas12i2 | CTTT | Sense | rArGrArArArUrCrCrGrUrCrUrUrUrCrArUr |
| exon2_target4 | | | | | UrGrArCrGrGrGrArArArCrArGrGrUrArArG |
| | | | | | rArCrArGrGrGrGrU (SEQ ID NO: 56) |
| Cas12i2_TRAC_ | TRAC | Cas12i2 | CTTT | Anti- | rArGrArArArUrCrCrGrUrCrUrUrUrCrArUr |
| exon3_target4 | | | | sense | UrGrArCrGrGrCrArGrGrArGrGrArGrGrArU |
| | | | | | rUrCrGrGrArArCrC (SEQ ID NO: 57) |
| SpCas9_TRAC_ | TRAC | SpCas9 | TGG | Anti- | mA*mA*mG*rUrUrCrCrUrGrUrGrArUrGrU |
| exon2_target1 | | | | sense | rCrArArGrCrGrUrUrUrUrArGrArGrCrUrAr |
| | | | | | GrArArArUrArGrCrArArGrUrUrArArArArU |
| | | | | | rArArGrGrCrUrArGrUrCrCrGrUrUrArUrCr |
| | | | | | ArArCrUrUrGrArArArArArGrUrGrGrCrArC |
| | | | | | rCrGrArGrUrCrGrGrUrGrCmU*mU*mU*rU |
| | | | | | (SEQ ID NO: 58) |

r-RNA base
*-phosphorothioated
m-2’ O-methyl

For “effector only” controls, variant Cas12i2 of SEQ ID NO: 4 or SpCas9 of SEQ ID NO: 5 were mixed with Protein Storage Buffer (25 mM Tris, pH 7.5, 250 mM NaCl, 1 mM TCEP, 50% glycerol) at the same volume ratio as the crRNA or sgRNA, respectively. Additional controls were also included such as SpCas9 (purchased from Aldevron) with either transfection control guide Lethal #1, pooled CD3, or ROSA26 sgRNAs and SpCas9 (purchased from Horizon) with either transfection control guide Lethal #1, pooled CD3, or ROSA26 sgRNAs. RNP complexations were incubated at 37 degrees Celsius for 30-60 minutes. Following incubation, RNPs were diluted to 20 μM, 50 μM, 100 μM, or 160 μM effector concentration for variant Cas12i2 of SEQ ID NO: 4 and 20 μM or 50 μM for SpCas9.

Diluted complexed reactions were dispensed at 2 μL per well into a 384 electroporation plate. Cell suspensions were collected and counted using an automated cell counter. Cell density was adjusted to 1.1e7 cells/mL in P3 primary cell buffer (from Lonza #VXP-3032) and was dispensed at 2e5 cells/reaction (18 μL). Final concentration of variant Cas12i2 RNPs was 2 μM, 5 μM, 10 μM, or 16 μM. Final concentration of SpCas9 RNPs was 2 or 5 μM. The following controls were set up: unelectroporated cells only, cells in P3 primary cell buffer (from Lonza #VXP-3032) only, cells in Protein Storage Buffer only. The plate was electroporated using an electroporation device (program EO-115-AA, Lonza HT), excluding the unelectroporated conditions. Each well was split into four 96-well editing plates (containing 200 μL total volume) using robotics (from StarLab Hamilton). Editing plates were incubated for 7 days at 37 degrees Celsius with 100 μL media replacement at day 4.

After 7 days, plates were spun down and the supernatant was removed. Pellets were resuspended in 200 μL of PBS. 100 μL of sample was collected and stained with either anti-TRAC antibody or anti-CD3E antibody (transfection control guide lethal #1, pooled CD3E, ROSA26, Protein Storage Buffer and unelectroporated for SpCas9 (SEQ ID NO: 5) controls). All cells were stained with DAPI to assess viability. Remaining cell suspension was transferred to a 96-well PCR plate and pelleted at 500× g for 5 min. Supernatants were removed and pellets were frozen at −80 degrees Celsius.

For genomic DNA extraction, pellets were thawed to room temperature and resuspended in appropriate volume of DNA extraction buffer (from QuickExtract) to give final concentration of 1000 cells/μL. Samples were then cycled in PCR machine at 65 degrees Celsius for 15 min, 68 degrees Celsius for 15 min, 98 degrees Celsius for 10 min. Samples were then frozen at −20 degrees Celsius.

Samples for Next Generation Sequencing (NGS) were prepared by rounds of PCR. The first round (PCR I) was used to amplify the genomic regions flanking the target site and add NGS adapters. The second round (PCR II) was used to add NGS indexes. Reactions were then pooled, purified by column purification, and quantified on a fluorometer (Qubit). Sequencing runs were done using a 150 cycle NGS instrument (NextSeq v2.5) mid or high output kit and run on an NGS instrument (NextSeq 550).

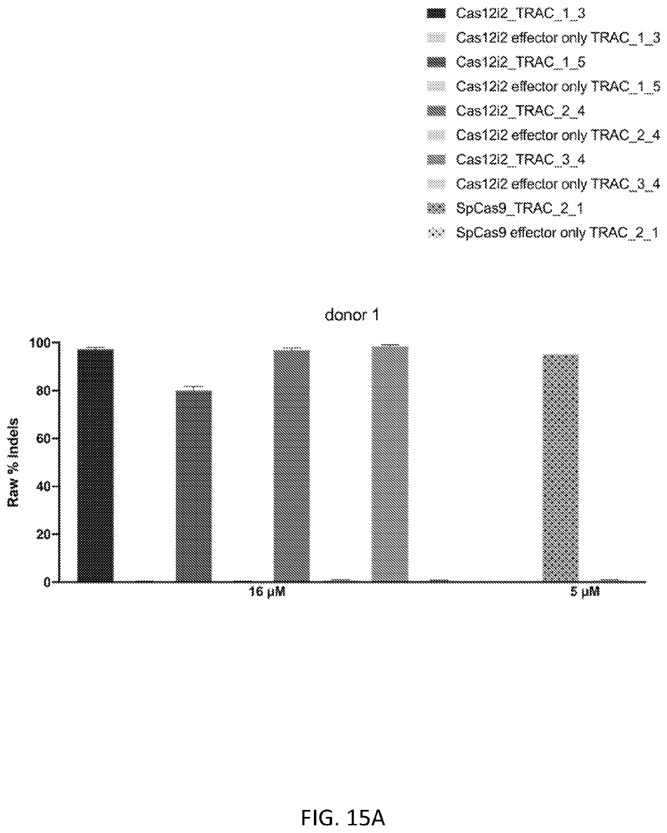

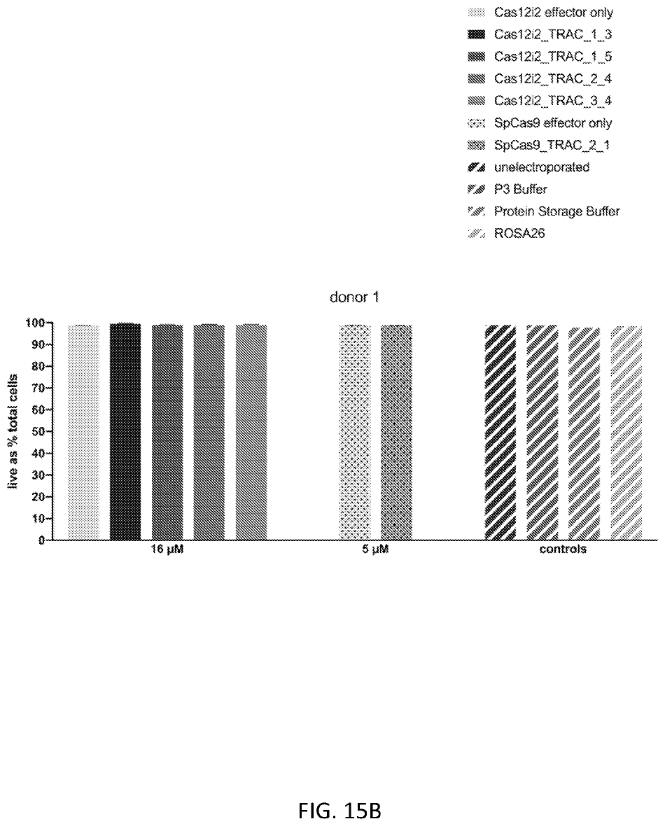

FIG. 15A and FIG. 15B illustrate the results of this example. As shown in FIG. 15A, TRAC-targeting RNP complexes comprising variant Cas12i2 and different crRNAs resulted in indel activity in primary T cells. FIG. 15B shows the viability of modified cells, as measured by DAPI staining, seven days after the targeting of TRAC in the primary T cells by the variant Cas12i2.

The study in this example showed that variant Cas12i2 of the disclosure comprises gene editing activity in primary T cells and can be used to generate modified T cells.

### Example 6—Modified T Cells Generated by Cas12i2 Editing of PDCD1 in Primary T Cells

This example demonstrates generation of modified T cells with variant Cas12i2. For this study, human primary T cells were transfected with PDCD1-targeting RNPs comprising variant Cas12i2 of SEQ ID NO: 4 and different crRNAs. The modified T cells were analyzed by FACS staining and indel assessment at the PDCD1 target. RNPs comprising SpCas9 protein (SEQ ID NO: 5) and TRAC-targeting sgRNA were used as controls.

CD3+ T cells from three individual human donors were revived and counted using an automated cell counter. A sample from each donor was collected and stained for CD3e and DAPI for flow cytometry analysis of surface expression and viability, respectively. Cell density was adjusted to 1e6 cells/mL and cells were stimulated for 3 days with a cocktail of anti-CD3:CD28 antibodies.

RNP Complexation Reactions: Variant Cas12i2 RNP complexes were prepared by mixing purified variant Cas12i2 of SEQ ID NO: 4 (400 μM) with different crRNAs (1 mM in 250 mM NaCl) at a 1:1 Cas12i2 effector:crRNA volume ratio (corresponding to 2.5:1 crRNA:Cas12i2 effector molar ratio). SpCas9 RNP complexes were prepared by mixing purified SpCas9 (62 μM) with single guide RNA (sgRNA) (1 mM in water) at a 6.45:1 SpCas9 effector: sgRNA volume ratio (corresponding to 2.5:1 sgRNA: SpCas9 effector molar ratio). SpCas9 protein (SEQ ID NO: 5) was purchased from Aldevron. Sequences of crRNAs and sgRNA are shown in Table 21.

TABLE 21
Sequences of PDCD1-targeting crRNAs (for variant Cas12i2) and sgRNA (for SpCas9)
used for RNP complexes

| | | | | DNA | |
|---|---|---|---|---|---|
| Guide Name | Gene | Effector | PAM | Strand | crRNA/sgRNA |
| Cas12i2_PDCD1_ | PDCD1 | Cas12i2 | GTTC | Anti- | rArGrArArArUrCrCrGrUrCrUrUrUrCrArUr |
| exon1_targetl | | | | sense | UrGrArCrGrGrUrUrArGrGrUrArGrGrUrGrG |
| | | | | | rGrGrUrCrGrGrCrG (SEQ ID NO: 59) |
| Cas12i2_PDCD1_ | PDCD1 | Cas12i2 | CTTC | Anti- | rArGrArArArUrCrCrGrUrCrUrUrUrCrArUr |
| exon2_target7 | | | | sense | UrGrArCrGrGrCrCrCrGrArGrGrArCrCrGrCr |
| | | | | | ArGrCrCrArGrCrC (SEQ ID NO: 60) |
| Cas12i2_PDCD1_ | PDCD1 | Cas12i2 | CTTC | Anti- | rArGrArArArUrCrCrGrUrCrUrUrUrCrArUr |
| exon2_target8 | | | | sense | UrGrArCrGrGrCrGrUrGrUrCrArCrArCrArA |
| | | | | | rCrUrGrCrCrCrArA (SEQ ID NO: 61) |
| Cas12i2_PDCD1_ | PDCD1 | Cas12i2 | CTTC | Anti- | rArGrArArArUrCrCrGrUrCrUrUrUrCrArUr |
| exon2_target9 | | | | sense | UrGrArCrGrGrCrArCrArUrGrArGrCrGrUrG |
| | | | | | rGrUrCrArGrGrGrC (SEQ ID NO: 62) |
| SpCas9_PDCD1_ | PDCD1 | SpCas9 | AGG | Anti- | mU*mC*mC*rArGrGrCrArUrGrCrArGrArU |
| exonl_target1 | | | | sense | rCrCrCrArCrGrUrUrUrUrArGrArGrCrUrAr |
| | | | | | GrArArArUrArGrCrArArGrUrUrArArArArU |
| | | | | | rArArGrGrCrUrArGrUrCrCrGrUrUrArUrCr |
| | | | | | ArArCrUrUrGrArArArArArGrUrGrGrCrArC |
| | | | | | rCrGrArGrUrCrGrGrUrGrCmU*mU*mU*rU |
| | | | | | (SEQ ID NO: 63) |

r-RNA base
*-phosphorothioated
m-2’ O-methyl

For “effector only” controls, variant Cas12i2 of SEQ ID NO: 4 or SpCas9 of SEQ ID NO: 5 were mixed with Protein Storage Buffer (25 mM Tris, pH 7.5, 250 mM NaCl, 1 mM TCEP, 50% glycerol) at the same volume ratio as the crRNA or sgRNA, respectively. Additional controls were also included such as SpCas9 (purchased from Aldevron) with either transfection control guide Lethal #1, pooled CD3, or ROSA26 sgRNAs and SpCas9 (purchased from Horizon) with either transfection control guide Lethal #1, pooled CD3, or ROSA26 sgRNAs. RNP complexations were incubated at 37 degrees Celsius for 30-60 minutes. Following incubation, RNPs were diluted to 20 μM, 50 μM, 100 μM, or 160 μM effector concentration for variant Cas12i2 of SEQ ID NO: 4 and 20 μM or 50 μM for SpCas9.

Diluted complexed reactions were dispensed at 2 μL per well into a 384 electroporation plate. Cell suspensions were collected and counted using an automated cell counter. Cell density was adjusted to 1.1e7 cells/mL in P3 primary cell buffer (from Lonza #VXP-3032) and was dispensed at 2e5 cells/reaction (18 μL). Final concentration of variant Cas12i2 RNPs was 2 μM, 5 μM, 10 μM, or 16 μM. Final concentration of SpCas9 RNPs was 2 or 5 μM. The following controls were set up: unelectroporated cells only, cells in P3 primary cell buffer (from Lonza #VXP-3032) only, cells in Protein Storage Buffer only. The plate was electroporated using an electroporation device (program EO-115-AA, Lonza HT), excluding the unelectroporated conditions. Each well was split into four 96-well editing plates (containing 200 μL total volume) using robotics (from StarLab Hamilton). Editing plates were incubated for 7 days at 37 degrees Celsius with 100 μL media replacement at day 4.

After 7 days, plates were spun down and the supernatant was removed. Pellets were resuspended in 200 μL of PBS. 100 μL of sample was collected and stained with either anti-TRAC antibody or anti-CD3E antibody (transfection control guide lethal #1, pooled CD3E, ROSA26, Protein Storage Buffer and unelectroporated for SpCas9 (SEQ ID NO: 5) controls). All cells were stained with DAPI to assess viability. Remaining cell suspension was transferred to a 96-well PCR plate and pelleted at 500× g for 5 min. Supernatants were removed and pellets were frozen at −80 degrees Celsius.

For genomic DNA extraction, pellets were thawed to room temperature and resuspended in appropriate volume of DNA extraction buffer (from QuickExtract) to give final concentration of 1000 cells/μL. Samples were then cycled in PCR machine at 65 degrees Celsius for 15 min, 68 degrees Celsius for 15 min, 98 degrees Celsius for 10 min. Samples were then frozen at −20 degrees Celsius.

Samples for Next Generation Sequencing (NGS) were prepared by rounds of PCR. The first round (PCR I) was used to amplify the genomic regions flanking the target site and add NGS adapters. The second round (PCR II) was used to add NGS indexes. Reactions were then pooled, purified by column purification, and quantified on a fluorometer (Qubit). Sequencing runs were done using a 150 cycle NGS instrument (NextSeq v2.5) mid or high output kit and run on an NGS instrument (NextSeq 550).

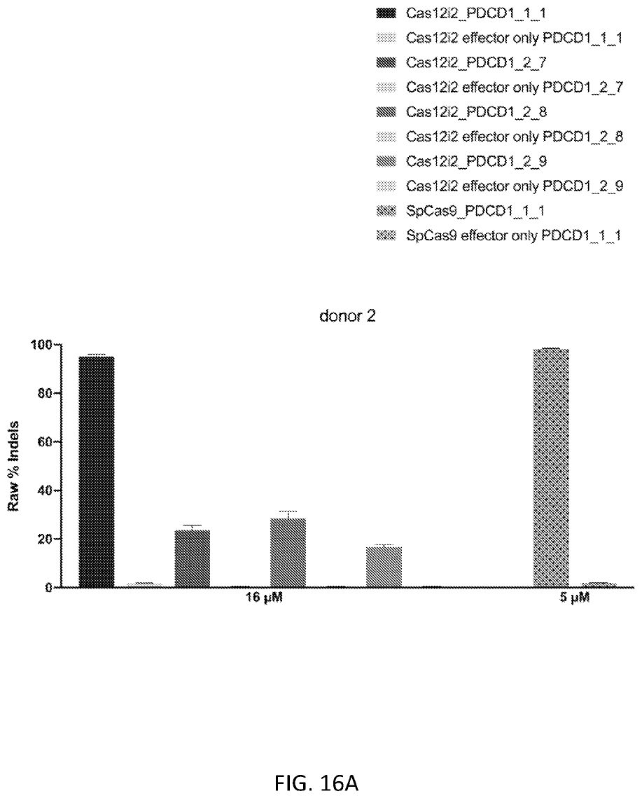

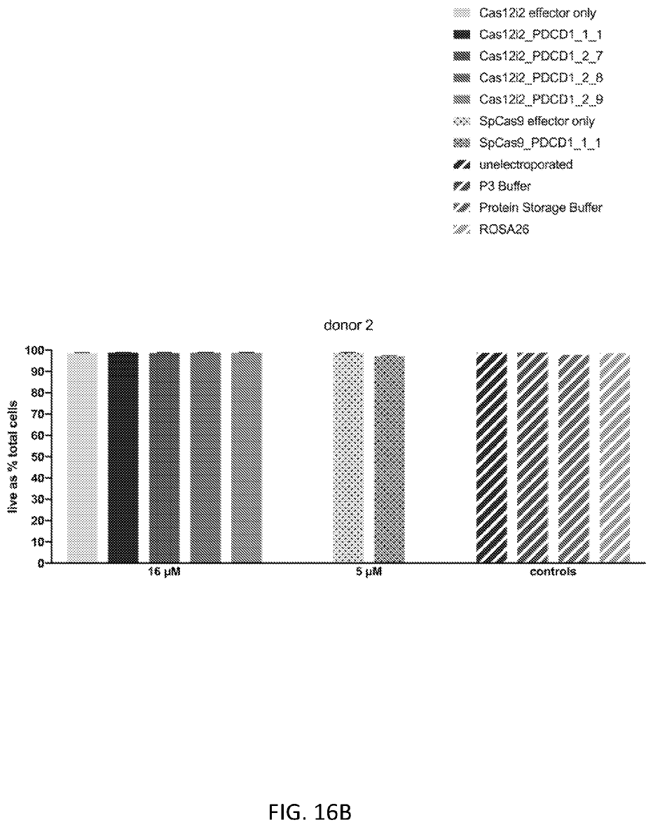

FIG. 16A and FIG. 16B illustrate the results of this example. As shown in FIG. 16A, PDCD1-targeting RNP complexes comprising variant Cas12i2 and different crRNAs resulted in indel activity in primary T cells. FIG. 16B shows the viability of modified cells, as measured by DAPI staining, seven days after the targeting of PDCD1 in the primary T cells by the variant Cas12i2.

This example showed that variant Cas12i2 of the disclosure comprises gene editing activity in primary T cells and can be used to generate modified T cells.

### Example 7—Modified T Cells Generated by Cas12i2 Editing of BCL11A Intronic Erythroid Enhancer in Primary CD34+ HSPCs

This example describes generation of modified CD34+ hematopoietic stem/progenitor cells (HSPC) with variant Cas12i2. For this study, human primary CD34+ HSPCs were transfected with BCL11A intronic erythroid enhancer-targeting RNPs comprising variant Cas12i2 of SEQ ID NO: 4 and crRNA. The modified CD34+ HSPCs were analyzed by FACS staining and indel assessment at the BCL11A intronic erythroid enhancer target.

Two frozen human bone marrow CD34+ cell vials per cell lot were thawed (Day 0), washed and assessed for cell number and viability by acridine orange/propidium iodide (AO/PI) staining using a cell counter. CD34+ cells were cultured in serum-free expansion media (from StemCell Technologies) with the appropriate supplement for approximately 48 hours.

RNP Complexation Reactions: Variant Cas12i2 RNP complexes were prepared by mixing purified variant Cas12i2 of SEQ ID NO: 4 (400 μM) with different crRNAs (1 mM in 250 mM NaCl) at a 1:1 Cas12i2 effector:crRNA volume ratio (corresponding to 2.5:1 crRNA:Cas12i2 effector molar ratio). SpCas9 RNP complexes were prepared by mixing purified SpCas9 (62 μM) with single guide RNA (sgRNA) (1 mM in water) at a 6.45:1 SpCas9 effector: sgRNA volume ratio (corresponding to 2.5:1 sgRNA: SpCas9 effector molar ratio). SpCas9 protein (SEQ ID NO: 5) was purchased from Aldevron. Sequences of crRNAs and sgRNA are shown in Table 22.

TABLE 22
Sequences of BCL11A intronic erythroid enhancer-targeting crRNAs (for variant
Cas12i2) and sgRNA (for SpCas9) used for RNP complexes

| | | | | DNA | |
|---|---|---|---|---|---|
| Guide Name | Gene | Effector | PAM | Strand | crRNA/sgRNA |
| Cas12i2_BCL11A_ | BCL11A_ | Cas12i2 | CTTT | Anti- | rArGrArArArUrCrCrGrUrCrUrUrUrCrAr |
| enh_T1 | enhancer | | | sense | UrUrGrArCrGrGrGrArArGrCrUrArGrUr |
| | | | | | CrUrArGrUrGrCrArArGrC (SEQ ID NO: |
| | | | | | 64) |
| Cas12i2_BCL11A_ | BCL11A_ | Cas12i2 | CTTC | Sense | rArGrArArArUrCrCrGrUrCrUrUrUrCrAr |
| enh_T4 | enhancer | | | | UrUrGrArCrGrGrCrUrGrGrArGrCrCrUr |
| | | | | | GrUrGrArUrArArArArGrC (SEQ ID NO: |
| | | | | | 65) |
| Cas12i2_BCL11A_ | BCL11A_ | Cas12i2 | CTTC | Sense | rArGrArArArUrCrCrGrUrCrUrUrUrCrAr |
| enh_T5 | enhancer | | | | UrUrGrArCrGrGrUrArCrCrCrCrArCrCrC |
| | | | | | rArCrGrCrCrCrCrCrArC (SEQ ID NO: |
| | | | | | 66) |
| SpCas9_BCL11A_ | BCL11A_ | SpCas9 | AGG | Anti- | mC*mU*mA*rArCrArGrUrUrGrCrUrUr |
| enh_T1 | enhancer | | | sense | UrUrArUrCrArCrGrUrUrUrUrArGrArGr |
| | | | | | CrUrArGrArArArUrArGrCrArArGrUrUr |
| | | | | | ArArArArUrArArGrGrCrUrArGrUrCrCr |
| | | | | | GrUrUrArUrCrArArCrUrUrGrArArArAr |
| | | | | | ArGrUrGrGrCrArCrCrGrArGrUrCrGrGr |
| | | | | | UrGrCmU*mU*mU*rU (SEQ ID NO: 67) |

r-RNA base
*-phosphorothioated
m-2’ O-methyl

For effector only controls, variant Cas12i2 or SpCas9 were mixed with protein storage buffer (25 mM Tris, pH 7.5, 250 mM NaCl, 1 mM TCEP, 50% glycerol) at the same volume ratio as the crRNA or sgRNA, respectively. Complexations were incubated at 37 degrees Celsius for 30-60 minutes. Following incubation, RNPs were diluted to 18.75 μM, 50 μM, 100 μM, or 160 μM effector concentration for variant Cas12i2 and 18.75 μM or 50 μM for SpCas9. For multiplexing, separate RNPs were mixed together prior to electroporation.

On Day 2, approximately 1e5 cells per electroporation reaction, plus 20% extra, were harvested and counted. Cells were washed once with PBS and resuspended in buffer+ supplement (from Lonza #VXP-3032)+1 mM transfection enhancer oligo (to bring concentration to 4.28 μM in P3 buffer). Concentration of resuspended cells was approximately 5,555 cells/μL.

18 μL of resuspended cells (˜1e5 cells) were mixed with 2 μL of individual or multiplexed RNP complexes to bring final concentration of variant Cas12i2 RNPs to 1.875 μM, 5 μM, 10 μM or 16 μM. Final concentration of SpCas9 RNPs was 1.875 μM or 5 μM. The following controls were set up: unelectroporated cells only, cells in protein storage buffer only. The plate was electroporated using an electroporation device, excluding the unelectroporated conditions. Each electroporation reaction was transferred into 24-well culture plate well containing pre-warmed serum-free media and the appropriate supplement. Cultures were incubated at 37 degrees Celsius, 5% CO₂ for 3 days.

A portion of cell samples (approximately 20 μL) from each test condition was collected at 24, 48, and 72 h post electroporation. Viability was evaluated using AO/PI stain on a cell counter.

On Day 3, cell pellets were prepared from cells remaining after viability testing. Approximately 5e4 cells from each sample were harvested and transferred to a microcentrifuge tube. Cells were pelleted at 1500 rpm for 5 min. Supernatants were removed and pellets were frozen at −80° C.

For genomic DNA extraction, pellets were thawed to room temperature and resuspended in appropriate volume of DNA extraction buffer (from Lucigen) to give final concentration of 1000 cells/μL. Samples were then cycled in PCR machine at 65° C. for 15 min, 68° C. for 15 min, 98° C. for 10 min. Samples were then frozen at −20° C.

Samples for Next Generation Sequencing (NGS) were prepared by rounds of PCR. The first round (PCR I) was used to amplify the genomic regions flanking the target site and add NGS adapters. The second round (PCR II) was used to add NGS indexes. Reactions were then pooled, purified by column purification, and quantified on a fluorometer (Qubit). Sequencing runs were done using a 300 or 150 cycle NGS instrument (NextSeq v2.5) mid or high output kit and run on an NGS instrument (NextSeq 550).

For NGS analysis, the indel mapping function used a sample's fastq file, the amplicon reference sequence, and the forward primer sequence. For each read, a kmer-scanning algorithm was used to calculate the edit operations (match, mismatch, insertion, deletion) between the read and the reference sequence. In order to remove small amounts of primer dimer present in some samples, the first 30 nucleotides of each read were required to match the reference and reads where over half of the mapping nucleotides are mismatches were filtered out as well. Up to 50,000 reads passing those filters were used for analysis, and reads were counted as an indel read if they contained an insertion or deletion. The indel % was calculated as the number of indel-containing reads divided by the number of reads analyzed (reads passing filters up to 50,000). The QC standard for the minimum number of reads passing filters was 10,000. Indels were further assessed for disruption of the GATAA motif sequence by searching for TTATC (reverse complement of GATAA sequence, on the forward strand) sequence in each indel.

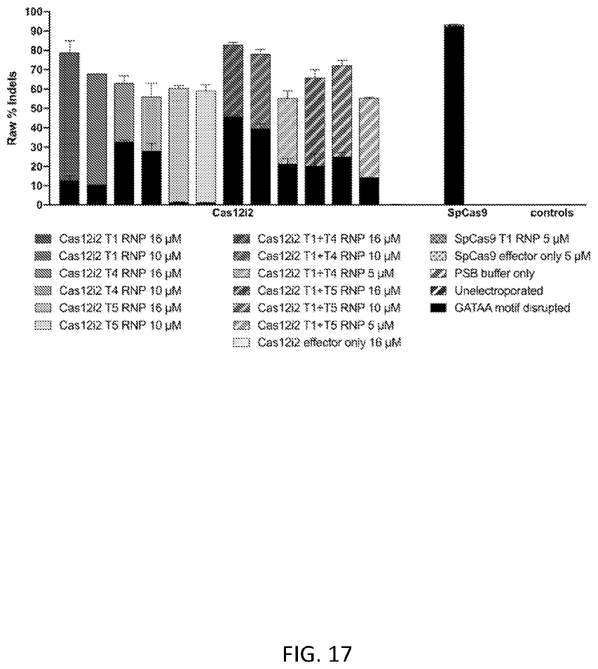

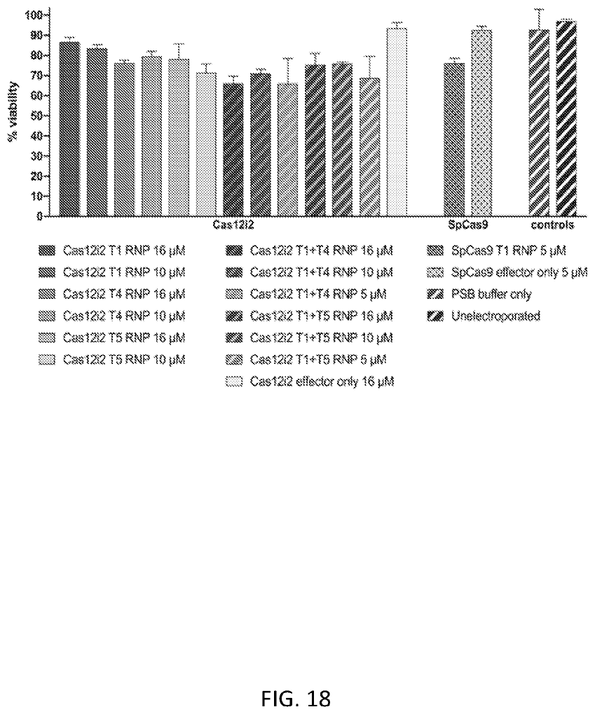

FIG. 17 and FIG. 18 demonstrate the results of this example. As shown in FIG. 17, BCL11A intronic erythroid enhancer-targeting RNP complexes comprising variant Cas12i2 and crRNA resulted in indel activity in primary CD34+ HSPCs. The data showed that at least 50% of variant Cas12i2-induced indels partially or fully disrupted the GATAA motif of BCL11A intronic erythroid enhancer region.

FIG. 18 illustrates that modified CD34+ HSPCs generated with variant Cas12i2 editing of BCL11A intronic erythroid enhance were viable at least 72 hours after treatment of primary CD34+ HSPCs with variant Cas12i2 RNP complexes.

This example demonstrated that variant Cas12i2 comprises robust indel activity. Variant Cas12i2 RNPs that targeted BCL11A intronic erythroid enhancer region-targeting were used to generate modified CD34+ HSPCs and resulted in at least about 50% partial or complete disruption of the GATAA motif in the modified cells.

SEQUENCE LISTING

| SEQ ID | |
|---|---|
| NO | Sequence |
| 1 | atgagcagcg cgatcaaaag ctacaagagc gttctgcgtc cgaacgagcg taagaaccaa |
| | ctgctgaaaa gcaccattca gtgcctggaa gacggtagcg cgttcttttt caagatgctg |
| | caaggcctgt ttggtggcat caccccggag attgttcgtt tcagcaccga acaggagaaa |
| | cagcaacagg atatcgcgct gtggtgcgcg gttaactggt tccgtccggt gagccaagac |
| | agcctgaccc acaccattgc gagcgataac ctggtggaga agtttgagga atactatggt |
| | ggcaccgcga gcgacgcgat caaacagtac ttcagcgcga gcattggcga aagctactat |
| | tggaacgact gccgtcaaca gtactatgat ctgtgccgtg agctgggtgt tgaggtgagc |
| | gacctgaccc atgatctgga gatcctgtgc cgtgaaaagt gcctggcggt tgcgaccgag |
| | agcaaccaga acaacagcat cattagcgtt ctgtttggca ccggcgaaaa agaggaccgt |
| | agcgtgaaac tgcgtatcac caagaaaatt ctggaggcga tcagcaacct gaaagaaatc |
| | ccgaagaacg ttgcgccgat tcaagagatc attctgaacg tggcgaaagc gaccaaggaa |
| | accttccgtc aggtgtatgc gggtaacctg ggtgcgccga gcaccctgga gaaatttatc |
| | gcgaaggacg gccaaaaaga gttcgatctg aagaaactgc agaccgacct gaagaaagtt |
| | attcgtggta aaagcaagga gcgtgattgg tgctgccagg aagagctgcg tagctacgtg |
| | gagcaaaaca ccatccagta tgacctgtgg gcgtggggcg aaatgttcaa caaagcgcac |
| | accgcgctga aaatcaagag cacccgtaac tacaactttg cgaagcaacg tctggaacag |
| | ttcaaagaga ttcagagcct gaacaacctg ctggttgtga agaagctgaa cgactttttc |
| | gatagcgaat ttttcagcgg cgaggaaacc tacaccatct gcgttcacca tctgggtggc |
| | aaggacctga gcaaactgta taaggcgtgg gaggatgatc cggcggaccc ggaaaacgcg |
| | attgtggttc tgtgcgacga tctgaaaaac aactttaaga aagagccgat ccgtaacatt |
| | ctgcgttaca tcttcaccat tcgtcaagaa tgcagcgcgc aggacatcct ggcggcggcg |
| | aagtacaacc aacagctgga tcgttataaa agccaaaagg cgaacccgag cgttctgggt |
| | aaccagggct ttacctggac caacgcggtg atcctgccgg agaaggcgca gcgtaacgac |
| | cgtccgaaca gcctggatct gcgtatttgg ctgtacctga aactgcgtca cccggacggt |
| | cgttggaaga aacaccatat cccgttctac gatacccgtt tcttccaaga aatttatgcg |
| | gcgggcaaca gcccggttga cacctgccag tttcgtaccc cgcgtttcgg ttatcacctg |
| | ccgaaactga ccgatcagac cgcgatccgt gttaacaaga aacatgtgaa agcggcgaag |
| | accgaggcgc gtattcgtct ggcgatccaa cagggcaccc tgccggtgag caacctgaag |
| | atcaccgaaa ttagcgcgac catcaacagc aaaggtcaag tgcgtattcc ggttaagttt |
| | gacgtgggtc gtcaaaaagg caccctgcag atcggtgacc gtttctgcgg ctacgatcaa |
| | aaccagaccg cgagccacgc gtatagcctg tgggaagtgg ttaaagaggg tcaataccat |
| | aaagagctgg gctgctttgt tcgtttcatc agcagcggtg acatcgtgag cattaccgag |
| | aaccgtggca accaatttga tcagctgagc tatgaaggtc tggcgtaccc gcaatatgcg |
| | gactggcgta agaaagcgag caagttcgtg agcctgtggc agatcaccaa gaaaaacaag |
| | aaaaaggaaa tcgtgaccgt tgaagcgaaa gagaagtttg acgcgatctg caagtaccag |
| | ccgcgtctgt ataaattcaa caaggagtac gcgtatctgc tgcgtgatat tgttcgtggc |
| | aaaagcctgg tggaactgca acagattcgt caagagatct ttcgtttcat tgaacaggac |
| | tgcggtgtta cccgtctggg cagcctgagc ctgagcaccc tggaaaccgt gaaagcggtt |
| | aagggtatca tttacagcta ttttagcacc gcgctgaacg cgagcaagaa caacccgatc |
| | agcgacgaac agcgtaaaga gtttgatccg gaactgttcg cgctgctgga aaagctggag |
| | ctgattcgta cccgtaaaaa gaaacaaaaa gtggaacgta tcgcgaacag cctgattcag |
| | acctgcctgg agaacaacat caagttcatt cgtggtgaag gcgacctgag caccaccaac |
| | aacgcgacca agaaaaaggc gaacagccgt agcatggatt ggttggcgcg tggtgttttt |
| | aacaaaatcc gtcaactggc gccgatgcac aacattaccc tgttcggttg cggcagcctg |
| | tacaccagcc accaggaccc gctggtgcat cgtaacccgg ataaagcgat gaagtgccgt |
| | tgggcggcga tcccggttaa ggacattggc gattgggtgc tgcgtaagct gagccaaaac |
| | ctgcgtgcga aaaacatcgg caccggcgag tactatcacc aaggtgttaa agagttcctg |
| | agccattatg aactgcagga cctggaggaa gagctgctga agtggcgtag cgatcgtaaa |
| | agcaacattc cgtgctgggt gctgcagaac cgtctggcgg agaagctggg caacaaagaa |
| | gcggtggttt acatcccggt tcgtggtggc cgtatttatt ttgcgaccca caaggtggcg |
| | accggtgcgg tgagcatcgt tttcgaccaa aaacaagtgt gggtttgcaa cgcggatcat |
| | gttgcggcgg cgaacatcgc gctgaccgtg aagggtattg gcgaacaaag cagcgacgaa |
| | gagaacccgg atggtagccg tatcaaactg cagctgacca gc |
| 2 | MSSAIKSYKS VLRPNERKNQ LLKSTIQCLE DGSAFFFKML QGLFGGITPE IVRFSTEQEK |
| | QQQDIALWCA VNWFRPVSQD SLTHTIASDN LVEKFEEYYG GTASDAIKQY FSASIGESYY |
| | WNDCRQQYYD LCRELGVEVS DLTHDLEILC REKCLAVATE SNQNNSIISV LFGTGEKEDR |
| | SVKLRITKKI LEAISNLKEI PKNVAPIQEI ILNVAKATKE TFRQVYAGNL GAPSTLEKFI |
| | AKDGQKEFDL KKLQTDLKKV IRGKSKERDW CCQEELRSYV EQNTIQYDLW AWGEMFNKAH |
| | TALKIKSTRN YNFAKQRLEQ FKEIQSLNNL LVVKKLNDFF DSEFFSGEET YTICVHHLGG |
| | KDLSKLYKAW EDDPADPENA IVVLCDDLKN NFKKEPIRNI LRYIFTIRQE CSAQDILAAA |
| | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF DVGRQKGTLQ IGDRFCGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVTVEAK EKFDAICKYQ PRLYKFNKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTTN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNIGTGE YYHQGVKEFL SHYELQDLEE ELLKWRSDRK |
| | SNIPCWVLQN RLAEKLGNKE AVVYIPVRGG RIYFATHKVA TGAVSIVFDQ KQVWVCNADH |
| | VAAANIALTV KGIGEQSSDE ENPDGSRIKL QLTS |
| 3 | MSSAIKSYKS VLRPNERKNQ LLKSTIQCLE DGSAFFFKML QGLFGGITPE IVRFSTEQEK |
| | QQQDIALWCA VNWFRPVSQD SLTHTIASDN LVEKFEEYYG GTASDAIKQY FSASIGESYY |
| | WNDCRQQYYD LCRELGVEVS DLTHDLEILC REKCLAVATE SNQNNSIISV LFGTGEKEDR |
| | SVKLRITKKI LEAISNLKEI PKNVAPIQEI ILNVAKATKE TFRQVYAGNL GAPSTLEKFI |
| | AKDGQKEFDL KKLQTDLKKV IRGKSKERDW CCQEELRSYV EQNTIQYDLW AWGEMFNKAH |
| | TALKIKSTRN YNFAKQRLEQ FKEIQSLNNL LVVKKLNDFF DSEFFSGEET YTICVHHLGG |
| | KDLSKLYKAW EDDPADPENA IVVLCDDLKN NFKKEPIRNI LRYIFTIRQE CSAQDILAAA |
| | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVTVEAK EKFDAICKYQ PRLYKFNKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTTN |
| | NATKKKANSR SMDWLARGVF NKIRQLAPMH NITLFGCGSL YTSHQDPLVH RNPDKAMKCR |
| | WAAIPVKDIG RWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVFDQ KQVWVCNADH |
| | VAAANIALTG KGIGEQSSDE ENPDGSRIKL QLTS |
| 4 | MSSAIKSYKS VLRPNERKNQ LLKSTIQCLE DGSAFFFKML QGLFGGITPE IVRFSTEQEK |
| | QQQDIALWCA VNWFRPVSQD SLTHTIASDN LVEKFEEYYG GTASDAIKQY FSASIGESYY |
| | WNDCRQQYYD LCRELGVEVS DLTHDLEILC REKCLAVATE SNQNNSIISV LFGTGEKEDR |
| | SVKLRITKKI LEAISNLKEI PKNVAPIQEI ILNVAKATKE TFRQVYAGNL GAPSTLEKFI |
| | AKDGQKEFDL KKLQTDLKKV IRGKSKERDW CCQEELRSYV EQNTIQYDLW AWGEMFNKAH |
| | TALKIKSTRN YNFAKQRLEQ FKEIQSLNNL LVVKKLNDFF DSEFFSGEET YTICVHHLGG |
| | KDLSKLYKAW EDDPADPENA IVVLCDDLKN NFKKEPIRNI LRYIFTIRQE CSAQDILAAA |
| | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVTVEAK EKFDAICKYQ PRLYKFNKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTTN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVFDQ KQVWVCNADH |
| | VAAANIALTG KGIGEQSSDE ENPDGSRIKL QLTS |
| 5 | MDKKYSIGLD IGTNSVGWAV ITDEYKVPSK KFKVLGNTDR HSIKKNLIGA LLFDSGETAE |
| | ATRLKRTARR RYTRRKNRIC YLQEIFSNEM AKVDDSFFHR LEESFLVEED KKHERHPIFG |
| | NIVDEVAYHE KYPTIYHLRK KLVDSTDKAD LRLIYLALAH MIKFRGHFLI EGDLNPDNSD |
| | VDKLFIQLVQ TYNQLFEENP INASGVDAKA ILSARLSKSR RLENLIAQLP GEKKNGLFGN |
| | LIALSLGLTP NFKSNFDLAE DAKLQLSKDT YDDDLDNLLA QIGDQYADLF LAAKNLSDAI |
| | LLSDILRVNT EITKAPLSAS MIKRYDEHHQ DLTLLKALVR QQLPEKYKEI FFDQSKNGYA |
| | GYIDGGASQE EFYKFIKPIL EKMDGTEELL VKLNREDLLR KQRTFDNGSI PHQIHLGELH |
| | AILRRQEDFY PFLKDNREKI EKILTFRIPY YVGPLARGNS RFAWMTRKSE ETITPWNFEE |
| | VVDKGASAQS FIERMTNFDK NLPNEKVLPK HSLLYEYFTV YNELTKVKYV TEGMRKPAFL |
| | SGEQKKAIVD LLFKTNRKVT VKQLKEDYFK KIECFDSVEI SGVEDRFNAS LGTYHDLLKI |
| | IKDKDFLDNE ENEDILEDIV LTLTLFEDRE MIEERLKTYA HLFDDKVMKQ LKRRRYTGWG |
| | RLSRKLINGI RDKQSGKTIL DFLKSDGFAN RNFMQLIHDD SLTFKEDIQK AQVSGQGDSL |
| | HEHIANLAGS PAIKKGILQT VKVVDELVKV MGRHKPENIV IEMARENQTT QKGQKNSRER |
| | MKRIEEGIKE LGSQILKEHP VENTQLQNEK LYLYYLQNGR DMYVDQELDI NRLSDYDVDH |
| | IVPQSFLKDD SIDNKVLTRS DKNRGKSDNV PSEEVVKKMK NYWRQLLNAK LITQRKFDNL |
| | TKAERGGLSE LDKAGFIKRQ LVETRQITKH VAQILDSRMN TKYDENDKLI REVKVITLKS |
| | KLVSDFRKDF QFYKVREINN YHHAHDAYLN AVVGTALIKK YPKLESEFVY GDYKVYDVRK |
| | MIAKSEQEIG KATAKYFFYS NIMNFFKTEI TLANGEIRKR PLIETNGETG EIVWDKGRDF |
| | ATVRKVLSMP QVNIVKKTEV QTGGFSKESI LPKRNSDKLI ARKKDWDPKK YGGFDSPTVA |
| | YSVLVVAKVE KGKSKKLKSV KELLGITIME RSSFEKNPID FLEAKGYKEV KKDLITKLPK |
| | YSLFELENGR KRMLASAGEL QKGNELALPS KYVNFLYLAS HYEKLKGSPE DNEQKQLFVE |
| | QHKHYLDEII EQISEFSKRV ILADANLDKV LSAYNKHRDK PIREQAENII HLFTLTNLGA |
| | PAAFKYFDTT IDRKRYTSTK EVLDATLIHQ SITGLYETRI DLSQLGGD |
| 6 | TGTCCCCCCAAGTTTTGGAC |
| 7 | AGAAAUCCGUCUUUCAUUGACGGUGUCCCCCCAAGUUUUGGAC |
| 8 | GCTTTTGTCCCCCCAAGTTT |
| 9 | GCUUUUGUCCCCCCAAGUUUGTTTTAGAGCTAGAAATAGCAAGTTAAAATAAGGCTAGTCCGTTATCA |
| | ACTTGAAAAAGTGGCACCGAGTCGGTGCUUUUUU |
| 10 | GGAGAGGTGAGGGACTTGGG |
| 11 | AGAAAUCCGUCUUUCAUUGACGGGGAGAGGUGAGGGACUUGGG |
| 12 | GGAGAGGTGAGGGACTTGGG |
| 13 | GGAGAGGUGAGGGACUUGGGGTTTTAGAGCTAGAAATAGCAAGTTAAAATAAGGCTAGTCCGTTATCA |
| | ACTTGAAAAAGTGGCACCGAGTCGGTGCUUUUUU |
| 14 | GTGAGAATGGTGCGTCCTAG |
| 15 | AGAAAUCCGUCUUUCAUUGACGGGUGAGAAUGGUGCGUCCUAG |
| 16 | TTGTGAGAATGGTGCGTCCT |
| 17 | UUGUGAGAAUGGUGCGUCCUGTTTTAGAGCTAGAAATAGCAAGTTAAAATAAGGCTAGTCCGTTATCA |
| | ACTTGAAAAAGTGGCACCGAGTCGGTGCUUUUUU |
| 18 | AACTGGCCCTGGCTTTGGCA |
| 19 | AGAAAUCCGUCUUUCAUUGACGGAACUGGCCCUGGCUUUGGCA |
| 20 | GCTTTAACTGGCCCTGGCTT |
| 21 | GCUUUAACUGGCCCUGGCUUGTTTTAGAGCTAGAAATAGCAAGTTAAAATAAGGCTAGTCCGTTATCA |
| | ACTTGAAAAAGTGGCACCGAGTCGGTGCUUUUUU |
| 22 | GGATGGCGACTTCAGGCACA |
| 23 | AGAAAUCCGUCUUUCAUUGACGGGGAUGGCGACUUCAGGCACA |
| 24 | TGGATGGCGACTTCAGGCAC |
| 25 | UGGAUGGCGACUUCAGGCACGTTTTAGAGCTAGAAATAGCAAGTTAAAATAAGGCTAGTCCGTTATCA |
| | ACTTGAAAAAGTGGCACCGAGTCGGTGCUUUUUU |
| 26 | ATGTGATTGATGCCCAAAGG |
| 27 | AGAAAUCCGUCUUUCAUUGACGGAUGUGAUUGAUGCCCAAAGG |
| 28 | TTTATGTGATTGATGCCCAA |
| 29 | UUUAUGUGAUUGAUGCCCAAGTTTTAGAGCTAGAAATAGCAAGTTAAAATAAGGCTAGTCCGTTATCA |
| | ACTTGAAAAAGTGGCACCGAGTCGGTGCUUUUUU |
| 30 | GGGGAGGCCTGGAGTCATGG |
| 31 | AGAAAUCCGUCUUUCAUUGACGGGGGGAGGCCUGGAGUCAUGG |
| 32 | TTTGGGGAGGCCTGGAGTCA |
| 33 | UUUGGGGAGGCCUGGAGUCAGTTTTAGAGCTAGAAATAGCAAGTTAAAATAAGGCTAGTCCGTTATCA |
| | ACTTGAAAAAGTGGCACCGAGTCGGTGCUUUUUU |
| 34 | TGGGGGTGACCGCCGGAGCG |
| 35 | AGAAAUCCGUCUUUCAUUGACGGUGGGGGUGACCGCCGGAGCG |
| 36 | TGGGGGTGACCGCCGGAGCG |
| 37 | UGGGGGUGACCGCCGGAGCGGTTTTAGAGCTAGAAATAGCAAGTTAAAATAAGGCTAGTCCGTTATCA |
| | ACTTGAAAAAGTGGCACCGAGTCGGTGCUUUUUU |
| 38 | GTTGACATTGTCCACACCTG |
| 39 | AGAAAUCCGUCUUUCAUUGACGGGUUGACAUUGUCCACACCUG |
| 40 | TTGTTGACATTGTCCACACC |
| 41 | UUGUUGACAUUGUCCACACCGTTTTAGAGCTAGAAATAGCAAGTTAAAATAAGGCTAGTCCGTTATCA |
| | ACTTGAAAAAGTGGCACCGAGTCGGTGCUUUUUU |
| 42 | GGAAATCTATTGAGGCTCTG |
| 43 | AGAAAUCCGUCUUUCAUUGACGGGGAAAUCUAUUGAGGCUCUG |
| 44 | TTGGAAATCTATTGAGGCTC |
| 45 | UUGGAAAUCUAUUGAGGCUCGTTTTAGAGCTAGAAATAGCAAGTTAAAATAAGGCTAGTCCGTTATCA |
| | ACTTGAAAAAGTGGCACCGAGTCGGTGCUUUUUU |
| 46 | MSSAIKSYKS VLRPNERKNQ LLKSTIQCLE DGSAFFFKML QGLFGGITPE IVRFSTEQEK |
| | QQQDIALWCA VNWFRPVSQD SLTHTIASDN LVEKFEEYYG GTASDAIKQY FSASIGESYY |
| | WNDCRQQYYD LCRELGVEVS DLTHDLEILC REKCLAVATE SNQNNSIISV LFGTGEKEDR |
| | SVKLRITKKI LEAISNLKEI PKNVAPIQEI ILNVAKATKE TFRQVYAGNL GAPSTLEKFI |
| | AKDGQKEFDL KKLQTDLKKV IRGKSKERDW CCQEELRSYV EQNTIQYDLW AWGEMFNKAH |
| | TALKIKSTRN YNFAKQRLEQ FKEIQSLNNL LVVKKLNDFF DSEFFSGEET YTICVHHLGG |
| | KDLSKLYKAW EDDPADPENA IVVLCDDLKN NFKKEPIRNI LRYIFTIRQE CSAQDILAAA |
| | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELGCFVRFI SSGDIVSITE NRGNQFDQLS YEGLAYPQYA |
| | DWRKKASKFV SLWQITKKNK KKEIVTVEAK EKFDAICKYQ PRLYKFNKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTTN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVFDQ KQVWVCNADH |
| | VAAANIALTG KGIGEQSSDE ENPDGGRIKL QLTS |
| 47 | MSSAIKSYKS VLRPNERKNQ LLKSTIQCLE DGSAFFFKML QGLFGGITPE IVRFSTEQEK |
| | QQQDIALWCA VNWFRPVSQD SLTHTIASDN LVEKFEEYYG GTASDAIKQY FSASIGESYY |
| | WNDCRQQYYD LCRELGVEVS DLTHDLEILC REKCLAVATE SNQNNSIISV LFGTGEKEDR |
| | SVKLRITKKI LEAISNLKEI PKNVAPIQEI ILNVAKATKE TFRQVYAGNL GAPSTLEKFI |
| | AKDGQKEFDL KKLQTDLKKV IRGKSKERDW CCQEELRSYV EQNTIQYDLW AWGEMFNKAH |
| | TALKIKSTRN YNFAKQRLEQ FKEIQSLNNL LVVKKLNDFF DSEFFSGEET YTICVHHLGG |
| | KDLSKLYKAW EDDPADPENA IVVLCDDLKN NFKKEPIRNI LRYIFTIRQE CSAQDILAAA |
| | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELRCRVRFI SSGDIVSITE NRGNQFDQLS YEGLAYPQYA |
| | DWRKKASKFV SLWQITKKNK KKEIVTVEAK EKFDAICKYQ PRLYKFNKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTTN |
| | NATKKKANSR SMDWLARGVF NKIRQLAPMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVFDQ KQVWVCNADH |
| | VAAANIALTG KGIGRQSSDE ENPDGGRIKL QLTS |
| 48 | MSSAIKSYKS VLRPNERKNQ LLKSTIQCLE DGSAFFFKML QGLFGGITPE IVRFSTEQEK |
| | QQQDIALWCA VNWFRPVSQD SLTHTIASDN LVEKFEEYYG GTASDAIKQY FSASIGESYY |
| | WNDCRQQYYD LCRELGVEVS DLTHDLEILC REKCLAVATE SNQNNSIISV LFGTGEKEDR |
| | SVKLRITKKI LEAISNLKEI PKNVAPIQEI ILNVAKATKE TFRQVYAGNL GAPSTLEKFI |
| | AKDGQKEFDL KKLQTDLKKV IRGKSKERDW CCQEELRSYV EQNTIQYDLW AWGEMFNKAH |
| | TALKIKSTRN YNFAKQRLEQ FKEIQSLNNL LVVKKLNDFF DSEFFSGEET YTICVHHLGG |
| | KDLSKLYKAW EDDPADPENA IVVLCDDLKN NFKKEPIRNI LRYIFTIRQE CSAQDILAAA |
| | KYNQQLDRYK SQKANPSVLG NQGFTWTNAV ILPEKAQRND RPNSLDLRIW LYLKLRHPDG |
| | RWKKHHIPFY DTRFFQEIYA AGNSPVDTCQ FRTPRFGYHL PKLTDQTAIR VNKKHVKAAK |
| | TEARIRLAIQ QGTLPVSNLK ITEISATINS KGQVRIPVKF RVGRQKGTLQ IGDRFCGYDQ |
| | NQTASHAYSL WEVVKEGQYH KELRCRVRFI SSGDIVSITE NRGNQFDQLS YEGLAYPQYA |
| | DWRKKASKFV SLWQITKKNK KKEIVTVEAK EKFDAICKYQ PRLYKFNKEY AYLLRDIVRG |
| | KSLVELQQIR QEIFRFIEQD CGVTRLGSLS LSTLETVKAV KGIIYSYFST ALNASKNNPI |
| | SDEQRKEFDP ELFALLEKLE LIRTRKKKQK VERIANSLIQ TCLENNIKFI RGEGDLSTTN |
| | NATKKKANSR SMDWLARGVF NKIRQLATMH NITLFGCGSL YTSHQDPLVH RNPDKAMKCR |
| | WAAIPVKDIG DWVLRKLSQN LRAKNRGTGE YYHQGVKEFL SHYELQDLEE ELLKWRSDRK |
| | SNIPCWVLQN RLAEKLGNKE AVVYIPVRGG RIYFATHKVA TGAVSIVFDQ KQVWVCNADH |
| | VAAANIALTG KGIGRQSSDE ENPDGGRIKL QLTS |
| 49 | rArGrArArArUrCrCrGrUrCrUrUrUrCrArUrUrGrArCrGrGrArArUrGrUrCrGrGrArUrG |
| | rGrArUrGrArArArCrC |
| 50 | rArGrArArArUrCrCrGrUrCrUrUrUrCrArUrUrGrArCrGrGrCrUrArUrCrUrCrUrUrGrU |
| | rArCrUrArCrArCrUrG |
| 51 | rArGrArArArUrCrCrGrUrCrUrUrUrCrArUrUrGrArCrGrGrArCrArCrGrGrCrArGrGrC |
| | rArUrArCrUrCrArUrC |
| 52 | rArGrArArArUrCrCrGrUrCrUrUrUrCrArUrUrGrArCrGrGrGrUrCrArCrArGrCrCrCrA |
| | rArGrArUrArGrUrUrA |
| 53 | mG*mG*mC*rCrGrArGrArUrGrUrCrUrCrGrCrUrCrCrGrGrUrUrUrUrArGrArGrCrUrAr |
| | GrArArArUrArGrCrArArGrUrUrArArArArUrArArGrGrCrUrArGrUrCrCrGrUrUrArUr |
| | CrArArCrUrUrGrArArArArArGrUrGrGrCrArCrCrGrArGrUrCrGrGrUrGrCmU*mU*mU* |
| | rU |
| 54 | rArGrArArArUrCrCrGrUrCrUrUrUrCrArUrUrGrArCrGrGrArArGrArGrCrArArCrArG |
| | rUrGrCrUrGrUrGrGrC |
| 55 | rArGrArArArUrCrCrGrUrCrUrUrUrCrArUrUrGrArCrGrGrArArCrArArCrArGrCrArU |
| | rUrArUrUrCrCrArGrA |
| 56 | rArGrArArArUrCrCrGrUrCrUrUrUrCrArUrUrGrArCrGrGrGrArArArCrArGrGrUrArA |
| | rGrArCrArGrGrGrGrU |
| 57 | rArGrArArArUrCrCrGrUrCrUrUrUrCrArUrUrGrArCrGrGrCrArGrGrArGrGrArGrGrA |
| | rUrUrCrGrGrArArCrC |
| 58 | mA*mA*mG*rUrUrCrCrUrGrUrGrArUrGrUrCrArArGrCrGrUrUrUrUrArGrArGrCrUrAr |
| | GrArArArUrArGrCrArArGrUrUrArArArArUrArArGrGrCrUrArGrUrCrCrGrUrUrArUr |
| | CrArArCrUrUrGrArArArArArGrUrGrGrCrArCrCrGrArGrUrCrGrGrUrGrCmU*mU*mU* |
| | rU |
| 59 | rArGrArArArUrCrCrGrUrCrUrUrUrCrArUrUrGrArCrGrGrUrUrArGrGrUrArGrGrUrG |
| | rGrGrGrUrCrGrGrCrG |
| 60 | rArGrArArArUrCrCrGrUrCrUrUrUrCrArUrUrGrArCrGrGrCrCrCrGrArGrGrArCrCrG |
| | rCrArGrCrCrArGrCrC |
| 61 | rArGrArArArUrCrCrGrUrCrUrUrUrCrArUrUrGrArCrGrGrCrGrUrGrUrCrArCrArCrA |
| | rArCrUrGrCrCrCrArA |
| 62 | rArGrArArArUrCrCrGrUrCrUrUrUrCrArUrUrGrArCrGrGrCrArCrArUrGrArGrCrGrU |
| | rGrGrUrCrArGrGrGrC |
| 63 | mU*mC*mC*rArGrGrCrArUrGrCrArGrArUrCrCrCrArCrGrUrUrUrUrArGrArGrCrUrAr |
| | GrArArArUrArGrCrArArGrUrUrArArArArUrArArGrGrCrUrArGrUrCrCrGrUrUrArUr |
| | CrArArCrUrUrGrArArArArArGrUrGrGrCrArCrCrGrArGrUrCrGrGrUrGrCmU*mU*mU* |
| | rU |
| 64 | rArGrArArArUrCrCrGrUrCrUrUrUrCrArUrUrGrArCrGrGrGrArArGrCrUrArGrUrCrU |
| | rArGrUrGrCrArArGrC |
| 65 | rArGrArArArUrCrCrGrUrCrUrUrUrCrArUrUrGrArCrGrGrCrUrGrGrArGrCrCrUrGrU |
| | rGrArUrArArArArGrC |
| 66 | rArGrArArArUrCrCrGrUrCrUrUrUrCrArUrUrGrArCrGrGrUrArCrCrCrCrArCrCrCrA |
| | rCrGrCrCrCrCrCrArC |
| 67 | mC*mU*mA*rArCrArGrUrUrGrCrUrUrUrUrArUrCrArCrGrUrUrUrUrArGrArGrCrUrAr |
| | GrArArArUrArGrCrArArGrUrUrArArArArUrArArGrGrCrUrArGrUrCrCrGrUrUrArUr |
| | CrArArCrUrUrGrArArArArArGrUrGrGrCrArCrCrGrArGrUrCrGrGrUrGrCmU*mU*mU* |
| | rU |

What is claimed is:

1. A modified cell comprising a Cas12i-induced genomic deletion, wherein
(a) the deletion starts within about 5 nucleotides to about 15 nucleotides downstream of a 5′-NTTN-3′ sequence, wherein N is any nucleotide;
(b) wherein the deletion is greater than about 15 nucleotides in length; and
(c) wherein the modified cell substantially lacks expression of the gene.

2. The modified cell of claim 1, wherein the 5′-NTTN-3′ sequence is on a sense strand of the gene.

3. The modified cell of claim 1, wherein the 5′-NTTN-3′ sequence is on an antisense strand of the gene.

4. The modified cell of claim 1, wherein the deletion starts within about 5 nucleotides to about 10 nucleotides downstream of the 5′-NTTN-3′ sequence.

5. The modified cell of claim 1, wherein the deletion starts within about 10 nucleotides to about 15 nucleotides downstream of the 5′-NTTN-3′ sequence.

6. The modified cell of claim 1, wherein the deletion ends within about 30 nucleotides to about 50 nucleotides downstream of the 5′-NTTN-3′ sequence.

7. The modified cell of claim 1, wherein the deletion ends within about 30 nucleotides to about 40 nucleotides downstream of the 5′-NTTN-3′ sequence.

8. The modified cell of claim 1, wherein the deletion ends within about 20 to about 30 nucleotides downstream of the 5′-NTTN-3′ sequence.

9. The modified cell of claim 1, wherein
(a) the deletion starts within about 5 to about 15 nucleotides downstream of the 5′-NTTN-3′ sequence on the sense strand;
(b) wherein the deletion ends within about 5 to about 15 nucleotides of a 5′-NAAN-3′ sequence on the sense strand, wherein N is any nucleotide; and
(c) wherein the 5′-NAAN-3′ sequence is downstream of the 5′-NTTN-3′ sequence.

10. The modified cell of claim 1, wherein
(a) the deletion starts within about 5 to about 15 nucleotides downstream of the 5′-NTTN-3′ sequence on the antisense strand;
(b) wherein the deletion ends within about 5 to about 15 nucleotides of a 5′-NAAN-3′ sequence on the antisense strand, wherein N is any nucleotide; and
(c) wherein the 5′-NAAN-3′ sequence is downstream of the 5′-NTTN-3′ sequence.

11. The modified cell of claim 9 or claim 10, wherein the deletion is greater than about 40 nucleotides in length.

12. The modified cell of claim 1, wherein the deletion is in an exon of the gene.

13. The modified cell of claim 1, wherein the deletion overlaps with a mutation in the gene.

14. The modified cell of claim 1, wherein the deletion overlaps with an insertion in the gene.

15. The modified cell of claim 1, wherein the deletion removes at least a portion of a repeat expansion of the gene.

16. The modified cell of claim 1, wherein the deletion disrupts one or both alleles of the gene.

17. The modified cell of claim 1, wherein the modified cell comprises two or more deletions.

18. The modified cell of claim 1, wherein an unmodified cell lacks the deletion.

19. The modified cell of claim 18, wherein the unmodified cell expresses the gene.

20. The modified cell of claim 18, wherein the unmodified cell is a wild-type cell.

21. The modified cell of claim 1, wherein the 5′-NTTN-3′ sequence is 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′, wherein Y is C or T, B is any nucleotide except for A, D is any nucleotide except for C, and R is A or G.

22. The modified cell of claim 1, wherein the 5′-NTTN-3′ sequence is 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′.

23. The modified cell of claim 1, wherein the modified cell is a eukaryotic cell.

24. The modified cell of claim 1, wherein the modified cell is an animal cell or a cell derived from an animal cell.

25. The modified cell of claim 1, wherein the modified cell is a mammalian cell or derived from a mammalian cell.

26. The modified cell of claim 1, wherein the modified cell is a human cell or derived from a human cell.

27. The modified cell of claim 1, wherein the modified cell is a stem cell (e.g., a totipotent/omnipotent stem cell, a pluripotent stem cell, a multipotent stem cell, an oligopotent stem cell, or an unipotent stem cell), a differentiated cell, or a terminally differentiated cell.

28. The modified cell of claim 1, wherein the modified cell is a primary cell.

29. The modified cell of claim 1, wherein the modified cell is from a cell line.

30. The modified cell of claim 1, wherein the modified cell is a T cell, B cell, or NK cell.

31. The modified cell of claim 1, wherein the modified cell comprises a modification in a gene selected from the group consisting of: BCL11A intronic erythroid enhancer, CD3, B2M, TRAC, PDCD1, PDL1, CIITA, TTR, LDHA, and HAO1.

32. Progeny of the modified cell of claim 1.

33. A method of obtaining a plurality of cells, wherein the method comprises isolating and culturing the modified cell of claim 1.

34. A method of obtaining a plurality of cells, wherein the method comprises culturing the modified cell of claim 1.

35. A plurality of cells comprising the modified cell or a plurality of cells derived from the modified cell of claim 1.

36. A modified cell comprising a deletion, wherein the deletion is adjacent to a 5′-NTTN-3′ sequence, wherein N is any nucleotide.

37. The modified cell of claim 36, wherein the deletion is up to about 40 nucleotides in length.

38. The modified cell of claim 36, wherein the deletion is from about 4 nucleotides to about 40 nucleotides in length.

39. The modified cell of claim 36, wherein the deletion is from about 4 nucleotides to about 25 nucleotides in length.

40. The modified cell of claim 36, wherein the deletion is from about 10 nucleotides to about 25 nucleotides in length.

41. The modified cell of claim 36, wherein the deletion is from about 10 nucleotides to about 15 nucleotides in length.

42. The modified cell of claim 36, wherein the deletion is downstream of the 5′-NTTN-3′ sequence.

43. The modified cell of claim 36, wherein the deletion starts within about 5 nucleotides to about 15 nucleotides of the 5′-NTTN-3′ sequence.

44. The modified cell of claim 36, wherein the deletion starts within about 5 nucleotides to about 10 nucleotides of the 5′-NTTN-3′ sequence.

45. The modified cell of claim 36, wherein the deletion starts within about 10 nucleotides to about 15 nucleotides of the 5′-NTTN-3′ sequence.

46. The modified cell of claim 36, wherein the deletion starts within about 5 nucleotides to about 15 nucleotides downstream of the 5′-NTTN-3′ sequence.

47. The modified cell of claim 36, wherein the deletion starts within about 5 nucleotides to about 10 nucleotides downstream of the 5′-NTTN-3′ sequence.

48. The modified cell of claim 36, wherein the deletion starts within about 10 nucleotides to about 15 nucleotides downstream of the 5′-NTTN-3′ sequence.

49. The modified cell of claim 36, wherein the deletion ends within about 20 nucleotides to about 30 nucleotides of the 5′-NTTN-3′ sequence.

50. The modified cell of claim 36, wherein the deletion ends within about 20 nucleotides to about 25 nucleotides of the 5′-NTTN-3′ sequence.

51. The modified cell of claim 36, wherein the deletion ends within about 25 nucleotides to about 30 nucleotides of the 5′-NTTN-3′ sequence.

52. The modified cell of claim 36, wherein the deletion ends within about 20 nucleotides to about 30 nucleotides downstream of the 5′-NTTN-3′ sequence.

53. The modified cell of claim 36, wherein the deletion ends within about 20 nucleotides to about 25 nucleotides downstream of the 5′-NTTN-3′ sequence.

54. The modified cell of claim 36, wherein the deletion ends within about 25 nucleotides to about 30 nucleotides downstream of the 5′-NTTN-3′ sequence.

55. The modified cell of claim 36, wherein the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

56. The modified cell of claim 36, wherein the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

57. The modified cell of claim 36, wherein the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

58. The modified cell of claim 36, wherein the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

59. The modified cell of claim 36, wherein the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

60. The modified cell of claim 36, wherein the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

61. The modified cell of claim 36, wherein the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

62. The modified cell of claim 36, wherein the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

63. The modified cell of claim 36, wherein the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

64. The modified cell of claim 36, wherein the deletion is in a genome of the modified cell.

65. The modified cell of claim 36, wherein the deletion is in an exon of the gene.

66. The modified cell of claim 36, wherein the deletion overlaps with a mutation in the gene.

67. The modified cell of claim 36, wherein the deletion overlaps with an insertion in the gene.

68. The modified cell of claim 36, wherein the deletion removes at least a portion of a repeat expansion of the gene.

69. The modified cell of claim 36, wherein the deletion disrupts one or both alleles of the gene.

70. The modified cell of claim 36, wherein the modified cell comprises two or more deletions.

71. The modified cell of claim 36, wherein an unmodified cell lacks the deletion.

72. The modified cell of claim 71, wherein the unmodified cell is a wild-type cell.

73. The modified cell of claim 36, wherein a number of nucleotides deleted in the modified cell is greater than a number of nucleotides deleted in a second modified cell, wherein the second modified is generated by treating an unmodified cell with a Cas9 polypeptide of SEQ ID NO: 5.

74. A modified cell comprising a DNA insertion, wherein the DNA insertion is adjacent to a 5′-NTTN-3′ sequence, wherein N is any nucleotide.

75. The modified cell of claim 74, wherein the insertion is 1 nucleotide in length.

76. The modified cell of claim 74, wherein the insertion is from 2 nucleotides to about 9 nucleotides in length.

77. The modified cell of claim 74, wherein the insertion is greater than about 9 nucleotides in length.

78. The modified cell of claim 74, wherein the insertion is downstream of the 5′-NTTN-3′ sequence.

79. The modified cell of claim 74, wherein the insertion starts within about 15 nucleotides to about 35 nucleotides of the 5′-NTTN-3′ sequence.

80. The modified cell of claim 74, wherein the insertion starts within about 18 nucleotides to about 30 nucleotides of the 5′-NTTN-3′ sequence.

81. The modified cell of claim 74, wherein the insertion starts within about 20 nucleotides to about 28 nucleotides of the 5′-NTTN-3′ sequence.

82. The modified cell of claim 74, wherein the insertion starts within about 20 nucleotides to about 25 nucleotides downstream of the 5′-NTTN-3′ sequence.

83. The modified cell of claim 74, wherein the insertion is in a genome of the modified cell.

84. The modified cell of claim 74, wherein the insertion is in an exon of the gene.

85. The modified cell of claim 74, wherein the insertion overlaps with a mutation in the gene.

86. The modified cell of claim 74, wherein the insertion overlaps with a deletion in the gene.

87. The modified cell of claim 74, wherein the insertion corrects a frameshift in the gene.

88. The modified cell of claim 74, wherein the insertion disrupts one or both alleles of the gene.

89. The modified cell of claim 74, wherein an unmodified cell lacks the DNA insertion.

90. The modified cell of claim 89, wherein the unmodified cell is a wild-type cell.

91. The modified cell of claim 36 or claim 74, wherein the 5′-NTTN-3′ sequence is 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′, wherein Y is C or T, B is any nucleotide except for A, D is any nucleotide except for C, and R is A or G.

92. The modified cell of claim 36 or claim 74, wherein the 5′-NTTN-3′ sequence is 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′.

93. The modified cell of claim 36 or claim 74, wherein the modified cell is a eukaryotic cell or a prokaryotic cell.

94. The modified cell of claim 36 or claim 74, wherein the modified cell is an animal cell, a plant cell, or a fungal cell or the cell is derived from an animal cell, a plant cell, or a fungal cell.

95. The modified cell of claim 36 or claim 74, wherein the modified cell is a mammalian cell or derived from a mammalian cell.

96. The modified cell of claim 36 or claim 74, wherein the modified cell is a human cell or derived from a human cell.

97. The modified cell of claim 36 or claim 74, wherein the modified cell is a stem cell (e.g., a totipotent/omnipotent stem cell, a pluripotent stem cell, a multipotent stem cell, an oligopotent stem cell, or an unipotent stem cell), a differentiated cell, or a terminally differentiated cell.

98. The modified cell of claim 36 or claim 74, wherein the modified cell is a primary cell.

99. The modified cell of claim 36 or claim 74, wherein the modified cell is from a cell line.

100. The modified cell of claim 36 or claim 74, wherein the modified cell is a T cell, B cell, or NK cell.

101. The modified cell of claim 36 or claim 74, wherein the modified cell comprises a modification in a gene selected from the group consisting of: BCL11A intronic erythroid enhancer, CD3, B2M, TRAC, PDCD1, PDL1, CIITA, TTR, LDHA, and HAO1.

102. Progeny of the modified cell of claim 36 or claim 74.

103. A method of obtaining a plurality of cells, wherein the method comprises isolating and culturing the modified cell of claim 36 or claim 74.

104. A method of obtaining a plurality of cells, wherein the method comprises culturing the modified cell of claim 36 or claim 74.

105. A plurality of cells comprising the modified cell or a plurality of the modified cell of claim 36 or claim 74.

106. A plurality of cells, wherein at least 70% of the cells comprise a deletion in a gene, wherein the deletion is adjacent to a 5′-NTTN-3′ sequence.

107. The plurality of cells of claim 106, wherein at least 80% of the cells comprise the deletion.

108. The plurality of cells of claim 106, wherein at least 90% of the cells comprise the deletion.

109. The plurality of cells of claim 106, wherein each of the cells comprises the deletion.

110. The plurality of cells of claim 106, wherein the deletion is at least about 5 nucleotides in length in about 90% of the cells having the deletion.

111. The plurality of cells of claim 106, wherein the deletion is from about 4 nucleotides to about 40 nucleotides in length in the cells having the deletion.

112. The plurality of cells of claim 106, wherein the deletion is at least about 10 nucleotides in length in about 75% of the cells having the deletion.

113. The plurality of cells of claim 106, wherein the deletion is at least about 15 nucleotides in length in about 50% of the cells having the deletion.

114. The plurality of cells of claim 106, wherein the deletion is at least about 20 nucleotides in length in about 25% of the cells having the deletion.

115. The plurality of cells of claim 106, wherein the deletion is at least about 25 nucleotides in length in about 25% of the cells having the deletion.

116. The plurality of cells of claim 106, wherein the deletion is at least about 5 nucleotides or longer in about 90% of the cells having the deletion.

117. The plurality of cells of claim 106, wherein the deletion is about 10 nucleotides or longer in about 75% of the cells having the deletion.

118. The plurality of cells of claim 106, wherein the deletion is about 15 nucleotides or longer in about 50% of the cells having the deletion.

119. The plurality of cells of claim 106, wherein the deletion is about 20 nucleotides or longer in about 25% of the cells having the deletion.

120. The plurality of cells of claim 106, wherein the deletion is about 25 nucleotides or longer in about 25% of the cells having the deletion.

121. The plurality of cells of claim 106, wherein the deletion is downstream of the 5′-NTTN-3′ sequence.

122. The plurality of cells of claim 106, wherein the deletion starts within about 5 nucleotides to about 15 nucleotides of the 5′-NTTN-3′ sequence.

123. The plurality of cells of claim 106, wherein the deletion starts within about 5 nucleotides to about 10 nucleotides of the 5′-NTTN-3′ sequence.

124. The plurality of cells of claim 106, wherein the deletion starts within about 10 nucleotides to about 15 nucleotides of the 5′-NTTN-3′ sequence.

125. The plurality of cells of claim 106, wherein the deletion starts within about 5 nucleotides to about 15 nucleotides downstream of the 5′-NTTN-3′ sequence.

126. The plurality of cells of claim 106, wherein the deletion starts within about 5 nucleotides to about 10 nucleotides downstream of the 5′-NTTN-3′ sequence.

127. The plurality of cells of claim 106, wherein the deletion starts within about 10 nucleotides to about 15 nucleotides downstream of the 5′-NTTN-3′ sequence.

128. The plurality of cells of claim 106, wherein the deletion ends within about 20 nucleotides to about 30 nucleotides of the 5′-NTTN-3′ sequence.

129. The plurality of cells of claim 106, wherein the deletion ends within about 20 nucleotides to about 25 nucleotides of the 5′-NTTN-3′ sequence.

130. The plurality of cells of claim 106, wherein the deletion ends within about 25 nucleotides to about 30 nucleotides of the 5′-NTTN-3′ sequence.

131. The plurality of cells of claim 106, wherein the deletion ends within about 20 nucleotides to about 30 nucleotides downstream of the 5′-NTTN-3′ sequence.

132. The plurality of cells of claim 106, wherein the deletion ends within about 20 nucleotides to about 25 nucleotides downstream of the 5′-NTTN-3′ sequence.

133. The plurality of cells of claim 106, wherein the deletion ends within about 25 nucleotides to about 30 nucleotides downstream of the 5′-NTTN-3′ sequence.

134. The plurality of cells of claim 106, wherein the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

135. The plurality of cells of claim 106, wherein the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

136. The plurality of cells of claim 106, wherein the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

137. The plurality of cells of claim 106, wherein the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

138. The plurality of cells of claim 106, wherein the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

139. The plurality of cells of claim 106, wherein the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

140. The plurality of cells of claim 106, wherein the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

141. The plurality of cells of claim 106, wherein the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

142. The plurality of cells of claim 106, wherein the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

143. The plurality of cells of claim 106, wherein the deletion is in an exon of the gene.

144. The plurality of cells of claim 106, wherein the deletion overlaps with a mutation in the gene.

145. The plurality of cells of claim 106, wherein the deletion overlaps with an insertion in the gene.

146. The plurality of cells of claim 106, wherein the deletion removes at least a portion of a repeat expansion of the gene.

147. The plurality of cells of claim 106, wherein the deletion disrupts one or both alleles of the gene.

148. A plurality of cells, wherein at least 70% of the cells comprise an insertion in a gene, wherein the insertion is adjacent to a 5′-NTTN-3′ sequence.

149. The plurality of cells of claim 148, wherein at least 80% of the cells comprise the insertion.

150. The plurality of cells of claim 148, wherein at least 90% of the cells comprise the insertion.

151. The plurality of cells of claim 148, wherein 100% of the cells comprises the insertion.

152. The plurality of cells of claim 148, wherein the insertion is 1 nucleotide in length.

153. The plurality of cells of claim 148, wherein the insertion is from 2 nucleotides to about 9 nucleotides in length.

154. The plurality of cells of claim 148, wherein the insertion is greater than about 9 nucleotides in length.

155. The plurality of cells of claim 148, wherein the insertion is downstream of the 5′-NTTN-3′ sequence.

156. The plurality of cells of claim 148, wherein the insertion starts within about 15 nucleotides to about 35 nucleotides of the 5′-NTTN-3′ sequence.

157. The plurality of cells of claim 148, wherein the insertion starts within about 18 nucleotides to about 30 nucleotides of the 5′-NTTN-3′ sequence.

158. The plurality of cells of claim 148, wherein the insertion starts within about 20 nucleotides to about 28 nucleotides of the 5′-NTTN-3′ sequence.

159. The plurality of cells of claim 148, wherein the insertion starts within about 20 nucleotides to about 25 nucleotides downstream of the 5′-NTTN-3′ sequence.

160. The plurality of cells of claim 148, wherein the insertion is in an exon of the gene.

161. The plurality of cells of claim 148, wherein the insertion overlaps with a mutation in the gene.

162. The plurality of cells of claim 148, wherein the insertion overlaps with a deletion in the gene.

163. The plurality of cells of claim 148, wherein the insertion corrects a frameshift in the gene.

164. The plurality of cells of claim 148, wherein the insertion disrupts one or both alleles of the gene.

165. A plurality of cells, wherein
(a) at least about 20% of the cells comprise a deletion adjacent to a 5′-NTTN-3′ sequence; and
(b) less than about 3% of the cells comprise an insertion adjacent to the 5′-NTTN-3′ sequence.

166. The plurality of cells of claim 165, wherein at least about 30% of the cells comprise the deletion.

167. The plurality of cells of claim 165, wherein at least about 40% of the cells comprise the deletion.

168. The plurality of cells of claim 165, wherein at least about 50% of the cells comprise the deletion.

169. The plurality of cells of claim 165, wherein at least about 60% of the cells comprise the deletion.

170. The plurality of cells of claim 165, wherein at least about 70% of the cells comprise the deletion.

171. The plurality of cells of claim 165, wherein at least about 80% of the cells comprise the deletion.

172. The plurality of cells of claim 165, wherein at least about 90% of the cells comprise the deletion.

173. The plurality of cells of claim 165, wherein less than about 2% of the cells comprise the insertion.

174. The plurality of cells of claim 165, wherein less than about 1% of the cells comprise the insertion.

175. The plurality of cells of claim 165, wherein less than about 0.5% of the cells comprise the insertion.

176. The plurality of cells of claim 165, wherein less than about 0.1% of the cells comprise the insertion.

177. The plurality of cells of claim 165, wherein the deletion is downstream of the 5′-NTTN-3′ sequence.

178. The plurality of cells of claim 165, wherein the deletion starts within about 5 nucleotides to about 15 nucleotides of the 5′-NTTN-3′ sequence.

179. The plurality of cells of claim 165, wherein the deletion starts within about 5 nucleotides to about 10 nucleotides of the 5′-NTTN-3′ sequence.

180. The plurality of cells of claim 165, wherein the deletion starts within about 10 nucleotides to about 15 nucleotides of the 5′-NTTN-3′ sequence.

181. The plurality of cells of claim 165, wherein the deletion starts within about 5 nucleotides to about 15 nucleotides downstream of the 5′-NTTN-3′ sequence.

182. The plurality of cells of claim 165, wherein the deletion starts within about 5 nucleotides to about 10 nucleotides downstream of the 5′-NTTN-3′ sequence.

183. The plurality of cells of claim 165, wherein the deletion starts within about 10 nucleotides to about 15 nucleotides downstream of the 5′-NTTN-3′ sequence.

184. The plurality of cells of claim 165, wherein the deletion ends within about 20 nucleotides to about 30 nucleotides of the 5′-NTTN-3′ sequence.

185. The plurality of cells of claim 165, wherein the deletion ends within about 20 nucleotides to about 25 nucleotides of the 5′-NTTN-3′ sequence.

186. The plurality of cells of claim 165, wherein the deletion ends within about 25 nucleotides to about 30 nucleotides of the 5′-NTTN-3′ sequence.

187. The plurality of cells of claim 165, wherein the deletion ends within about 20 nucleotides to about 30 nucleotides downstream of the 5′-NTTN-3′ sequence.

188. The plurality of cells of claim 165, wherein the deletion ends within about 20 nucleotides to about 25 nucleotides downstream of the 5′-NTTN-3′ sequence.

189. The plurality of cells of claim 165, wherein the deletion ends within about 25 nucleotides to about 30 nucleotides downstream of the 5′-NTTN-3′ sequence.

190. The plurality of cells of claim 165, wherein the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

191. The plurality of cells of claim 165, wherein the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

192. The plurality of cells of claim 165, wherein the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

193. The plurality of cells of claim 165, wherein the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

194. The plurality of cells of claim 165, wherein the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

195. The plurality of cells of claim 165, wherein the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

196. The plurality of cells of claim 165, wherein the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

197. The plurality of cells of claim 165, wherein the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

198. The plurality of cells of claim 165, wherein the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

199. The plurality of cells of claim 165, wherein the insertion is 1 nucleotide in length.

200. The plurality of cells of claim 165, wherein the insertion is from 2 nucleotides to about 9 nucleotides in length.

201. The plurality of cells of claim 165, wherein the insertion is greater than about 9 nucleotides in length.

202. The plurality of cells of claim 165, wherein the insertion is downstream of the 5′-NTTN-3′ sequence.

203. The plurality of cells of claim 165, wherein the insertion starts within about 15 nucleotides to about 35 nucleotides of the 5′-NTTN-3′ sequence.

204. The plurality of cells of claim 165, wherein the insertion starts within about 18 nucleotides to about 30 nucleotides of the 5′-NTTN-3′ sequence.

205. The plurality of cells of claim 165, wherein the insertion starts within about 20 nucleotides to about 28 nucleotides of the 5′-NTTN-3′ sequence.

206. The plurality of cells of claim 165, wherein the insertion starts within about 20 nucleotides to about 25 nucleotides downstream of the 5′-NTTN-3′ sequence.

207. The plurality of cells of claim 106, claim 148, or claim 165, wherein the 5′-NTTN-3′ sequence is 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′, wherein Y is C or T, B is any nucleotide except for A, D is any nucleotide except for C, and R is A or G.

208. The plurality of cells of claim 106, claim 148, or claim 165, wherein the 5′-NTTN-3′ sequence is 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′.

209. The plurality of cells of claim 106, claim 148, or claim 165, wherein the plurality of cells are eukaryotic cells or prokaryotic cells.

210. The plurality of cells of claim 106, claim 148, or claim 165, wherein the plurality of cells are animal cells, plant cells, or fungal cells or the cells derived from animal cells, plant cells, or fungal cells.

211. The plurality of cells of claim 106, claim 148, or claim 165, wherein the plurality of cells are mammalian cells or derived from mammalian cells.

212. The plurality of cells of claim 106, claim 148, or claim 165, wherein the plurality of cells are human cells or derived from human cells.

213. The plurality of cells of claim 106, claim 148, or claim 165, wherein the plurality of cells are stem cells (e.g., totipotent/omnipotent stem cells, pluripotent stem cells, multipotent stem cells, oligopotent stem cells, or unipotent stem cells), differentiated cells, or terminally differentiated cells.

214. The plurality of cells of claim 106, claim 148, or claim 165, wherein the plurality of cells are primary cells.

215. The plurality of cells of claim 106, claim 148, or claim 165, wherein the plurality of cells are cells of a cell line.

216. The plurality of cells of claim 106, claim 148, or claim 165, wherein the plurality of cells comprise two or more cell types.

217. The plurality of cells of claim 106, claim 148, or claim 165, wherein the plurality of cells are T cells, B cells, or NK cells.

218. The plurality of cells of claim 106, claim 148, or claim 165, wherein the plurality of cells comprise a modification in a gene selected from the group consisting of: BCL11A intronic erythroid enhancer, CD3, B2M, TRAC, PDCD1, PDL1, CIITA, TTR, LDHA, and HAO1.

219. A plurality of modified cells, wherein at least about 0.1% of the modified cells comprise an insertion adjacent to a 5′-NTTN-3′ sequence, wherein N is any nucleotide.

220. The plurality of modified cells of claim 219, wherein at least about 0.5% of the modified cells comprise the insertion.

221. The plurality of modified cells of claim 219, wherein at least about 1.0% of the modified cells comprise the insertion.

222. The plurality of modified cells of claim 219, wherein at least about 2.0% of the modified cells comprise the insertion.

223. The plurality of modified cells of claim 219, wherein at least about 3.0% of the modified cells comprise the insertion.

224. The plurality of modified cells of claim 219, wherein the insertion is 1 nucleotide in length.

225. The plurality of modified cells of claim 219, wherein the insertion is from 2 nucleotides to about 9 nucleotides in length.

226. The plurality of modified cells of claim 219, wherein the insertion is greater than about 9 nucleotides in length.

227. The plurality of modified cells of claim 219, wherein the insertion is downstream of the 5′-NTTN-3′ sequence.

228. The plurality of modified cells of claim 219, wherein the insertion starts within about 15 nucleotides to about 35 nucleotides of the 5′-NTTN-3′ sequence.

229. The plurality of modified cells of claim 219, wherein the insertion starts within about 18 nucleotides to about 30 nucleotides of the 5′-NTTN-3′ sequence.

230. The plurality of modified cells of claim 219, wherein the insertion starts within about 20 nucleotides to about 28 nucleotides of the 5′-NTTN-3′ sequence.

231. The plurality of modified cells of claim 219, wherein the insertion starts within about 20 nucleotides to about 25 nucleotides downstream of the 5′-NTTN-3′ sequence.

232. The plurality of modified cells of claim 219, wherein the 5′-NTTN-3′ sequence is 5′-NTTY-3′,5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′, wherein Y is C or T, B is any nucleotide except for A, D is any nucleotide except for C, and R is A or G.

233. The plurality of modified cells of claim 219, wherein the 5′-NTTN-3′ sequence is 5′-ATTA-3′,5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′.

234. The plurality of modified cells of claim 219, wherein the insertion is in an exon of the gene.

235. The plurality of modified cells of claim 219, wherein the insertion overlaps with a mutation in the gene.

236. The plurality of modified cells of claim 219, wherein the insertion overlaps with a deletion in the gene.

237. The plurality of modified cells of claim 219, wherein the insertion corrects a frameshift in the gene.

238. The plurality of modified cells of claim 219, wherein the insertion disrupts one or both alleles of the gene.

239. The plurality of modified cells of claim 219, wherein the plurality of modified cells are eukaryotic cells or prokaryotic cells.

240. The plurality of modified cells of claim 219, wherein the plurality of modified cells are animal cells, plant cells, or fungal cells or the cells derived from animal cells, plant cells, or fungal cells.

241. The plurality of modified cells of claim 219, wherein the plurality of modified cells are mammalian cells or derived from mammalian cells.

242. The plurality of modified cells of claim 219, wherein the plurality of modified cells are human cells or derived from human cells.

243. The plurality of modified cells of claim 219, wherein the plurality of modified cells are stem cells (e.g., totipotent/omnipotent stem cells, pluripotent stem cells, multipotent stem cells, oligopotent stem cells, or unipotent stem cells), differentiated cells, or terminally differentiated cells.

244. The plurality of modified cells of claim 219, wherein the plurality of modified cells are primary cells.

245. The plurality of modified cells of claim 219, wherein the plurality of modified cells are cells of a cell line.

246. The plurality of modified cells of claim 219, wherein the plurality of modified cells comprise two or more cell types.

247. A composition or formulation comprising the modified cell of claim 1, claim 36 or claim 74, a plurality of cells of claim 106, claim 148, or claim 165, or a plurality of modified cells of claim 219.

248. A composition or formulation comprising a modified cell or a plurality of cells comprising a deletion, wherein the deletion is adjacent to a 5′-NTTN-3′ sequence, wherein N is any nucleotide.

249. The composition or formulation of claim 248, wherein at least 70% of the plurality of cells comprise the deletion.

250. The composition or formulation of claim 248, wherein at least 80% of the plurality of cells comprise the deletion.

251. The composition or formulation of claim 248, wherein at least 90% of the plurality of cells comprise the deletion.

252. The composition or formulation of claim 248, wherein 100% of the plurality of cells comprise the deletion.

253. The composition or formulation of claim 248, wherein the deletion is up to about 40 nucleotides in length.

254. The composition or formulation of claim 248, wherein the deletion is between about 4 nucleotides and 40 nucleotides in length.

255. The composition or formulation of claim 248, wherein the deletion is between about 4 nucleotides and 25 nucleotides in length.

256. The composition or formulation of claim 248, wherein the deletion is between about 10 nucleotides and 25 nucleotides in length.

257. The composition or formulation of claim 248, wherein the deletion is between about 10 nucleotides and 15 nucleotides in length.

258. The composition or formulation of claim 248, wherein the deletion starts within about 5 nucleotides to about 15 nucleotides of the 5′-NTTN-3′ sequence.

259. The composition or formulation of claim 248, wherein the deletion starts within about 5 nucleotides to about 10 nucleotides of the 5′-NTTN-3′ sequence.

260. The composition or formulation of claim 248, wherein the deletion starts within about 10 nucleotides to about 15 nucleotides of the 5′-NTTN-3′ sequence.

261. The composition or formulation of claim 248, wherein the deletion starts within about 5 nucleotides to about 15 nucleotides downstream of the 5′-NTTN-3′ sequence.

262. The composition or formulation of claim 248, wherein the deletion starts within about 5 nucleotides to about 10 nucleotides downstream of the 5′-NTTN-3′ sequence.

263. The composition or formulation of claim 248, wherein the deletion starts within about 10 nucleotides to about 15 nucleotides downstream of the 5′-NTTN-3′ sequence.

264. The composition or formulation of claim 248, wherein the deletion ends within about 20 nucleotides to about 30 nucleotides of the 5′-NTTN-3′ sequence.

265. The composition or formulation of claim 248, wherein the deletion ends within about 20 nucleotides to about 25 nucleotides of the 5′-NTTN-3′ sequence.

266. The composition or formulation of claim 248, wherein the deletion ends within about 25 nucleotides to about 30 nucleotides of the 5′-NTTN-3′ sequence.

267. The composition or formulation of claim 248, wherein the deletion ends within about 20 nucleotides to about 30 nucleotides downstream of the 5′-NTTN-3′ sequence.

268. The composition or formulation of claim 248, wherein the deletion ends within about 20 nucleotides to about 25 nucleotides downstream of the 5′-NTTN-3′ sequence.

269. The composition or formulation of claim 248, wherein the deletion ends within about 25 nucleotides to about 30 nucleotides downstream of the 5′-NTTN-3′ sequence.

270. The composition or formulation of claim 248, wherein the deletion starts within about 5 nucleotides to about 15 nucleotides downstream of the 5′-NTTN-3′ sequence and ends within about 20 nucleotides to about 30 nucleotides downstream of the 5′-NTTN-3′ sequence.

271. The composition or formulation of claim 248, wherein the deletion starts within about 5 nucleotides to about 15 nucleotides downstream of the 5′-NTTN-3′ sequence and ends within about 20 nucleotides to about 25 nucleotides downstream of the 5′-NTTN-3′ sequence.

272. The composition or formulation of claim 248, wherein the deletion starts within about 5 nucleotides to about 15 nucleotides downstream of the 5′-NTTN-3′ sequence and ends within about 25 nucleotides to about 30 nucleotides downstream of the 5′-NTTN-3′ sequence.

273. The composition or formulation of claim 248, wherein the deletion starts within about 5 nucleotides to about 10 nucleotides downstream of the 5′-NTTN-3′ sequence and ends within about 20 nucleotides to about 30 nucleotides downstream of the 5′-NTTN-3′ sequence.

274. The composition or formulation of claim 248, wherein the deletion starts within about 5 nucleotides to about 10 nucleotides downstream of the 5′-NTTN-3′ sequence and ends within about 20 nucleotides to about 25 nucleotides downstream of the 5′-NTTN-3′ sequence.

275. The composition or formulation of claim 248, wherein the deletion starts within about 5 nucleotides to about 10 nucleotides downstream of the 5′-NTTN-3′ sequence and ends within about 25 nucleotides to about 30 nucleotides downstream of the 5′-NTTN-3′ sequence.

276. The composition or formulation of claim 248, wherein the deletion starts within about 10 nucleotides to about 15 nucleotides downstream of the 5′-NTTN-3′ sequence and ends within about 20 nucleotides to about 30 nucleotides downstream of the 5′-NTTN-3′ sequence.

277. The composition or formulation of claim 248, wherein the deletion starts within about 10 nucleotides to about 15 nucleotides downstream of the 5′-NTTN-3′ sequence and ends within about 20 nucleotides to about 25 nucleotides downstream of the 5′-NTTN-3′ sequence.

278. The composition or formulation of claim 248, wherein the deletion starts within about 10 nucleotides to about 15 nucleotides downstream of the 5′-NTTN-3′ sequence and ends within about 25 nucleotides to about 30 nucleotides downstream of the 5′-NTTN-3′ sequence.

279. The composition or formulation of claim 248, wherein the deletion is in an exon of the gene.

280. The composition or formulation of claim 248, wherein the deletion overlaps with a mutation in the gene.

281. The composition or formulation of claim 248, wherein the deletion overlaps with an insertion in the gene.

282. The composition or formulation of claim 248, wherein the deletion removes at least a portion of a repeat expansion of the gene.

283. The composition or formulation of claim 248, wherein the deletion disrupts one or both alleles of the gene.

284. A composition or formulation comprising a modified cell or a plurality of modified cells comprising an insertion, wherein the insertion is adjacent to a 5′-NTTN-3′ sequence, wherein N is any nucleotide.

285. The composition or formulation of claim 284, wherein the insertion is 1 nucleotide in length.

286. The composition or formulation of claim 284, wherein the insertion is from 2 nucleotides to about 9 nucleotides in length.

287. The composition or formulation of claim 284, wherein the insertion is greater than about 9 nucleotides in length.

288. The composition or formulation of claim 284, wherein the insertion is downstream of the 5′-NTTN-3′ sequence.

289. The composition or formulation of claim 284, wherein the insertion starts within about 15 nucleotides to about 35 nucleotides of the 5′-NTTN-3′ sequence.

290. The composition or formulation of claim 284, wherein the insertion starts within about 18 nucleotides to about 30 nucleotides of the 5′-NTTN-3′ sequence.

291. The composition or formulation of claim 284, wherein the insertion starts within about 20 nucleotides to about 28 nucleotides of the 5′-NTTN-3′ sequence.

292. The composition or formulation of claim 284, wherein the insertion starts within about 20 nucleotides to about 25 nucleotides downstream of the 5′-NTTN-3′ sequence.

293. The composition or formulation of claim 284, wherein the insertion is in an exon of the gene.

294. The composition or formulation of claim 284, wherein the insertion overlaps with a mutation in the gene.

295. The composition or formulation of claim 284, wherein the insertion overlaps with a deletion in the gene.

296. The composition or formulation of claim 284, wherein the insertion corrects a frameshift in the gene.

297. The composition or formulation of claim 284, wherein the insertion disrupts one or both alleles of the gene.

298. The composition or formulation of claim 284, wherein the 5′-NTTN-3′ sequence is 5′-NTTY-3′, 5′-NTTC-3′,5′-NTTT-3′,5′-NTTA-3′,5′-NTTB-3′,5′-NTTG-3′,5′-CTTY-3′,5‘-DTTR’3′,5′-CTTR-3′,5′-DTTT-3′,5′-ATTN-3′, or 5′-GTTN-3′, wherein Y is C or T, B is any nucleotide except for A, D is any nucleotide except for C, and R is A or G.

299. The composition or formulation of claim 284, wherein the 5′-NTTN-3′ sequence is 5′-ATTA-3′, 5′-ATTT-3′,5′-ATTG-3′,5′-ATTC-3′,5′-TTTA-3′,5′-TTTT-3′,5′-TTTG-3′,5′-TTTC-3′,5′-GTTA-3′,5′-GTTT-3′,5′-GTTG-3′,5′-GTTC-3′,5′-CTTA-3′,5′-CTTT-3′,5′-CTTG-3′, or 5′-CTTC-3′.

300. The composition or formulation of claim 284, wherein at least about 0.1% of the plurality of modified cells comprise the insertion.

301. The composition or formulation claim 284, wherein at least about 0.5% of the plurality of modified cells comprise the insertion.

302. The composition or formulation claim 284, wherein at least about 1.0% of the plurality of modified cells comprise the insertion.

303. The composition or formulation claim 284, wherein at least about 2.0% of the plurality of modified cells comprise the insertion.

304. The composition or formulation claim 284, wherein at least about 3.0% of the plurality of modified cells comprise the insertion.

305. The composition or formulation claim 284, wherein at least about 70% of the plurality of modified cells comprise the insertion.

306. The composition or formulation of claim 284, wherein at least about 80% of the plurality of modified cells comprise the insertion.

307. The composition or formulation of claim 284, wherein at least about 90% of the plurality of modified cells comprise the insertion.

308. The composition or formulation of claim 284, wherein 100% of the plurality of modified cells comprise the insertion.

309. The composition or formulation of claim 284, wherein the modified cell or a cell of the plurality is a eukaryotic cell or a prokaryotic cell.

310. The composition or formulation of claim 284, wherein the modified cell or a cell of the plurality is an animal cell, a plant cell, or a fungal cell or the cell is derived from an animal cell, a plant cell, or a fungal cell.

311. The composition or formulation of claim 284, wherein the modified cell or a cell of the plurality is a mammalian cell or derived from a mammalian cell.

312. The composition or formulation of claim 284, wherein the modified cell or a cell of the plurality is a human cell or derived from a human cell.

313. The composition or formulation of claim 284, wherein the modified cell or a cell of the plurality is a stem cell (e.g., a totipotent/omnipotent stem cell, a pluripotent stem cell, a multipotent stem cell, an oligopotent stem cell, or an unipotent stem cell), a differentiated cell, or a terminally differentiated cell.

314. The composition or formulation of claim 284, wherein the modified cell or a cell of the plurality is a primary cell.

315. The composition or formulation of claim 284, wherein the modified cell or a cell of the plurality is a cell from a cell line.


SEQUENCE LISTING (67 sequences)

SEQ ID NO: 1 - DNA, 3162 bp, Artificial Sequence
  Synthetic Construct
atgagcagcgcgatcaaaagctacaagagcgttctgcgtccgaacgagcgtaagaaccaactgctgaaaagcaccattcagtgcctggaagacggtagcgcgttctttttcaagatgctgcaaggcctgtttggtggcatcaccccggagattgttcgtttcagcaccgaacaggagaaacagcaacaggatatcgcgctgtggtgcgcggttaactggttccgtccggtgagccaagacagcctgacccacaccattgcgagcgataacctggtggagaagtttgaggaatactatggtggcaccgcgagcgacgcgatcaaacagtacttcagcgcgagcattggcgaaagctactattggaacgactgccgtcaacagtactatgatctgtgccgtgagctgggtgttgaggtgagcgacctgacccatgatctggagatcctgtgccgtgaaaagtgcctggcggttgcgaccgagagcaaccagaacaacagcatcattagcgttctgtttggcaccggcgaaaaagaggaccgtagcgtgaaactgcgtatcaccaagaaaattctggaggcgatcagcaacctgaaagaaatcccgaagaacgttgcgccgattcaagagatcattctgaacgtggcgaaagcgaccaaggaaaccttccgtcaggtgtatgcgggtaacctgggtgcgccgagcaccctggagaaatttatcgcgaaggacggccaaaaagagttcgatctgaagaaactgcagaccgacctgaagaaagttattcgtggtaaaagcaaggagcgtgattggtgctgccaggaagagctgcgtagctacgtggagcaaaacaccatccagtatgacctgtgggcgtggggcgaaatgttcaacaaagcgcacaccgcgctgaaaatcaagagcacccgtaactacaactttgcgaagcaacgtctggaacagttcaaagagattcagagcctgaacaacctgctggttgtgaagaagctgaacgactttttcgatagcgaatttttcagcggcgaggaaacctacaccatctgcgttcaccatctgggtggcaaggacctgagcaaactgtataaggcgtgggaggatgatccggcggacccggaaaacgcgattgtggttctgtgcgacgatctgaaaaacaactttaagaaagagccgatccgtaacattctgcgttacatcttcaccattcgtcaagaatgcagcgcgcaggacatcctggcggcggcgaagtacaaccaacagctggatcgttataaaagccaaaaggcgaacccgagcgttctgggtaaccagggctttacctggaccaacgcggtgatcctgccggagaaggcgcagcgtaacgaccgtccgaacagcctggatctgcgtatttggctgtacctgaaactgcgtcacccggacggtcgttggaagaaacaccatatcccgttctacgatacccgtttcttccaagaaatttatgcggcgggcaacagcccggttgacacctgccagtttcgtaccccgcgtttcggttatcacctgccgaaactgaccgatcagaccgcgatccgtgttaacaagaaacatgtgaaagcggcgaagaccgaggcgcgtattcgtctggcgatccaacagggcaccctgccggtgagcaacctgaagatcaccgaaattagcgcgaccatcaacagcaaaggtcaagtgcgtattccggttaagtttgacgtgggtcgtcaaaaaggcaccctgcagatcggtgaccgtttctgcggctacgatcaaaaccagaccgcgagccacgcgtatagcctgtgggaagtggttaaagagggtcaataccataaagagctgggctgctttgttcgtttcatcagcagcggtgacatcgtgagcattaccgagaaccgtggcaaccaatttgatcagctgagctatgaaggtctggcgtacccgcaatatgcggactggcgtaagaaagcgagcaagttcgtgagcctgtggcagatcaccaagaaaaacaagaaaaaggaaatcgtgaccgttgaagcgaaagagaagtttgacgcgatctgcaagtaccagccgcgtctgtataaattcaacaaggagtacgcgtatctgctgcgtgatattgttcgtggcaaaagcctggtggaactgcaacagattcgtcaagagatctttcgtttcattgaacaggactgcggtgttacccgtctgggcagcctgagcctgagcaccctggaaaccgtgaaagcggttaagggtatcatttacagctattttagcaccgcgctgaacgcgagcaagaacaacccgatcagcgacgaacagcgtaaagagtttgatccggaactgttcgcgctgctggaaaagctggagctgattcgtacccgtaaaaagaaacaaaaagtggaacgtatcgcgaacagcctgattcagacctgcctggagaacaacatcaagttcattcgtggtgaaggcgacctgagcaccaccaacaacgcgaccaagaaaaaggcgaacagccgtagcatggattggttggcgcgtggtgtttttaacaaaatccgtcaactggcgccgatgcacaacattaccctgttcggttgcggcagcctgtacaccagccaccaggacccgctggtgcatcgtaacccggataaagcgatgaagtgccgttgggcggcgatcccggttaaggacattggcgattgggtgctgcgtaagctgagccaaaacctgcgtgcgaaaaacatcggcaccggcgagtactatcaccaaggtgttaaagagttcctgagccattatgaactgcaggacctggaggaagagctgctgaagtggcgtagcgatcgtaaaagcaacattccgtgctgggtgctgcagaaccgtctggcggagaagctgggcaacaaagaagcggtggtttacatcccggttcgtggtggccgtatttattttgcgacccacaaggtggcgaccggtgcggtgagcatcgttttcgaccaaaaacaagtgtgggtttgcaacgcggatcatgttgcggcggcgaacatcgcgctgaccgtgaagggtattggcgaacaaagcagcgacgaagagaacccggatggtagccgtatcaaactgcagctgaccagc

SEQ ID NO: 2 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFDVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNIGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTVKGIGEQSSDEENPDGSRIKLQLTS

SEQ ID NO: 3 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGRWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 4 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGSRIKLQLTS

SEQ ID NO: 5 - protein, 1368 aa, Artificial Sequence [sequence omitted]

SEQ ID NO: 6 - DNA, 20 bp, Artificial Sequence
  Synthetic Construct
tgtccccccaagttttggac

SEQ ID NO: 7 - RNA, 43 nt, Artificial Sequence
  Synthetic Construct
agaaauccgucuuucauugacgguguccccccaaguuuuggac

SEQ ID NO: 8 - DNA, 20 bp, Artificial Sequence
  Synthetic Construct
gcttttgtccccccaagttt

SEQ ID NO: 9 - DNA, 102 bp, Artificial Sequence
  Synthetic Construct
gcuuuuguccccccaaguuugttttagagctagaaatagcaagttaaaataaggctagtccgttatcaacttgaaaaagtggcaccgagtcggtgcuuuuuu

SEQ ID NO: 10 - DNA, 20 bp, Artificial Sequence
  Synthetic Construct
ggagaggtgagggacttggg

SEQ ID NO: 11 - RNA, 43 nt, Artificial Sequence
  Synthetic Construct
agaaauccgucuuucauugacggggagaggugagggacuuggg

SEQ ID NO: 12 - DNA, 20 bp, Artificial Sequence
  Synthetic Construct
ggagaggtgagggacttggg

SEQ ID NO: 13 - DNA, 102 bp, Artificial Sequence
  Synthetic Construct
ggagaggugagggacuuggggttttagagctagaaatagcaagttaaaataaggctagtccgttatcaacttgaaaaagtggcaccgagtcggtgcuuuuuu

SEQ ID NO: 14 - DNA, 20 bp, Artificial Sequence
  Synthetic Construct
gtgagaatggtgcgtcctag

SEQ ID NO: 15 - RNA, 43 nt, Artificial Sequence
  Synthetic Construct
agaaauccgucuuucauugacgggugagaauggugcguccuag

SEQ ID NO: 16 - DNA, 20 bp, Artificial Sequence
  Synthetic Construct
ttgtgagaatggtgcgtcct

SEQ ID NO: 17 - DNA, 102 bp, Artificial Sequence
  Synthetic Construct
uugugagaauggugcguccugttttagagctagaaatagcaagttaaaataaggctagtccgttatcaacttgaaaaagtggcaccgagtcggtgcuuuuuu

SEQ ID NO: 18 - DNA, 20 bp, Artificial Sequence
  Synthetic Construct
aactggccctggctttggca

SEQ ID NO: 19 - RNA, 43 nt, Artificial Sequence
  Synthetic Construct
agaaauccgucuuucauugacggaacuggcccuggcuuuggca

SEQ ID NO: 20 - DNA, 20 bp, Artificial Sequence
  Synthetic Construct
gctttaactggccctggctt

SEQ ID NO: 21 - DNA, 102 bp, Artificial Sequence
  Synthetic Construct
gcuuuaacuggcccuggcuugttttagagctagaaatagcaagttaaaataaggctagtccgttatcaacttgaaaaagtggcaccgagtcggtgcuuuuuu

SEQ ID NO: 22 - DNA, 20 bp, Artificial Sequence
  Synthetic Construct
ggatggcgacttcaggcaca

SEQ ID NO: 23 - RNA, 43 nt, Artificial Sequence
  Synthetic Construct
agaaauccgucuuucauugacggggauggcgacuucaggcaca

SEQ ID NO: 24 - DNA, 20 bp, Artificial Sequence
  Synthetic Construct
tggatggcgacttcaggcac

SEQ ID NO: 25 - DNA, 102 bp, Artificial Sequence
  Synthetic Construct
uggauggcgacuucaggcacgttttagagctagaaatagcaagttaaaataaggctagtccgttatcaacttgaaaaagtggcaccgagtcggtgcuuuuuu

SEQ ID NO: 26 - DNA, 20 bp, Artificial Sequence
  Synthetic Construct
atgtgattgatgcccaaagg

SEQ ID NO: 27 - RNA, 43 nt, Artificial Sequence
  Synthetic Construct
agaaauccgucuuucauugacggaugugauugaugcccaaagg

SEQ ID NO: 28 - DNA, 20 bp, Artificial Sequence
  Synthetic Construct
tttatgtgattgatgcccaa

SEQ ID NO: 29 - DNA, 102 bp, Artificial Sequence
  Synthetic Construct
uuuaugugauugaugcccaagttttagagctagaaatagcaagttaaaataaggctagtccgttatcaacttgaaaaagtggcaccgagtcggtgcuuuuuu

SEQ ID NO: 30 - DNA, 20 bp, Artificial Sequence
  Synthetic Construct
ggggaggcctggagtcatgg

SEQ ID NO: 31 - RNA, 43 nt, Artificial Sequence
  Synthetic Construct
agaaauccgucuuucauugacggggggaggccuggagucaugg

SEQ ID NO: 32 - DNA, 20 bp, Artificial Sequence
  Synthetic Construct
tttggggaggcctggagtca

SEQ ID NO: 33 - DNA, 102 bp, Artificial Sequence
  Synthetic Construct
uuuggggaggccuggagucagttttagagctagaaatagcaagttaaaataaggctagtccgttatcaacttgaaaaagtggcaccgagtcggtgcuuuuuu

SEQ ID NO: 34 - DNA, 20 bp, Artificial Sequence
  Synthetic Construct
tgggggtgaccgccggagcg

SEQ ID NO: 35 - RNA, 43 nt, Artificial Sequence
  Synthetic Construct
agaaauccgucuuucauugacggugggggugaccgccggagcg

SEQ ID NO: 36 - DNA, 20 bp, Artificial Sequence
  Synthetic Construct
tgggggtgaccgccggagcg

SEQ ID NO: 37 - DNA, 102 bp, Artificial Sequence
  Synthetic Construct
ugggggugaccgccggagcggttttagagctagaaatagcaagttaaaataaggctagtccgttatcaacttgaaaaagtggcaccgagtcggtgcuuuuuu

SEQ ID NO: 38 - DNA, 20 bp, Artificial Sequence
  Synthetic Construct
gttgacattgtccacacctg

SEQ ID NO: 39 - RNA, 43 nt, Artificial Sequence
  Synthetic Construct
agaaauccgucuuucauugacggguugacauuguccacaccug

SEQ ID NO: 40 - DNA, 20 bp, Artificial Sequence
  Synthetic Construct
ttgttgacattgtccacacc

SEQ ID NO: 41 - DNA, 102 bp, Artificial Sequence
  Synthetic Construct
uuguugacauuguccacaccgttttagagctagaaatagcaagttaaaataaggctagtccgttatcaacttgaaaaagtggcaccgagtcggtgcuuuuuu

SEQ ID NO: 42 - DNA, 20 bp, Artificial Sequence
  Synthetic Construct
ggaaatctattgaggctctg

SEQ ID NO: 43 - RNA, 43 nt, Artificial Sequence
  Synthetic Construct
agaaauccgucuuucauugacggggaaaucuauugaggcucug

SEQ ID NO: 44 - DNA, 20 bp, Artificial Sequence
  Synthetic Construct
ttggaaatctattgaggctc

SEQ ID NO: 45 - DNA, 102 bp, Artificial Sequence
  Synthetic Construct
uuggaaaucuauugaggcucgttttagagctagaaatagcaagttaaaataaggctagtccgttatcaacttgaaaaagtggcaccgagtcggtgcuuuuuu

SEQ ID NO: 46 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELGCFVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGEQSSDEENPDGGRIKLQLTS

SEQ ID NO: 47 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLAPMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 48 - protein, 1054 aa, Artificial Sequence
  Synthetic Construct
MSSAIKSYKSVLRPNERKNQLLKSTIQCLEDGSAFFFKMLQGLFGGITPEIVRFSTEQEKQQQDIALWCAVNWFRPVSQDSLTHTIASDNLVEKFEEYYGGTASDAIKQYFSASIGESYYWNDCRQQYYDLCRELGVEVSDLTHDLEILCREKCLAVATESNQNNSIISVLFGTGEKEDRSVKLRITKKILEAISNLKEIPKNVAPIQEIILNVAKATKETFRQVYAGNLGAPSTLEKFIAKDGQKEFDLKKLQTDLKKVIRGKSKERDWCCQEELRSYVEQNTIQYDLWAWGEMFNKAHTALKIKSTRNYNFAKQRLEQFKEIQSLNNLLVVKKLNDFFDSEFFSGEETYTICVHHLGGKDLSKLYKAWEDDPADPENAIVVLCDDLKNNFKKEPIRNILRYIFTIRQECSAQDILAAAKYNQQLDRYKSQKANPSVLGNQGFTWTNAVILPEKAQRNDRPNSLDLRIWLYLKLRHPDGRWKKHHIPFYDTRFFQEIYAAGNSPVDTCQFRTPRFGYHLPKLTDQTAIRVNKKHVKAAKTEARIRLAIQQGTLPVSNLKITEISATINSKGQVRIPVKFRVGRQKGTLQIGDRFCGYDQNQTASHAYSLWEVVKEGQYHKELRCRVRFISSGDIVSITENRGNQFDQLSYEGLAYPQYADWRKKASKFVSLWQITKKNKKKEIVTVEAKEKFDAICKYQPRLYKFNKEYAYLLRDIVRGKSLVELQQIRQEIFRFIEQDCGVTRLGSLSLSTLETVKAVKGIIYSYFSTALNASKNNPISDEQRKEFDPELFALLEKLELIRTRKKKQKVERIANSLIQTCLENNIKFIRGEGDLSTTNNATKKKANSRSMDWLARGVFNKIRQLATMHNITLFGCGSLYTSHQDPLVHRNPDKAMKCRWAAIPVKDIGDWVLRKLSQNLRAKNRGTGEYYHQGVKEFLSHYELQDLEEELLKWRSDRKSNIPCWVLQNRLAEKLGNKEAVVYIPVRGGRIYFATHKVATGAVSIVFDQKQVWVCNADHVAAANIALTGKGIGRQSSDEENPDGGRIKLQLTS

SEQ ID NO: 49 - RNA, 43 nt, Artificial Sequence
  Synthetic Construct
agaaauccgucuuucauugacggaaugucggauggaugaaacc

SEQ ID NO: 50 - RNA, 43 nt, Artificial Sequence
  Synthetic Construct
agaaauccgucuuucauugacggcuaucucuuguacuacacug

SEQ ID NO: 51 - RNA, 43 nt, Artificial Sequence
  Synthetic Construct
agaaauccgucuuucauugacggacacggcaggcauacucauc

SEQ ID NO: 52 - RNA, 43 nt, Artificial Sequence
  Synthetic Construct
agaaauccgucuuucauugacgggucacagcccaagauaguua

SEQ ID NO: 53 - RNA, 100 nt, Artificial Sequence
  Synthetic Construct
  modified_base (1)..(2): gm
  misc_feature (1)..(4): The linkage between nucleotides at positions 1, 2, 3, and 4 are phosphorothioate linkages
  modified_base (3)..(3): cm
  modified_base (97)..(99): um
  misc_feature (97)..(100): The linkage between nucleotides at positions 97, 98, 99, and 100 are phosphorothioate linkages
ggccgagaugucucgcuccgguuuuagagcuagaaauagcaaguuaaaauaaggcuaguccguuaucaacuugaaaaaguggcaccgagucggugcuuuu

SEQ ID NO: 54 - RNA, 43 nt, Artificial Sequence
  Synthetic Construct
agaaauccgucuuucauugacggaagagcaacagugcuguggc

SEQ ID NO: 55 - RNA, 43 nt, Artificial Sequence
  Synthetic Construct
agaaauccgucuuucauugacggaacaacagcauuauuccaga

SEQ ID NO: 56 - RNA, 43 nt, Artificial Sequence
  Synthetic Construct
agaaauccgucuuucauugacgggaaacagguaagacaggggu

SEQ ID NO: 57 - RNA, 43 nt, Artificial Sequence
  Synthetic Construct
agaaauccgucuuucauugacggcaggaggaggauucggaacc

SEQ ID NO: 58 - RNA, 100 nt, Artificial Sequence
  Synthetic Construct
  modified_base (1)..(2): 2′-O-methyladenosine
  misc_feature (1)..(4): The linkage between nucleotides at positions 1, 2, 3, and 4 are phosphorothioate linkages
  modified_base (3)..(3): gm
  modified_base (97)..(99): um
  misc_feature (97)..(100): The linkage between nucleotides at positions 97, 98, 99, and 100 are phosphorothioate linkages
aaguuccugugaugucaagcguuuuagagcuagaaauagcaaguuaaaauaaggcuaguccguuaucaacuugaaaaaguggcaccgagucggugcuuuu

SEQ ID NO: 59 - RNA, 43 nt, Artificial Sequence
  Synthetic Construct
agaaauccgucuuucauugacgguuagguagguggggucggcg

SEQ ID NO: 60 - RNA, 43 nt, Artificial Sequence
  Synthetic Construct
agaaauccgucuuucauugacggcccgaggaccgcagccagcc

SEQ ID NO: 61 - RNA, 43 nt, Artificial Sequence
  Synthetic Construct
agaaauccgucuuucauugacggcgugucacacaacugcccaa

SEQ ID NO: 62 - RNA, 43 nt, Artificial Sequence
  Synthetic Construct
agaaauccgucuuucauugacggcacaugagcguggucagggc

SEQ ID NO: 63 - RNA, 100 nt, Artificial Sequence
  Synthetic Construct
  modified_base (1)..(1): um
  misc_feature (1)..(4): The linkage between nucleotides at positions 1, 2, 3, and 4 are phosphorothioate linkages
  modified_base (2)..(3): cm
  modified_base (97)..(99): um
  misc_feature (97)..(100): The linkage between nucleotides at positions 97, 98, 99, and 100 are phosphorothioate linkages
uccaggcaugcagaucccacguuuuagagcuagaaauagcaaguuaaaauaaggcuaguccguuaucaacuugaaaaaguggcaccgagucggugcuuuu

SEQ ID NO: 64 - RNA, 43 nt, Artificial Sequence
  Synthetic Construct
agaaauccgucuuucauugacgggaagcuagucuagugcaagc

SEQ ID NO: 65 - RNA, 43 nt, Artificial Sequence
  Synthetic Construct
agaaauccgucuuucauugacggcuggagccugugauaaaagc

SEQ ID NO: 66 - RNA, 43 nt, Artificial Sequence
  Synthetic Construct
agaaauccgucuuucauugacgguaccccacccacgcccccac

SEQ ID NO: 67 - RNA, 100 nt, Artificial Sequence
  Synthetic Construct
  modified_base (1)..(1): cm
  misc_feature (1)..(4): The linkage between nucleotides at positions 1, 2, 3, and 4 are phosphorothioate linkages
  modified_base (2)..(2): um
  modified_base (3)..(3): 2′-O-methyladenosine
  modified_base (97)..(99): um
  misc_feature (97)..(100): The linkage between nucleotides at positions 97, 98, 99, and 100 are phosphorothioate linkages
cuaacaguugcuuuuaucacguuuuagagcuagaaauagcaaguuaaaauaaggcuaguccguuaucaacuugaaaaaguggcaccgagucggugcuuuu